(19) United States
(12) Patent Application Publication
(10) Pub. No.: US 2012/0237481 A1
(43) Pub. Date: Sep. 20, 2012

(54) Incorporation of the B18R gene to enhance antitumor effect of virotherapy

(76) Inventors: Xiaoliu Zhang, Houston, TX (US); Xinping Fu, Houston, TX (US)

(21) Appl. No.: 13/420,734

(22) Filed: Mar. 15, 2012

Related U.S. Application Data
(60) Provisional application No. 61/453,887, filed on Mar. 17, 2011.

(51) Int. Cl. A61K 35/76; C07K 14/555; A61P 35/00; C12N 5/09; C12N 7/01

(52) U.S. Cl. 424/93.2; 435/375; 435/235.1; 530/351

(57) ABSTRACT

The present invention relates to a novel composition and method to potentiate the antitumor effect of an oncolytic virus by providing for resistance against a host's innate interferon response. Particularly, a B18R gene is incorporated into an oncolytic virus. During treatment of a host with the modified oncolytic virus, the oncolytic virus retains its phenotype, and the host's innate immune response has a minimal affect on viral replication.

### CROSS-REFERENCES TO RELATED APPLICATIONS

This application claims priority to U.S. provisional application No. 61/453,887, filed on Mar. 17, 2011, which is herein incorporated by reference in its entirety.

### GOVERNMENTAL SPONSORSHIP

The U.S. Government has a paid-up license in this invention and the rights in limited circumstances to require the patent owners to license others on reasonable terms as provided for by the terms of grant No. 7R01CA132792-03 awarded by the National Cancer Institute.

### FIELD OF THE INVENTION

The present invention relates to a novel composition and method to potentiate the antitumor effect of an oncolytic virus by providing for resistance against a host's innate interferon response.

### BACKGROUND OF THE INVENTION

Tumor virotherapy involves the application of a natural or genetically modified virus that can specifically replicate in cancer cells for the treatment of malignant diseases. Extensive preclinical studies and early stage clinical trials have shown that these so-called oncolytic viruses are safe for in vivo administration, and in many instances, have shown great potential for clinical applications. However, despite the demonstrated potency in lysing tumor cells in vitro, the success of virotherapy in vivo can be limited by the complex interplay of virus replication and host resistance factors. One of the major resistance factors is the host's immune defense system, which can restrict the ability of the virus to replicate and spread within tumors. More specifically, the host's innate immune system is rapidly activated during replication of the oncolytic virus in vivo. Thus, since the antitumor effect of an oncolytic virus is mainly generated during the acute phase of virus replication, the innate immune system may play a more pivotal role in dictating or inhibiting the initial extent of virus replication and spread in the tumor tissues than the classical adaptive immune responses of T and B lymphocytes.

Among a host's first lines of innate immune defense against the oncolytic activity of virotherapy are the interferons (IFNs). IFNs compose three major classes: type 1 (IFN-α and -β), type II (IFN-γ) and type III (IFN-λ). Upon viral infection, IFN release can be induced almost instantly, and the IFNs then bind to their receptors to activate signal transducer and activator of transcription (STAT) complexes. This activation triggers expression of a series of interferon-responsive genes such as protein kinase R (PKR) and 2′-5′-OAS/RNaseL, which convert cells into an antiviral state. The antiviral effect of IFNs is potent and rapid. Consequently, many viruses have developed diverse strategies to counteract the interferon activity. These include direct prevention of interferon synthesis, blockage of the effect of downstream signaling events triggered by receptor binding, and inhibition of the functions of antiviral effectors induced by IFNs. For example, herpes simplex virus (HSV) has employed diverse mechanisms to counteract the antiviral effect of IFNs. Several viral gene products, including ICP0 and ICP27 act by inhibiting the function of interferon regulatory factors (IRF) 3 and 7. Other HSV gene products, such as ICP34.5 and Us11, have been found to interact directly with the effector protein PKR and prevent its downstream effect—phosphorylation of eIF-2α. Vaccinia, another large DNA virus, also contains several genes whose products, through distinct mechanisms, function to limit the antiviral effect of IFNs. One of these viral products is the B18R gene, which is a secreted molecule that acts as a decoy receptor to intercept type I IFNs from various species, thus preventing these IFNs from binding to their receptors.

Despite HSV's reported ability to evade the antiviral effect of IFNs, the outcome of in vivo HSV infection is still largely dictated by the interferon status of the host. This has been demonstrated in several animal experiments (Conrady, C. D., Halford, W. P., and Carr, D. J. (2011). Loss of the type I interferon pathway increases vulnerability of mice to genital herpes simplex virus 2 infection. J Virol 85: 1625-1633; Carr, D. J., Al-khatib, K., James, C. M., and Silverman, R. (2003). Interferon-beta suppresses herpes simplex virus type 1 replication in trigeminal ganglion cells through an RNase L-dependent pathway. Journal of neuroimmunology 141: 40-46; Sainz, B., Jr., and Halford, W. P. (2002). Alpha/Beta interferon and gamma interferon synergize to inhibit the replication of herpes simplex virus type 1. J Virol 76: 11541-11550). Furthermore, clinical observations of patients with a genetic defect in the intracellular protein UNC-93B, which results in impaired cellular interferon-α/β and -λ antiviral responses, has shown that these patients are prone to more severe infections, such as HSV encephalitis (Casrouge, A., Zhang, S. Y., Eidenschenk, C., Jouanguy, E., Puel, A., Yang, K., et al. (2006). Herpes simplex virus encephalitis in human UNC-93B deficiency. Science 314: 308-312). Together, these indicate that strategies enabling an oncolytic HSV to ward off the host's interferon antiviral effect will enhance the therapeutic effect of oncolytic virotherapy. Thus, there is need in the art for a modified oncolytic virus that provides for resistance against the antiviral effects of a host's interferon response.

### SUMMARY OF THE INVENTION

In one embodiment of the invention, a medicament containing a modified oncolytic virus is administered to a host. The oncolytic virus is modified by having incorporated the B18R gene into the genome of the oncolytic virus. The B18R gene (DCT.VV.Orf107) was obtained from the National Institute of Allergy and Infectious Diseases, and was stored at Addgene.

Another embodiment of the invention discloses a method of generating a modified oncolytic virus. The modified oncolytic virus is generated by incorporating a B18R gene into the oncolytic virus.

In a further embodiment of the invention, a modified oncolytic virus includes an incorporated B18R gene.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the present disclosure. It should be understood, however, that the invention is not limited to the precise arrangements and instrumentalities shown.

FIG. 1. Shows that B18R is correctly expressed once the gene is inserted into the genome of an oncolytic HSV and that incorporation of the B18R gene into an oncolytic HSV (Synco-2D) does not change its phenotype (fusogenic phenotype).

FIG. 2. Shows that B18R released from Synco-B18R can effectively antagonize IFN-α to improve viral replication in human colon and liver cancer cells.

FIG. 3. Shows that B18R released from Synco-B18R can effectively antagonize both IFN-α and IFN-β to improve viral replication.

FIG. 4. Shows that B18R released from Synco-B18R can potentiate the oncolytic HSV replication in resistant tumor cells.

FIG. 5. Shows that incorporation of B18R can increase the killing ability of oncolytic HSV against permissive human cancer cells in the presence of high concentration IFN-α.

FIG. 6. Shows that incorporation of B18R can increase the killing ability of oncolytic HSV against resistant cancer cells (LL/2).

FIG. 7. Shows that incorporation of B18R can increase the killing ability of oncolytic HSV against semi-permissive Hepa1-6 (7A) and ID8 (7B) tumor cells.

FIG. 8. Shows that incorporation of B18R can significantly potentiate the antitumor efficacy of oncolytic HSV against established tumors.

### DETAILED DESCRIPTION OF THE INVENTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. It should be understood that any one of the features of the invention may be used separately or in combination with other features. Other systems, methods, features, and advantages of the invention will be or become apparent to one with skill in the art upon examination of the drawings and the detailed description. It is intended that all such additional systems, methods, features, and advantages be included within this description, be within the scope of the present invention, and be protected by the accompanying claims.

The present invention is directed to compositions and methods to potentiate the antitumor effect of an oncolytic virus. According to the present invention, incorporation of an interferon-antagonizing molecule into an oncolytic virus can improve the antitumor effect of the oncolytic virus (the B18R gene starting materials (DCT.VV.Orf107) disclosed herein were obtained from the National Institute of Allergy and Infectious Diseases, and were stored at Addgene). In one embodiment, B18R gene from vaccinia virus is incorporated into an oncolytic HSV to significantly potentiate its antitumor effects. This is because the interferon-antagonizing genes of HSV (e.g., ICP0, ICP34.5) mainly act intracellularly, while B18R is secreted to the outside of cells and its decoy effect on interferon works mainly extracellularly. Thus incorporating B18R gene into an oncolytic HSV provides the virus with additional ability to antagonize the interferon antiviral effect from outside of the cells, allowing the virus to replicate and spread more effectively within the tumor tissues for a maximal antitumor effect.

In another embodiment, Synco-B18R (exemplary genome sequence provided in SEQ ID NO: 2) is constructed by inserting the B18R gene into the internal repeat region of the genome of Synco-2D (exemplary genome sequence provided in SEQ ID NO: 1), an HSV-1-based oncolytic virus. In vitro data shown herein demonstrate that the tumor-killing ability of Synco-B18R is largely maintained in the presence of high levels of type I interferons, while the oncolytic effect of the parental virus (Synco-2D) is severely compromised. In vivo data shown herein demonstrate that Synco-B18R has a significantly superior antitumor activity than Synco-2D against established tumors. Our results thus confirm that the incorporation of the B18R gene of vaccinia virus can potentiate the antitumor effect of an oncolytic HSV, and such a strategy may be applicable to other types of oncolytic viruses.

In another embodiment of the present invention, the B18R gene is inserted into the genome of an oncolytic herpes simplex virus. The B18R gene was cut out from DCT.VV.Orf107 (obtained from the National Institute of Allergy and Infectious Diseases, and stored at Addgene). Add and cloned into pSZ-EGFP, which contains the green fluorescent protein marker gene (GFP) in addition to the repeat sequence of HSV in the internal junction region. The DNA sequence from the repeated region of HSV would allow the plasmid to recombine to the viral genome, and the GFP marker would facilitate the screening for positive recombinant virus. The new plasmid was transfected into Vero cells, which were then infected with Synco-2D. Virus was harvested 24 h later and GFP-positive plaques (green plaques) were picked up during the subsequent passage of the virus in Vero cells in 10 cm 6-well plates. The virus pick-up was subjected to another 5 more rounds of plaque purification until the GFP-positive virus reached 100% homogeneity.

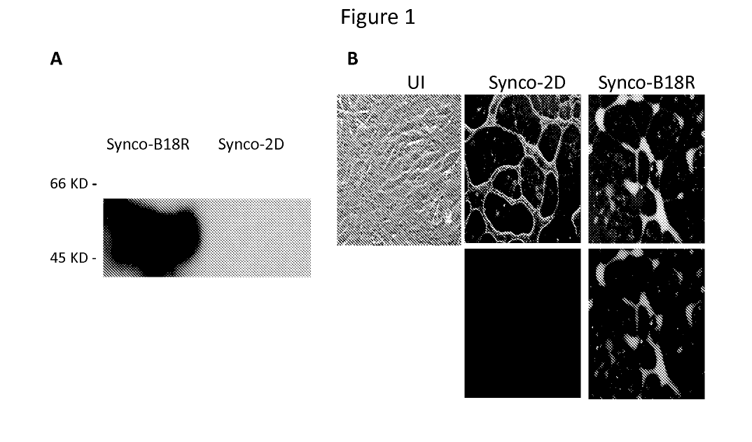

In another preferred embodiment of the present invention, insertion of the vaccinia B18R gene into the Synco-2D genome does not change the fusogenic phenotype of the virus. The parental Synco-2D is a fusogenic oncolytic HSV—infection of tumor cells by the virus induces a widespread membrane fusion and thus syncytia formation (Fu, X., Tao, L., Jin, A., Vile, R., Brenner, M., and Zhang, X. (2003). Expression of a fusogenic membrane glycoprotein by an oncolytic herpes simplex virus provides potent synergistic anti-tumor effect. Mol. Ther. 7: 748-754; Nakamori, M., Fu, X., Pettaway, C. A., and Zhang, X. (2004). Potent antitumor activity after systemic delivery of a doubly fusogenic oncolytic herpes simplex virus against metastatic prostate cancer. Prostate 60: 53-60). To determine if the fusogenic phenotype of the virus has been changed by the insertion of the B18R gene, Vero cells are infected with Synco-B18R at 0.1 plaque-forming unit (pfu) per cell. FIG. 1A shows a strong band of the right size for B18R in lysates of Synco-B18R infected cells, but not in the sample infected with Synco-2D. Like the parental Synco-2D, Synco-B18R infection also showed a typical syncytial phenotype. For example, FIG. 1B shows a typical micrograph taken 24 hours after infection. In FIG. 1B, the uninfected cells (labeled as UI) are seen as a monolayer without any cytopathic effect. However, the cells infected by Synco-2D and Synco-B18R both show clear membrane fusion (syncytium formation) under the phase contrast field (top panel). Under the dark field (bottom panel), only the cells infected with Synco-B18R are visible since the green fluorescent protein (GFP) gene was inserted into the viral genome together with the B18R gene. In contrast, only a dark field is visible in the cells infected with Synco-2D as it does not contain the GFP gene. Thus, insertion of the vaccinia B18R gene into the Synco-2D genome does not change the fusogenic phenotype of the virus.

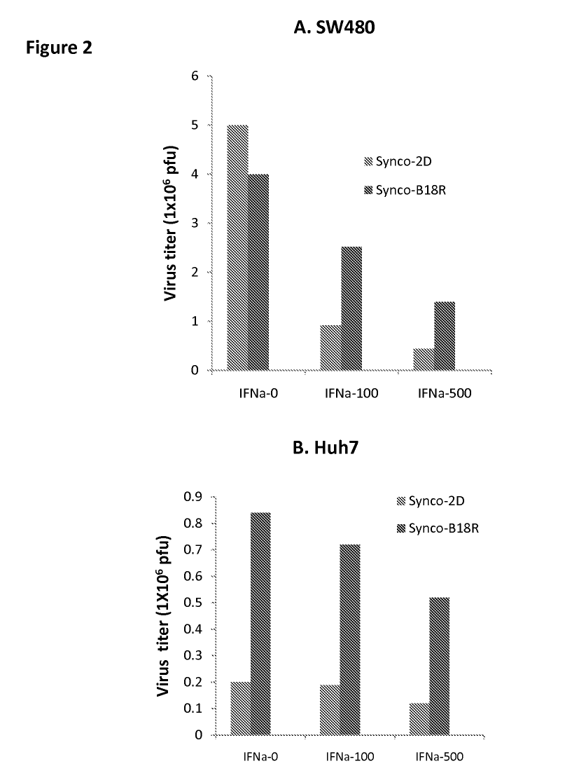

In a further embodiment of the present invention, incorporation of the B18R gene into an oncolytic HSV provides the virus with the ability to ward off the inhibitory effect of type I interferons. To show that Synco-B18R resists the inhibitory effect of interferons, Synco-B18R is directly compared with the parental Synco-2D for their ability to replicate in human tumor cells in the presence of externally added type I interferons. Human tumor cell lines SW480 (a human colon cancer cell line) and Huh7 (a human hepatocellular carcinoma line) were infected with either Synco-2D or Synco-B18R at 0.1 pfu/cell with or without an increasing amount of IFN-α in the medium. The viruses were harvested 24 h later and quantitated by plaque assay. As show in FIGS. 2A and 2B, the replication of Synco-2D was severely affected by the added IFN-α in both cell lines. However, the inhibitory effect of IFN-α on the replication of Synco-B18R was much less severe. In Huh7 cells, the titer of Synco-B18R was reduced by less than 1 fold even where IFN-α was added to a very high concentration (500 units).

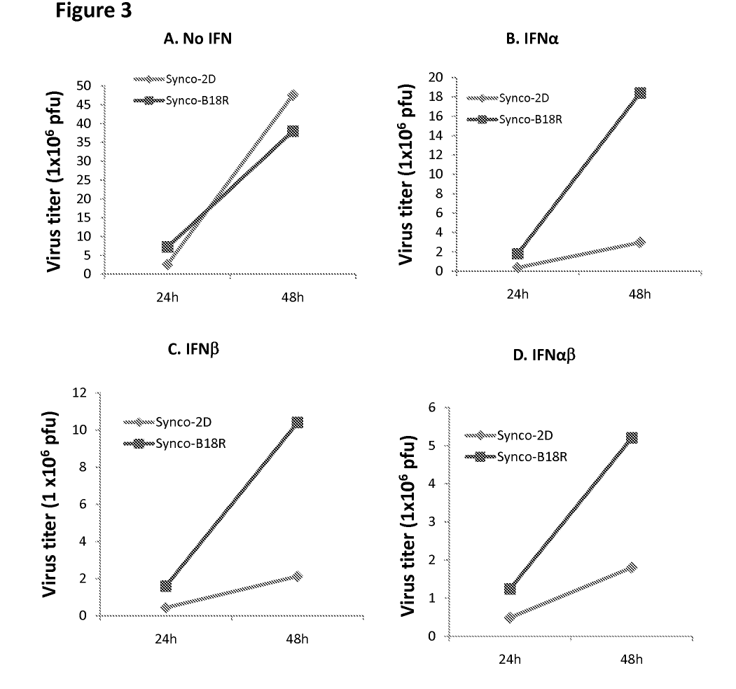

In one embodiment, incorporation of the B18R gene into an oncolytic HSV provides the virus with the ability to ward off both IFN-α and IFN-β, either individually or in combination. To show the effects of both IFN-α and IFN-β, either individually or in combination, on the replication of Synco-2D and Synco-B18R, an experiment is conducted on a time course, in which the virus is harvested either at 24 h or 48 h after infection. In the presence of IFN-α and IFN-β, either individually or in combination at a relatively high dose (500 units), Synco-B18R titer was significantly higher than that of Synco-2D. The difference was particularly dramatic at 48 h after infection, when the titer of Synco-B18R was 5 to 9 fold higher than that of Synco-2D (FIGS. 3B, C and D). Together, these results clearly demonstrate that incorporation of the B18R gene into an oncolytic HSV renders the virus with the ability to ward off the inhibitory effect of type I interferons.

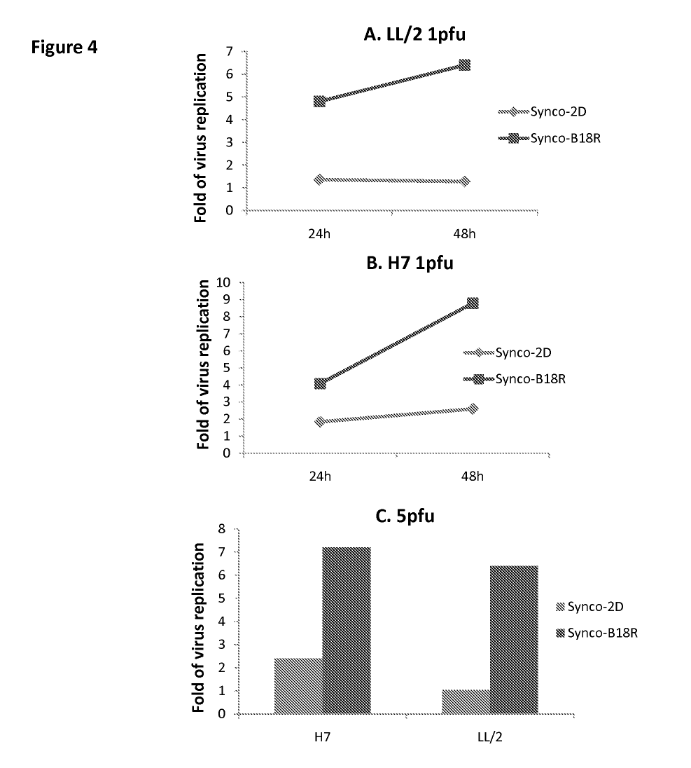

In another embodiment of the present invention, incorporation of B18R into an oncolytic HSV allows the oncolytic virus to replicate more efficiently in resistant tumor cells, such as LL/2 (a murine lung cancer cell line) and H7 (a murine pancreatic cancer line). Some tumor cells are more resistant than other tumor cells (e.g., those used in FIGS. 2 and 3) to HSV replication. Usually this resistance is due to the host's innate defense mechanisms, including the effects of IFNs. Referring to FIG. 4, the replication of Synco-2D and Synco-B18R is compared in two tumor cell lines that are more resistant to virus replication than SW480 or Huh7. Among them, LL/2 is a murine lung cancer cell line (Lewis lung cancer cell line) and H7 is a murine pancreatic cancer line. Due to their relative resistance, these cells were infected with the viruses at relatively higher virus doses (at 1 pfu and 5 pfu per cell, respectively), and the viruses were harvested at 24 h and 48 h after infection. Viral replication was calculated by dividing the virus yield at 24 h or 48 h after infection with the input virus (1 h after infection). The results show that, despite the resistant nature of these tumor cells, Synco-B18R had replicated by almost 10-fold, while Synco-2D had only less than a 2-fold replication.

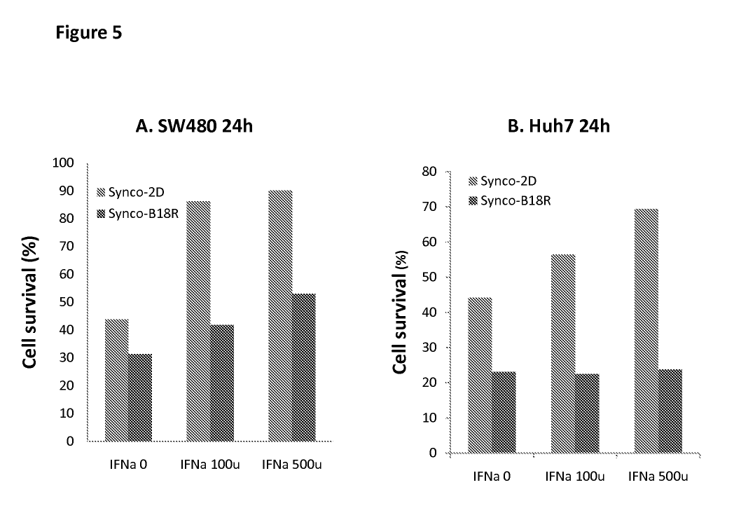

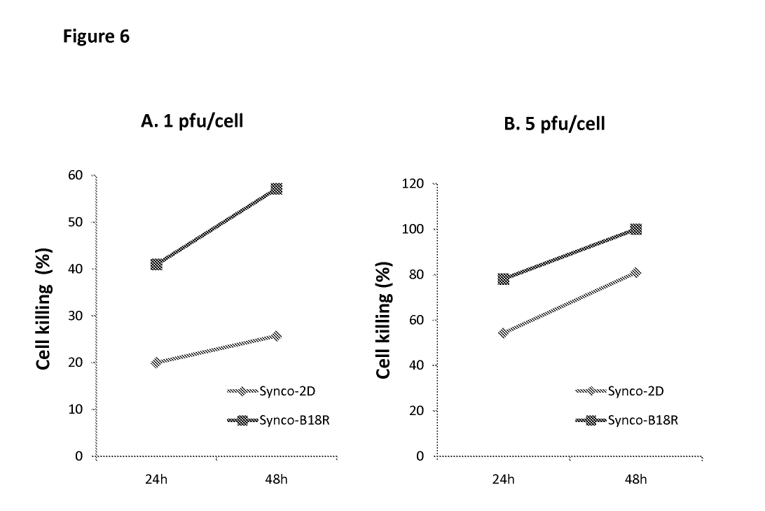

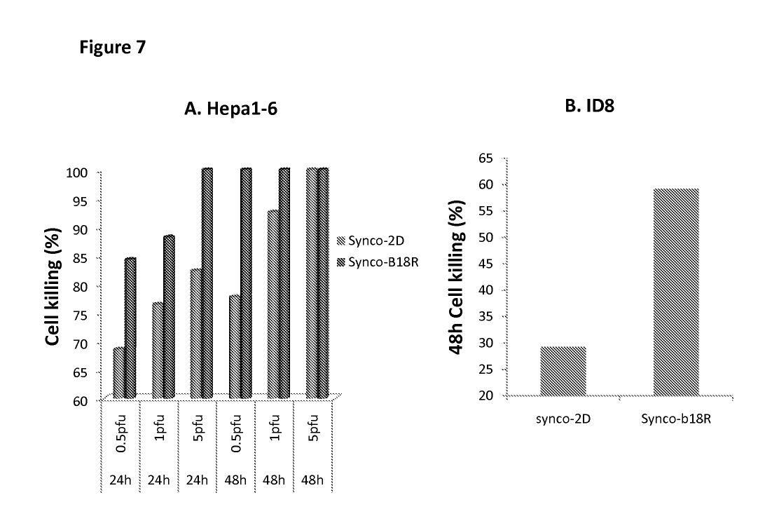

Another preferred embodiment of the present invention teaches that the incorporation of the B18R gene into an oncolytic HSV dramatically increases the virus replication and killing effect in tumor cells, in some cases even in the presence of high amount of IFNs in the culture medium. To demonstrate that the incorporation of B18R increases the killing ability of oncolytic HSV, it is determined if the increased replication ability seen in Synco-B18R transforms into an increased killing ability against tumor cells. The killing activity of Synco-2D and Synco-B18R is directly compared in a series of tumor cells. These include: SW480 human colon cancer cells (FIG. 5A) and human hepatocellular carcinoma cells (FIG. 5B), murine Lewis lung carcinoma (FIG. 6), murine hepatocellular carcinoma cell line Hepa1-6 (FIG. 7A) or murine ovarian cancer cell line ID8 (FIG. 7B). In all these tumor cells, Synco-B18R killed cells much more effectively than Synco-2D, with or without the presence of added interferons in the medium. These results, together with the results shown in FIGS. 2, 3, and 4 demonstrate that incorporation of B18R gene into an oncolytic HSV dramatically increases the virus replication and killing effect in tumor cells even in the presence of high amounts of IFNs in the culture medium.

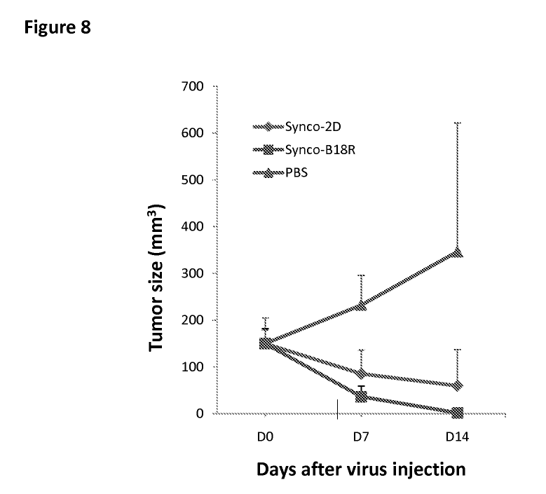

Another preferred embodiment of the present invention teaches that the incorporation of B18R gene into an oncolytic virus significantly enhances its antitumor effect (and hence its therapeutic effect), due to its ability to antagonize the host's interferon antiviral effect. Hepa1-6 murine hepatocellular carcinoma cells were implanted into immune competent B6 mice. Once the tumor reached the approximate size of 5 mm in diameter, mice were randomly divided into 3 groups and were treated with 1) PBS (as a negative control), 2) 1×10⁷ pfu of Synco-2D or 3) 1×10⁷ pfu of Synco-B18R. The tumor size was measured on a regular basis and the tumor size was determined by the formula: tumor volume [mm³]=(length [mm])×(width [mm])²×0.52. The results show that, despite the effective antitumor effect of Synco-2D, Synco-B18R was even better than Synco-2D at all of the time points after the mice received the virotherapy treatment (see FIG. 8). Moreover, 4/5 mice in the group treated with Synco-B18R became tumor-free at day 14 after virotherapy, while no tumor-free animal was recorded in other groups (See Table 1 Below). These results demonstrate that the incorporation of the B18R gene into an oncolytic virus (in this case a type I herpes simplex virus based oncolytic virus) significantly enhances its antitumor effect, due to its ability to antagonize the host's interferon antiviral effect.

TABLE 1
Incorporation of B18R can significantly potentiate the antitumor
efficacy of oncolytic HSV against established tumors.

| Treatment | Tumor-free animals¹ |
|---|---|
| PBS | 0/5 |
| Synco-2D | 0/5 |
| Synco-B18R | 4/5 |

¹Recorded on day 14 after the start of virotherapy.

While the invention described here specifically focuses on a novel method to significantly increase the antitumor effect of HSV-based oncolytic virus by incorporating the B18R gene, one of ordinary skills in the art, with the benefit of this disclosure, would recognize the extension of the approach to other types of oncolytic viruses. Furthermore, for the sake of convenience, the B18R gene was inserted into the internal repeat region of the viral genome during the construction of Synco-B18R. A person of ordinary skill in the art would understand that the B18R gene can be inserted into any region of the viral genome, as long as it is sufficiently expressed, and that the expression of B18R from genes inserted in any locus of the viral genome will satisfy the ultimate goal of antagonizing the interferon antiviral mechanism.

It will be appreciated by those skilled in the art that changes could be made to the embodiments described above without departing from the broad inventive concept thereof. It is understood, therefore, that the invention disclosed herein is not limited to the particular embodiments disclosed, but it is intended to cover modifications within the spirit and scope of the present invention as defined by the appended claims.

### EXAMPLES

### Example 1

### B18R Expression and Phenotypic Characterization of Synco-B18R

Synco-B18R is constructed by inserting the B18R gene into the genome of an oncolytic HSV (Synco-2D), which has a fusogenic phenotype. Exemplary genome sequences of Synco-2D and Synco-B18R are provided in SEQ ID NO: 1 and SEQ ID NO: 2 respectively. The B18R gene is located in the region between nucleotides 1 and 7409. To measure the expression of B18R from Synco-B18R, vero cells were infected with either Synco-B18R or Synco-2D for 24 h. Cells were then collected and lysed for detection of B18R by far western blotting analysis. This was done by first incubating the separated proteins on the membrane with 10 μg/ml of IFNβ for 1 h, and then by incubating with DSS crosslinking agent. The membrane was washed three times with PBS and incubated overnight with goat anti-human IFNβ antibody. After two washes with PBS, 1:1000 dilution of HRP-conjugated donkey anti-goat IgG was added and incubated for 1 h. After wash, band detection was performed by using ECL Plus Western Blotting Detection system. To phenotypically characterize Synco-B18R, Vero cells were infected with Synco-B18R at a multiplicity of infection (MOI) of 0.1 pfu/cell. Micrographs were taken 24 h after infection (original magnification: 200×). The results (FIG. 1) show that incorporation of the B18R gene into an oncolytic HSV (Synco-2D) does not change its phenotype (fusogenic phenotype).

### Example 2

### B18R Released from Synco-B18R can Effectively Antagonize IFN-α to Improve Viral Replication in Human Colon and Liver Cancer Cells

Sw480 (Colon Cancer) and Huh7 (liver Cancer) cells were seeded into 24-well plates and were infected with either Synco-2D or Synco-B18R at 0.1 pfu/cell. The infected cells were then cultured with medium with or without the indicated amount of IFN-α (100 or 500 units). The virus was harvested 24 h later and titrated by plaque assay. While the added IFN-α severely reduced the replication of the parental Synco-2D, it had much less inhibitory effect on the replication of Synco-B18R. These results (FIG. 2) show that B18R can help the virus ward off the antiviral effects of IFN-α.

### Example 3

### B18R Released from Synco-B18R can Effectively Antagonize Both IFN-α and IFN-β to Improve Viral Replication

SW480 cells were seeded into 24-well plates and were infected with either Synco-2D or Synco-B18R at 0.1 pfu/cell. The infected cells were then cultured with medium with or without 500 units of either IFN-α or IFN-β alone or in combination (labeled as IFNαβ). The virus was harvested 24 h and 48 h later and titrated by plaque assay. While the added IFN-α and IFN-β severely reduced the replication of the parental Synco-2D, they had much less inhibitory effect on the replication of Synco-B18R. These results (FIG. 3) show that B18R can help the virus ward off the antiviral effects of both IFN-α and IFN-β.

### Example 4

### B18R Released from Synco-B18R can Potentiate the Oncolytic HSV Replication in Resistant Tumor Cells

As compared with other tumor cells, LL/2 and H7 cells are more resistant to oncolytic HSV replication. This resistance is mainly due to the innate interferon antiviral effect. Thus, Synco-B18R is tested to determine if it could replicate better than Synco-2D in these cells. LL/2 or H7 cells seeded in 96-well plates were infected with Synco-2D or Synco-B18R at 1 pfu/cell (FIGS. 4A and 4B) or 5 pfu/cell (FIG. 4C). The virus was harvested at 24 h or 48 h after infection and quantitated by plaque assay. The fold of virus replication was calculated by dividing the virus yield at 24 h or 48 h after infection with the input virus (1 h after infection). While the parental Synco-2D hardly grows in these resistant cells, the yield of Synco-B18R was significantly increased in these cells. These data thus indicate that expression of B18R from the virus can reverse the resistance of these tumor cells to the replication of oncolytic HSV.

### Example 5

### Incorporation of B18R can Increase the Killing Ability of Oncolytic HSV Against Permissive Human Cancer Cells in the Presence of High Concentration IFN-α

SW480 (FIG. 5A) and Huh7 (FIG. 5B) cells seeded in 24-well plates were infected with either Synco-2D or Synco-B18R at 0.1 pfu/cell and cultured in medium with or without the indicated amount of IFN-α. Cells were harvested 24 h later and cell viability was determined by trypan blue staining. The percentage of cell survival was calculated by dividing the number of viable cells from the infected well with that from a non-infected well. The results show that B18R can increase the killing effect of the oncolytic virus against these tumor cells in the presence of high concentration of type I interferon.

### Example 6

### Incorporation of B18R can Increase the Killing Ability of Oncolytic HSV Against Resistant Cancer Cells (LL/2)

LL/2 cells seeded in 24-well plates were infected with Synco-2D or Synco-B18R at either 1 pfu/cell (FIG. 6A) or 5 pfu/cell (FIG. 6B). Cells were harvested 24 h later and cell viability was determined by trypan blue staining. The percentage of cell killing was calculated by the following formula: Cell killing (%)=(1−(viable cell number in infected well)/(viable cell number in control well))×100. The results show that B18R can facilitate the oncolytic HSV to efficiently kill tumor cells that are otherwise resistant to the oncolytic effect of the virus.

### Example 7

### Incorporation of B18R can Increase the Killing Ability of Oncolytic HSV Against Semi-Permissive Hepa1-6 and ID8 Tumor Cells

Cells seeded in 24-well plates were infected with Synco-2D or Synco-B18R at 0.05 or 1 pfu/cell (FIG. 7A) or at 5 pfu/cell (FIG. 7B). Cells were harvested either at either 24 h or 48 h (FIG. 7A) or at 48 h after infection (FIG. 7B) and cell viability was determined by trypan blue staining. The percentage of cell killing was calculated by the following formula: Cell killing (%)=(1−(viable cell number in infected well)/(viable cell number in control well))×100. The results show that B18R can enhance the killing effect of oncolytic HSV against tumor cells that are only semi-permissive to the oncolytic effect of the virus.

### Example 8

### Incorporation of B18R can Significantly Potentiate the Antitumor Efficacy of Oncolytic HSV Against Established Tumors

Murine hepatocellular carcinoma was established at the right flank of immune-competent B6 mice by subcutaneous implantation of Hepa1-6 cells. Once the tumor reached the approximate size of 5 mm in diameter, mice received intratumoral injection of either PBS (as a negative control), the Synco-2D or Synco-B18R viruses. Tumor size was measured at the indicated time and tumor volume determined by the formula: tumor volume [mm³]=(length [mm])×(width [mm])²×0.52. The results (FIG. 8) show that B18R can further increase the therapeutic efficacy of a potent oncolytic HSV such as Synco-2D.

What is claimed is:

1. A method of treating cancer, comprising:
administering to a host a medicament including an oncolytic virus, wherein the oncolytic virus is modified by the incorporation of a B18R gene.

2. The method of claim 1, wherein incorporation of the B18R gene potentiates an antitumor effect of the oncolytic virus.

3. The method of claim 1, wherein the B18R gene is derived from a vaccinia virus.

4. The method of claim 1, wherein the B18R is driven by a strict late viral promoter so that its expression is tailored to the ability of the oncolytic virus to replicate in tumor cells.

5. The method of claim 1, wherein the B18R gene is inserted into the genome of the oncolytic virus.

6. The method of claim 1, wherein the oncolytic virus is a Herpes Simplex Virus compound.

7. The method of claim 1, wherein the incorporation of the B18R gene into the oncolytic virus does not alter a phenotype of the oncolytic virus.

8. The method of claim 1, wherein the oncolytic virus is capable of warding off the host's innate immune response.

9. The method of claim 8, wherein the innate immune response comprises interferons.

10. The method of claim 9, wherein the interferons are one or more of the following: type I interferons, type II interferons, or type III interferons.

11. The method of claim 1, wherein the medicament is administered to resistant tumor cells.

12. The method of claim 1, wherein the modified oncolytic virus antagonizes the host's interferon antiviral effect.

13. A method of generating an oncolytic virus, comprising:
incorporating a B18R gene into the oncolytic virus.

14. The method of claim 13, wherein incorporation of the B18R gene potentiates an antitumor effect of the oncolytic virus.

15. The method of claim 13, wherein the B18R gene is derived from a vaccinia virus.

16. The method of claim 13, wherein the B18R gene is inserted into the genome of the oncolytic virus.

17. The method of claim 13, wherein the oncolytic virus is a Herpes Simplex Virus compound.

18. The method of claim 13, wherein the incorporation of the B18R gene into the oncolytic virus does not alter a phenotype of the oncolytic virus.

19. The method of claim 13, wherein the oncolytic virus is capable of warding off the host's innate immune response.

20. The method of claim 19, wherein the innate immune response comprises interferons.

21. The method of claim 20, wherein the interferons are one or more of the following: type I interferons, type II interferons, or type III interferons.

22. The method of claim 13, wherein the oncolytic virus is administered to resistant tumor cells.

23. The method of claim 13, wherein the modified oncolytic virus antagonizes the host's interferon antiviral effect.

24. An oncolytic virus, comprising:
a B18R gene incorporated into the oncolytic virus.

25. The method of claim 24, wherein incorporation of the B18R gene potentiates an antitumor effect of the oncolytic virus.

26. The oncolytic virus of claim 24, wherein the B18R gene is derived from a vaccinia virus.

27. The oncolytic virus of claim 24, wherein the B18R gene is inserted into the genome of the oncolytic virus.

28. The oncolytic virus of claim 24, wherein the oncolytic virus is a Herpes Simplex Virus compound.

29. The oncolytic virus of claim 24, wherein the incorporated B18R gene does not alter a phenotype of the oncolytic virus.

30. The oncolytic virus of claim 24, wherein the oncolytic virus is capable of warding off the host's innate immune response.

31. The innate immune response of claim 30, further comprising interferons.

32. The interferons of claim 31, wherein the interferons are one or more of the following: type I interferons, type II interferons, or type III interferons.

33. The oncolytic virus of claim 24, wherein the oncolytic virus selectively kills tumor cells.

34. The oncolytic virus of claim 24, wherein the modified oncolytic virus antagonizes the host's interferon antiviral effect.


SEQUENCE LISTING (2 sequences)

SEQ ID NO: 1 - DNA, 163251 bp, Artificial Sequence
  Synco-2D is a fusogenic oncolytic herpes simplex virus (HSV)
agcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacctccacgcccaactcggaacccgcggtcaggagcgcgcccgcggccgccccgccgccgccccccgccggtgggcccccgccttcttgttcgctgctgctgcgccagtggctccacgttcccgagtccgcgtccgacgacgacgatgacgacgactggccggacagccccccgcccgagccggcgccagaggcccggcccaccgccgccgccccccggccccggcccccaccgcccggcgtgggcccggggggcggggctgacccctcccaccccccctcgcgccccttccgccttccgccgcgcctcgccctccgcctgcgcgtcaccgcggagcacctggcgcgcctgcgcctgcgacgcgcgggcggggagggggcgccggagccccccgcgacccccgcgacccccgcgacccccgcgacccccgcgacccccgcgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaacgttacacccgaggcggcctgggtcttccgcggagctcccgggagctccgcaccaagccgctctccggagagacgatggcaggagccgcgcatatatacgcttggagccagcccgccctcacagggcgggccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccccatggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggtgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacaaggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcgggggggggagagggggaactcgtgggtgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccacccggagcccaccacggacgaggatgacgacgacctggacgacggtgaggcggggggcggcaaggaccctgggggaggaggaggaggagggggggggagggaggaataggcgggcgggcgaggaaagggcgggccggggagggggcgtaacctgatcgcgccccccgttgtctcttgcagcagactacgtaccgcccgccccccgccggacgccccgcgcccccccacgcagaggcgccgccgcgccccccgtgacgggcggggcgtctcacgcagccccccagccggccgcggctcggacagcgcccccctcggcgcccatcgggccacacggcagcagtaacaccaacaccaccaccaacagcagcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggccccccgcgtccgcggccgcgtcgggacccgcgcgcccccgcgcggccgtggccccgtgcgtgcgagcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtgggcacgggccctcccgcggcgccgccccctccggcgccgccccgctcccctccgccgcctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgacccccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagactatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataagggacgccccccgtgtttgtggggaggggggggtcgggcgctgggtggtctctggccgcgcccactacaccagccaatccgtgtcggggaggggaaaagtgaaagacacgggcaccacacaccagcgggtcttttgtgttggccctaataaaaaaaaactcaggggatttttgctgtctgttgggaaataaaggtttacttttgtatcttttccctgtctgtgttggatgtatcgcggggatgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgccatgttgggcaggctctggtgttaaccacagagccgcggcccgggctgcctgaccaccgatccccgaaagcatcctgccactggcatggagccagaaccacagtgggttgggtgtgggtgttaagtttccgcgagcgcctgcccgcccggactgacctggcctctggccgccacaaagggcggggggggttaactacactatagggcaacaaaggatgggaggggtggcggggcgggacggggcgcccaaaagggggtcggccacaccacagacgtgggtgttggggggtggggcggaggggtgggggggggggagacagaaacaggaacatagttagaaaacaagaatgcggtgcagccagagaatcacaggagacgaggggatgggcgtgttggttaccaacccacacccaggcatgctcggtggtatgaaggagggggggcggtgcttcttagagaccgccgggggacgtggggttggtgtgcaaaggcacgcgcacccgcgcggccaggtgggccggtactccatccccccctcccccgacccttcccacccccgcgtgccagagatcaccccggtcccccggcacccgccactcctccgtatcctcgctttaggaacaactttagggggggtacacacgcgccgtgcatttccttccacaccccccctcccccgcactccccccccccaggcagtaagacccaagcatagagagccaggcacaaaaacacaggcggggtgggacacatgccttcttggagtacgtgggtcattggcgtggggggttacagcgacaccggccgaccccctggcggtcttccagccggcccttagataagggggcagttggtggtcggacgggtaagtaacagagtctgactaagggtgggagggggggaaaagaacgggctggtgtgctgtaacacgagcccacccgcgagtggcgtggccgaccttagcctctggggcgccccctgtcgtttgggtcccccccctctattggggagaagcaggtgtctaacctacctggaaacgcggcgtctttgttgaacgacaccggggcgccctcgacgagtgggataacgggggaggaagggagggaggagggtactgggggtgaagaaggggggggggagaagcgagaacaggaaaggcgatggagcccggcagaacaccgaggaaaaaaaaaccacagcgcatgcgccgggccgttgtggggccccgggccggggccccttgggtccgccggggccccgggccgggccgccacgggggccggccgttggcggtaaccccgagtgttcatctcaggccccgggccgggaacccggaaaagcctccggggggcctttttcgcgtcgcgtgccggcgagcgggtccggacggggcccggaccgccgcggtcgggggcccctcgtcccgggccgtacgcggccttcgccccgtgaggggacagacgaacgaaacattccggcgacggaacgaaaaacaccccagacgggttaaagaaacagaaaccgcaacccccaccacccccgaaacggggaaaacgaaaaaacagaccagcggccggccggcgcttagggggaggatgtcgccgacgccccttggccgccccggctgcaggggggcccggagagccgcggcacccggacgcgcccggaaagtctttcgcaccacccgcgatcggcacggccgcgcccccgcttttataaaggctgagatgacgcagcaaaaacaggccacagcaccacgtgggtaggtgatgtaattttattttcctcgtctgcggcctaatggatttccgggcgcggtgcccctgtctgcagagcacttaacggattgatatctcgcgggcacgcgcgcccttaatggaccggcgcggggcggggggccggatacccacacgggcggggggggggtgtcgcgggccgtctgctggcccgcggccacataaacaatgactctgggcctttctgcctctgccgcttgtgagtgcgcgcgccggctctgcggtgtcggcggcggctgcggcggctgcggcggccgccgtgttcggtctcggtagccggccggcgggtggactcgcggggggccggagggtggaaggcaggggggtgtaggatgggtatcaggacttccacttcccgtccttccatcccccgttcccctcggttgttcctcgcctcccccaacaccccgccgctttccgttggggttgttattgttgtcgggatcgtgcgggccgggggtcgccggggcaggggcgggggcgtgggcgggggtgctcgtcgatcgaccgggctcagtgggggcgtggggtgggtgggagaaggcgaggagactggggtgggggtgtcggtgggtggttgttttttgtggttgtttttgtgtctgttcccgtcccccgtcaccccctccctccgtcccctccgtccccccgtcgcgggtgtttgtgtttgtttattccgacattggtttatttaaataaacacagccgttctgcgtgtctgttcttgcgtgtggctgggggcttatatgtggggtcccgggggcgggatggggtttagcggcggggggcggcgcgccggacggggcgctggagataacggcccccggggaacgggggaccggggctgggtatcccgaggtgggtgggtgggcggcggtggccgggccgggccgggccgggccgggccgggtgggcggggtttggaaaaacgaggaggaggaggagaaggcgggggggggggagacggggggaaagcaaggacacggcccggggggtgggagcgcgggccgggccgctcgtaagagccgcgacccggccgccggggagcgttgtcgccgtcggtctgccggcccccgtccctcccttttttgaccaaccagcgccccccccccccctcaccaccattcctactaccaccaccaccaccaccaccgacacctcccgcgcacccccgcccacatccccccccaacccgcaccaccagcacgggttgggggtagcaggggatcaaaggggggcaaagcggcggggcggttcggggggggggggggggggcgggaaaccaagtaggcccgcccatccgcggcccctcccggcagccacgcccccagcgtcgggtgtcacggggaaagagcagaggggagaggggagagggggggagaggggagagggggggagaggggagagggggggagaggggagagggggggagaggggagagggggggagaggggagagggggggagaggggagagggggggagaggggagagggggggagaggggagagggggggagaggggagagggggggagagggggtatataaaccaacgaaaagcgcgggaacggggatacggggcttgtgtggcacgacgtcgtggttgtgttactgggcaaacacttggggactgtaggtttctgtgggtgccgaccctaggcgctatggggattttgggttgggtcgggcttattgccgttggggttttgtgtgtgcgggggggcttgccttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttttggaggacttgagttaccccgcgtttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcagccgaagcccctcaccacgccgccgcccatcatcgccacgtcggaccccaccccgcgacgggacgccgccacaaaaagcagacgccgacgaccccactcccggcgcctctaacgatgcctcgacggaaacccgtccgggttcggggggcgaaccggccgcctgtcgctcgtcagggccggcggcgctcctcgccgccctagaggctggtcccgctggtgtgacgttttcctcgtccgcgccccccgaccctcccatggatttaacaaacgggggggtgtcgcctgcggcgacctcggcgcctctggactggaccacgtttcggcgtgtgtttctgatcgacgacgcgtggcggcccctgatggagcctgagctggcgaaccccttaaccgcccacctcctggccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggcggccgtcaagaactgttatcccgaggcacggatgagcggccacggttgcctggaaaagtgggcgcgggacggcgtcctgttactaaacacgaccctgaccgtcaagcgcggggcggcggcgtcccactctagaatcggttgggaccgtttcgtgggcggagttatccgccggttggccgcgcgccgccccggcctggtgtttatgctctggggcacacacgcccagaatgccatcaggccggaccctcgggtccattgcgtcctcaagttttcgcacccgtcgcccctctccaaggttccgttcggaacctgccagcatttcctcgtggcgaaccgatacctcgagacccggtcgatttcacccatcgactggtcggtttgaaaggcatcgacgtccggggtttttgtcggtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcggggatatcgccagatacgactcccgcagattccattcggggtgccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgccccccccccatgccaccctcactgcccgtcgcgcgtgtttgatgttaataaataacacataaatttggctggttgtttgttgtctttaatggaccgcccgcaagggggggggggcatttcagtgtcgggtgacgagcgcgatccggccgggatcctaggaccccaaaagtttgtctgcgtattccagggcggggctcagttgaatctcccgcagcacctctaccagcaggtccgcggtgggctggagaaactcggccgtcccggggcaggcggttgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtggaactgtaaggggacggcgggttaatagacaatgaccacgttcggatcgcgcagagccgatagtatgtgctcactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgtagtcggcgtttatggccatggccagctccgtagagtcaatggactcgacaaactcgctgatgttggtgttgacgacggacatgaagccgtgttggtcccgcaagaccacgtaaggcaggggggcctcttccagtaactcggccacgttggccgtcgcgtgccgcctccgcagctcgtccgcaaaggcaaacacccgtgcgtacgtgtatcccatgagcgtataattgtccgtctgcagggcgacggacatcagccccccgcgcggcgagccggtcagcatctcgcagccccggaagataacgttgtccacgtacgtgctaaagggggcgacttcaaatgcctccccgaagagctcttggaggattcggaatctcccgaggaaggcccgcttcagcagcgcaaactgggtgtgaacggcggcggtggtctccggttccccgggggtgtagtggcagtaaaacacgtcgagctgttgttcgtccagccccgcgaaaataacgtcgaggtcgtcgtcgggaaaatcgtccgggcccccgtcccgcggccccagttgcttaaaatcaaacgcacgctcgccgggggcgcctgcgtcggccattaccgacgcctgcgtcggcacccccgaagatttggggcgcagagacagaatctccgccgttagttctcccatgcgggcgtaggcgagggtcctctgggtcgcatccaggcccgggcgctgcagaaagttgtaaaaggagataagcccgctaaatatgagccgcgacaggaacctgtaggcaaactccaccgaagtctccccctgagtctttacaaagctgtcgtcacgcaacactgcctcgaaggcccggaacgtcccactaaacccaaaaaccagttttcgcaggcgcgcggtcaccgcgatctggctgttgaggacgtaagtgacgtcgttgcgggccacgaccagctgctgtttgctgtgcacctcgcagcgcatgtgccccgcgtcctggtcctggctctgcgagtagttggtgatgcggctggcgttggccgtgagccacttttcaatcgtcaggccgggctggtgtgtcagccgtcggtattcgtcaaactccttgaccgacacgaacgtaagcacggggagggtgaacacgacgaactccccctcacgggtcaccttcaggtaggcgtggagcttggccatgtacgcgctcacctctttgtgggaggagaacagccgcgtccagccggggaggttggcggggttggtgatgtagttttccgggacgacgaagcgatccacgaactgcatgtgctcctcggtgatgggcaggccgtactccagcaccttcatgaggttaccgaactcgtgctcgacgcaccgtttgttgttaataaaaatggcccagctatacgagaggcgggcgtactcgcgcagcgtgcggttgcagatgaggtacgtgagcacgttctcgctctggcggacggaacaccgcagtttctggtgctcgaaggtcgactccagggacgccgtctgcgtcggcgagcccacacacaccaacacgggccgcaggcgggccgcgtactggggggtgtggtacagggcgttaatcatccaccagcaatacaccacggccgtgaggaggtgacgcccaaggagcccggcctcgtcgatgacgatcacgttgctgcgggtaaaggccggcagcgccccgtgggtggccggggccaaccgcgtcagggcgccctcggccaaccccagggtccgttccagggcggccagggcgcgaaactcgttccgcaactcctcgcccccggaggcggccagggcgcgcttcgtgaggtccaaaatcacctcccagtagtacgtcagatctcgtcgctgcaggtcctccagcgaggcggggttgctggtcagggtgtacgggtactgtcccagttgggcctggacgtgattcccgcgaaacccaaattcatgaaagatggtgttgatgggtcggctgagaaaggcgcccgagagtttggcgtacatgttttgggccgcaatgcgcgtggcgcccgtcaccacacagtccaagacctcgttgattgtctgcacgcacgtgctctttccggagccagcgttgccggtgataagatacaccgcgaacggaaactccctgaggggcaggcctgcgggggactctaaggccgccacgtcccggaaccactgcagatggggcacttgcgctccgtcgagctgttgttgcgagagctctcggatgcgcttaaggattggctgcaccccgtgcatagacgtaaaatttaaaaaggcctcggccctccctggaacggctggtcggtccccgggttgctgaaggtgcggcgggccgggtttctgtccgtctagctggcgctccccgccggccgccgccatgaccgcaccacgctcgcgggcccccactacgcgtgcgcggggggacacggaagcgctgtgctcccccgaggacggctgggtaaaggttcaccccagccccggtacgatgctgttccgcgagattctccacgggcagctggggtataccgagggccagggggtgtacaacgtcgtccggtccagcgaggcgaccacccggcagctgcaggcggcgatctttcacgcgctcctcaacgccaccacttaccgggacctcgaggcggactggctcggccacgtggcggcccgcggtctgcagccccaacggctggttcgccggtacaggaacgcccgggaggcggatatcgccggggtggccgagcgggtgttcgacacgtggcggaacacgcttaggacgacgctgctggactttgcccacgggttggtcgcctgctttgcgccgggcggcccgagcggcccgtcaagcttccccaaatatatcgactggctgacgtgcctggggctggtccccatattacgcaagcgacaagaagggggtgtgacgcagggtctgagggcgtttctcaagcagcacccgctgacccgccagctggccacggtcgcggaggccgcggagcgcgccggccccgggttttttgagctggcgctggccttcgactccacgcgcgtggcggactacgaccgcgtgtatatctactacaaccaccgccggggcgactggctcgtgcgagaccccatcagcgggcagcgcggagaatgtctggtgctgtggccccccttgtggaccggggaccgtctggtcttcgattcgcccgtccagcggctgtttcccgagatcgtcgcgtgtcactccctccgggaacacgcgcacgtctgccggctgcgcaataccgcgtccgtcaaggtgctgctggggcgcaagagcgacagcgagcgcggggtggccggtgccgcgcgggtcgttaacaaggtgttgggggaggacgacgagaccaaggccgggtcggccgcctcgcgcctcgtgcggcttatcatcaacatgaagggcatgcgccacgtaggcgacattaacgacaccgtgcgttcctacctcgacgaggccggggggcacctgatagacgccccggccgtcgacggtaccctccctggattcggcaagggcggaaacagccgcgggtctgcgggccaggaccagggggggcgggcgccgcagcttcgccaggccttccgcacggccgtggttaacaacatcaacggcgtgttggagggctatataaataacctgtttggaaccatcgagcgcctgcgcgagaccaacgcgggcctggcgacccaattgcaggagcgcgaccgcgagctccggcgcgcaacagcgggggccctggagcgccagcagcgcgcggccgacctggcggccgagtccgtgaccggtggatgcggcagccgccctgcgggggcggacctgctccgggccgactatgacattatcgacgtcagcaagtccatggacgacgacacgtacgtcgccaacagctttcagcacccgtacatcccttcgtacgcccaggacctggagcgcctgtcgcgcctctgggagcacgagctggtgcgctgttttaaaattctgtgtcaccgcaacaaccagggccaagagacgtcgatctcgtactccagcggggcgatcgccgcattcgtcgccccctactttgagtcagtgcttcgggccccccgggtaggcgcgcccatcacgggctccgatgtcatcctgggggaggaggagttatgggatgcggtgtttaagaaaacccgcctgcaaacgtacctgacagacatcgcggccctgttcgtcgcggacgtccagcacgcagcgctgcccccgcccccctccccggtcggcgccgatttccggcccggcgcgtccccgcggggccggtccagatcgcggtcgcccggaagaactgcgcgaggcgcgccggaccagggcgggggcatcgggcaccgggatggccgccgcgacggccgacgatgaggggtcggccgccaccatcctcaagcaggccatcgccggggaccgcagcctggtcgaggcggccgaggcgattagccagcagacgctgctccgcctggcctgcgaggtgcgccaggtcggcgaccgccagccgcggtttaccgccaccagcatcgcgcgcgtcgacgtcgcgcctgggtgccggttgcggttcgttctggacgggagtcccgaggacgcctatgtgacgtcggaggattactttaagcgctgctgcggccagtccagttatcgcggcttcgcggtggcggtcctgacggccaacgaggaccacgtgcacagcctggccgtgccccccctcgttctgctgcaccggttctccctgttcaaccccagggacctcctggactttgagcttgcctgtctgctgatgtacctggagaactgcccccgaagccacgccaccccgtcgacctttgccaaggttctggcgtggctcggggtcgcgggtcgccgcacgtccccattcgaacgcgttcgctgccttttcctccgcagttgccactgggtcctaaacacactcatgttcatggtgtacgtaaaaccgttcgacgacgagttcgtcctgccccactggtacatggcccggtacctgctggccaacaacccgccccccgttctctcggccctgttctgtgccaccccgacgagctcctcattccggctgccggggccgcccccccgctccgactgcgtggcctataaccccgccgggatcatggggagctgctgggcgtcggaggaggtgcgcgcgcctctggtctattggtggctttcggagaccccaaaacgacagacgtcgtcgctgttttatcagttttgttgaattttaggaaataaacccggttttgtttctgtggcctcccgacggatgcgcgtgtccttactccgtcttggtgggtgggtggctgtgtatggcgtcccatctgtgcggggaggggggcaagtcggcacgtattcggacagactcaagcacacacgggggagcgctcttgtctcagggcaatgtttttattggtcaaactcaggcaaacagaaacgacatcttgtcgtcaaagggatacacaaacttccccccctcgccccatactcccgccagcaccccggtaaacaccaactcaatctcgcgcaggatttcgcgcaggtgatgagcgcagtccacgggggggagcacaaggggccgcgggtatagatcgacggggacgccgaccgactccccgcctccgggacagacacgcacgacgcgccgccagtagtgctctgcgtccagcaaggcgccgccgcggaaggcagtggggggcaaggggtcgctggcctcaaagggggacacccgaacgctccagtactccgcgtccaaccgtttattaaacgcgtccaagataaggcggtcgcaggcgtcctccataaggccccgggccgtgagtgcgtcctcctccggcacgcatgccgttgtcaggcccaggacccgtcgcagcgtgtcgcgtacgacccctgccgccgtggtgtacgcgggcccgcggagaggaaatcccccaagatggtcagtgttgtcgcgggagttccagaaccacactcccgcctggctccaggcgactgcgtgggtgtagacgccctcgagggccaggcacagtgggtgccgcagccggacggcgttggccctaagcacggctcccacggccgtctcgatggcccgccgggcgtcctcgatcaccccggaagccgcatccgcgtcttgggggtccacgttaaagacaccccagaacgcacccccatcgcccccgcagaccgcgaacttcaccgagctggccgtctcctcgatctgcaggcagacggcggccattaccccacccaggagctgccgcagcgcagggcaggcgttgcacgtgtccgggaccaggcgctccaagacggccccggcccagggctctgagggagcggccaccaccagcgcgtccagtcttgctaggcccgtccggccgtgggggtccgccagcccgctccccccgaggtcggccagggccgccaggagctgggcgcgaagtccggggaagcaaaaccgcgccgtccagacgggcccgacggccgcgggcgggtctaacagttggatgattttagtggcgggatgccaccgcgccaccgcctcccgcaccgcgggcaggaggcatccggctgccgccgaggccacgccgggccaggctcgcggggggaggacgaccctggcccccaccgcgggccaggcccccaggagcgcggcgtaagcggccgcggccccgcgcaccaggtcccgtgccgactcggccgtggccggcacggtgaacgtgggccaacccggaaaccccaggacggcaaagtacgggacgggtcccccccggacctcaaactcgggccccagaaaggcaaagacgggggccagggccccgggggcggcgtggaccgtggtatgccactgccggaaaagggcgacgagcgccggcgcggagaacttctcgccggcgcttacaaagtagtcgtaatcgcggggcagcagcacccgtgccgtgactcgttgcgggtgcccgcgtggccgcaggcccacctcgcacacctcgaccaggtccccgaacgcgccctccttcttgatcggcggaaacgcaagagtctggtattcgcgcgcaaatagcgcggttccggtggtgatgttaacggtcagcgaagcggcggacgcgcactggggggtgtcgcgaatggccgccaggcgcgcccacgccagccgcgcgtcgggatgctcggcaacgcgcgccgccagggccatagggtcgatgtcaatgttggcctccgcgaccaggagagcggcgcgaggggcggcgggcgggccccacgacgctctctcaactttcaccaccagtcccgtgcgtgggtccgagccgatacgcagcggggcgaacagggccaccggcccggtctggcgctccagggccgccaggacgcacgcgtacagcgcccgccacagagtcgggttctccaggggctccagcggggaggcggccggcgtcgtcgcggcgcgggcggccgccacgacggcctggacggagacgtccgcggagccgtagaaatcccgcagctccgtcgcggtgacggagacctccgcaaagcgcgcgcgaccctcccctgcggcgttgcgacatacaaaatacaccagggcgtggaagtactcgcgagcgcgggggggcagccataccgcgtaaagggtaatggcgctgacgctctcctccacccacacgatatctgcggtgtccatcgcacggcccctaaggatcacgggcggtctgtgggtcccatgctgccgtgcctggccgggcccggtgggtcgcggaaaccggtgacggggggggggcggtttttggggttggggtgggggtgggaaacggcccgggtccgggggccaacttggcccctcggtgcgttccggcaacagcgccgccggtccgcggacgaccacgtaccgaacgagtgcggtcccgagacttatagggtgctaaagttcaccgccccctgcatcatgggccaggcctcggtggggagctccgacagcgccgcctccaggatgatgtcagcgttggggttggcgctggatgagtgcgtgcgcaaacagcgcccccacgcgggcacgcgtagcttgaagcgcgcgcccgcaaactcccgcttgtgggccataagcagggcgtacagctgcctgtgggtccggcaggcgctgtggtcgatgtggtgggcgtccaacaaccccacgattgtctgtttggtgaggtttttaacgcgccccgccccgggaaacgtctgcgtgcttttggccatctgcacgccaaacagttcgccccagattatcttgaacagcgccaccgcgtggtccgtctcgctaacggacccgcgcgggggacagccgcttagggcgtcggcgacgcgcttgacggcttcctccgagagcagaagtccgtcggttacgttacagtggcccagttcgaacaccagctgcatgtagcggtcgtagtggggggtcagtaggtccagcacgtcatcggggccgaaggtcctcccagatcccccggccgccgagtcccaatgcaggcgcgcggccatggtgctgcacaggcacaacagctcccagacgggggttacgttcagggtggggggcagggccacgagctccagctctccggtgacgttgatcgtggggatgacgcccgtggcgtagtggtcatagatccgccgaaatatggcgctgctgcgggtggccatgggaacgcggagacaggcctccagcaacgccaggtaaataaaccgcgtgcgtcccatcaggctgttgaggttgcgcatgagcgcgacaatttccgccggcgcgacatcggaccggaggtatttttcgacgaaaagacccacctcctccgtctcggcggcctgggccggcagcgacgcctcgggatcccggcaccgcagctcccgtagatcgcgctgggccctgagggcgtcgaaatgtacgccccgcaaaaacagacagaagtcctttggggtcagggtatcgtcgtgtccccagaagcgcacgcgtatgcagtttagggtcagcagcatgtgaaggatgttaaggctgtccgagagacacgccagcgtgcatctctcaaagtagtgtttgtaacggaatttgttgtagatgcgcgacccccgccccagcgacgtgtcgcatgccgacgcgtcacagcgccccttgaaccggcgacacagcaggtttgtgacctgggagaactgcgcgggccactggccgcaggaactgaccacgtgattaaggagcatgggcgtaaagacgggctccgagcgcgccccggagccgtccatgtaaatcagtagctcccccttgcggagggtgcgcacccgtcccagggactggtacacggacaccatgtccggtccgtagttcatgggtttcacgtaggcgaacatgccatcaaagtgcaggggatcgaagctgaggcccacggttacgaccgtcgtgtatataaccacgcggtattggccccacgtggtcacgtccccgaggggggtgagcgagtgaagcaacagcacgcggtccgtaaactgacggcagaaccgggccacgatctccgcgaaggagaccgtcgacgaaaaaatgcagatgttatcgcccccgccaaggcgcgcttccagctccccaaagaacgtggccccccgggcctccggagaggcgtccggagacgggccgctcggcggcccgggcgggcgcagggcagcctgcaggagctcggtccccagacgcgggagaaacaggcaccggcgcgccgaaaacccgggcatggcgtactcgccgaccaccacatgcacgtttttttcgccccggagaccgcacaggaagtccaccaactgcgcgttggcggttgcgtccatggcgatgatccgaggacagatgcgcagcaggcgtagcattaacgcatccacgcggcccagttgctgcatcgttggcgaatagagctggcccagcgtcgacataacctcgtccagaacgaggacgtcgtagttgttcagaaggttggggcccacgcgatgaaggctttccacctggacgataagtcggtggaaggggcggtcgttcataatgtaattggtggatgagaagtaggtgacaaagtcgaccaggcctgactcagcgaaccgcgtcgctagggtctgggtaaaactccgacgacaggagacgacgagcacactcgtgtccggagagtggatcgcttcccgcagccagcggatcagcgcggtagtttttcccgaccccattggcgcgcggaccacagtcacgcacctggccgtcggggcgctcgcgttggggaaggtgacgggtccgtgctgctgccgctcgatcgttgttttcgggtgaacccggggcacccattcggccaaatcccccccgtacaacatccgcgctagcgatacgctcgacgtgtactgttcgcactcgtcgtccccaatgggacgcccggcccccagaggatctcccgactccgcgccccccacgaaaggcatgaccggggcgcggacggcgtggtgggtctggtgtgtgcaggtggcgacgtttgtggtctctgcggtctgcgtcacggggctcctcgtcctggcctctgtgttccgggcacggtttccctgcttttacgccacggcgagctcttatgccggggtgaactccacggccgaggtgcgcgggggtgtagccgtgcccctcaggttggacacgcagagccttgtgggcacttatgtaatcacggccgtgttgttgttggccgtggccgtgtatgccgtggtcggcgccgtgacctcccgctacgaccgcgccctggacgcgggccgccgtctggctgcggcccgcatggccatgccgcacgccacgctgatcgccggaaacgtctgctcttggttgctgcagatcaccgtcctgttgctggcccatcgcatcagccagctggcccacctggtttacgtcctgcactttgcgtgtctggtgtattttgcggcccatttttgcaccaggggggtcctgagcgggacgtatctgcgtcaggtgcacggcctgatggagctggccccgacccatcatcgcgtcgtcggcccggctcgcgccgtgctgacaaacgccttgctgttgggcgtcttcctgtgcacggccgacgccgcggtatccctgaataccatcgccgcgttcaactttaatttttcggccccgggcatgctcatctgcctgaccgtgctgttcgccattctcgtcgtatcgctgttgttggtggtcgagggggtgttgtgtcactacgtgcgcgtgttggtgggcccccacctgggggccgtggccgccacgggcatcgtcggcctggcctgcgagcactattacaccaacggctactacgttgtggagacgcagtggccgggggctcagacgggagtccgcgtcgccctcgccctggtcgccgcctttgccctcggcatggccgtgctccgctgcacccgcgcctatctgtatcacaggcggcaccacaccaaattttttatgcgcatgcgcgacacgcgacaccgcgcacattccgccctcaagcgcgtacgcagttccatgcgcggatcgcgagacggccgccacaggcccgcacccggcagcccgcccgggattcccgaatatgcggaagacccctacgcgatctcatacggcggccagctcgaccggtacggagattccgacggggagccgatttacgacgaggtggcggacgaccaaaccgacgtattgtacgccaagatacaacacccgcggcacctgcccgacgacgatcccatctatgacaccgttggggggtacgaccccgagcccgccgaggaccccgtgtacagcaccgtccgccgttggtagctgtttggttccgttttaataaaccgtttgtgtttaacccgaccgtggtgtatgtctggtgtgtggcgtccgatcccgttactatcaccgtccccccccccccctcaaccccggcgattgtgggttttttaaaaacgacacgcgtgcgaccgtatacagaacattgttttggtttttattcgctatcggacatggggggtggaaactgggtggcggggcaggcgcctccgggggtccgccggtgagtgtggcgcgagggggggtccgatgaacgcaggcgctgtctccccggggcccgcgtaaccccgcgcatatccgggggcacgtagaaattaccttcctcttcggactcgatatccacgacgtcaaagtcgtgggcggtcagcgagacgacctccccgtcgtcggtgatgaggacgttgtttcggcagcagcagggccgggccccggagaacgagaggcccatagctcggcgagcgtgtcgtcgaatgccaggcggctgcttcgctggatggccttatagatctccggatcgatgcggacgggggtaatgatcagggcgatcggaacggcctggttcgggagaatggacgccttgctgggtcctgcggccccgagagccccggcgccgtcctccaggcggaacgttacgccctcctccgcgctggtgcggtgcctgccgataaacgtcaccagatgcgggtggggggggcagtcggggaagtggctgtcgagcacgtagccctgcaccaagatctgcttaaagttcgggtgacgggggttcgcgaagacgggctcgcggcggaccagatccccggagctccaggacacgggggagatggtgtggcgtccgaggtcgggggcgccaaacagaagcacctccgagacaacgccgctatttaactccaccaaggcccgatccgcggcggagcaccgccttttttcgcccgaggcgtgggcctctgaccaggcctggtcttgcgtgacgagagcctcctccgggccggggacgcgcccgggcgcgaagtatcgcacgctgggcttcgggatcgaccggataaatgcccggaacgcctccggggaccggtgtgccatcaagtcctcgtacgcggaggccgtggggtcgctggggtccatggggtcgaaagcgtacttggcccggcatttgacctcgtaaaaggccaggggggtcttggggactggggccaggtagccgtgaatgtcccgaggacagacgagaatatccagggacgccccgaccatccccgtgtgaccgtccatgaggaccccacacgtatgcacgttctcttcggcgaggtcgctgggttcgtggaagataaagcgccgcgtgtcggcgccggcctcgccgccgtcgtccgcgcggcccacgcagtagcgaaacagcaggcttcgggccgtcggctcgttcacccgcccgaacatcaccgccgaagactgtacatccggccgcaggctggcgttgtgcttcagccactggggcgagaaacacggaccctgggggccccagcggagggtggatgcggtcgtgaggccccgccggagcagggcccatagctggcagtcggcctggttttgcgtggccgcctcgtaaaaccccatgaggggccggggcgccacggcgtccgcggcggccgggggcccgcggcgcgtcaggcgccataggtgccgaccgagtccgcggtccaccatacccgcctcctcgaggaccacggccagggaacacagataatccaggcgggcccagaggggaccgatggccagaggggcgcggacgccgcgcagcaacccgcgcaggtggcgctcgaacgtctcggctagtatatgggagggcagcgcgttggggatcaccgacgccgaccacatagagtcaaggtccggggagtcgggatcggcgtccgggtcgcgggcgtgggtgcccccaggagatagcggaatgtctggggtcggaggccctgaggcgtcagaaagtgccggcgacgcggcccggggcttttcgtctgcggtgtcggtggcgtgctgatcacgtggggggttaacgggcgaatgggagctcgggtccacagctgatgtcgtctggggtggggggggcaggggacggaaggtggttgtcagcggaagactgttagggcgggggcgcttgggggggctgtcggggccacgaggggtgtcctcggccagggcccagggacgcttagtcacggtgcgtcccggcggacatgctgggcctaccgtggactccatttccgagacgacgtggggggagcggtggttgagcgcgccgccgggtgaacgctgattctcacgacagcgcgtgccgcgcgcacgggttggtgtgacacaggcgggacaccagcaccaggagaggcttaagctcgggaggcagcgccaccgacgacagtatcgccttgtgtgtgtgctggtaatttatacaccgatccgtaaacgcgcgccgaatcttgggattgcggaggtggcgccggatgccctctgggacgtcatacgccaggccgtgggtgttggtctcggccgagttgacaaacagggctgggtgcagcacgcagcgataggcgagcagggccagggcgaagtccggcgacagctggttgttaaaatactggtaaccgggaaaccgggtcacgggtacgcccaggctcggggcgacgtacacgctaaccaccaactccagcagcgtctggcccagggcgtacaggtcaaccgctaacccgacgtcgtgcttcaggcggtggttggtaaattcggcccgttcgttgttaaggtatttcaccaacagctccgggggctggttatacccgtgacccaccagggtgtgaaagttggctgtggttagggcggtgggcatgccaaacatccggggggacttgaggtccggctcctggaggcaaaactgcccccgggcgatcgtggagttggagttgagggtgacgaggctaaagtcggcgaggacggcccgccggagcgagacggcgtccgaccgcagcatgacgaggatgttggcgcacttgatatccaggtggctgatcccgcaggtggtgtttaaaaacacaacggcgcgggccagctccgtgaagcactggtggagggccgtcgagaccgaggggtttgttgtgcgcagggacgccagttggccgatatacttaccgaggtccatgtcgtacgcggggaacactatctgtcgttgttgcagcgagaacccgaggggcgcgatgaagccgcggatgttgtgggtgcggccggcgcgtagaacgcactccccgaccaacagggtcgcgatgagctcaacggcaaaccactccttttcctttatggtcttaacggcaagcttatgttcgcgaatcagttggacgtcaccgtatcccccagaccccccgaagcttcgggccccggggatctcgagggtcgtgtagtgtagggcggggttgatggcgaacacggggctgcatagcttgcggatgcgcgtgagggtgaggatgtgcgagggggacgaggggggtgcggttaacgccgcctgggatctgcgcaggggcgggcggttcagtttggccgccgtaccgggcgtctcgggggacgcgcggcgatgagacgagcggctcattcgccatcgggatagtcccgcgcgaagccgctcgcggaggccggatcggtggcgggacccgtgggaggagcgggagacggcggcgtcctggagagaggggccgctggggcgcccggaggccccgtgggggttggagtgtacgtaggatgcgagccaatccttgaaggaccgttggcgtgcaccttgggggctgaggttagctgccacatgaccagcaggtcgctgtctgcgggactcatccatccttcggccaggtcgccgtctccccacagagaagcgttggtcgctgcttcctcgagttgctcctcctggtccgcaagacgatcgtccacggcgtccaggcgctcaccaagcgccggatcgaggtaccgtcggtgtgcggttagaaagtcacgacgcgccgcttgctcctccacgcgaattttaacacaggtcgcgcgctgtcgcatcatctctaagcgcgcgcgggactttagccgcgcctccaattccaagtgggccgcctttgcagccataaaggcgccaacaaaccgaggatcttgggtgctgacgccctcccggtgcagctgcagggtctggtccttgtaaatctcggctcggaggtgcgtctcggccaggcgtcggcgcagggccgcgtgggcggcatctcggtccattccgccaccctgcgggcgacccggggggtgctctgatagtctcgcgtgcccaaggcccgtgatcggggtacttcgccgccgcgacccgccacccggtgtgcgcgatgtttggtcagcagctggcgtccgacgtccagcagtacctggagcgcctcgagaaacagaggcaacttaaggtgggcgcggacgaggcgtcggcgggcctcaccatgggcggcgatgccctacgagtgccctttttagatttcgcgaccgcgacccccaagcgccaccagaccgtggtccctggcgtcgggacgctccacgactgctgcgagcactcgccgctcttctcggccgtggcgcggcggctgctgtttaatagcctggtgccggcgcaactaaaggggcgtgatttcgggggcgaccacacggccaagctggaattcctggcccccgagttggtacgggcggtggcgcgactgcggtttaaggagtgcgcgccggcggacgtggtgcctcagcgtaacgcctactatagcgttctgaatacgtttcaggccctccaccgctccgaagcctttcgccagctggtgcactttgtgcgggactttgcccagctgctcaaaacctccttccgggcctccagcctcacggagaccacgggcccccccaaaaaacgggccaaggtggacgtggccacccacggccggacgtacggcacgctggagctgttccaaaaaatgatccttatgcacgccacctactttctggccgccgtgctcctcggggaccacgcggagcaggtcaacacgttcctgcgtctcgtgtttgagatccccctgtttagcgacgcggccgtgcgccacttccgccagcgcgccaccgtgtttctcgtcccccggcgccacggcaagacctggtttctggtgcccctcatcgcgctgtcgctggcctcctttcgggggatcaagatcggctacacggcgcacatccgcaaggcgaccgagccggtgtttgaggagatcgacgcctgcctgcggggctggttcggttcggcccgagtggaccacgttaaaggggaaaccatctccttctcgtttccggacgggtcgcgcagtaccatcgtgtttgcctccagccacaacacaaacgtaagtcctcttttctttcgcatggctctcccaaggggccccgggtcgacccgacccacacccacccacccacatacacacacaaccagacgcgggaggaaagtctgccccgtgggcactgatttttattcgggatcgcttgaggaggcccgggcaacggcccgggcaacggtggggcaactcgtagcaaataggcgactgatgtacgaagagaagacacacaggcgccacccggcgctggtcggggggatgttgtccgcgccgcaccgtcccccgacgacctcttgcagacggtccgtgatgcaaggacggcggggggcctgcagcagggtgaccgtatccacgggatggccaaagagaagcggacacaggctagcatccccctggaccgccagggtacactgggccatcttggcccacagacacggggcgacgcagggacaggactccgttacgacggaggagagccacagtgcgttggcggaatcgatgtggggcggcggggcgcaggactcgcagccccccgggtggttggtgatcctggccaggagccatcccagatggcgggccctgcttcccggtggacagagcgaccccaggtcgctgtccatggcccagcagtagatctggccgctggggaggtgccaccaggcccccgggcccaaggcgcagcacgcgcccggctccgggggggtcttcgcggggaccagatacgcgccatccagctcgccgaccactggctcctccgcgagctgttcggtggttgggtcgggggtttcctccgggggggtggccgcccgtatgcgtgcgaacgtgagggtgcacaggagcggggtcagggggtgcgtcacgctccggaggtggacgatcgcgcagtagcggcgctcgcggttaaagaaaaagagggcaaagaaggtgttcgggggcaaccgcagcgccttggggcgcgtcagatacagaaaaatctcgcagaagagggcgcgcccggggtctgggttaggaagggccacctgacacagaggctcggtgaggaccgttagacaccgaaagatcttgagccgctcgtccgcccgaacgacgcgccacacaaagacggagttgacaatgcgcgcgatagagtcgacgtccgtccccaggtcgtcgactctatcgcgcgtgccgcgagctccggcccgggaatccggccggggcaaggtccccgggggaccaggcggcgccaggggccgccggggtcccagctgcgccatgccgggggcggggggagggcaaaccccagaggcgggggccaacggcgcggggaggagtgggtgggcgaggtggccgggggaaggcgcccgctagcgagaccggccgttcccggacgacaccttgcgacaaaacctaaggacagcggcccgcgcgacggggtccgagaggctaaggtaggccgcgatgttaatggtgaacgcaaagccgccgggaaagacaactatgccacagaggcggcgattaaaccccaggcagaggtaggcgtagctttccccgggcaggtattgctcgcagaccctgcgtggggctgtggaggggacggcctccatgaagcgacatttactctgctcgcgtttactgacgtcaccatccatcgccacggcgattggacgattgttaagccgcagcgtgtctccgcttgtgctgtagtagtcaaaaacgtaatggccgtcggagtcggcaaagcgggccgggaggtcgtcgccgagcgggacgacccgccgcccccgaccgccccgtccccccaggtgtgccaggacggccagggcatacgcggtgtgaaaaaaggcgtcgggggcggtcccctcgacggcgcgcatcaggttctcgaggagaatggggaagcgcctggtcacctcccccaaccacgcgcgttggtcggggccaaagtcatagcgcaggcgctgtgagattcgcgggccgccctgaagcgcggcccggatggcctggcccagggcccggaggcacgccagatgtatgcgcgcggtaaaggcgacctcggcggcgatgtcaaagggcggcaggacggggcgcgggtggcgcaggggcacctcgagcgcgggaaagcgtagcagcagctccgcctgcccagcgggagacagctggtgggggcgcacgacgcgttctgcggcgcaggcctcggtcagggccgtggccagcgccgaggacagcagcggagggcgggcgcgtcgcccgccccacgccacggagttctcgtaggagacgacgacgaagcgctgcttggttccgtagtggtggcgcaggaccacggagatagaacgacggctccacagccagtccggccggtcgccgccggccagggcttcccatccgcgatccaaccactcgaccagcgaccgcggctttgcggtaccaggggtaagggttagaacgtcgttcaggatgtcctcgcccccgggcccgtggggcgctggggccacaaagcggcccccgccggggggctccagacccgccagcaccgcatctgcgtcagccgcccccatggcgcccccgctgacggcctggtgaaccagggcgccctggcgtagccccgatgcaacgccacaggccgcacgcccggtccgcgctcggaccgggtggcggcgggtgacgtcctgcactgcccgctgaaccaacgcgaggatctcctcgttctcctgtgcgatggacacgtcctgggccgcggtcgtgtcgccgccgggggccgtcagctgctcctccggggagatgggggggtcggacgccccgacgatgggcgggtctgcgggcgcccccgcgtggggccgggccaagggctgcggacgcggggacgcgctttcccccagacccatggacaggtgggccgcagcctccttcgcggccggcggggcggcggcgccaagcagagcgacgtagcggcacaaatgccgacagacgcgcatgatgcgcgtgctgtcggccgcgtagcgcgtgttgggggggacgagctcgtcgtaactaaacagaatcacgcgggcacagctcgcccccgagccccacgcaaggcgcagcgccgccacggcgtacgggtcatagacgccctgcgcgtcacacaccacgggcagggagacgaacaaccccccggcgctggacgcacgcggaaggaggccagggtgtgccggcacgacgggggccagaagctcccccaccgcatccgcgggcacgtaggcggcaaacgccgtgcaccacggggtacagtcgccggtggcatgagcccgagtctggatttcgacctggaagtttgcggccgtcccgagtccggggcggccgcgcatcagggcggccagagggattcccgcggccgccaggcactcgctggatatgatgacgtgaaccaaagaccgagggccgacccgggccgtggccgagatcgtctggacctcgttggccaagtgcgcgttcatggttcgggggtgggtgtgggtgtgtaggcgatgcgggtcccccgagtccgcgggaagggcgtgggtttggcgcgcgtatgcgtattcgccaacggaggcgtgcgtgcttatgcgcggcgcgtttcttctgtctctagggaatccgaggccaggactttaacctgctctttgtcgacgaggccaactttattcgcccggatgcggtccagacgattatgggctttctcaaccaggccaactgcaagattatcttcgtgtcgtccaccaacaccgggaaggccagtacgagctttttgtacaacctccgcggggccgcagacgagcttctcaacgtggtgacctatatatgcgatgatcacatgccgagggtggtgacgcacacaaacgccacggcctgttcttgttatatcctcaacaagcccgttttcatcacgatggacggggcggttcgccggaccgccgatttgtttctggccgattccttcatgcaggagatcatcgggggccaggccagggagaccggcgacgaccggcccgttctgaccaagtctgcgggggagcggtttctgttgtaccgcccctcgaccaccaccaacagcggcctcatggcccccgatttgtacgtgtacgtggatcccgcgttcacggccaacacccgagcctccgggaccggcgtcgctgtcgtcgggcggtaccgcgacgattatatcatcttcgccctggagcacttttttctccgcgcgctcacgggctcggcccccgccgacatcgcccgctgcgtcgtccacagtctgacgcaggtcctggccctgcatcccggggcgtttcgcggcgtccgggtggcggtcgagggaaatagcagccaggactcggccgtcgccatcgccacgcacgtgcacacagagatgcaccgcctactggcctcggagggggccgacgcgggctcgggccccgagcttctcttctaccactgcgagcctcccgggagcgcggtgctgtacccctttttcctgctcaacaaacagaagacgcccgcctttgaacactttattaaaaagtttaactccgggggcgtcatggcctcccaggagatcgtttccgcgacggtgcgcctgcagaccgacccggtcgagtatctgctcgagcagctaaataacctcaccgaaaccgtctcccccaacactgacgtccgtacgtattccggaaaacggaacggcgcctcggatgaccttatggtcgccgtcattatggccatctacctcgcggcccaggccggacctccgcacacattcgctcctatcacacgcgtctcgtgagcgcccaataaacacacccaggtatgctacgcacgaccacggtgtcgtctgttaaggggggggggggaagggggtgttggcgggaagcgtgggaacacgggggattctctcacgaccggcaccagtaccacccccctgtgaacacagaaaccccaacccaaatcccataaacatacgacacacaggcatattttggaatttcttaggtttttatttatttaggtatgctggggtttctccctggatgcccacccccacccccccgtgggtctagccgggccttagggatagcgtataacgggggccatgtctccggaccgcacaacggccgcgccgtcaaaggtgcacacccgaaccacgggagccagggccaaggtgtctcctagttggcccgcgtgggtcagccaggcgacgagcgcctcgtaaagcggcagccttcgctctccatcctgcatcagggccggggcttcggggtgaatgagctgggcggcctcccgcgtgacactctgcatctgcagtagagcgttcacgtacccgtcctgggcacttagcgcaaagagccgggggattagcgtaaggatgatggtggttccctccgtgatcgagtaaaccatgttaaggaccagcgatcgcagctcggcgtttacgggaccgagttgttggacgtccgccagcagcgagaggcgactcccgttgtagtacagcacgttgaggtctggcagccctccggggtttctggggctggggttcaggtcccggatgcccctggccacgagccgcgccacgatttcgcgcgccaggggcgatggaagcggaacgggaaaccgcaacgtgaggtccagcgaatccaggcgcacgtccgtcgcttggccctcgaacacgggcgggacgaggctgatggggtccccgttacagagatctacgggggaggtgttgcgaaggttaacggtgccggcgtgggtgaggcccacgtccagggggcaggcgacgattcgcgtgggaagcacccgggtgatgaccgcggggaagcgccttcggtacgccagcaacaaccccaacgtgtcgggactgacgcctccggagacgaaggattcgtgcgccacgtcggccagcgtcagttgccggcggatggtcggcaggaataccacccgcccttcgcagcgctgcagcgccgccgcatcggggcgcgagatgcccgagggtatcgcgatgtcagtttcaaagccgtccgccagcatggcgccgatccacgcggcagggagtgcagtggtggttcgggtggcgggaggagcgcggtgggggtcagcggcgtagcagagacgggcgaccaacctcgcataggacggggggtgggtcttagggggttgggaggcgacagggaccccagagcatgcgcggggaggtctgtcgggcccagacgcaccgagagcgaatccgtccgcggagtcccggcttgggttttatggggcccggccctcggaatcgcggcttgtcggcggggacaaagggggcggggctaggggcttgcggaaacagaagacgcgtgggataaaagaatcgcactaccccaaggaagggcggggcggtttattacagagccagtcccttgagcggggatgcgtcatagacgagatactgcgcgaagtgggtctcccgcgcgtgggcttccccgttgcgggcactgcggaggagggcggggtcgctggcgcaggtgagcgggtaggcctcctgaaacaggccacacgggtcctccacgagttcgcggcaccccggggggcgcttaaactgtacgtcgctggcggcggtggccgtggacaccgccgaacccgtctccacgatcaggcgctccaggcagcgatgtttggcggcgatgtcggccgacgtaaagaacttaaagcaggggctgagcaccggcgaggccccgttgaggtggtaggccccgttatagagcaggtccccgtacgaaaatcgctgcgacgcccacgggttggccgtggccgcgaaggcccgggacgggtcgctctggccgtggtcgtacatgagggcggtgacatccccctccttgtcccccgcgtaaacgcccccggcggcgcgtccccgggggttgcagggccggcggaagtagttgacgtcggtcgacacgggggtggcgataaactcacacacggcgtcctggccgtggtccatccctgcgcgccgcggcacctgggcgcacccgaacacggggacgggctgggccggccccaggcggtttcccgccacgaccgcgttccgcaggtacacggctgccgcgttgtccaggagagggggagccccgcggcccaggtaaaagttttggggaaggttgcccatgtcggtgacggggttgcggacggttgccgtggccacgacggcggtgtagcccacgcccaggtccacgttcgcgcgcggctgggtgagcgtgaagtttacccccccgccagtttcgtgccgggccacctggagctggcccaggaagtacgcctccgacgcgcgctccgagaacagcacgttctcagtcacaaagcggtcctgtcggacgacggtgaacccaaacccgggatggaggcccgtcttgagctgatgatgcaaggccacgggactgatcttgaagtaccccgccatgagcgcgtaggtcagcgcgttctccccggccgcgctctcgcggacgtgctgcacgacgggctgtcggatcgacgaaaagtagttggcccccagagccggggggaccagggggacctgccgcgacaggtcgcgcagggccggggggaaattgggcgcgttcgccacgtggtcggccccggcgaacagcgcgtggacggggagggggtaaaaatagtcgccattttggatggtatggtccagatgctggggggccatcagcaggattccggcgtgcaacgccccgtcgaatatgcgcatgttggtggtggacgcggtgttggcgcccgcgtcgggcgccgccgagcagagcagcgccgttgtgcgttcggccatgttgtgggccagcacctgcagcgtgagcatggcgggcccgtccactaccacgcgcccgttgtgaaacatggcgttgaccgtgttggccaccagattggccgggtgcagggggtgcgcggggtccgtcacggggtcgctggggcactcctcgccgggggcgatctccgggaccaccatgttctgcagggtggcgtatacgcggtcgaagcgaacccccgcggtgcagcagcggccccgcgagaaggcgggcaccatcacgtagtagtaaatcttgtggtgcacggtccagtccgccccccggtgcggccggtcatccgcggcgtccgcggctcgggcctgggtgttgtgcagcagctggccgtcgttgcggttgaagtccgcggtcgccacgttacatgccgccgcgtacacggggtcgtggccccccgcgctaacccggcagtcgcgatggcggtccagggccgcgcgccgcatcagggcgtcacagtcccacacgaggggtggcagcagcgccgggtctcgcattaggtgattcagctcggcttgcgcctgcccgcccagctccgggccggtcagggtaaagtcatcaaccagctgggccagggcctcgacgtgcgccaccaggtcccggtacacggccatgcactcctcgggaaggtctcccccgaggtaggtcacgacgtacgagaccagcgagtagtcgttcacgaacgccgcgcaccgcgtgttgttccagtagctggtgatgcactggaccacgagccgggccagggcgcagaagacgtgctcgctgccgtgtatggcggcctgcagcaggtaaaacaccgccgggtagttgcggtcgtcgaacgccccgcgaacggcggcgatggtggcgggggccatggcgtggcgtcccacccccagctccaggccccgggcgtcccggaacgccgccggacatagcgccaggggcaagttgccgttcaccacgcgccaggtggcctggatctcccccgggccggccgggggaacgtccccccccggcagctccacgtcggccacccccacaaagaagtcgaacgcggggtgcagctcaagagccaggttggcgttgtcgggctgcataaactgctccggggtcatctggccttccgcgacccatcggacccgcccgtgggccaggcgctgcccccaggcgttcaaaaacagctgctgcatgtctgcggcggggccggccggggccgccacgtacgccccgtacggattggcggcttcgacggggtcgcggttaaggcccccgaccgccgcgtcaacgttcatcagcgaagggtggcacacggtcccgatcgcgtgttccagagacaggcgcagcacctggcggtccttcccccaaaaaaacagctggcggggcgggaaggcgcggggatccgggtggccgggggcggggactaggtccccggcgtgcgcggcaaaccgttccatgaccggattgaacaggcccaggggcaggacgaacgtcaggtccatggcgcccaccagggggtagggaacgttggtggcggcgtagatgcgcttctccagggcctccagaaagaccagcttctcgccgatggacaccagatccgcgcgcacgcgcgtcgtctggggggcgctctcgagctcgtccagcgtctgccggttcaggtcgagctgctcctcctgcatctccagcaggtggcggcccacgtcgtccagacttcgcacggccttgcccatcacgagcgccgtgaccaggttggccccgttcaggaccatctcgccgtacgtcaccggcacgtcggcttcggtgtcctccactttcaggaaggactgcaggaggcgctgtttgatcggggcggtggtgacgagcaccccgtcgaccggccgcccgcgcgtgtcggcatgcgtcagacggggcacggccacggagggctgcgtggccgtggtgaggtccacgagccaggcctcgacggcctcccggcggtggcccgccttgcccaggaaaaagctcgtctcgcagaagcttcgctttagctcggcgaccagggtcgcccgggccaccctggtggccaggcggccgttgtccaggtatcgttgcatcggcaacaacaaagccaggggcggcgccttttccagcagcacgtgcagcatctggtcggccgtgccgcgctcaaacgccccgaggacggcctggacgttgcgagcgagctgttggatggcgcgcaactggcgatgcgcgccgatacccgtcccgtccagggcctcccccgtgagcagggcgatggcctcggtggccaggctgaaggcggcgttcagggcccggcggtcgataatcttggtcatgtaattgtgtgtgggttgctcgatggggtgcgggccgtcgcgggcaatcagcggctggtggacctcgaactgtacgcgcccctcgttcatgtaggccagctccggaaacttggtacacacgcacgccaccgacaacccgagctccagaaagcgcacgagcgacagggtgttgcaatacgaccccagcagggcgtcgaactcgacgtcgtacaggctgtttgcatcggagcgcacgcgggaaaaaaaatcaaacaggcgtcgatgcgacgccacctcgatcgtgctaaggagggacccggtcggcaccatggccgcggcataccggtatcccggagggtcgcggttgggagcggccatggggtcgcgtggagatcggctgtctctagcgatattggcccggggaggctaagatccaccccaacgcccggccacccgtgtacgtgcccgacggcccaaggtccaccgaaagacacgacgggcccggacccaaaaaggcgggggatgctgtgtgagaggccgggtgccggtcgggggggaaaggcaccgggagaaggctgcggcctcgttccaggagaacccagtgtccccaacagacccggggacgtgggatcccaggccttatatacccccccccccgccccacccccgttagaacgcgacgggtgcattcaagatggccctggtccaaaagcgtgccaggaagaaattggcagaggcggcaaagctgtccgccgccgccacccacatcgaggccccggccgcgcaggctatccccagggcccgtgtgcgcaggggatcggtgggcggcagcatttggttggtggcgataaagtggaaaagcccgtccggactgaaggtctcgtgggcggcggcgaacaaggcacacagggccgtgcctcccaaaaacacggacatcccccaaaacacgggcgccgacaacggcagacgatccctcttgatgttaacgtacaggaggagcgcccgcaccgcccacgtaacgtagtagccgacgatggcggccaggatacaggccggcgccaccacccttccggtcagcccgtaatacatgcccgctgccaccatctccaacggcttcaggaccaaaaacgaccaaaggaacagaatcacgcgctttgaaaagaccggctgggtatggggcggaagacgcgagtatgccgaactgacaaaaaaatcagaggtgccgtacgaggacaatgaaaactgttcctccagcggcagttctccctcctcccccccgaaggcggcctcgtcgaccagatctcgatccaccagaggaaggtcatcccgcatggtcatggggtgtgcggtggaggtggggagaccgaaaccgcaaagggtcgcttacgtcagcaggatcccgagatcaaagacacccgggttcttgcacaaacaccacccgggttgcatccgcggaggcgagtgttttgataaggccgttccgcgccttgatataacctttgatgttgaccacaaaacccggaatttacgcctacgccccaatgcccacgcaagatgaggtaggtaacccccccgtgggtgtgacgttgcgtttagttcattggaggccaaggggaaaaatggggtggggaggaaacggaaaacccagtaggccgtgtcgggaacacgcccggggttgtcctcaaaaggcagggtccatactacggaagccgtcgttgtattcgagacctgcctgtgcaacgcacgtcggggttgcctgtgtccggttcggcccccaccgcgtgcggcacgcacgaggacgagtccgcgtgctttattggcgttccaagcgttgccctccagtttctgttgtcggtgttcccccatacccacgcccacatccaccgtagggggcctctgggccgtgttacgtcgccgcccgcgatggagcttagctacgccaccaccatgcactaccgggacgttgtgttttacgtcacaacggaccgaaaccgggcctactttgtgtgcggggggtgtgtttattccgtggggcggccgtgtgcctcgcagcccggggagattgccaagtttggtctggtcgttcgagggacaggcccagacgaccgcgtggtcgccaactatgtacgaagcgagctccgacaacgcggcctgcaggacgtgcgtcccattggggaggacgaggtgtttctggacagcgtgtgtcttctaaacccgaacgtgagctccgagctggatgtgattaacacgaacgacgtggaagtgctggacgaatgtctggccgagtactgcacctcgctgcgaaccagcccgggtgtgctaatatccgggctgcgcgtgcgggcgcaggacagaatcatcgagttgtttgaacacccaacgatagtcaacgtttcctcgcactttgtgtataccccgtccccatacgtgttcgccctggcccaggcgcacctcccccggctcccgagctcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgactcgagttccctggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctagccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctgtttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagccacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgtctcgctcgctcccaacacggacagtctgtgtaaaagaccccaataaacgtatatcgctactacacccttgtgtgtcaatggacgcctctccgggggggggggagggaaagcaaagaggggctgggggagcggcaccaccggggcctgaacaaacaaaccacagacacggttacagtttattcggtcgggcggagaaacggccgaagccacgcccactttattcgcgtctccaaaaaaacgggacacttgtccggagaacctttaggatgccagccagggcggcggtaatcataaccacgcccagcgcagaggcggccagaaacccgggcgcaattgcggccacgggctgcgtgtcaaaggctagcaaatgaatgacggttccgtttggaaatagcaacaaggccgtggacggcacgtcgctcgaaaacacgcttggggcgccctccgtcggcccggcggcgatttgctgctgtgtgttgtccgtatccaccagcaacacagacatgacctccccggccggggtgtagcgcataaacacggcccccacgagccccaggtcgcgctggttttgggtgcgcaccagccgcttggactcgatatcccgggtggagccttcgcatgtcgcggtgaggtaggttaggaacagtgggcgtcggacgtcgacgccggtgagcttgtagccgatcccccggggcagaggggagtgggtgacgacgtagctggcgttgtgggtgatgggtaccaggatccgtggctcgacgttggcagactgccccccgcaccgatgtgaggcctcagggacgaaggcgcggatcagggcgttgtagtgtgcccaacgcgtcagggtcgaggcgaggccgtgggtctgctgggccaggacttcgaccggggtctcggatcgggtggcttgagccagcgcgtccaggataaacacgctctcgtctagatcaaagcgcagggaggccgcgcatggcgaaaagtggtccggaagccaaaagagggttttctggtggtcggcccgggccagcgcggtccggaggtcggcgttggtcgctgcggcgacgtcggacgtacacagggccgaggctatcagaaggctccggcgggcgcgttcccgctgcaccgccgaggggacgccagccaagaacggctgccggaggacagccgaggcgtaaaatagcgcccggtggacgaccggggtggtcagcacgcggccccctagaaactcggcatacagggcgtcgatgagatgggctgcgctgggcgccactgcgtcgtacgccgaggggctatccagcacgaaggccagctgatagcccagcgcgtgtaatgccaagctctgttcgcgctccagaatctcggccaccaggtgctggagccgagcctctagctgcaggcgggccgtgggatccaagactgacacattaaaaaacacagaatccgcggcacagcccgcggccccgcgggcggccaacccggcaagcgcgcgcgagtgggccaaaaagcctagcaggtcggagaggcagaccgcgccgtttgcgtgggcggcgttcacgaaagcaaaacccgacgtcgcgagcagccccgttaggcgccagaagagaggggggcgcgggccctgctcggcgcccgcgtcccccgagaaaaactccgcgtatgcccgcgacaggaactgggcgtagttcgtgccctcctccgggtagccgcccacgcggcggagggcgtccagcgcggagccgttgtcggcccgcgtcagggaccctaggacaaagacccgataccgggggccgcccgggggcccgggaagagcccccggggggttttcgtccgcggggtccccgacccgatctagcgtctggcccgcggggaccaccatcacttccaccggagggctgtcgtgcatggatatcacgagccccatgaattcccgcccgtagcgcgcgcgcaccagcgcggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggtcgggggggcggtgtatcacgcggcccgctcgggtggctcgccgtcgccacgttgtctccccgcgggaacgtcagggcctcggggtcagggacggccgaaaacgttacccaggcccgggaacgcagcaacacggaggcggctggattgtgcaagagacccttaaggggggcgaccgaggggggaggctgggcggtcggctcgaccgtggtgggggcgggcaggctcgcgttcgggggccggccgagcaggtaggtcttcgggatgtaaagcagctggccggggtcccgcggaaactcggccgtggtgaccaatacaaaacaaaagcgctcctcgtaccagcgaagaaggggcagagatgccgtagtcaggtttagttcgtccggcggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggcccgtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggcagccggcgggcgattggtcgtaatccaggataaagacatgcatgggacggaggcgtttggccaagacgtccaaagcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgcccccgaaagctgtccccaatcctcccgccacgacccgccgccctgcagataccgcaccgtattggcaagcagcccataaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccaacacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccaggcggacttccgtggctttttgttgccggcgagggcgcaacgccgtacgtcggttgttatggccgcgagaacgcgcagcctggtcgaacgcagacgcgtgttgatggcaggggtacgaagccatacgcgcttctacaaggcgctggccgaagaggtgcgggagtttcacgccaccaagatctgcggcacgctgttgacgctgttaagcgggtcgctgcagggtcgctcggtattcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccattgagcctaggacccagcgagcccgtcgccgccgcggcggcgccgcccgggggtctgcgagcagaccgaaaaggtcacactctggggcgcgcgacccgcccgagtcagcggcccgccagttaccacccgccgaccaaacccccacctccacggagggcgggggggtgcttaagaggatcgcggcgctcttctgcgtgcccgtggccaccaagaccaaaccccgagccgcctccgaatgagagtgtttcgttccttccccctccccccgcgtcagacaaaccctaaccaccgcttaagcggcccccgcgaggtccgaagactcatttggatccggcgggagccacccgacaacagcccccgggttttcccacgccagacgccggtccgctgtgccatcgcgccccctcatcccaccccccatcttgtccccaaataaaacaaggtctggtagttaggacaacgaccgcagttctcgtgtgttattttcgctctccgcctctcgcagatggacccgtactgcccatttgacgctctggacgtctgggaacacaggcgcttcatagtcgccgattcccgaaacttcatcacccccgagttcccccgggacttttggatgtcgcccgtctttaacctcccccgggagacggcggcggagcaggtggtcgtcctacaggcccagcgcacagcggctgccgctgccctggagaacgccgccatgcaggcggccgagctccccgtcgatatcgagcgccggttacgcccgatcgaacggaacgtgcacgagatcgcaggcgccctggaggcgctggagacggcggcggccgccgccgaagaggcggatgccgcgcgcggggatgagccggcgggtgggggcgacgggggggcgcccccgggtctggccgtcgcggagatggaggtccagatcgtgcgcaacgacccgccgctacgatacgacaccaacctccccgtggatctgctacacatggtgtacgcgggccgcggggcgaccggctcgtcgggggtggtgttcgggacctggtaccgcactatccaggaccgcaccatcacggactttcccctgaccacccgcagtgccgactttcgggacggccgtatgtccaagaccttcatgacggcgctggtactgtccctgcaggcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcggtgatcgggaccgagggcggccggccacagtaccgctaccgcgacgacaagctccccaagacgcagttcgcggccggcgggggccgctacgaacacggagcgctggcgtcgcacatcgtgatcgccacgctgatgcaccacggggtgctcccggcggccccgggggacgtcccccgggacgcgagtacccacgttaaccccgacggcgtggcgcaccacgacgacataaaccgcgccgccgccgcgttcctcagccggggccacaacctattcctgtgggaggaccagactctgctgcgggcaaccgcgaacaccataacggccctgggcgttatccagcggctcctcgcgaacggcaacgtgtacgcggaccgcctcaacaaccgcctgcagctgggcatgctgatccccggagccgtcccttcggaggccatcgcccgtggggcctccgggtccgactcgggggccatcaagagcggagacaacaatctggaggcgctatgtgccaattacgtgcttccgctgtaccgggccgacccggcggtcgagctgacccagctgtttcccggcctggccgccctgtgtcttgacgcccaggcggggcggccggtcgggtcgacgcggcgggtggtggatatgtcatcgggggcccgccaggcggcgctggtgcgcctcaccgccctggaactcatcaaccgcacccgcacaaaccccacccctgtgggggaggttatccacgcccacgacgccctggcgatccaatacgaacaggggcttggcctgctggcgcagcaggcacgcattggcttgggctccaacaccaagcgtttctccgcgttcaacgttagcagcgactacgacatgttgtactttttatgtctggggttcattccacagtacctgtcggcggtttagtgggtggtgggcgaggggggagggggcattagggagaaaaaacaagagcctccgttgggttttctttgtgcctgtactcaaaaggtcataccccgtaaacggcgggctccagtcccggcccggcggttggcgtgaacgcaacggcgggagctgggttagcgtttagtttagcattcgctctcgcctttccgcccgccccccgaccgttgcgcctttttttttttcgtccaccaaagtctctgtgggtgcgcgcatggcagccgatgccccgggagaccggatggaggagcccctgcccgacagggccgtgcccatttacgtggctgggtttttggccctgtatgacagcggggactcgggcgagttggcattggatccggatacggtgcgggcggccctgcctccggataacccactcccgattaacgtggaccaccgcgctggctgcgaggtggggcgggtgctggccgtggtcgacgacccccgcgggccgttttttgtggggctgatcgcctgcgtgcagctggagcgcgtcctcgagacggccgccagcgctgcgattttcgagcgccgcgggccgccgctctcccgggaggagcgcctgttgtacctgatcaccaactacctgccctcggtctccctggccacaaaacgcctggggggcgaggcgcaccccgatcgcacgctgttcgcgcacgtcgcgctgtgcgcgatcgggcggcgcctcggcactatcgtcacctacgacaccggtctcgacgccgccatcgcgccctttcgccacctgtcgccggcgtctcgcgagggggcgcggcgactggccgccgaggccgagctcgcgctgtccgggcgcacctgggcgcccggcgtggaggcgctgacccacacgctgctttccaccgccgttaacaacatgatgctgcgggaccgctggagcctggtggccgagcggcggcggcaggccgggatcgccggacacacctacctccaggcgagcgaaaaattcaaaatgtggggggcggagcctgtttccgcgccggcgcgcgggtataagaacggggccccggagtccacggacataccgcccggctcgatcgctgccgcgccgcagggtgaccggtgcccaatcgtccgtcagcgcggggtcgccttgtccccggtactgccccccatgaaccccgttccgacatcgggcaccccggcccccgcgccgcccggcgacgggagctacctgtggatcccggcctcccattacaaccagctcgtcgccggccatgccgcgccccaaccccagccgcattccgcgtttggtttcccggctgcggcggggtccgtggcctatgggcctcacggtgcgggtctttcccagcattaccctccccacgtcgcccatcagtatcccggggtgctgttctcgggacccagcccactcgaggcgcagatagccgcgttggtgggggccatagccgcggaccgccaggcgggcggtcagccggccgcgggagaccctggggtccgggggtcgggaaagcgtcgccggtacgaggcggggccgtcggagtcctactgcgaccaggacgaaccggacgcggactacccgtactaccccggggaggctcgaggcgcgccgcgcggggtcgactcccggcgcgcggcccgccattctcccgggaccaacgagaccatcacggcgctgatgggggcggtgacgtctctgcagcaggaactggcgcacatgcgggctcggaccagcgccccctatggaatgtacacgccggtggcgcactatcgccctcaggtgggggagccggaaccaacaacgacccacccggccctttgtcccccggaggccgtgtatcgccccccaccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaataagacccattggtgtatgttctttttttattgggaggcgcgggtaggcgggtagctttacaatgcaaaagccttcgacgtggaggaaggcgtggggggggggggaatcggcactgaccaagggggtccgttttgtcacgggaaaggaaagaggaaacaggccgcggacacccgggggagtttgtgttcccttttctttcttcccacacacacaaaaggcgtaccaaacaaacaaaccaaaagatgcacatgcggtttaacacccgtggtttttatttacaacaaaccccccatcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggcagacaccagggccatgtaccgtatcatctcccgggcctcggctagcttggcctcgtcaaagtcgccgccctcctcgccctccccggacgcgtccgggttggtggggttcttgagctccttggtggttagcgggtacagggccttcatggggttgctctgcagccgcatgacgtagcgaaaggcgaagaaggccgccgccaggccggccaggaccaacagacccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcaaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggtgatgttgaggtcgatgaaggtgctgacggtggtgatgtcggcgcggctcagctggtgggagtacgcgtactcctcgaagtacacgtagcccccaccgaaggtgaagtagcgccggtgtcccacggtgcacggctcgatcgcatcgcgcgtcagccgcagctcgttgttctcccccagctgcccctcgaccaacgggccctggtcttcgtaccgaaagctgaccagggggcggctgtagcaggccccgggccgcgagctgatgcgcatcgagttttggacgatcacgttgtccgcggcgaccggcacgcacgtggagacggccatcacgtcgccgagcatccgcgcgctcacccgccggcccacggtggccgaggcgatggcgttggggttcagcttgcgggcctcgttccacagggtcagctcgtgattctgcagctcgcaccacgcgatggcaacgcggcccaacatatcgttgacatggcgctgtatgtggttgtacgtaaactgcagcctggcgaactcgatggaggaggtggtcttgatgcgctccacggacgcgttggcgctggccccgggcggcgggggcgtggggtttgggggcttgcggctctgctcgcggaggtgttcccgcacgtacagctccgcgagcgtgttgctgagaaggggctggtacgcgatcagaaagcccccattggccaggtagtactgcggctggcccaccttgatgtgcgtcgcgttgtacctgcgggcgaagatgcggtccatggcgtcgcgggcgtccttgccgatgcagtcccccaggtccacgcgcgagagcgggtactcggtcaggttggtggtgaaggtggtggatatggcgtcggaagagaatcggaaggagccgccgtactcggagcgcagcatctcgtccacctcctgccacttggtcatggtgcagaccgacgggcgctttggcacccagtcccaggccacggtgaacttgggggtcgtgagcaggttccgggtggtcggcgccgtggcccgggccttggtggtgaggtcgcgcgcgtagaagccgtcgacctgcttgaagcggtcggcggcgtagctggtgtgttcggtgtgcgacccctcccggtagccgtaaaacggggacatgtacacaaagtcgccagtcgccaacacaaactcgtcgtacgggtacaccgagcgcgcgtccacctcctcgacgatgcagtttaccgtcgtcccgtaccggtggaacgcctccacccgcgaggggttgtacttgaggtcggtggtgtgccagccccggctcgtgcgggtcgcggcgttggccggtttcagctccatgtcggtctcgtggtcgtcccggtgaaacgcggtggtctccaggttgttgcgcacgtacttggccgtggaccgacagacccccttggcgttgatcttgtcgatcacctcctcgaaggggacgggggcgcggtcctcaaagatccccataaactgggagtagcggtggccgaaccacacctgcgaaacggtgacgtctttgtagtacatggtggccttgaacttgtacggggcgatgttctccttgaagaccaccgcgatgccctccgtgtagttctgaccctcgggccgggtcgggcagcggcgcggctgctcgaactgcaccaccgtggcgcccgtggggggtgggcacacgtaaaagtttgcatcggtgttctccgccttgatgtcccgcaggtgctcgcgcagggtggcgtggcccgcggcgacggtcgcgttgtcgccggcggggcgcggcggctttgggggtttcggttttctgttcttcttcggtttcgggtcccccgttgggggggcgccaggggcgggcggcgccggagtggcagggcccccgttcgccgcctgggtcgcggccgcgaccccaggcgtgccgggggaactcggagccgccgacgccaccaggacccccagcgtcaaccccaagagcgcccatacgacgaaccaccggcgcccccgcgcgggggcgccctggcgcatggcgggactacgggggcccgtcgtgccccccgtcaggtagcctgggggcgaggtgctggaggaccgagtagaggatcgagaaaacgtctcggtcgtagaccacgaccgaccgggggccgatacagccgtcgggggcgctctcgacgatggccaccagcggacagtcggagtcgtacgtgagatatacgccgggcgggtaacggtaacgaccttcggaggtcgggcggctgcagtccgggcggcgcaactcgagctccccgcaccggtagaccgaggcaaagagtgtggtggcgataatcagctcgcgaatatatcgccaggcggcgcgctgagtgggcgttattccggaaatgccgtcaaaacagtaaaacctctgaaattcgctgacggcccaatcagcacccgagccccccgcccccatgatgaaccgggcgagctcctccttcaggtgcggcaggagccccacgttctcgacgctgtaatacagcgcggtgttggggggctgggcgaagctgtgggtggagtgatcaaagaggggcccgttgacgagctcgaagaagcgatgggtgatgctggggagcagggccgggtccacctggtgtcgcaggagagacgctcgcatgaaccggtgcgcgtcgaacacgcccggcgccgagcggttgtcgatgaccgtgcccgcgcccgccgtcagggcgcagaagcgcgcgcgcgccgcaaagccgttggcgaccgcggcgaacgtcgcgggcagcacctcgccgtggacgctgacccgcagcatcttctcgagctccccgcgctgctcgcggacgcagcgccccaggctggccaacgaccgcttcgtcaggcggtccgcgtacagccgccgtcgctcccgcacgtccgcggccgcttgcgtggcgatgtccccccacgtctcgggcccctgccccccgggcccgcggcgacggtcttcgtcctcgcccccgcccccgggagctcccaacccccgtgccccttcctctacggcgacacggtccccgtcgtcgtcggggcccgcgccgcccttgggcgcgtccgccgcgccccccgcccccatgcgcgccagcacgcgacgcagcgcctcctcgtcgcactgttcggggctgacgaggcgccgcaagagcggcgtcgtcaggtggtggtcgtagcacgcgcggatgagcgcctcgatctgatcgtcgggtgacgtggcctgaccgccgattattagggcgtccaccatatccagcgccgccaggtggctcccgaacgcgcgatcgaaatgctccgcccgccgcccgaacagcgccagttccacggccaccgcggcggtctcctgctgcaactcgcgccgcgccagcgcggtcaggttgctggcaaacgcgtccatggtggtctggccggcgcggtcgccggacgcgagccagaatcgcaattcgctgatggcgtacaggccgggcgtggtggcctgaaacacgtcgtgcgcctccagcagggcgtcggcctccttgcggaccgagtcgttctcgggcgacgggtggggctgcccgtcgccccccgcggtccgggccagcgcatggtccaacacggagagcgcccgcgcgcggtcggcgtccgacagcccggcggcgtggggcaggtaccgccgcagctcgttggcgtccagccgcacctgcgcctgctgggtgacgtggttacagatacggtccgccaggcggcgggcgatcgtcgccccctggttcgccgtcacacacagttcctcgaaacagaccgcgcaggggtgggacgggtcgctaagctccggggggacgataaggcccgaccccaccgcccccaccataaactcccgaacgcgctccagcgcggcggtggcgccgcgcgagggggtgatgaggtggcagtagtttagctgctttagaaagttctcgacgtcgtgcaggaaacacagctccatatggacggtcccgccatacgtatccagcctgacccgttggtgatacggacagggtcgggccaggcccatggtctccgtgaaaaacgccgcgacgtctcccgcggtcgcgaacgtctccaggctgcccaggagccgctcgccctcgcgccacgcgtactctagcagcaactccagggtgaccgacagcggggtgagaaaggccccggcctgggcctccaggcccggcctcagacgacgccgcagcgcccgcacctgaagcgcgttcagcttcagttgggggagcttcccccgtccgatgtgggggtcgcaccgccggagcagctctatctgaaacacataggtctgcacctgcccgagcagggctaacaacttttgacgggccacggtgggctcggacaccggggcggccatctcgcggcgccgatctgtaccgcggccggagtatgcggtggaccgaggcggtccgtacgctacccggcgtctggctgagccccggggtccccctcttcggggcggcctcccgcgggcccgccgaccggcaagccgggagtcggcggcgcgtgcgtttctgctctattcccagacaccgcggagaggaatcacggcccgcccagagatatagacacggaacacaaacaagcacggatgtcgtagcaataatttattttacacacattccccgccccgccctaggttcccccaccccccaacccctcacagcatatccaacgtcaggtctccctttttgtcggggggcccctccccaaacgggtcatccccgtggaacgcccgtttgcggccggcaaatgccggtcccggggcccccgggccgccgaacggcgtcgcgttgtcgtcctcgcagccaaaatccccaaagttaaacacctccccggcgttgccgagttggctgactagggcctcggcctcgtgcgccacctccagggccgcgtccgtcgaccactcgccgttgccgcgctccagggcacgcgcggtcagctccatcatctcctcgcttaggtactcgtcctccaggagcgccagccagtcctcgatctgcagctgctgggtgcggggccccaggcttttcacggtcgccacgaacacgctactggcgacggccgccccgccctcggagataatgccccggagctgctcgcacagcgagctttcgtgcgctccgccgccgaggcttgaggccgcgcacacaaacccggcccggggacaggccaggacgaacttgcgggtgcggtcaaaaataaggagcgggcacgcgtttttgccgcccatcaggctggcccagttcccggcctgaaacacacggtcgttgccggccatgccgtagtacttgctgatgctcaaccccaacacgaccatggggcgcgccgccatgacgggccgcagcaggttgcagctggcgaacatggacgtccacgcgcccggatgcgcgtccacggcgtccatcagcgcgcgggccccggcctccaggcccgccccgccctgcgcggaccacgcggccgcagcctgcacgctggggggacggcgggaccccgcgatgatggccgtaagggtgttgatgaagtatgtcgagtgatcgcagtaccgcagaatctggtttgccatgtagtacatcgccagctcgctcacgttgttgggggccaggttaataaagtttatcgcgccgtagtccagggaaaactttttaatgaacgcgatggtctcgatgtcctcgcgcgacaggagccgggcgggaagctggttgcgttggagggccgtccagaaccactgcgggttcggctggttggaccccgggggcttgccgttggggaagatggccgcgtggaactgcttcagcagaaagcccagcggtccgaggaggatgtccacgcgcttgtcgggcttctggtaggcgctctggaggctggcgacccgcgccttggcggcctcggacgcgttggcgctcgcgcccgcgaacaacacgcggctcttgacgcgcagctccttgggaaaccccagggtcacgcgggcaacgtcgccctcgaagctgctctcggcgggggccgtctggccggccgttaggctgggggcgcagatagccgccccctccgagagcgcgaccgtcagcgttttggccgacagaaacccgttgttaaacatgtccatcacgcgccgccgcagcaccggttggaattgattgcgaaagttgcgcccctcgaccgactgcccggcgaacaccccgtggcactgactcagggccaggtcctggtacacggcgaggttggatcgccgcccgagaagctgaagcagggggcacggcccgcacgcgtacgggtccagcgtcagggacatggcgtggttggcctcgcccagaccgtcgcgaaacttgaagttcctcccctccaccaggttgcgcatcagctgctccacctcgcggtccacgacctgcctgacgttgttcaccaccgtatgcagggcctcgcggttggtgatgatggtctccagccgccccatggccgtggggaccgcctggtccacgtactgcagggtctcgagttcggccatgacgcgctcggtcgccgcgcggtacgtctcctgcatgatggtccgggcggtctcggatccgtccgcgcgcttcagggccgagaaggcggcgtagtttcccagcacgtcgcagtcgctgtacatgctgttcatggtcccgaagacgccgatggctccgcgggcggcgctggcgaactttggatggcgcgcccggaggcgcatgagcgtcgtgtgtacgcaggcgtggcgcgtgtcgaaggtgcataggttacagggcacgtcggtctggttggagtccgcgacgtatcgaaacacgtccatctcctggcgcccgacgatcacggcgccgtcgcagcgctccaggtaaaacagcatcttggccagcagcgccggggaaaacccacacagcatggccaggtgctcgccggcaaattcctgggttccgccgacgaggggcgcggtgggccgaccctcgaacccgggcaccacgtgtccctcgcggtccacctgtgggttggccgccacgtgggtcccgggcacgaggaagaagcggtaaaaggagggtttgctgtggtcctttgggtccgccgggccggcgtcgtccacctcggtgagatggagggccgagttggtgctaaataccatggcccccacgagtcccgcggcgcgcgccaggtacgccccgacggcgttggcgcgggccgcggccgtgtcctggccctcgaacagcggccacgcggagatgtcggtgggcggctcgtcaaagacggccatcgacacgatagactcgagggccagggcggcgtctccggccatgacggaggccaggcgctgttcgaacccgcccgccgcgcccttgccgccgccgtcgcgcccgccccgcggggtcttaccctggctggcttcgaaggccgtgaacgtaatgtcggcggggagggcggcgccctcgtggttttcgtcaaacgccaggtgggcggccgcgcgggccacggcgtccacgtttcggcatcgcagtgccacggcggcgggtcccacgaccgcctcgaacaggaggcggttgagggggcggttaaaaaacggaagcgggtaggtaaatttctccccgatcgatcggtggttggcgttgaacggctctgcgatgacacggctaaaatccggcatgaacagctgcaacgggtacacgggtatgcggtgcacctccgccccgcctatggttaccttgtccgagcctcccaggtgcagaaaggtgttgttgatgcacacggcctccttgaagccctcggtaacgaccagatacaggagggcgcggtccgggtccaggccgaggcgctcacacagcgcctcccccgtcgtctcgtgtttgaggtcgccgggccggggggtgtagtccgaaaagccaaaatggcggcgtgcccgctcgcaaagtcgcgtcaggttcggggcctgggtgctggggtccaggtgccggccgccgtgaaagacgtacacggacgagctgtagtgcgagggcgtcagtttcagggacaccgcggtacccccgagccccgtcgtgcgagaacccacgaccacggccacgttggcctcaaagccgctctccacggtcaggcccacgaccaggggcgccacggcgacgtcggaatcgccgctgcgtgccgacagtaacgccagaagctcgatgccttcggacggacacgcgcgagcgtacacgtatcccaggggcccgggggggaccttgatggtggttgccgtcttgggctttgtctccatgtccttttgtcaatcggtccgcgaacggaggtaatcccggcacgacgacggacgcccgacaaggtatgtctcccgagcgtcaaaatccgggggggggcggcgacggtcaaggggagggttggagaccggggttggggaatgaatccctccccttcaccgacaaccccccgggtaaccacggggtcgccgatgaaccccggcggccggcaacgcggggtccctgcgagaggcacagatgcttacggtcaggtgctccgggtcgggtgcgtctggtatgcggttggtatatgtacactttacctgggggcgtgccggtccgccccagcccctcccacgccccgcgcgtcatcagccggtgggcgtggccgctattataaaaaaagtgagaacgcgaagcgttcgcactttgtcctaataatatatatattattaggacaaagtgcgaacgcttcgcgttctcactttttttataatagcggccacgcccaccggctacgtcactctcctgtcggccgccggcggtccataagcccggccggccgggccgacgcgaataaaccgggccgccggccggggcgccgcgcagcagctcgccgcccggatccgccagacaaacaaggcccttgcacatgccggcccgggcgagcctgggggtccggtaattttgccatcccacccaagcggctttttgggtttttctcttcccccctccccacattcccctctttaggggttcgggtgggaacaaccgcgatgttttccggtggcggcggcccgctgtcccccggaggaaagtcggcggccagggcggcgtccgggttttttgcgcccgccggccctcgcggagccagccggggacccccgccttgtttgaggcaaaacttttacaacccctacctcgccccagtcgggacgcaacagaagccgaccgggccaacccagcgccatacgtactatagcgaatgcgatgaatttcgattcatcgccccgcgggtgctggacgaggatgcccccccggagaagcgcgccggggtgcacgacggtcacctcaagcgcgcccccaaggtgtactgcgggggggacgagcgcgacgtcctccgcgtcgggtcgggcggcttctggccgcggcgctcgcgcctgtggggcggcgtggaccacgccccggcggggttcaaccccaccgtcaccgtctttcacgtgtacgacatcctggagaacgtggagcacgcgtacggcatgcgcgcggcccagttccacgcgcggtttatggacgccatcacaccgacggggaccgtcatcacgctcctgggcctgactccggaaggccaccgggtggccgttcacgtttacggcacgcggcagtacttttacatgaacaaggaggaggtcgacaggcacctacaatgccgcgccccacgagatctctgcgagcgcatggccgcggccctgcgcgagtccccgggcgcgtcgttccgcggcatctccgcggaccacttcgaggcggaggtggtggagcgcaccgacgtgtactactacgagacgcgccccgctctgttttaccgcgtctacgtccgaagcgggcgtgtgctgtcgtacctgtgcgacaacttctgcccggccatcaagaagtacgagggtggggtcgacgccaccacccggttcatcctggacaaccccgggttcgtcaccttcggctggtaccgtctcaaaccgggccggaacaacacgctagcccagccggcggccccgatggccttcgggacatccagcgacgtcgagtttaactgtacggcggacaacctggccatcgaggggggcatgagcgacctaccggcatacaagctcatgtgcttcgatatcgaatgcaaggcggggggggaggacgagctggcctttccggtggccgggcacccggaggacctggtcatccagatatcctgtctgctctacgacctgtccaccaccgccctggagcacgtcctcctgttttcgctcggttcctgcgacctccccgaatcccacctgaacgagctggcggccaggggcctgcccacgcccgtggttctggaattcgacagcgaattcgagatgctgttggccttcatgacccttgtgaaacagtacggccccgagttcgtgaccgggtacaacatcatcaacttcgactggcccttcttgctggccaagctgacggacatttacaaggtccccctggacgggtacggccgcatgaacggccggggcgtgtttcgcgtgtgggacataggccagagccacttccagaagcgcagcaagataaaggtgaacggcatggtgaacatcgacatgtacgggattataaccgacaagatcaagctctcgagctacaagctcaacgccgtggccgaagccgtcctgaaggacaagaagaaggacctgagctatcgcgacatccccgcctactacgccgccgggcccgcgcaacgcggggtgatcggcgagtactgcatacaggattccctgctggtgggccagctgttttttaagtttttgccccatctggagctctcggccgtcgcgcgcttggcgggtattaacatcacccgcaccatctacgacggccagcagatccgcgtctttacgtgcctgctgcgcctggccgaccagaagggctttattctgccggacacccaggggcgatttaggggcgccgggggggaggcgcccaagcgtccggccgcagcccgggaggacgaggagcggccagaggaggagggggaggacgaggacgaacgcgaggagggcgggggcgagcgggagccggagggcgcgcgggagaccgccggcaggcacgtggggtaccagggggccagggtccttgaccccacttccgggtttcacgtgaaccccgtggtggtgttcgactttgccagcctgtaccccagcatcatccaggcccacaacctgtgcttcagcacgctctccctgagggccgacgcagtggcgcacctggaggcgggcaaggactacctggagatcgaggtgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtgtacgggttcacgggagtgcagcacggactcctgccgtgcctgcacgttgccgcgacggtgacgaccatcggccgcgagatgctgctcgcgacccgcgagtacgtccacgcgcgctgggcggccttcgaacagctcctggccgatttcccggaggcggccgacatgcgcgcccccgggccctattccatgcgcatcatctacggggacacggactccatctttgtgctgtgccgcggcctcacggccgccgggctgacggccgtgggcgacaagatggcgagccacatctcgcgcgcgctgtttctgccccccatcaaactcgagtgcgaaaagacgttcaccaagctgctgctgatcgccaagaaaaagtacatcggcgtcatctacgggggtaagatgctcatcaagggcgtggatctggtgcgcaaaaacaactgcgcgtttatcaaccgcacctccagggccctggtcgacctgctgttttacgacgataccgtctccggagcggccgccgcgttagccgagcgccccgcggaggagtggctggcgcgacccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtattacaagctcatggcccgccgcgcgcaggtcccgtccatcaaggaccggatcccgtacgtgatcgtggcccagacccgcgaggtagaggagacggtcgcgcggctggccgccctccgcgagctagacgccgccgccccaggggacgagcccgccccccccgcggccctgccctccccggccaagcgcccccgggagacgccgtcgcctgccgaccccccgggaggcgcgtccaagccccgcaagctgctggtgtccgagctggccgaggatcccgcatacgccattgcccacggcgtcgccctgaacacggactattacttctcccacctgttgggggcggcgtgcgtgacattcaaggccctgtttgggaataacgccaagatcaccgagagtctgttaaaaaggtttattcccgaagtgtggcaccccccggacgacgtggccgcgcggctccggaccgcagggttcggggcggtgggtgccggcgctacggcggaggaaactcgtcgaatgttgcatagagcctttgatactctagcatgagccccccgtcgaagctgatgtccctcattttacaataaatgtctgcggccgacacggtcggaatctccgcgtccgtgggtttctctgcgttgcgccggaccacgagcacaaacgtgctctgccacacgtgggcgacgaaccggtaccccgggcacgcggtgagcatccggtctatgagccggtagtgcaggtgggcggacgtgccgggaaagatgacgtacagcatgtggcccccgtaagtggggtccgggtaaaacaacagccgcgggtcgcacgccccgcctccgcgcaggatcgtgtggacgaaaaaaagctcgggttggccaagaatcccggccaagaggtcctggaggggggcgttgtggcggtcggccaacacgaccaaggaggccaggaaggcgcgatgctcgaatatcgtgttgatctgctgcacgaaggccaggattagggcctcgcggctggtggcggcgaaccgcccgtctcccgcgttgcacgcgggacagcaacccccgatgcctaggtagtagcccatcccggagagggtcaggcagttgtcggccacggtctggtccagacagaagggcagcgagacgggagtggtcttcaccaggggcaccgagagcgagcgcacgatggcgatctcctcggagggcgtctgggcgagggcggcgaaaaggccccgatagcgctggcgctcgtgtaaacacagctcctgtttgcgggcgtgaggcggcaggctcttccgggaggcccgacgcaccacgcccagagtcccgccggccgcagaggagcgcgaccgccggcgctccttgccgtgatagggcccgggccgggagccgcggcgatgggggtcggtgtcatacataggtacacagggtgtgctccagggacaggagcgagatcgagtggcgtctaagcagcgcgcccgcctcacggacaaatgtggcgagcgcggtgggctttggtacaaatacctgatacgtcttgaaggtgtagatgagggcacgcaacgctatgcagacacgcccctcgaactcgttcccgcaggccagcttggccttgtggagcagcagctcgtcgggatgggtggcggggggatggccgaacagaacccaggggtcaacctccatctccgtaatggcgcacatggggtcacagaacatgtgcttaaagatggcctcgggccccgcggcccgaagcaggctcacaaaccggcccccgtccccgggctgcgtctcggggtcagcctcgagctggtcgacgacgggtacgatacagtcgaagaggctcgtgttgttttccgagtagcggaccacggaggcccggagtctgcgcagggccagccagtaagcacgcaccagtaacaggttacacagcaggcattctccgccggtgcgcccgcgcccccggccgtgtttcagcacggtggccatcagagggcccaggtcgaggtcgggctgggcatcgggttcggtaaactgcgcaaagcgcggagccacgtcgcgcgtgcgtgccccgcgatgcgcttcccaggactggcggaccgtggcgcgacgggcctccgcggcagcgcgcagctggggccccgactcccagacggcgggggtgccggcgaggagcaacaggaccagatccgcgtacgcccacgtatccggcgactcctccggctcgcggtccccggcgaccgtctcgaattccccgttgcgagcggcggcgcgagtacagcagctgtccccgcccccgcgccgaccctccgtgcagtccaggagacgggcgcaatccttccagttcatcagcgcggtggtgagcgacggctgcgtgccggatcccgccgccgaccccgccccctcctcgcccccggaggccaaggttccgatgagggcccgggtggcagactgcgccaggaacgagtagttggagtactgcaccttggcggctcccggggagggcgagggcttgggttgcttctgggcatgccgcccgggcaccccgccgtcggtacggaagcagcagtggagaaaaaagtgccggtggatgtcgtttatggtgagggcaaagcgtgcgaaggagccgaccagggtcgccttcttggtgcgcagaaagtggcggtccatgacgtacacaaactcgaacgcggccacgaagatgctagcggcgcagtggggcgcccccaggcatttggcacagagaaacgcgtaatcggccacccactgaggcgagaggcggtaggtttgcttgtacagctcgatggtgcggcagaccagacagggccggtccagcgcgaaggtgtcgatggccgccgcggaaaagggcccggtgtccaaaagcccctccccacagggatccgggggcgggttgcggggtcctccgcgcccgcccgaaccccctccgtcgcccgcccccccgcgggcccttgagggggcggtgaccacgtcggcggcgacgtcctcgtcgagcgtaccgacgggcggcacacctatcacgtgactggccgtcaggagctcggcgcagagagcctcgttaagagccaggaggctgggatcgaaggccacatacgcgcgctcgaacgcccccgccttccagctgctgccgggggactcttcgcacaccgcgacgctcgccaggaccccggggggcgaagttgccatggctgggcgggaggggcgcacgcgccagcgaactttacgggacacaatccccgactgcgcgctgcggtcccagaccctggagagtctagacgcgcgctacgtctcgcgagacggcgcgcatgacgcggccgtctggttcgaggatatgacccccgccgagctggaggttgtcttcccgactacggacgccaagctgaactacctgtcgcggacgcagcggctggcctccctcctgacgtacgccgggcctataaaagcgcccgacgacgccgccgccccgcagaccccggacaccgcgtgtgtgcacggcgagctgctcgccgccaagcgggaaagattcgcggcggtcattaaccggttcctggacctgcaccagattctgcggggctgacgcgcgtgctgttgggcgggacggttcgcgaaccctttggtgggtttacgcgggcacgcacgctcccatcgcgggcgccatggcgggactgggcaagccctacaccggccacccaggtgacgccttcgagggtctcgttcagcgaattcggcttatcgtcccatctacgttgcggggcggggacggggaggcgggcccctactctccctccagcctcccctccaggtgcgcctttcagtttcatggccatgacgggtccgacgagtcgtttcccatcgagtatgtactgcggcttatgaacgactgggccgaggtcccgtgcaacccttacctgcgcatacagaacaccggcgtgtcggtgctgtttcaggggttttttcatcgcccacacaacgcccccgggggcgcgattacgccagagcggaccaatgtgatcctgggctccaccgagacgacggggctgtccctcggcgacctggacaccatcaaggggcggctcggcctggatgcccggccgatgatggccagcatgtggatcagctgctttgtgcgcatgccccgcgtgcagctcgcgtttcggttcatgggccccgaagatgccggacggacgagacggatcctgtgccgcgccgccgagcaggctattacccgtcgccgccgaacccggcggtcccgggaggcgtacggggccgaggccgggctgggggtggccggaacgggtttccgggccaggggggacggttttggcccgctccccttgttaacccaagggccctcccgcccgtggcaccaggccctgcggggtcttaagcacctacggattggcccccccgcgctcgttttggcggcgggactcgtcctgggggccgctatttggtgggtggttggtgctggcgcgcgcctataaaaaaggacgcaccgccgccctaatcgccagtgcgttccggacgccttcgccccacacagccctcccgaccgacacccccatatcgcttcccgacctccggtcccgatggccgtcccgcaatttcaccgccccagcaccgttaccaccgatagcgtccgggcgcttggcatgcgcgggctcgtcttggccaccaataactctcagtttatcatggataacaaccacccgcacccccagggcacccaaggggccgtgcgggagtttctccgcggtcaggcggcggcgctgacggaccttggtctggcccacgcaaacaacacgtttaccccgcagcctatgttcgcgggcgacgccccggccgcctggttgcggcccgcgtttggcctgcggcgcacctattcaccgtttgtcgttcgagaaccttcgacgcccgggaccccgtgaggcccggggagttccttctggggtgttttaatcaataaaagaccacaccaacgcacgagccttgcgtttaatgtcgtgtttattcaagggagtgggatagggttcgacggttcgaaacttaacacaccaaataatcgagcgcgtctagcccagtaacatgcgcacgtgatgtaggctggtcagcacggcgtcgctgtgatgaagcagcgcccggcgggtccgctgtaactgctgttgtaggcggtaacaggcgcggatcagtaccgccagggcgctacgaccggtgcgttgcacgtagcgtcgcgacagaactgcgtttgccgatacgggcggggggccgaattgtaagcgcgtcacctcttgggagtcatcggcggataacgcactgaatggttcgttggttatgggggagtgtggttccccagggagtgggtcgagcgcctcggcctcggaatccgagaggaacaacgaggtggcgtcggagtcttcgtcgtcagagacatacagggtctgaagcagcgacacgggcgggggggtagcgtcgatgtgtagcgcgagggaggatgcccacgaagacaccccagacaaggagctgcccgtgcgtggatttgtggaagacgcggaagccgggacggatgggcggttttgcggtgcccggaaccgaaccgccggatactccccgggtgctacatgcccgttttggggctggggttggggctggggttggggctggggttggggctggggttggggctggggttggggctggggttggggttggggttggggctggggttggggttggggctggggctggggctggggctggggctggggctggggctggggctggggctggggctggggctggggctggggctggggctggggctggggctggggttggggcgcggacaggcggctgacggtcaaatgcccccgggggcgcgcagatgtggtgggcgtggccaccggctgccgtgtagtggggcggcgggaaaccgggcctccgggcgcaacaccgccctccagcgtcaagtatgtggggggcgggcctgacgtcgggggcggggcgacgggttggaccgcgggaggcgggggagagggacctgcgggagaggatgaggtcggctcggccgggttgcggcctaaaacaggggccgtggggtcggcggggtcccagggtgaagggagggattcccgcgattcggacagcgacgcgacagcggggcgcgtaaggcgccgctgcggcccgcctacgggaaccctggggggggttggcgcgggacccgaggttagcggggggcggcggttttcgcccccgggcaaaaccgtgccggttgcgaccgggggcggaacgggatcgatagggagagcgggagaagcctggccggcggcctggggcccgagcgggaggggcacaccagacaccaaagcgtggggcgctggctctgggggtttgggaggggccggggggcgcgcgaaatcggtaaccggggcgaccgtgtcggggagggcaggcggccgccaaccctgggtggtcgcggaagcctgggtggcgcgcgccagggagcgtgcccggcggtgtcggcgcgcgcgcgacccggacgaagaagcggcagaagcgcgggaggaggcgggggggcggggggcggtggcatcggggggcgccggggaactttggggggacggcaagcgccggaagtcgtcgcgggggcccacgggcgccggccgcgtgctttcggccgggacgcccggtcgtgcttcgcgagccgtgactgccggcccagggggccgcggtgcacactgggacgtggggacggactgatcggcggtgggcgaaagggggtccggggcaaggaggggcgcggggccgccggagtcgtcagacgcgagctcctccaggccgtgaatccatgcccacatgcgaggggggacgggctcgccgggggtggcgtcggtgaatagcgtgggggccaggcttccgggccccaacgagccctccgccccaacaaggtccacagggccgggggtcgggtttgggaccgaggggctctggtcgtcgggggcgcgctggtacaccggatgccccgggaatagctcccccgacaggagggaggcgtcgaacggccgcccgaggatagctcgcgcgaggaaggggtcctcgtcggtggcgctggcggcgaggacgtcctcgccgcccgccacaaacgggagctcctcggtggcctcgctgccaacaaaccgcacgtcgggggggccgggggggtccgggttttcccacaacaccgcgaccggggtcatggagatgtccacgagcaccagacacggcgggccccgggcgaggggccgctcggcgatgagcgcggacaggcgcgggagctgtgccgccagacacgcgttttcgatcgggttcaggtcggcgtgcaggaggcggacggcccacgtctcgatgtcggacgacacggcatcgcgcaaggcggcgtccggcccgcgagcgcgtgagtcaaacagcgtgagacacagctccagctccgactcgcgggaaaaggccgtggtgttgcggagcgccacgacgacgggcgcgcccaggagcactgccgccagcaccaggtccatggccgtaacgcgcgccgcgggggtgcggtgggtggcggcggccggcacggcgacgtgctggcccgtgggccggtagagggcgttggggggagcggggggtgacgcctcgcgcccccccgaggggctcagcgtctgcccagattccagacgcgcggtcagaagggcgtcgaaactgtcatactctgtgtagtcgtccggaaacatgcaggtccaaagagcgaccagagcggtgcttgggagacacatgcgcccgaggacgctcaccgccgccagcgcctgggcgggactcagctttcccagcgcggcgccgcgctcggttcccagctcggggaccgagcgccagggcgccagggggtcggtttcggacaacttgccgcggcgccagtctgccagccgcgtgccgaacatgaggccccgggtcggagggcctccggccgaaaacgctggcagcacgcggatgcgggcgtctggatgcggggtcaggcgctgcacgaatagcatggaatctgctgcgttctgaaacgcacgggggagggtgagatgcatgtactcgtgttggcgaaccagatccaggcgccaaaaggtgtaaatgtgttccggggagctggccaccagcgccaccagcacgtcgttctcgttaaaggaaacgcggtgcctagtggagctctggggtccgagcggcggccccggggccgccgcgtcacccccccattccagctgggcccagcgacacccaaactcgcgcgtgagagtggtcgcgacgagggcgacgtagagctcggccgccgcatccatcgaggccccccatctcgcctggcggtggcgcacaaagcgtccgaagagctgaaagttggcggcctgggcgtcgctgagggccagctgaagccggttgatgacggtgaggacgtacatggccgtgacggtcgaggccgactccagggtgtccgtcggaagcggggggcgaatgcatgccgcctcgggacacatcagcagcgcgccgagcttgtcggtcacggccgggaagcagagcgcgtactgcagtggcgttccatccgggaccaaaaagctgggggcgaacggccgatccagcgtactggtggcctcgcgcagcaccaggggccccgggcctccgctcactcgcaggtacgcctcgccccggcggcgcagcatctgcgggtcggcctcttggccgggtggggcggacgcccgggcgcgggcgtctagggcgcgaagatccacgagcaggggcgcgggcgcggccgccgcgcccgcgcccgtctggcctgtggccttggcgtacgcgctatataagcccatgcggcgttggatgagctcccgcgcgccccggaactcctccaccgcccatggggccaggtccccggccaccgcgtcgaattccgccaacaggccccccagggtgtcaaagttcatctcccaggccacccttggcaccacctcgtcccgcagccgggcgctcaggtcggcgtgttgggccacgcgccccccgagctcctccacggccccggcccgctcggcgctcttggcgcccaggacgccctggtacttggcgggaaggcgctcgtagtcccgctgggctcgcagccccgacacagtgttggtggtgtcctgcagggcgcgaagctgctcgcatgccgcgcgaaatccctcgggcgatttccaggcccccccgcgaacgcggccgaagcgaccccatacctcgtcccactccgcctcggcctcctcgagagacctccgcagggcctcgacgcggcgacgggtgtcgaagagcgcctgcaggcgcgcgccctgtcgcgtcaggaggcccgggccgtcgccgctggccgcgcttagcgggtgcgtctcaaaggtacgctgggcatgttccaaccaggcgaccgcctgcacgtcgagctcgcgcgccttctccgtctggtccaccagaatttcgacctgatccgcgatctcctccgccgagcgcgcctggtccagcgtcttggccacggtcgccgggacggcgaccaccttcagcagggtcttcagattggccagaccctcggcctcgagctgggcccggcgctcgcgcgcggccagcacctcccgcagccccgccgtgacccgctcggtggcttcggcgcgctgctgtttggcgcgcaccacggcgtccttggtatcggccaggtcctgtcgggtcacgaatgcgacgtagtgcgcgtacgccgtgtccttcacggggctctggtccacgcgctccagcgccgccacgcacgccaccagcgcgtcctcgctcgggcagggcagggtgacccctgcccggacaagctcggcggccgccgccgggtcgttgcgcaccgcggatatctcctccgcggcggcggccaggtccagcgccacgcttccgatcgcgcgccgcgcgtcggcccggagggcgtccaggcgatcgcggatatccacgtactcggcgtagcccttttgaaaaaacggcacgtactggcgcagggccggcacgccccccaagtcttccgacaggtgtaggacggcctcgtggtagtcgataaacccgtcgttcgcctgggcccgctccagcagcccccccgccagccgcagaagccgcgccaggggctcggtgtccacccgaaacatgtcggcgtacgtgtcggccgcggccccgaaggccgcgctccagtcgatgcggtgaatggctgcgagcggggggagcatggggtggcgctggttctcgggggtgtatgggttaaacgcaagggccgtctccagggcaagggtcaccgccttggcgttggttcccagcgcctgttcggcccgctttcggaagtcccgggggttgtagccgtgcgtgcccgccagcgcctgcaggcgacggagctcgaccacgtcaaactcggcaccgctttccacgcggtccagcacggcctccacgtcggcggcccagcgctcgtggctactgcgggcgcgctgggccgccatcttctctctgaggtcggcggtggcggcctcaagttcgtcggcgcggcgtcgcgtggcgccgatgacctttcccagctcctgcagggcgcgcccgctgggggagtggtccccggccgtcccttcggcgtgcaacaggcccccgaacctgccctcgtggcccgcgaggctttcccgcgcgccggtggtcgcgcgcgtcgcggcctggatcagggaggcatgctctccctccggttggttggcggcccggcgcacctggacgacaaggtcggcggcagccgaccctaaggtcgtgagctgggcgatggccacccgcgcgtccagggccaaccgagtcgccttgacgtatcccgcggcgctgtcggccatggccgctaggaaggccaggggggaggccgggtcgctggcggccgcgcccagggccgtcaccgcgtcgaccaggacgcggtgcgcccgcacggccgcatccaccgtcgacgcagggtctgccgttgcgacggcggcgctgccggcgttgatggcgttcgagacggcgtgggctatgatcggggcgtgatcggcgaagaactgcaagagaaacggagtctctggggcgtcggcgaacaggttcttcagcaccaccacgaagctgggatgcaagccagacagagccgtcgccgtgtccggagtcgggtgctccagggcatctcggtactgccccagcagcccccacatgtccgcccgcagcgccgccgtaacctcagggggcgccccccgaacggcctcggggaggtccgaccagcccgccggcagggaggcccgcagggtcgccaggacggccggacaggcctttagccccacaaagtcagggagggggcgcaggaccccctggagtttgtgcaagaacttctcccgggcgtcgcgggccaccttcgcccgctcccgcgctccctcgagcattgcctccagggagcgcgcgcgctcccgcaaacgggcacgcgcatcgggggcgagctctgccgtcagcttggcggcatccatggcccgcgcctgccgcagcgcttcctcggccatgcgcgtggcctctggcgacagcccgccgtcgtcggggtagggcgacgcgccgggcgcaggaacaaaggccgcgtcgctgtccagctgctggcccagggccgcatctagggcgtcgaagcgccgcagctcggccagacccgagctgcggcgcgcctgttggtcgttaatgtcgcggatgctgcgcgccagctcgtccagtggcttgcgttctatcagcccttggttggcggcgtccgtcaggacggagagccaggccgccaggtcctcgggggcgtccagcgtctggccccgctggatcagatcccgcaacaggatggccgtggggctggtcgcgatcgggggcggggcgggaatggcggcgcgctgcgcgatgtcccgcgtgtgctggtcgaagacaggcagggactcgagcagctggaccacgggcacgacggcggccgaagccacgtgaaaccggcggtcgttgttgtcgctggcctgtagagccttggcgctgtatacggccccccggtaaaagtactccttaaccgcgccctcgatcgcccgacgggcctgggtccgcacctcctccagccgaacctgaacggcctcggggcccagggggggtgggcgcggagccccctgcggggccgccccggccggggcgggcattacgccgaggggcccggcgtgctgtgagaccgcgtcgaccccgcgagcgagggcgtcgagggcctcgcgcatctggcgatcctccgcctccaccctaatctcttcgccacgggcaaatttggccagagcctggactctatacagaagcggttctgggtgcgtcggggtggcgggggcaaaaagggtgtccgggtgggcctgcgagcgctccagaagccactcgccgaggcgtgtatacagattggccggcggggccgcgcgaagctgcagctccaggtccgcgagttccccgtaaaaggcgtccgtctcccgaatgacatccctagccacaaggatcagcttcgccagcgccaggcgaccgatcagagagttttcgtccagcacgtgctggacgaggggcagatgggcggccacgtcggccaggctcaggcgcgtggaggccagaaagtcccccacggccgttttccagggcagcatgttcagggtaaactccagcagggcggcggccgggccggccaccccggcctgggtgtgcgtccgggccccgttctcgatgagaaaggcgaggacgcgttcaaagaaaaaaataacacagagctccagcagccccggagaggccggatacggcgaccgtaaggcgctgatggtgagccgcgaacacgcggcgacctcgcgggccagggcggcggagcacgcggtgaacttaaccgccgtggcggccacgtttgggtgggcctcgaacagctgggcgaggtctgcgcccgggggctcgggcgagcggcgagtcttcagcgcctcgagggcctgtgaggacgccggaaccgtgggcccgtcgtcctcgcccgcctcggcgaccggcggcccggccgggtcggggggtgccgaggcgaggacaggctccggaacggaggcggggaccgcggccccgacgggggttttgcctttgggggtggatttcttcttggttttggcagggggggccgagcgtttcgttttctcccccgaagtcaggtcttcgacgctggaaggcggagtccaggtgggtcggcggcgcttgggaaggccggccgagtagcgtgcccggtgccgaccaaccgggacgacgcccatctccaggacccgcatgtcgtcgtcatcttcttcggccgcctctgcggcgggggtcttgggggcggagggaggcggtggtgggatcgcggagggtgggtcggcggagggtgggtcggcggaggggggatccgtgggtggggtacccttcagggccaccgcccatacatcgtcgggcgcccgattcgggcgcttggcctctggttttgccgacggaccggccgtcccccgggatgtctcggaggccctgtcgtcgcgacgggcccgggtcggtggcggcgactgggcggctgtgggcgggtgtggccccgggccccctaccccctcccgggggcccacgccgacgcagggctcccccaggcccgcgatctcgccccgcagggggtgcgtgatggccacgcgccgttcgctgaacgcttcgtcctgcaggtaagtctcgctggccccgtaaagatgcagagccgcggccgtcaagtccgcaggagccgcgggttccgggcccgacggcacgaaaaacaccatggctcccgcccaccgtacgtccgggcgatcgcgggtgtaatacgtcaggtatggatacatgtcccccgcccgcactttggcgatgaacgcgggggtgccctccggaaggccgtgcgggtcaaaaaggtatgcggtgtcgccgtccctgaacagccccatccctagggggccaatggttaggagcgtgtacgacagggggcgcagggcccacgggccggcgaagaacgtgtgtgcggggcattgtgtctccagcaggcccgccgcgggctccccgaagaagcccacctcgccgtatacgcgcgagaagacacagcgcagtccgccgcgcgcccctgggtactcgaggaagttggggagctcgacgatcgaacacatgcgcggcggcccagggcccgcggtcgcgcgcgtccactcgcccccctcgaccaaacatccctcgatggcctccgcggacaggacgtcgcgagggcccacatcaaatatgaggctgagaaaggacagcgacgagcgcatgcacgataccgacccccccggctccaggtcgggcgcgaactggttccgagcaccggtgaccacgatgtcgcgatcccccccgcgttccatcgtggagtgcggtggggtgcccgcgatcatatgtgccctgctggccagagacccggcctgtttatggaccggacccccggggttagtgttgtttccgccacccatgcccccgtaccatggccccggttcccctgattaggctacgagtcgcggtgatcgcttcccaaaaaccgagctgcgtttgtctgtcttggtcttccacccccccccccgcccgcccgcacaccataacaccgagaacaacacacgggggtgggcgtaacataataaagctttattggtaactagttaacggcaagtccgtgggtggcgcgacggtgtcctccgggatcatctcgtcgtcctcgacgggggtgttggaatgaggcgccccctcgcggtccgcctggcgtgggccgtgcccataggcctccggcttctgtgcgtccatgggcataggcgcggggagactgtttccggcgtcgcggacctccaggtccctgggagactccggtccggctaacggacgaaacgcggaagcgcgaaacacgccgtcggtgacccgcaggagctcgttcatcagtaaccaatccatactcagcgtaacggccagcccctggcgagacagatccacggagtccggaaccgcggtcgtctggcccagggggccgaggctgtagtccccccaggcccctaggtcgcgacggctcgtaagcacgacgcggtcggccgcggggctttgcgggggggcgtcctcgggcgcatgcgccattacctctcggatggccgcggcgcgctggtcggccgagctgaccaagggcgccacgaccacggcgcgctccgtctgcaggcccttccacgtgtcgtggagttcctggacaaactcggccacgggctcgggtcccgcggccgcgcgcgcggcttgatagcaggccgacagacgccgccagcgcgctagaaactgacccatgaagcaaaacccggggacctggtctcccgacagcagcttcgacgcccgggcgtgaatgccggacacgacggacagaaacccgtgaatttcgcgccggaccacggccagcacgttgtcctcgtgcgacacctgggccgccagctcgtcgcacacccccaggtgcgccgtggtttcggtgatgacggaacgcaggctcgcgagggacgcgaccagcgcgcgcttggcgtcgtgatacatgctgcagtactgactcaccgcgtcccccatggcctcggggggccagggccccaggcggtcgggcgtgtccccgaccaccgcatacaggcggcgcccgtcgctctcgaaccgacactcgaaaaaggcggagagcgtgcgcatgtgcagccgcagcagcacgatggcgtcctccagttggcgaatcagggggtctgcgcgctcggcgaggtcctgcagcaccccccgggcggccagggcgtacatgctaatcaacaggaggctggtgcccacctcggggggcggggggggctgcagctggaccaggggccgcagctgctcgacggcacccctggagatcacgtacagctcccggagcagctgctctatgttgtcggccatctgcatagtggggccgaggccgccccgggcggccggttcgaggagggtgatcagcgcgcccagtttggtgcgatggccctcaaccgtggggagatagcccagcccaaagtcccgggcccaggccaacacacgcagggcgaactcgaccgggcggggaaggtaggccgcgctacacgtggccctcagcgcgtccccgaccaccagggccagaacgtaggggacgaagcccgggtcggcgaggacgttggggtgaatgccctcgagggcggggaagcggatctgggtcgccgcggccaggtggacagagggggcgtggctgggctgcccgacggggagaagcgcggacagcggcgtggccggggtggtgggggtgatgtcccagtgggtctgaccatacacgtcgatccagatgagcgccgtctcgcggagaaggctgggttgaccggaactaaagcggcgctcggccgtctcaaactcccccacgagcgcccgccgcaggctcgccagatgttccgtcggcacggccggacccatgatacgcgccagcgtctggcttagaacgccccccgacaggccgaccgcctcgcagagccgcccgtgcgtgtgctcgctggcgccctggacccgcctgaaagtttttacgtagttggcatagtacccgtattcccgcgccaacccaaacacgttcgaccccgcgagggcaatgcacccaaagagctgctggacttcgccgagtccgtggccggtgggcgtccgcgcggggacgcccgccgccagaaacccctccagggccgaaaggtagtgcgtgcagtgcgagggcgtgaacccagcgtcgatcagggtgttgatcaccacggagggcgaattggtattctggatcaacgtccacgtctgctgcagcagagccagcagccgctgctgggcgccggcggagggctgctccccgagctgcagcaggctggagacggcaggctggaagactgccagtgccgacgaactcaggaacggcacgtcgggatcaaacacggccacgtccgtccgcacgcgcgccattagcgtccccgggggcgcacaggccgagcgcgggctgacgcggctgagggccgtcgacacgcgcacctcctcgcggctgcgaaccatcttgttggcctcgagcggcggaatcattatggccgggtcgatctcccgcacggtgtgctgaaactgcgccaacaggggcggcgggaccacagccccccgctcgggggtcgtcaggtactcgtccaccagggccaacgtaaagagggcccgtgtgaggggagtgagggtcgcgtcgtctatgcgctggaggtgcgccgagaacagcgtcacccgattactcaccagggccaagaaccggaggccctcttgcacgaacggggcggggaagagcaggctgtacgccggggtggtaaggttcgcgctgggctgccccaacgggaccggcgccatcttgagcgacgtctccccaagggcctcgatggaggtccgcgggctcatggccaagcagctcttggtgacggtttgccagcggtctatccactccacggcgcactggcggacgcggaccggccccagggccgccgcggtgcgcaggccggcggaatccagcgcatgggacgtgtcggagccggtgaccgcgaggatggtgtccttgatgacctccatctcccggaaggcctggtcgggggcctcggggagagccaccaccaagcggtgtacgagcaacccggggaggttctcggccaagagcgccgtctccggaagcccgtgggcccggtggagcgcgcacaggtgttccagcagcggccgccagcagtcccgcgcgtctgccggggcgatggccgttcccgacaacagaaacgccgccatggcggcgcgcagcttggccgtggccagaaacgccgggtcgtccgccccgtttgccgtctcggccgtgggggttggcggttggcgaaggccggctaggctcgccaataggcgctgcataggtccgtccgagggcggaccggcgggtgaggtcgtgacgacgggggcctcggacgggagaccgcggtctgccatgacgcccggctcgcgtgggtgggggacagcgtagaccaacgacgagaccgggcgggaatgactgtcgtgcgctgtagggagcggcgaattatcgatcccccgcggccctccaggacccccgcaggcgttgcgagtaccccgcgtcttcgcggggtgttatacggccacttaagtcccggcatcccgttcgcggacccaggcccgggggattgtccggatgtgcgggcagcccggacggcgtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgacccccccccccctccctcccccgggtcttgctaggtcgcgatctggggtcgcaatgaagaccaatccgctacccgcaaccccttccgtgtggggcgggagtaccgtggaactcccccccacaacacgcgataccgcgggacagggcctgcttcggcgcgtcctgcgccccccgatctctcgccgcgacggcccagggctccccagggggtcgggaccccggagggcggccagcacgctgtggttgcttggcctggacggcacagacgcgccccctggggcgctgacccccaacgacgataccgaacaggccctggacaagatcctgcggggcaccatgcgcgggggggcggccctgatcggctccccgcgccatcatctaacccgccaagtgatcctgacggatctgtgccaacccaacgcggatcgtgctgggacgctgcttctggcgctgcggcaccccgccgacctgcctcacctggcccaccagcgcgccccgccaggccggcagaccgagcggctgggcgaggcctggggccagctgatggaggcgaccgccctggggtcggggcgagccgagagcgggtgcacgcgcgcgggcctagtgtcgtttaacttcctggtggcggcgtgtgccgcctcgtacgacgcgcgcgacgccgccgatgcggtacgggcccacgtcacggccaactaccgcgggacgcgggtgggggcgcgcctggatcgtttttccgagtgtctgcgcgccatggttcacacgcacgtcttcccccacgaggtcatgcggtttttcggggggctggtgtcgtgggtcacccaggacgagctagcgagcgtcaccgccgtgtgcgccgggccccaggaggcggcgcacaccggccacccgggccggccccgctcggccgtgatcctcccggcatgtgcgttcgtggacctggacgccgagctggggctggggggcccgggcgcggcgtttctgtacctggtattcacttaccgccagcgccgggaccaggagctgtgttgtgtgtacgtgatcaagagccagctccccccgcgcgggttggagccggccctggagcggctgtttgggcgcctccggatcaccaacacgattcacggcaccgaggacatgacgcccccggccccaaaccgaaaccccgacttccccctcgcgggcctggccgccaatccccaaaccccgcgttgctcggctggccaggtcacgaacccccagttcgccgacaggctgtaccgctggcagccggacctgcgggggcgccccaccgcacgcacctgtacgtacgccgcctttgcagagctcggcatgatgcccgaggatagtccccgctgcctgcaccgcaccgagcgctttggggcggtcagcgtccccgttgtcattctggaaggcgtggtgtggcgccccggcgagtggcgggcatgcgcgtgagcgtagcaaacgccccgcccacacaacgctccgcccccaaccccttccccgctgtcactcgttgttcgttgacccgggcgtccgccaaataaagccactgaaacccgaaacgcgagtgttgtaacgtcctttgggcgggaggaagccacaaaatgcaaatgggatacatggaaggaacacacccccgtgactcaggacatcggtgtgtccttttgggtttcactgaaactggcccgcgccccacccctgcgcgatgtggataaaaagccagcgcgggtggtttagggtaccacaggtgggtgctttggaaacttgccggtcgccgtgctcctgtgagcttgcgtccctccccggtttcctttgcgctcccgccttccggacctgctctcgcctactcttctttggctctcggtgcgattcgtcaggcagcggccttgtcgaatctcgaccccaccactcgccggacccgccgacgtcccctctcgagcccgccgaaacccgccgcgtctgttgaaatggccagccgcccagccgcatcctctcccgtcgaagcgcgggccccggttgggggacaggaggccggcggccccagcgcagccacccagggggaggccgccggggcccctctcgcccacggccaccacgtgtactgccagcgagtcaatggcgtgatggtgctttccgacaagacgcccgggtccgcgtcctaccgcatcagcgataacaactttgtccaatgtggttccaactgcaccatgatcatcgacggagacgtggtgcgcgggcgcccccaggacccgggggccgcggcatcccccgctcccttcgttgcggtgacaaacatcggagccggcagcgacggcgggaccgccgtcgtggcattcgggggaaccccacgtcgctcggcggggacgtctaccggtacccagacggccgacgtccccaccgaggcccttgggggcccccctcctcctccccgcttcaccctgggtggcggctgttgttcctgtcgcgacacacggcgccgctctgcggtattcgggggggagggggatccagtcggccccgcggagttcgtctcggacgaccggtcgtccgattccgactcggatgactcggaggacacggactcggagacgctgtcacacgcctcctcggacgtgtccggcggggccacgtacgacgacgcccttgactccgattcgtcatcggatgactccctgcagatagatggccccgtgtgtcgcccgtggagcaatgacaccgcgcccctggatgtttgccccgggacccccggcccgggcgccgacgccggtggtccctcagcggtagacccacacgcgccgacgccagaggccggcgctggtcttgcggccgatcccgccgtggcccgggacgacgcggaggggctttcggacccccggccacgtctgggaacgggcacggcctaccccgtccccctggaactcacgcccgagaacgcggaggccgtggcgcgctttctgggagatgccgtgaaccgcgaacccgcgctcatgctggagtacttttgccggtgcgcccgcgaggaaaccaagcgtgtcccccccaggacattcggcagcccccctcgcctcacggaggacgactttgggcttctcaactacgcgctcgtggagatgcagcgcctgtgtctggacgttcctccggtcccgccgaacgcatacatgccctattatctcagggagtatgtgacgcggctggtcaacgggttcaagccgctggtgagccggtccgctcgcctttaccgcatcctgggggttctggtgcacctgcggatccggacccgggaggcctcctttgaggagtggctgcgatccaaggaagtggccctggattttggcctgacggaaaggcttcgcgagcacgaagcccagctggtgatcctggcccaggctctggaccattacgactgtctgatccacagcacaccgcacacgctggtcgagcgggggctgcaatcggccctgaagtatgaggagttttacctaaagcgttttggcgggcactacatggagtccgtcttccagatgtacacccgcatcgccggctttttggcctgccgggccacgcgcggcatgcgccacatcgccctggggcgagaggggtcgtggtgggaaatgttcaagttctttttccaccgcctctacgaccaccagatcgtaccgtcgacccccgccatgctgaacctggggacccgcaactactacacctccagctgctacctggtaaacccccaggccaccacaaacaaggcgaccctgcgggccatcaccagcaacgtcagtgccatcctcgcccgcaacgggggcatcgggctatgcgtgcaggcgtttaacgactccggccccgggaccgccagcgtcatgcccgccctcaaggtccttgactcgctggtggcggcgcacaacaaagagagcgcgcgtccgaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagtgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaacctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgccgaatttcaggacctgaacaaacacatcgccgaggtgatgctgctgtcggcgatgaagaccagcaacgcgctgtgcgttcgcggggcccgtcccttcaaccactttaagcgcagcatgtatcgcgccggccgctttcactgggagcgctttccggacgcccggccgcggtacgagggcgagtgggagatgctacgccagagcatgatgaaacacggcctgcgcaacagccagtttgtcgcgctgatgcccaccgccgcctcggcgcagatctcggacgtcagcgagggctttgcccccctgttcaccaacctgttcagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgctgatcgacctgtgtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcatgagctgcgcgctgtgaccgacaaaccccctccgcgccaggcccgccgccactgtcgtcgccgtcccacgctctcccctgctgccatggattccgcggccccagccctctcccccgctctgacggcccttacggaccagagcgcgacggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgcttttccacaacaacgaccaggcgcgccgcgagtacgtggccggtaccatcaaccacccggccatccgcgccaaggtggactggttggaagcgcgggtgcgggaatgcgcctccgttccggaaaagttcattctcatgatcctcatcgagggcatcttttttgccgcctcgtttgccgccatcgcctaccttcgcaccaacaaccttctgcgggtcacctgccagtcaaacgacctcatcagccgggacgaggccgtgcacacgacggcctcgtgttacatctacaacaactacctcggcgggcacgccaagcccccgcccgaccgcgtgtacgggctgttccgccaggcggtcgagatcgagatcggatttatccgatcccaggcgccgacggacagccatatcctgagcccggcggcgctggcggccatcgaaaactacgtgcgattcagcgcggatcgcctgttgggccttatccacatgaagccactgttttccgccccaccccccgacgccagctttccgctgagcctcatgtccaccgacaaacacaccaattttttcgagtgtcgcagcacctcctacgccggggcggtcgtcaacgatctgtgagtgtcgcggcgcgcttctacccgtgtttgcccataataaacctctgaaccaaactttgggtctcattgtgattcttgtcagggacgcgggggtgggagaggataaaaggcggcgcaaaaagcagtaaccaggtccgtccagattctgcgggcataggataccataattttattggtgggtcgtttgttcggggacaagcgcgctcgtctgacgtttgggctactcgtcccagaatttggccaggacgtccttgtagaacgcgggtgggggggcctgggtccgcaactgctccagaaacctgtcggcgatatcaggggccgtgatatgccgggtcacgatagatcgcgccaggttttcgtcgcggatgtcctggtagataggcaggcgtttcagaagagtccacggcccccgctccttggggccgataagcgatatgacgtacttaatgtagcggtgttccaccagctcggtgatggtcatgggatcggggagccagtccagggactctggggcgtcgtggatgacgtggcgtcgcgggttggccacataactgcggtgctcttccagcagctgcgcgttcgggacctggacgagctcgggcggggtgagtatctccgaggaggacgacctggggccggggtggcccccggtaacgtcccggggatccagggggaggtcctcgtcgtcttcgtatccgccggcgatctgttgggttagaatttcggtccacgagacgcgcgtctcggtgccgccggcggccggcggcagagggggcctggtttccgtggagcgcgagctggtgtgttcccggcggatggcccgccgggtctgagagcgactcgggggggtccagtgacattcgcgcagcacatcctccacggaggcgtaggtgttattgggatggaggtcggtgtggcagcggacaaagagggccaggaactgggggtagctcatcttaaagtactttagtatatcgcgacagttgatcgtgggaatgtagcaggcgctaatatccaacacaatatcacagcccatcaacaggaggtcagtgtccgtggtgtacacgtacgcgaccgtgttggtgtgatagaggttggcgcaggcatcgtccgcctccagctgacccgagttaatgtaggcgtaccccagggcccggagaacgcgaatacagaacagatgcgccagacgcagggccggcttcgagggcgcggcggacggcagcgcggctccggacccggccgtcccccgggtccccgaggccagagaggtgccgcgccggcgcatgttggaaaaggcagagctgggtctggagtcggtgatgggggaaggcggtggagaggcgtccacgtcactggcctcctcgtccgtccggcactgggccgtcgtgcgggccaggatggccttggctccaaacacaaccggctccatacaattgaccccgcgatcggtaacgaagatggggaaaagggacttttgggtaaacacctttaataagcgacagaggcagtgtagcgtaatggcctcgcggtcgtaactggggtatcggcgctgatatttgaccaccaacgtgtacatgacgttccacaggtccacggcaatgggggtgaagtacccggccggggccccaaggccccggcgcttgaccagatggtgtgtgtgggcaaacttcatcatcccgaacaaacccatgtcaggtcaattgtaactgcggatcggcctaactaaggcgtggttggtgcgacggtccgggacacccgagcctgtctctctgtgtatggtgacccagacaacaacaccgacacaagaggacaataatccgttaggggacgctctttataatttcgatggcccaactccacgcggattggtgcagcaccctgcatgcgccggtgcgggccaaccttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccatcaaaaccccaccctcgcagcggcatacgccccctccgcgtcccgcacccgagaccccggcccggctgccctcaccaccgaagcccacctcgtcactgtggggtgttcccagcccgcgttgggatgacggattcccctggcggtgtggcccccgcctcccccgtggaggacgcgtcggacgcgtccctcgggcagccggaggagggggcgccctgccaggtggtcctgcagggcgccgaacttaatggaatcctacaggcgtttgccccgctgcgcacgagccttctggactcgcttctggttatgggcgaccggggcatccttatccataacacgatctttggggagcaggtgttcctgcccctggaacactcgcaattcagtcggtatcgctggcgcggacccacggcggcgttcctgtctctcgtggaccagaagcgctccctcctgagcgtgtttcgcgccaaccagtacccggacctacgtcgggtggagttggcgatcacgggccaggccccgtttcgcacgctggttcagcgcatatggacgacgacgtccgacggcgaggccgttgagctagccagcgagacgctgatgaagcgcgaactgacgagctttgtggtgctggttccccagggaacccccgacgttcagttgcgcctgacgaggccgcagctcaccaaggtccttaacgcgaccggggccgatagtgccacgcccaccacgttcgagctcggggttaacggcaaattttccgtgttcaccacgagtacctgcgtcacctttgctgcccgcgaggagggcgtgtcgtccagcaccagcacccaggtccagatcctgtccaacgcgctcaccaaggcgggccaggcggccgccaacgccaagacggtgtacggggaaaatacccatcgcaccttctctgtggtcgtcgacgattgcagcatgcgggcggtgctccggcgactgcaggtcggcgggggcaccctcaagttcttcctcacgacccccgtccccagtctgtgcgtcaccgccaccggtcccaacgcggtatcggcggtatttctcctgaaaccccagaagatttgcctggactggctgggtcatagccaggggtctccttcagccgggagctcggcctcccgggcctctgggagcgagccaacagacagccaggactccgcgtcggacgcggtcagccacggcgatccggaagacctcgatggcgctgcccgggcgggagaggcgggggccttgcatgcctgtccgatgccgtcgtcgaccacgcgggtcactcccacgaccaagcgggggcgctcggggggcgaggatgcgcgcgcggacacggccctaaagaaacctaagacggggtcgcccaccgcacccccgcccgcagatccagtccccctggacacggaggacgactccgatgcggcggacgggacggcggcccgtcccgccgctccagacgcccggagcggaagccgttacgcgtgttactttcgcgacctcccgaccggagaagcaagccccggcgccttctccgccttccgggggggcccccaaaccccgtatggttttggattcccctgacggggcggggccttggcggccgcccaactctcgcaccatcccgggttaatgtaaataaacttggtattgcccaacactttcccgcgtgtcgcgtgtggttcatgtgtgtgcctggcgcccccaccctcgggttcgtgtatttcctttccctgtccttataaaagccgtatgtggggcgtgacggaaccaccccgcgtgccatcacggccaaggcgcgggatgctccgcaacgacagccaccgggccgtgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgaatgccgcgtctggggcgttcgccgtcgggtgcgccgtgctgggctttatgcgcgcaccccctcccctcgcgcggcccaccgcgcggatatacgcctggctcaaactggcggccggtggagcggcccttgttctgtggagtctcggggagcccggcacgcagccgggggccccggccccgggcccggccacccagtgcctggcactgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccctggccgcggcggtgttggcgggcccgggggcgaccaccgccagggactgcttttccagggcttgccccgaccaccgccgcgtctgtgtcatcaccgcaggcgagtctctttcccgccgccccccggaggacccagagcgacccggggttcccgggcccccgtcccccccgaccccccaacgatcccacgggccgccggccgatgaggtcgcaccggccagggtcgcgcggcccgaaaacgtctgggtgcccgtggtcacctttctgggggcgggcgcgcttgccgtcaagacggtgcgagaacatgcccggggaacgccgggcccgggcctgccgctgtggccccaggtgtttctcggaggccatgtggcggtggccctgacggagctgtgtcaggcgcttccgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggtgctgctgtgccctccggggtcaacgggcgggcggtcgggcgattgatatatttttcaataaaaggcattagtcccgaagaccgccggtgtgtgatgatttcgccataacacccaaaccccggatggggcccgggtataaattccggaaggggacacgggctaccctcactatcgagggcgcttggtcgggaggccgcatcgaacgcacacccccatccggtggtccgtgtggaggtcgttttcatgcccggtctcgctttgccgggaacgctagccgatccctcgcgagggggaggcgtcgggcatggccccggggcgggtgggccttgccgtggtcctgtggagcctgttgtggctcggggcgggggtgtccgggggctcggaaactgcctccaccgggcccacgatcaccgcgggagcggtgacgaacgcgagcgaggcccccacatcggggtcccccgggtcagccgccagcccggaggtcacccccacatcgaccccaaaccccaacaatgtcacacaaaacaaaaccacccccaccgagccggccagccccccaacaacccccaagcccacctccacgcccaaaagcccccccacgtccacccccgaccccaaacccaagaacaacaccacccccgccaagtcgggccgccccactaaaccccccgggcccgtgtggtgcgaccgccgcgacccattggcccggtacggctcgcgggtgcagatccgatgccggtttcggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctaacggacccccacgtgctctgggcggagggggccggcccgggcgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccaccaggtgtttaacccgggccagatcgacacgcagacgcacgagcaccccgacgggttcaccacagtctctaccgtgacctccgaggctgtcggcggccaggtccccccgcggaccttcacctgccagatgacgtggcatcgcgactccgtgacgttctcgcgacgcaatgccaccgggctggccctggtgctgccgcggccaaccatcaccatggaatttggggtccgcattgtggtctgcacggccggctgcgtccccgagggcgtgacgtttgcctggttcctgggggacgacccctcaccggcggctaagtcggccgttacggcccaggagtcgtgcgaccaccccgggctggctacggtccggtccaccctgcccatttcgtacgactacagcgagtacatctgtcggttgaccggatatccggccgggattcccgttctagaacaccacggcagtcaccagcccccacccagggaccccaccgagcggcaggtgatcgaggcgatcgagtgggtggggattggaatcggggttctcgcggcgggggtcctggtcgtaacggcaatcgtgtacgtcgtccgcacatcacagtcgcggcagcgtcatcggcggtaacgcaagacccccccgttacctttttaatatctatatagtttggtcccccctctatcccgcccaccgctgggcgctataaagccgccaccctctcttccctcaggtcatccttggtcgatcccgaacgacacacggcgtggagcaaaacgcctccccctgagccgctttcctaccaacacaacggcatgcctctgcgggcatcggaacacgcctaccggcccctgggccccgggacaccccccatgcgggctcggctccccgccgcggcctgggttggcgtcgggaccatcatcgggggagttgtgatcattgccgcgttggtcctcgtgccctcgcgggcctcgtgggcactttccccatgcgacagcggatggcacgagttcaacctcgggtgcatatcctgggatccgacccccatggagcacgagcaggcggtcggcggctgtagcgccccggcgaccctgatcccccgcgcggctgccaaacagctggccgccgtcgcacgcgtccagtcggcaagatcctcgggctactggtgggtgagcggagacggcattcgggcctgcctgcggctcgtcgacggcgtcggcggtattgaccagttttgcgaggagcccgcccttcgcatatgctactatccccgcagtcccgggggctttgttcagtttgtaacttcgacccgcaacgcgctggggctgccgtgaggcgcgtgtactgcggtctgtctcgtctcctcttctccccttccctccccctccgcatcccaggatcacaccggtcaacgagggttgggggggtccggcacggacccaaaataataaacacacaatcacgtgcgataaaaagaacacgcggtcccctgtggtgtttttggttatttttattaaatctcgtcgacaaacagggggaaaggggcgtggtctagcgacggcagcacgggcggaggcgttcaccggctccggcgtccttcgcgtttaagcttggtcaggagggcgctcagggcggcgacgttggtcgggccgtcgttggtcagggcgttggctcgatggcgggcgaggacgggcgaggggctcaacggcgggggcgggggcccggtgcggcccgggggggaaaatagggcggatcccccccagtcgtacaggggattttccgcctcaatgtacggggaggccggcgctgcattcgccgtgttcacgcagacgttttcgtagacccgcatccatggtatttcctcgtagacacgccccccgtcctcgctcaccgtctcgtatattgactcgtcgtcctcgtagggggcgtgccgttcgcgggccgaggcggcgtgggtggctttgcggcgggcgtcgtcgtcgtcgtcgtcggccgtcagatacgtggcttccatctggtcgggttctccctccggggcgggtccccacacccgtggccgatcgaggctccccagagacgcgcgccggacgaggagggggcacgtcgccgccggcggtcgcctgtcgggtcccgcgacgttacgggccgggaggcgcgggggcacctcccccatgtgcgtgtaatacgtggccggctgtgtggccgcagcggggggctcggcgaccgggtcgttcgcatccggaagcgggggccccgcgccgtccgcgcggcgcctccggaacctccgggtggacgcgggggtcgagtgtaggcgaggtcgggggaggggcgggggctcgttgtcgcgccgcgcccgctgaatcttttcccgacaggtcccaccccccgcgcgatgcccccccgggccgctggccatgtcgtccgggggaggccccgcggaccacgtcgtccggcgagacgccacgagccgcaggatggactcgtagtggagcgacggcgccccgttgcggagcagatccgcggccagggcggccccgaaccaagccttgatgctcaactccatccgggcccagctgggggcggtcatcgtggggaacaggggggcggtggtccgacagaaacgctcctggctgtccaccgcggcccgcagatactcgttgttcaggctgtcggtggcccagacgccgtacccggtgagggtcgcgttgatgatatactgggcgtggtgatggacgatcgacagaacctccaccgtggatacgacggtatccacggtcccgtacgtaccgccgctccgcttgccggtctgccacaggttggctaggcgcgtcaggtggcccaggacgtcgctgaccgccgccctgagcgccatgcactgcatggagccggtcgtgccgctgggaccccggtccagatggcgcgcgaacgtttccgcgggcgcctccgggctgccgccgagcgggaggaaccggcgattggagggactcagccggtgacatacgtgcttgtccgtcgtccacagcatccaggacgcccaccggtacagcacggagacgtaggccaggagctcgttgagccgcagtgcggtgtcggtgctggggcggcttgggtccgccgggcgcataaagaacatgtactgctgaatccgatggagggcgtcgcgcaggccggccacggtggcggcgtacttggccgccacggccccgctcttgaacggggtgcgcgccagcagctttggcgccagggtgggccgcagcagcacgtgaaggctggggtcgcagtcgcccacggggtcctcggggacgtccaggccgctgggcaccaccgtctgcaggtacttccagtactgcgtgaggatggcgcggctcaactggccgccgggcagctccacctcgcccagcgcctgggtggcggccgaagcgtagtgccggatgtactcgtagtgcgggtcgctggcgagcccgtccacgatcaaactctcgggaaccgtgttgtgttgccgcgcggccaaccggacgctgcgatcggtgcaggtcagaaacgccggctgcgcgtcgtcggagcgctgccgcaaggcgcccacggccgcgctaaggagcccctccggggtggggagcagacacccgccgaagatgcgccgctcgggaacgcccgcgttgtcgccgcggatcaggttggcaggcgtcaggcaccgcgccagccgcagggagctcgcgccgcgcgtccggcgctgcatggtgacgcccgttcggtcgggacccgccggtcggagttatgccgcgtccagggccatcggggcgctttttatcgggaggagcttatgggcgtggcgggcctcccagcccggtcgcgcgcctccccgacacgtgcgcccgcagggcggcggccccctcgtctcccatcagcagtttcctaaactgggacatgatgtccaccacgcggacccgcgggcccaacacggacccgccgcttacgggggcgggggggaagggctccaggtccttgagaagaaaggcggggtctgccgtcccggacacgggggcccggggcgctgaggaggcggggcgcagatccacgtgctccgcggccgcgcggacgtccgcccagaacttggcgggggtggtgcgcgcgtacaggggctgggtcgctcggaggacgcacgcgtagcgcaggggggtgtacgtgcccacctcgggggccgtgaatcccccgtcaaacgcggccagtgtcacgcacgccaccacggtgtcggcaaagcccagcagccgctgcaggacgagcccggcggccagaatggcgcgcgtggccgccgcgtcgtcccggcgccggtgcgcgtccccgcacgcccgggcgtactttaaggtcacggtcgccagggccgtgtgcagcgcgtacaccgcagcgcccagcacggcgttgagcccgctgttggcgagcagccggcgcgctgcggtgtcgcccagcgcctcgtgctcggcccccacgaccgcggggcttcccaggggcagggcgcgaaacagctcctcccgcgccacgtccgcaaaggcggggtggtgcacgtgcgggtgcaggcgcgcccccacgaccaccgagagccactggaccgtctgctccgccatcaccgccagcacatccagcacgcgccccaggaaggcggcctcccgcgtcaaaacgcaccggacggcgtcgggattgaagcgggcgagcagggccccggtggccaggtacgtcatgcggccggcatagcgggcggccacgcgacagtcgcggtccagcagcgcgcgcaccccgggccagtacagcagggaccccagcgagctgcggaacaccgcggcgtcggggccggattggggggacactaacccccccgcgctcagtaacggcacggccgcggccccgacgggacgcaacgccgtgaggctcgcgaactgccgcctcagctcggccgccctgtcgtccaggtcagacccgcgcgcctccgcgtgaaggcgcgtcccgcacacccacccgttgatggccagccgcacgacggcatccgccaaaaagctcatcgcctgggcggggctggtttttgttcgacgatccgtcaggtcaagaatcccatcgcccgtgatataccaggccaacgcctcgccctgctgcagggtttggcggaaaaacaccgcggggttgtcgggggaggcgaagtgcatgacccccacgcgcgataacccgaacgcgctatccggacacgggtaaaacccggccggatgccccagggctagggcggagcgcacggactcgtcccacacggcaacctgaggggccagtcgatccaacgggaatgccgcccggagctccgggcccggcacgcgtccctccagaacctccaccttgggcggggaacgggccccgccgccgtcctccggcccgacggcttccgggtagtcgtcctcctcgtactgcagctcctctaggaacagcggcgacggcgccacccgcgaaccgccgacccgccccaaaatagcccgcgcgtcgatcgatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagttatcgacttaattaagtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtagatctgctagcgtttaaacttaagcttggtaccgagctcggatccactagtccagtgtggtggaattcggctttaagcctggtaccgtaacaatccctcacccgttccaggtcggggatcaagtgcttgtcagacgccatcgacccagcagccttgagcctcggtggaaaggcccatacctggtgttgctgactaccccgaccgcggtaaaagtcgatggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaataagttagttcaattcatcaatgataggataagtgcatgttaaggcgcctagtgttgacaattaatcatcggcatagtatacggcatagtataatacgactcactataggagggccaccaagccgaattctgcagatatccagcacagtggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaataaacagtctctaactcgctagtctcggccgcggggggcccgggctgcccgccgccaccgctttaaagggccgcgcgcgacccccggggggtgtgttttggggggggcccgttttcggcgtctggccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcccgcggccccgccccccacgcccgccgcgcgcgcgcacgccgcccggaccgccgcccgccttttttgcgcgcgcgcgcgcccgcggggggcccgggctgccacaggtgaaaccaacagagcacggcgcactccgcacgtcacgtggactctagacttaattaaggatccggcgcgaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataattaactcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggacgggacccaggtatcccccctgccgggcctgcggaggaccgcggggaacctcatcatcatcgtccaggcgaccgcgcaccgactggctacgggccgcatcgggcccggggcgctgccgggacgctcggcgatgggatgtgggcggggcttccgacgcgcgccgtcgtcgggctcgcgggccttcccgtcgacggcgcacgggcggctcgtcgcccgccatctcctccagagcctctagctcgctgtcgtcatccccgcggaacaccgcacgcaggtaccccatgaaccccaccccatcgcccgctggctcgtccgccacgggcgaggcgcgggggcgggtggatgcgcgcctcctgcgccccgcgggttcgcgagccgacatggtggcgatagacgcgggttatcggatgtccgctaccccccaaaaaagaaaaagaccccacagcgcggatggaggccggggtaggtgccgccggaccccctcgcgatgggaatggacgggagcgacggggccggcgcaaaaaaacgcagtatctcccgcgaaggctacccgccgccccagcccccggccaaatgcggaaacggtcccgcgctctcgcctttatacgcgggccgccctgcgacacaatcacccgtccgtggtttcgaatctacacgacaggcccgcagacgcggctaacacacacgccggcaacccagaccccagtgggttggttgcgcggtcccgtctcctggctagttctttcccccaccaccaaataatcagacgacaaccgcaggttttgtaatgtatgtgctcgtgtttattgtggatacgaaccggtgacgggaggggaaaacccagacgggggatgcgggtccggtcgcgccccctacccaccgtactcgtcaattccaagggcatcggtaaacatctgctcaaactcgaagtcggccatatccagagcgccgtagggggcggagtcgtggggggtaaatcccggccccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcgtgttcccgcatgaccgcctcggagggcgaggtcgtgaagctggaatacgagtccaacttcgcccgaatcaacaccataaagtacccagaggcgcgggcctggttgccatgcagggtgggaggggtcgtcaacggcgcccctggctcctccgtagccgcgctgcgcaccagcgggaggttaaggtgctcgcgaatgtggtttagctcccgcagccggcgggcctcgattggcactccccggacggtgagcgctccgttgacgaacatgaagggctggaacagacccgccaactgacgccagctctccaggtcgcaacagaggcagtcaaacaggtcgggccgcatcatctgctcggcgtacgcggcccataggatctcgcgggtcaaaaatagatacaaatgcaaaaacaaaacacgcgccagacgagcggtctctcggtagtacctgtccgcgatcgtggcgcgcagcatttctcccaggtcgcgatcgcgtccgcgcatgtgcgcctggcggtgcagctgccggacgctggcgcgcaggtaccggtacagggccgagcagaagttggccaacacggttcgatagctctcctcccgcgcccgtagctcggcgtggaagaaacgagagagcgcttcgtagtagagcccgaggccgtcgcgggtggccggaagcgtagggaaggccacgtcgccgtgggcgcgaatgtcgatttgggtgcgttcggggacgtacgcgtccccccattccaccacatcgctgggcagcgttgataggaatttacactcccggtacaggtcggcgttggtcggtagcgccgaaaacagatcctcgttccaggtatcgagcatggtacatagcgcggggcccgcgctaaagcccaagtcgtcgaggagacggttaaagagggcggcgggggggacgggcatgggtggggagggcatgagctgggcctggctcaggcgccccgttgcgtacagcgggggggccgccggggtgtttttgggacccccggccgggcgggggggcggtggcgaagcgccgtccgcgttcatgtcggcaaacagctcgtcgaccaagaggtccattgggtggggttgatacgggaaagacgatatcgggcttttgatgcgatcgtccccgcccgcccagagagtgtgggacgcccgacggcgcgggaagagaaaacccccaaacgcgttagaggaccggacggaccttatggggggaagtgggcagcgggaaccccgtccgttcccgaggaatgacagcccgtggtcgccaccacgcatttaagcaacccgcacgggccgccccgtacctcgtgacttccccccacattggctcctgtcacgtgaaggcgaaccgagggcggctgtccaacccaccccccgccacccagtcccggtccccgtcggattgggaaacaaaggcacgcaacgccaacaccgaatgaacccctgttggtgctttattgtctgggtacggaagttttcactcgacgggccgtctggggcgagaagcggagcgggctggggctcgaggtcgctcggtggggcgcgacgccgcagaacgccctcgagtcgccgtggccgcgtcgacgtcctgcaccacgtctggattcaccaactcgttggcgcgctgaagcaggtttttgccctcgcagaccgtcacgcggatggtggtgatgccaaggagttcgttgaggtcttcgtctgtgcgcggacgcgacatgtcccagagctggaccgccgccatccgggcatgcatggccgccaggcgcccgaccgcggcgcagaagacgcgcttgttaaagccggccacccggggggtccatggcgcgtcggggtttgggggggcggtgctaaagtgcagctttctggccagcccctgcgcgggtgtcttggatcgggttggcgccgtcgacgcgggggcgtctgggagtgcggcggattctggctgggccgatttcctgccgcgggtggtctccgccgccggggccgcgggggccttagtcgccacccgctgggttcggggggcccggggggcggtggtgggtgtgcgtccggcccctccggacccagcgggtggcggaggcgcccgcgcaggccccgggccggacaaaaccgccccggaaacgggacgccgcgtccgggggacctccgggtgttcgtcgtcttcggatgacgagcccccgtagagggcataatccgactcgtcgtactggacgaaacggacctcgcccctctggcgcgagcgtgtctgtagggcgccacggcgggaggtgtcaggcggactatcgggactcgccatacctgaagacggggtgtagtacagatcctcgtactcatcgcgcggaacctcccgcggacccgacttcacggagcggcgagaggtcatggttccacgaacacgctagggtcggatgcgcggacaattaggcctgggttcggacggcgggggtggtgcaggtgtggagaggtcgagcgataggggcggcccgggagagaagagagggtccgcaaaacccactggggatgcgtgagtggccctctgtgggcggtgggggagagtcttataggaagtgcatataaccacaacccatgggtctaaccaatccccaggggccaagaaacagacacgccccaaacggtctcggtttccgcgaggaaggggaagtcctgggacaccctccacccccacccctcaccccacacagggcgggttcaggcgtgcccggcagccagtagcctctggcagatctgacagacgtgtgcgataatacacacgcccatcgaggccatgcctacataaaagggcaccagggcccccggggcagacatttggccagtgttttgggtctcgcaccgcgcgcccccgatcccatcgcgcccgccctcctcgccgggcggctccccgcgcgggcccgcgtctcccgccgctaaggcgacgagcaagacaaacaacaggcccgcccgacagacccttctgggggggcccatcgtccctaacaggaagatgagtcagtggggatccggggcgatccttgtccagccggacagcttgggtcgggggtacgatggcgactggcacacggccgtcgctactcgcgggggcggagtcgtgcaactgaacctggtcaacaggcgcgcggtggcttttatgccgaaggttagcggggactccggatgggccgtcgggcgcgtctctctggacctgcgaatggctatgccggctgacttttgcgcgattattcacgcccccgcgctagccagccccgggcaccacgtaatactgggtcttatcgactcggggtaccgcggaaccgttatggccgtggtcgtagcgcctaaaaggacgcgggaatttgcccccgggaccctgcgggtcgacgtgacgttcctggacatcctggcgacccccccggccctcaccgagccgatttccctgcggcagttcccgcaactggcgcccccccctccaaccggggccgggatacgcgaagatccttggttggagggggcgctcggggccccaagcgtgactacggccctaccggcgcgacgccgagggcggtccctcgtctatgccggcgagctgacgccggttcagacggaacacggggacggcgtacgagaagccatcgccttccttccaaaacgcgaggaggatgccggtttcgacattgtcgtccgtcgcccggtcaccgtcccggcaaacggcaccacggtcgtgcagccatccctccgcatgctccacgcggacgccgggcccgcggcctgctatgtgttggggcggtcgtcgctcaacgcccgcggcctcctggtcgttcctacgcgctggctccccgggcacgtatgtgcgtttgttgtttacaaccttacgggggttcctgtgaccctcgaggccggcgccaaggtcgcccagctcctggttgcgggggcggacgctcttccttggatccccccggacaactttcacgggaccaaagcgcttcgaaactaccccaggggtgttccggactcaaccgccgaacccaggaacccgccgctcctggtgtttacgaacgagtttgacgcggaggcccccccgagcgagcgcgggaccgggggttttggttctaccggtatttagcccacagctttgggttcgttccgggcaataaaaaacgtttgtatcgcatctttcctgtgtgtagttgtttatgttggatgcctgtgggtctatcacacccgcccctccatcccacaaacacaaaacacacgggttggatgaaaacacgcatttattgacccaaaacacacggagctgctcgagatgggccagggcgaggtgcggttggggaggctgtaggtctgggaacggacacgcggggacacgattccggtttggggtccgggagggcgtcgccgtttcgggcggcaggcgccagcgtaacctccgggggcggcgtgtgggggtgccccaaggagggcgcctcggtcaccccaatcccccccgaccgggttcccccggcaaccccgaaggcggagaggccaagggcccgttcggcgatggccacatcctccatgaccacgtcactctcggccatgctccgaatagcctgggagacgagcacatccgcggacttgtcagccgcccccacggacatgtacatctgcaggatggtggccatacacgtgtccgccaggcgccgcatcttgtcctgatgggccgccacggccccgtcgatcgtgggggcctcgaacccggggtggtggcgcgccagtcgttctaggttcaccatgcaggcgtggtacgtgcgggccaaggcgcgggccttcacgaggcgtcgggtgtcgtccagggaccccagggcgtcatcgagcgtgatgggggcgggaagtagcgcgttaacgaccgccagggcctcctgcagccgcggctccgcctccgagggcggaacggccgcgcggatcatctcatattgttcctcggggcgcgctccccagccacatatagccccgagaagagaagccatcgcgggcgggtactggcccttgggcgcgcggacgcaatggggcaggaagacgggaaccgcggggagaggcgggcggccgggactcccgtggaggtgaccgcgctttatgcgaccgacgggtgcgttattacctcttcgatcgccctcctcacaaactctctactgggggccgagccggtttatatattcagctacgacgcatacacgcacgatggccgtgccgacgggcccacggagcaagacaggttcgaagagagtcgggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgatcctggctctcaccgtggccatcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggtgcgcgtgcttgccgcgtaccgcagggcgtattatggaagcgcgcagagtcccttctggtttcttagcaaattcgggccggacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgctgcgtccctgacctcgtttgccgccatcacgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtacatctacctggcccattttgagtgtttcagccccccgcgcctagccacgcatcttcgggccgtgacgacccacgaccccaaccccgcggccagcacggagcagccctcgcccctgggcagggaggccgtggaacaatttttttgtcacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgatggggcgtttggcggacgccgaaaagctcctggtcccccgcgggtggcccgcgtttgcgcccgccagtcccggggaggacacggcgggcggcacgccgcccccacagacctgcggaattgtcaagcgcctcctgagactggccgccacggaacagcagggccccacacccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatattcaacggcgcgctggcaatcacaaacatcatcctggatctcgacatcgccctgaaggagcccgtcccctttcgccggctccacgaggccctgggccactttaggcgcggggctctggctgcggttcagctcctgtttcccgcggcccgcgtggaccccgacgcatatccctgttattttttcaaaagcgcatgtcggcccggcccggcgtccgtgggttccggcagcggactcggcaacgacgacgacggggactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggcacgccatgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccgtaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggctgccgatggtttgtcggacccccgccggcgtgcccccagaaccaatcgcaaccctgggggaccccgccccggggcggggtggacggacggccccggcgccccccatggcgaggcgtggcgcggcagtgagcagcccgacccacccggaggccagcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacgaccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggtacaataaaaacaaaacatttcaaacaaatcgcccctcgtgttgtccttctttgctcatggccggcggggcgtgggtcacggcagatggcgggggtgggcccggcgtacggcctgggtgggcggagggaactaacccaacgtataaatccgtccccgttccaaggccggtgtcatagtgcccttaggagcttcccgcccgggcgcatccccccttttgcactatgacagcgacccccctcaccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacacccaccacacccctccgctgattaccgagctccgcaactttgcggacctggttaaccacccgccggtcctacgcgaactggaggataagcgcggggtgcggctgcggtgtgcgcggccgtttagcgtcgggacgattaaggacgtctctgggtccggcgcgtcctcggcgggagagtacacgataaacgggatcgtgtaccactgccactgtcggtatccgttctcaaaaacatgctggatgggggcctccgcggccctacagcacctgcgctccatcagctccagcggcatggccgcccgcgcggcagagcatcgacgcgtcaagattaaaattaaggcgtgatctccaacccccccatgaatgtgtgtaaccccccccaaaaaaataaagagccgtaacccaaccaaaccaggcgtggtgtgagtttgtggacccaaagccctcagagacaacgcgacaggccagtatggaccgtgatacttttatttattaactcacaggggcgcttaccgccacaggaataccagaataatgaccaccacaatcgcgaccaccccaaatacagcatggcgccacaccacgccacaacagccctgtcgccggtatggggcatgatcagacgagccgcgcgccgcgcgttgggccctgtacagctcgcgcgaattgaccctaggaggccgccacgcgcccgagttttgcgttcgtcgctggtcgtcgggcgccaaagccccggacggctgttcggtcgaacgaacggccacgacagtggcataggttggggggtggtccgacatagcctcggcgtacgtcgggaggcccgacaagaggtcccttgtgatgtcgggtggggccacaagcctggtttccggaagaaacaggggggttgccaataacccgccagggccaaaactccggcgctgcgcacgtcgttcggcgcggcgccgggcgcgccgagcggctcgctgggcggcttggcgtgagcggccccgctccgacgcctcgccctctccggaggaggttggcggaattggcacggacaacaggggcccagcagagtacggtggaggtgggtccgtgggggtgtccagatcaataacgacaaacggcccctcgttcctaccagacaagctatcgtaggggggcgggggatcagcaaacgcgttccccgcgctccataaacccgcgtcgggttgcgccgcctccgaagccatggatgcgccccaaagccacgactcccgcgcgctaggtccttggggtaatggaaaaggccctactccccatccaagccagccaagttaacgggctacgccttcgggaatgggactggcaccccggcggattttgttgggctggcatgcgtcgcccaaccgagggccgcgtccacgggacgcgccttttataaccccgggggtcattcccaacgatcacatgcaatctaactggctcccctctccccccctctcccctctccccccctctcccctctccccccctctcccctctccccccctctcccctctccccccctctcccctctccccccctctcccctctccccccctctcccctctccccccctctcccctctccccccctctcccctctccccccctctcccctctcccctctgctctttccccgtgacacccgacgctgggggcgtggctgccgggaggggccgcggatgggcgggcctacttggtttcccgccccccccccccccccccgaaccgccccgccggctttgcccccctttgatcccctgctacccccaacccgtgctggtggtgcgggttggggggggatgtgggcgggggtgcgcgggaggtgtcggtggtggtggtggtggtggtagtaggaatggtggtgagggggggggggcgctggttggtcaaaaaagggagggacgggggccggcagaccgacggcgacaacgctccccggcggccgggtcgcggctcttacgagcggcccggcccgcgctcccaccccccgggccgtgtccttgctttccccccgtctcccccccccccgccttctcctcctcctcctcgtttttccaaaccccgcccacccggcccggcccggcccggcccggcccggccaccgccgcccacccacccacctcgggatacccagccccggtcccccgttccccgggggccgttatctccagcgccccgtccggcgcgccgccccccgccgctaaaccccatcccgcccccgggaccccacatataagcccccagccacacgcaagaacagacacgcagaacggctgtgtttatttaaataaaccaatgtcggaataaacaaacacaaacacccgcgacggggggacggaggggacggagggagggggtgacgggggacgggaacagacacaaaaacaaccacaaaaaacaaccacccaccgacacccccaccccagtctcctcgccttctcccacccaccccacgcccccactgagcccggtcgatcgacgagcacccccgcccacgcccccgcccctgccccggcgacccccggcccgcacgatcccgacaacaataacaaccccaacggaaagcggcggggtgttgggggaggcgaggaacaaccgaggggaacgggggatggaaggacgggaagtggaagtcctgatacccatcctacacccccctgccttccaccctccggccccccgcgagtccacccgccggccggctaccgagaccgaacacggcggccgccgcagccgccgcagccgccgccgacaccgcagagccggcgcgcgcactcacaagcggcagaggcagaaaggcccagagtcattgtttatgtggccgcgggccagcagacggcccgcgacaccccccccccgcccgtgtgggtatccggccccccgccccgcgccggtccattaagggcgcgcgtgcccgcgagatatcaatccgttaagtgctctgcagacaggggcaccgcgcccggaaatccattaggccgcagacgaggaaaataaaattacatcacctacccacgtggtgctgtggcctgtttttgctgcgtcatctcagcctttataaaagcgggggcgcggccgtgccgatcgcgggtggtgcgaaagactttccgggcgcgtccgggtgccgcggctctccgggcccccctgcagccggggcggccaaggggcgtcggcgacatcctccccctaagcgccggccggccgctggtctgttttttcgttttccccgtttcgggggtggtgggggttgcggtttctgtttctttaacccgtctggggtgtttttcgttccgtcgccggaatgtttcgttcgtctgtcccctcacggggcgaaggccgcgtacggcccgggacgaggggcccccgaccgcggcggtccgggccccgtccggacccgctcgccggcacgcgacgcgaaaaaggccccccggaggcttttccgggttcccggcccggggcctgagatgaacactcggggttaccgccaacggccggcccccgtggcggcccggcccggggccccggcggacccaaggggccccggcccggggccccacaacggcccggcgcatgcgctgtggtttttttttcctcggtgttctgccgggctccatcgcctttcctgttctcgcttctccccccccccttcttcacccccagtaccctcctccctcccttcctcccccgttatcccactcgtcgagggcgccccggtgtcgttcaacaaagacgccgcgtttccaggtaggttagacacctgcttctccccaatagaggggggggacccaaacgacagggggcgccccagaggctaaggtcggccacgccactcgcgggtgggctcgtgttacagcacaccagcccgttcttttccccccctcccacccttagtcagactctgttacttacccgtccgaccaccaactgcccccttatctaagggccggctggaagaccgccagggggtcggccggtgtcgctgtaaccccccacgccaatgacccacgtactccaagaaggcatgtgtcccaccccgcctgtgtttttgtgcctggctctctatgcttgggtcttactgcctggggggggggagtgcgggggagggggggtgtggaaggaaatgcacggcgcgtgtgtaccccccctaaagttgttcctaaagcgaggatacggaggagtggcgggtgccgggggaccggggtgatctctggcacgcgggggtgggaagggtcgggggagggggggatggagtaccggcccacctggccgcgcgggtgcgcgtgcctttgcacaccaaccccacgtcccccggcggtctctaagaagcaccgccccccctccttcataccaccgagcatgcctgggtgtgggttggtaaccaacacgcccatcccctcgtctcctgtgattctctggctgcaccgcattcttgttttctaactatgttcctgtttctgtctcccccccccccacccctccgccccaccccccaacacccacgtctgtggtgtggccgacccccttttgggcgccccgtcccgccccgccacccctcccatcctttgttgccctatagtgtagttaaccccccccgccctttgtggcggccagaggccaggtcagtccgggcgggcaggcgctcgcggaaacttaacacccacacccaacccactgtggttctggctccatgccagtggcaggatgctttcggggatcggtggtcaggcagcccgggccgcggctctgtggttaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcatccccgcgatacatccaacacagacagggaaaagatacaaaagtaaacctttatttcccaacagacagcaaaaatcccctgagtttttttttattagggccaacacaaaagacccgctggtgtgtggtgcccgtgtctttcacttttcccctccccgacacggattggctggtgtagtgggcgcggccagagaccacccagcgcccgacccccccctccccacaaacacggggggcgtcccttattgttttccctcgtcccgggtcgacgccccctgctccccggaccacgggtgccgagaccgcaggctgcggaagtccagggcgcccactagggtgccctggtcgaacagcatgttccccacgggggtcatccagaggctgttccactccgacgcgggggccgtcgggtactcggggggcatcacgtggttacccgcggtctcggggagcagggtgcggcggctccagccggggaccgcggcccgcagccgggtcgccatgtttcccgtctggtccaccaggaccacgtacgccccgatgttccccgtctccatgtccaggatgggcaggcagtcccccgtgatagtcttgttcacgtaaggcgacagggcgaccacgctagagacccccgagatgggcaggtagcgcgtgaggccgcccgcggggacggccccggaagtctccgcgtggcgcgtcttccgggcacacttcctcggcccccgcggcccagaagcagcgcgggggccgagggaggtttcctcttgtctccctcccagggcaccgacggccccgcccgaggaggcggaagcggaggaggacgcggccccggcggcggaagaggcggcccccgcgggggtcggggccgaggaggaagaggcagaggaggaagaggcggaggccgccgaggacgtcaggggggtcccgggcccaccctggccgcgcccccccggccctgagtcggagggggggtgcgtcgccgccctcttggcccctgccggcgcgagggggggacgcgtggactggggggaggggttttcctggcccgacccgcgcctcttcctcggacgcaccgccgcctcctgctcgacagaggcggcggaggggagcggggcggcgccggagggggcggcgccgcgggagggcccgtgcccaccctccacgcccggcccccccgagccgcgcgccaccgtcgcacgcgcccggcacagactctgttcttggttcgcggcctgagccagggacgagtgcgactggggcacacggcgcgcgtccgcggggcgggcggccggctccgccccgggggccggggcgcgggggccgggccccggaggcggcgctcgcacgcacggggccacggccgcgcgggggcgcgcgggtcccgacgcggccgcggacgcggggggcccggggcggggggcggagcctggcatgggcgccgcggggggcctgtggggagaggccgggggggagtcgctgatcactatggggtctctgttgtttgcaaggggggcgggtctgttgacaagggggcccgtccggcccctcggccgccccgcctccgcttcaacaaccccaaccccaaccccaacccccccggaggggccagacgccccccgcggcgccgcggctcgcgactggcgggagccgccgccgccgctgctgttggtggtggtgttggtgttactgctgccgtgtggcccgatgggcgccgaggggggcgctgtccgagccgcggccggctggggggctgcgtgagacgccccgcccgtcacggggggcgcggcggcgcctctgcgtgggggggcgcggggcgtccggcggggggcgggcggtacgtagtctgctgcaagagacaacggggggcgcgatcaggttacgccccctccccggcccgccctttcctcgcccgcccgcctattcctccctccccccccctcctcctcctcctcccccagggtccttgccgccccccgcctcaccgtcgtccaggtcgtcgtcatcctcgtccgtggtgggctccgggtgggtgggcgacagggccctcaccgtgtgcccccccagggtcaggtaccgcggggcgaaccgctgattgcccgtccagataaagtccacggccgtgcccgccctgacggcctcctcggcctccatgcgggtctgggggtcgttcacgatcgggatggtgctgaacgacccgctgggcgtcacgcccactatcaggtacaccagcttggcgttgcacagcgggcaggtgttgcgcaattgcatccaggttttcatgcacgggatgcagaagcggtgcatgcacgggaaggtgtcgcagcgcaggtggggcgcgatctcatccgtgcacacggcgcacacgtcgccctcgtcgctccccccgtcctctcgagggggggcgcccccgcaactgccggggtcttcctcgcggggggggctcccccccgagaccgcccccccatccacgccctgcggccccagcagccccgtctcgaacagttccgtgtccgtgctgtccgcctcggaggcggagtcgtcgtcatggtggtcggcgtccccccgcccccccacttcggtctccgcctcagagtcgctgctgtccggcaggtctcggtcgcagggaaacacccagacatccggggcgggctaaggggaaaaaaggggggcgggtaagaatggggggggatttcccgcgtcaatcagcacccacgagttccccctctcccccccccgcctcacaaagtcctgcccccctgctggcctcggaagaggggggagaaaggggtctgcaaccaaaggtggtctgggtccgtcctttggatcccgacccctcttcttccctcttctcccgccctccagacgcaccggagtcgggggtcccacggcgtcccccaaatatggcgggcggctcctccccacccccctagatgcgtgtgagtaaggggggcctgcgtatgagtcagtggggaccacgcccccaacacggcgaccccggtccttgtgtgtttgttgtgggggcgtgtctctgtgtatgagtcagggggtcccacggcgaccccgggccctgcgtctgagtcaaaggggccatgtgtatgtgttgggggtctgtatatataaagtcagggggtcacatggcgacccccaacagggcgaccccggtccctgtatatatagggtcagggggttccgcaccccctaacatggcgcccccggtccctgtatatatagtgtcacggggttccacgccccctaacatggcgccccaacatggcgcccggctcccgtgtatgagtgggggtcccccaacatggcggccggttccagtgtaagggtcgggggtcccccaacatggcgccccccaatatggcgccccccaatatggcgccccagacatggcgcccggcccctcacctcgcgctgggggcggccctcaggccggcgggtactcgctccggggcggggctccatgggggtcgtatgcggctggagggtcgcggacggagggtccctgggggtcgcaacgtaggcggggcttctgtggtgatgcggagagggggcggcccgagtctgcctggctgctgcgtctcgctccgagtgccgaggtgcaaatgcgaccagactgtcgggccagggctaacttataccccacgcctttcccctccccaaaggggcggcagtgacgattcccccaatggccgcgcgtcccaggggaggcaggcccaccgcggggcggccccgtccccggggaccaacccggcgcccccaaagaatatcattagcatgcacggcccggcccccgatttgggggcccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattaatgagtttctaattaccataccgggaagcggaacaaggcctcttgcaagtttttaattaccataccgggaagtgggcggcccggcccattgggcggtaactcccgcccaatgggccgggccccgaagactcggcggacgctggttggccgggccccgccgcgctggcggccgccgattggccagtcccgcccccgaggcggcccgccctgtgagggcgggctggctccaagcgtatatatgcgcggctcctgccatcgtctctccggagagcggcttggtgcggagctcccgggagctccgcggaagacccaggccgcctcgggtgtaacgttagaccgagttcgccgggccggctccgcgggccagggcccgggcacgggcctcgggccccaggcacggcccgatgaccgcctcggcctccgccacccggcgccggaaccgagcccggtcggcccgctcgcgggcccacgagccgcggcgcgccaggcgggcggccgaggcccagaccaccaggtggcgcacccggacgtggggcgagaagcgcacccgcgcgggggtcgcgggggtcgcgggggtcgcgggggtcgcgggggtcgcggggggctccggcgccccctccccgcccgcgcgtcgcaggcgcaggcgcgccaggtgctccgcggtgacgcgcaggcggagggcgaggcgcggcggaaggcggaaggggcgcgagggggggtgggaggggtcagccccgccccccgggcccacgccgggcggtgggggccggggccggggggcggcggcggtgggccgggcctctggcgccggctcgggcggggggctgtccggccagtcgtcgtcatcgtcgtcgtcggacgcggactcgggaacgtggagccactggcgcagcagcagcgaacaagaaggcgggggcccaccggcggggggcggcggcggggcggccgcgggcgcgctcctgaccgcgggttccgagttgggcgtggaggttacctgggactgtgcggttgggacggcgcccgtgggcccgggcggccgggggcggcgggggccgcgatggcggcggcggcgggccatggagacagagagcgtgccggggtggtagagtttgacaggcaagcatgtgcgtgcagaggcgagtagtgcttgcctgtctaactcgctagtctcggccgcggggggcccgggctgcccgccgccaccgctttaaagggccgcgcgcgacccccggggggtgtgttttggggggggcccgttttcggcgtctggccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcccgcggccccgccccccacgcccgccgcgcgcgcgcacgccgcccggaccgccgcccgccttttttgcgcgcgcgcgcgcccgcggggggcccgggctgccacaggtgaaaccaacagagcacggcgcactccgcacgtcacacgtcacgtcatccaccacacctgcccaacaacacaactcacagcgacaactcaccgcgcaacaactcctgttcctcatccacacgtcaccgcgcacctcccgctcctccagacgtaccccggcgcaacacaccgctcctgctacacaccaccgccccctccccagccccagccctccccagccccagccctccccggccccagccctccccggccccagccctccccggccccagccctccccggccccagccctccccggccccagccctccccggccccagccctccccggcgcgtcccgcgctccctcgggggggttcgggcatctctacctcagtgccgccaatctcaggtcagagatccaaaccctccgggggcgcccgcgcaccaccaccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgaataaacaacgctactgcaaaacttaatcaggttgttgccgtttattgcgtcttcgggtctcacaagcgccccgccccgtcccggcccgttacagcaccccgtccccctcgaacgcgccgccgtcgtcttcgtcccaggcgccttcccagtccacaacttcccgccgcgggggcgtggccaagcccgcctccgcccccagcacctccacggcccccgccgccgccagcacggtgccgctgcggcccgtggccgaggcccagcgaatcccgggcggcgccggcggcagggcccccgggccgtcgtcgtcgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcggcgtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgaagcgcgtgcgcacgcggtagcgcacgttgccgccgcggcacaggcgcagcggcggcgcgtcggggtacaggcgcgcgtgcgcggcctccacgcgcgcgaagacccccgggccgaacacgcggcccgaggccagcaccgtgcggcgcaggtcccgcgccgccggccagcgcacggcgcactgcacggcgggcagcagctcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgtgcgcccagcgccgacacgtcgggggcgccggtccaattgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttatcgtcgtcgtcgccgccgccgcacgcggcctgggcggcgggggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgtggctgggccccgggggctgccgccgccagccgcccagggggtcggggccctcggcgggccggcgcgacacggccacggggcgcgggcgggcctgcgccgcggcggcccggggcgccgcgggctgggcgggggcgggctcgggccccgggggcgtggaggggggcgcgggcgcggggaggggggcgcgggcgtccgagccgggggcgtccgcgccgctcttcttcgtcttcgggggtcgcgggccgccgcctccgggcggccgggccgggccgggactcttgcgcttgcgcccctcccgcggcgcggcggaggcggcggcggccgccagcgcgtcggcggcgtccggtgcgctggccgccgccgccagcagggggcgcaggctctggttgtcaaacagcaggtccgcggcggcggcggccgcggagctcggcaggcgcgggtcccgcggcagcgcggggcccagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggcccgcgggtccctccggccgcggggggctggcgggccgggccccggccagccccgggacggccgccaggtcgccgtcgaagccctcggccagcgcctccaggatcccgcggcaggcggccaggcactcgacggccacgcggccggcctgggcgcggcgcccggcgtcgtcgtcggcgtcggcgtggcgggcggcgtcggggtcgtcgccccccgcgggggaggcgggcgcggcggacagccgccccagggcggcgaggatccccgcggcgccgtacccggcgggcaccgcgcgctcgcccggtgcggcggcggcgacggcggcgaccccctcgtcatctgcgccggcgccggggctccccgcggcccccgtcagcgccgcgttctcgcgcgccaacaggggcgcgtaggcgcggcgcaggctggtcagcaggaagcccttctgcgcgcggtcgtatcggcggctcatggccacggcggccgccgcgtgcgccaggccccagccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccaccccatggcctccgcgtccggggtgtacagcagccgcgtgatcagggcgtactgctgcgcggcgtcgcccagctcgggcgcccacacggccgccggggcgcccgaggcctcgaaccggcgtcgcgcctcctccgcctcgggcgccccccagaggcccgggcggctgtcgcccaggccgccgtacagcacccgccccgggggcgggggcccggcgccgggccacggctccccgctgacgtacccgtcgcgatagcgcgcgtagaaggcgccggaggtcgcgtcggcgtccagctcgacccgccggggctgcccggccgtgaagcggcccgtggcgtcgcggccggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggctgccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccaccggccgtgacgacgtctccgcggcggctgggccggcgggcgcggcgacaggcggtccgtggggtccggatacgcgccgcgtagcggggcctcccgttcgcggccccgggccggggcccggtcgccggcggcgtcggctgcgtcgtcgtactcgtccccgtcatcgtcgtcggctcgaaaggcgggggtccggggcggcgaggccgcggggtcgggcgtcgggatcgtccggacggcctcctctaccatggaggccagcagagccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctccggggatacggctgcgacggcggacgtagcacggtaggtcacctacggactctcgatggggggagggggcgagacccacggaccccgacgacccccgccgtcgacgcggaactagcgcggaccggtcgatgcttgggtgggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccaggggccggcgggaaggaggagcagcggaggccgccggccccccgcccccccggcgggcccaccccgaacggaattccattatgcacgaccccgccccgacgccggcacgccgggggcccgtggccgcggcccgttggtcgaacccccggccccgcccatccgcgccatctgccatgggcggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggccccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgaggcgaccggcggcgaccgttgcgtggaccgcttcctgctcgtcgggcggggggaagccactgtggtcctccgggacgttttctggatggccgacatttccccaggcgcttttgcgccttgtgtaaaagcgcggcgtcccgctctccgatccccgcccctgggcacgcgcaagcgcaagcgcccttcccgccccctctcatcggagtctgaggtagaatccgatacagccttggagtctgaggtcgaatccgagacagcatcggattcgaccgagtctggggaccaggatgaagccccccgcatcggtggccgtagggccccccggaggcttggggggcggttttttctggacatgtcggcggaatccaccacggggacggaaacggatgcgtcggtgtcggacgaccccgacgacacgtccgactggtcttatgacgacattcccccacgacccaagcgggcccgggtaaacctgcggctcacgagctctcccgatcggcgggatggggttatttttcctaagatggggcgggtccggtctacccgggaaacgcagccccgggcccccaccccgtcggccccaagcccaaatgcaatgctacggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgtttgctgcaggtgtccggcggaacctggggcatgcacctgcgcaacaccatacgggaggtggaggctcgattcgacgccaccgcggaacccgtgtgcaagcttccttgtttggagaccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgccgcagaagaccgcaagtgtctggacggctgccggaaaatgcgcttctccaccgcctgcccctatccgtgcagcgacacgtttctccggccgtgagtccggtcgccccgacccccttgtatgtccccaaaataaaagaccaaaatcaaagcgtttgtcccagcgtcttaatggcgggaagggcggagagaaacagaccacgcggacatggggggtgtttgggggtttattggcaccgggggctaaagggtggtaaccggatagcagatgtgaggaagtcggggccgttcgccgcgaacggcgatcagagggtcagtttcttgcggaccacggcccggcgatgtgggttgctcgtctgggacctcgggcatgcccatacacgcacaacacggacgccgcaccggatgggacgtcgtaagggggcctggggtagctgggtggggtttgtgcagagcaatcagggaccgcagccagcgcatacaatcgcgctcccgtccgtttgtcccgggcagtaccacgccgtactggtattcgtaccggctgagcagggtctccagggggtggttgggggccgcggggaacggggtccacgccacggtccactcgggcaaaaaccgagtcggcacggcccacggttctcccacccacgcgtctggggtcttgatggcgataaatcttaccccgagccggattttttgggcgtattcgagaaacggcacacacagatccgccgcgcctaccacccacaagtggtagaggcgaggggggctgggttggtctcggtgcagcagtcggaagcacgccacggcgtccacgacctcggtgctctccaaggggctgtcctccgcaaacaggcccgtggtggtgtttggggggcagcgacaggacctagtgcgcacgatcgggcgggtgggtttgggtaagtccatcagcggctcggccaaccgtcgaaggttggccggacgaacgacgaccggggtacccaggggttctgatgccaaaatgcggcactgcctaagcaggaagctccacagggccgggcttgcgtcgacggaagtccggggcagggcgttgttctggtcaaggagggtcattacgttgacgacaacaacgcccatgttggtatattacaggcccgtgtccgatttggggcacttgcagatttgtaaggccacgcacggcggggagacaggccgacgcgggggctgctctaaaaatttaagggccctacggtccacagacccgccttcccgggggggcccttggagcgaccggcagcggaggcgtccgggggaggggagggtgatttacgggggggtaggtcagggggtgggtcgtcaaactgccgctccttaaaaccccggggcccgtcgttcggggtgctcgttggttggcactcacggtgcggcgaatggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccagcgaggccgagcgcctgtgtcatctgcaggagatccttgcccagatgtacggaaaccaggactaccccatagaggacgaccccagcgcggatgccgcggacgatgtcgacgaggacgccccggacgacgtggcctatccggaggaatacgcagaggagctttttctgcccggggacgcgaccggtccccttatcggggccaacgaccacatccctcccccgtgtggcgcatctccccccggtatacgacgacgcagccgggatgagattggggccacgggatttaccgcggaagagctggacgccatggacagggaggcggctcgagccatcagccgcggcggcaagcccccctcgaccatggccaagctggtgactggcatgggctttacgatccacggagcgctcaccccaggatcggaggggtgtgtctttgacagcagccatccagattacccccaacgggtaatcgtgaaggcggggtggtacacgagcacgagccacgaggcgcgactgctgaggcgactggaccacccggcgatcctgcccctcctggacctgcatgtcgtctccggggtcacgtgtctggtcctccccaagtaccaggccgacctgtatacctatctgagtaggcgcctgaacccactgggacgcccgcagatcgcagcggtctcccggcagctcctaagcgccgttgactacattcaccgccagggcattatccaccgcgacattaagaccgaaaatatttttattaacacccccgaggacatttgcctgggggactttggcgccgcgtgcttcgtgcagggttcccgatcaagccccttcccctacggaatcgccggaaccatcgacaccaacgcccccgaggtcctggccggggatccgtataccacgaccgtcgacatttggagcgccggtctggtgatcttcgagactgccgtccacaacgcgtccttgttctcggccccccgcggccccaaaaggggcccgtgcgacagtcagatcacccgcatcatccgacaggcccaggtccacgttgacgagttttccccgcatccagaatcgcgcctcacctcgcgctaccgctcccgcgcggccgggaacaatcgcccgccgtacacccgaccggcctggacccgctactacaagatggacatagacgtcgaatatctggtttgcaaagccctcaccttcgacggcgcgcttcgccccagcgccgcagagctgctttgtttgccgctgtttcaacagaaatgaccgccccctgggggcggtgctgtttgcgggttggcacaaaaagaccccgatccgcgtctgtggtgtttttggcatcatgtcgcagggcgccatgcgtgccgttgttcccattatcccattccttttggttcttgtcggtgtatcgggggttcccaccaacgtctcctccaccacccaaccccaactccagaccaccggtcgtccctcgcatgaagcccccaacatgacccagaccggcaccaccgactctcccaccgccatcagccttaccacgcccgaccacacaccccccatgccaagtattggactggaggaggaggaagaggaggagggggccggggacggcgaacatcttgaggggggagatgggacccgtgacaccctaccccagtccccgggcccagccttcccgttggctgaggacgtcgagaaggacaaacccaaccgtcccgtagtcccatcccccgatcccaacaactcccccgcgcgccccgagaccagtcgcccgaagacaccccccaccattatcgggccgctggcaactcgccccacgacccgactcacctcaaagggacgacccttggttccgacgcctcaacataccccgctgttctcgttcctcactgcctcccccgccctggacaccctcttcgtcgtcagcaccgtcatccacaccttatcgtttttgtgtattggtgcgatggcgacacacctgtgtggcggttggtccagacgcgggcgacgcacacaccctagcgtgcgttacgtgtgcctgccgtccgaacgcgggtagggtatggggcgggggatggggagagcccacatgcggaaagcaagaacaataaaggcggtggtatctagttgatatgcatctctgggtgtttttggggtgtggcggacgcggggcggtcattggacggggtgcagttaaatacatgcccgggacccatgaagcatgcgcgacttccgggcctcagaacccacccgaaacggccaacggacgtctgagccaggcctggctatccggagaaacagcacacgacttggcgttctgtgtgtcgcgatgtctctgcgcgcagtctggcatctggggcttttgggaagcctcgtgggggctgttcttgccgccacccatcggggacctgcggccaacacaacggaccccttaacgcacgccccagtgtcccctcaccccagccccctggggggctttgccgtccccctcgtagtcggtgggctgtgcgccgtagtcctgggggcggcatgtctgcttgagctcctgcgtcgtacgtgccgcgggtgggggcgttaccatccctacatggacccagttgtcgtataatttcccccccccccccccttctccgcgtgggtgatgtcgggtccaaactcccgacaccaccagctggcatggtataaatcaccggtgcgccccccaaaccatgtccggcagggggatgggggggcaatgcggagggcacccaacaacaccgggctaaccaggaaatccgtggccccggcccccaataaagatcgcggtagcccggccgtgtgacactatcgtccataccgaccacaccgacgaatcccccaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcagcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactagatacccccccttaatgggtgcgggggggtcaggtctgcggggttgggatgggaccttaactccatataaagcgagtctggaaggggggaaaggtggacagtcgataagtcggtagcgggggacgcgcacctgttccgcctgtcgcacccacagctttttttgcgaaccgtcccgttccgggatgccgtgccgcccgttgcagggcctggtgctcgtgggcctctgggtctgtgccaccagcctggttgtccgtggccccacggtcagtctggtatcaaactcatttgtggacgccggggccttggggcccgacggcgtagtggaggaagacctgcttattctcggggagcttcgctttgtgggggaccaggtcccccacaccacctactacgatgggggcgtagagctgtggcactaccccatgggacacaaatgcccacgggtcgtgcatgtcgtcacggtgaccgcgtgcccacgtcgccccgccgtggcattcgccctgtgtcgcgcgaccgacagcactcacagccccgcatatcccaccctggagctcaatctggcccaacagccgcttttgcgggtccagagggcaacgcgggactatgccggggtgtacgtgttacgcgtatgggtcggtgacgcgccaaacgccagcctgtttgtcctggggatggccatagccgccgaagggactctggcgtacaacggctcggcctatggctcctgcgacccgaaactgcttccgtcttcggccccgcgtctggccccggcgagcgtataccaacccgcccctaaccaggcctccaccccctcgaccaccacctccaccccctcgaccaccatccccgctccctcgaccaccatccccgctccccaagcatcgaccacgcccttccccacgggagatccaaaaccacaacctcccggggtcaaccacgaacccccatctaatgccacgcgagcgacccgcgactcgcgatacgcgctaacggtgacccagataatccagatagccatccccgcgtccatcatagccctggtgtttctggggagctgtatttgctttatacacagatgtcaacgccgctaccgacgctcccgtcgcccgatttacagcccccagatgcccacgggcatctcatgcgcggtgaacgaagcggccatggcccgcctcggagccgagctcaaatcgcatccgagcaccccccccaaatcccggcgccggtcgtcacgcacgccaatgccctccctgacggccatcgccgaagagtcggagcccgctggggcggctgggcttccgacgccccccgtggaccccacgacacccaccccaacgcctcccctgttggtataggtccacggccactggccgggagcaccacataaccgaccgcagtccctgagttgggaataaaccggtattatttacctatatccgtgtatgtcgatttctttccccccctccccggaaaccaaagaaggaagcaaagaatggatgggaggagttcaggaagccggggagagggcccgcggcgcatttaaggcgttgttgtgttgactttgcctcttctggcgggttggtgcggtgctgtttgttgggctcccattttacccgaagatcggctgctatccccgggacatggatcgcggggcggtggtggggtttcttctcggtgtttgtgttgtatcgtgcttggcgggaacgcccaaaacgtcctggagacgggtgagtgtcggcgaggacgtttcgttgcttccagctccggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacgaacggacttcgtgtggcaggagcgcgcggccgtggttaaccggagtctggttattcacggggtccgagagacggacagcggcctgtataccctgtccgtgggcgacataaaggacccggctcgccaagtggcctcggtggtcctggtggtgcaaccggccccagttccgaccccacccccgaccccagccgattacgacgaggatgacaatgacgagggcgaggacgaaagtctcgccggcactcccgccagcgggaccccccggctcccgcctccccccgcccccccgaggtcttggcccagcgcccccgaagtctcacatgtgcgtggggtgaccgtgcgtatggagactccggaagctatcctgttttcccccggggagacgttcagcacgaacgtctccatccatgccatcgcccacgacgaccagacctactccatggacgtcgtctggttgaggttcgacgtgccgacctcgtgtgccgagatgcgaatatacgaatcgtgtctgtatcacccgcagctcccagaatgtctgtccccggccgacgcgccgtgcgccgcgagtacgtggacgtctcgcctggccgtccgcagctacgcggggtgttccagaacaaaccccccaccgcgctgttcggccgaggctcacatggagcccgtcccggggctggcgtggcaggcggcctccgtcaatctggagttccgggacgcgtccccacaacactccggcctgtatctgtgtgtggtgtacgtcaacgaccatattcacgcctggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcactggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatacccccgtagcgatgggcatcaatctcgccgccagctcacaacctttggatccggaaggcccgatcgccgttactcccaggcctccgattcgtccgtcttctggtaaggcgccccatcccgaggccccacgtcggtcgccgaactgggcgaccgccggcgaggtggacgtcggagacgagctaatcgcgatttccgacgaacgcggacccccccgacatgaccgcccgcccctcgccacgtcgaccgcgccctcgccacacccgcgacccccgggctacacggccgttgtctccccgatggccctccaggctgtcgacgccccctccctgtttgtcgcctggctggccgctcggtggctccggggggcttccggcctgggggccgtcctgtgtgggattgcgtggtatgtgacgtcaattgcccgaggcgcataaagggccggtggtccgcctagccgcagcaaattaaaaatcgtgagtcacagcgaccgcaacttcccacccggagctttcttccggcctcgatgacgtcccggctctccgatcccaactcctcagcgcgatccgacatgtccgtgccgctttatcccacggcctcgccagtttcggtcgaagcctactactcggaaagcgaagacgaggcggccaacgacttcctcgtacgcatgggccgccaacagtcggtattaaggcgtcgacgcagacgcacccgctgcgtcggcatggtgatcgcctgtctcctcgtggccgttctgtcgggcggatttggggcgctcctgatgtggctgctccgctaaaagaccgcatcgacacgcgcgtccttcttgtcgtctctcttcccccccatcaccccgcaatttgcacccagcctttaactacattaaattgggttcgattggcaatgttgtctcccggttgatttttgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtggcaaggaagaaacaagcccgaccaccagacagaaaatgtaaccatacccaaaccgactctgggggctgtttgtggggtcggaaccataggatgaacaaaccaccccgtaccacccgcacccaagggtgcggtggctcatcggcatctgtccggtatgggttgttccccacccactcgcgttcggacgtcttagaatcatggcggttttctatgccgacatcggttttctcccccgcaataagacacgatgcgataaaatctgtttgtaaaatttattaagggtacaaattgccctagcacaggggtggggttagggccgggtccccacacccaaacgcaccaaacagatgcaggcagtgggtcgagtacagccccgcgtacgaacacgtcgatgcgtgtgtcagacagcaccagaaagcacaggccatcaacaggtcgtgcatgtgtcggtgggtttggacgcggggggccatggtggtgataaagttaatggccgccgtccgccagggccacaggggcgacgtctcttggttggcccggagccactgggtgtggaccagccgcgcgtggcggcccaacatggcccctgtagccgggggcgggggatcgcgcacgtttgcagcgcacatgcgagacacctcgaccacggttcgaaagaaggcccggtggtccgcgggcaacatcaccaggtgcgcaagcgcccgggcgtccagagggtagagccctgagtcatccgaggttggctcatcgcccgggtcttgccgcaagtgcgtgtgggttgggcttccggtgggcgggacgcgaaccgcggtgtggatcccgacgcgggcccgagcgtatgctccatgttgtggggagaaggggtctgggctcgccaggggggcatacttgcccgggctatacagacccgcgagccgtacgtggttcgcggggggtgcgtggggtccggggctcccggggagaccggggctcccggggagaccggggctccctgggagaccggggttgtcgtggatccctggggtcacgcggtaccctggggtctctgggagctcgcggtactctgggttccctaggttctcggggtggtcgcggaacccggggctcccggggaacacgcggtgtcctggggattgttggcggtcggacggcttcagatggcttcgagatcgtagtgtccgcaccgactcgtagtagacccgaatctccacattgccccgccgcttgatcattatcaccccgttgcgggggtccggagatcatgcgcgggtgtcctcgaggtgcgtgaacacctctggggtgcatgccggcggacggcacgccttttaagtaaacatctgggtcgcccggcccaactggggccgggggttgggtctggctcatctcgagagccacgggggggaaccaccctccgcccagagactcgggtgatggtcgtacccgggactcaacgggttaccggattacggggactgtcggtcacggtcccgccggttcttcgatgtgccacacccaaggatgcgttgggggcgatttcgggcagcagcccgggagagcgcagcaggggacgctccgggtcgtgcacggcggttctggccgcctcccggtcctcacgcccccttttattgatctcatcgcgtacgtcggcgtacgtcctgggcccaacccgcatggtgtccaggaaggtgtccgccatttccagggcccacgacatgctcccccccgacgagcaggaagcggtccacgcaacggtcgccgccggtcgcctcgacgaggacgttcctcctgcgggaaggcacgaacgcgggtgagccccctcctccgcccccgcgtcccccctcctccgcccccgcgtcccccctcctccgcccccgcgtcccccctcctccgcccccgcgtcccccctcctccgcccccgcgtcccccctcctccgcccccgcgtcccccctcctccacccccgcgtccccccctcctccgcccacccaaggtgcttacccgtgcaaaaaaggcggaccggtgggtttctgtcgtcggaggcccccggggtgcgtcccctgtgtttcgtgggtggggtgggcgggtctttcccccccgcgtccgcgtgtccctttccgatgcgatcccgatcccgagccggggcgtcgcgatgccgacgccgtccgctccgacggccctctgcgactcccgctcccggtccgcgtgctccgcagccgctcccgtcgttcgtggccggcgccgtctgcgggcgtcggtcgcgccgggcctttatgtgcgccggagagacccgccccccgccgcccgggcccgcccccggggccggcgcggagtcgggcacggcgccagtgctcgcacttcgccctaataatatatatatattgggacgaagtgcgaacgcttcgcgttctcacttcttttacccggcggccccgcccccttggggcggtcccgcccgccggccaatgggggggcggcaaggcgggcggcccttgggccgcccgccgtcccgttggtcccggcgtccggcgggcgggaccggggggcccggggacggccaacgggcgcgcggggctcgtatctcattaccgccgaaccgggaagtcggggcccgggccccgccccctgcccgttcctcgttagcatgcggaacggaagcggaaaccgccggatcgggcggtaatgagatgccatgcggggcggggcgcggacccacccgccctcgcgccccgcccatggcagatggcgcggatgggcggggccgggggttcgaccaacgggccgcggccacgggcccccggcgtgccggcgtcggggcggggtcgtgcataatggaattccgttcggggtgggcccgccgggggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttcccacccaagcatcgaccggtccgcgctagttccgcgtcgacggcgggggtcgtcggggtccgtgggtctcgccccctccccccatcgagagtccgtaggtgacctaccgtgctacgtccgccgtcgcagccgtatccccggaggatcgccccgcatcggcgatggcgtcggagaacaagcagcgccccggctccccgggccccaccgacgggccgccgcccaccccgagcccagaccgcgacgagcggggggccctcgggtggggcgcggagacggaggagggcggggacgaccccgaccacgaccccgaccacccccacgacctcgacgacgcccggcgggacgggagggcccccgcggcgggcaccgacgccggcgaggacgccggggacgccgtctcgccgcgacagctggctctgctggcctccatggtagaggaggccgtccggacgatcccgacgcccgaccccgcggcctcgccgccccggacccccgcctttcgagccgacgacgatgacggggacgagtacgacgacgcagccgacgccgccggcgaccgggccccggcccggggccgcgaacgggaggccccgctacgcggcgcgtatccggaccccacggaccgcctgtcgccgcgcccgccggcccagccgccgcggagacgtcgtcacggccggtggcggccatcggcgtcatcgacctcgtcggactccgggtcctcgtcctcgtcgtccgcatcctcttcgtcctcgtcgtccgacgaggacgaggacgacgacggcaacgacgcggccgaccacgcacgcgaggcgcgggccgtcgggcggggtccgtcgagcgcggcgccggcagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgagctggacgccgacgcgacctccggcgccttctacgcgcgctatcgcgacgggtacgtcagcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggcggccgtgtgggcgcccgagctgggcgacgccgcgcagcagtacgccctgatcacgcggctgctgtacaccccggacgcggaggccatggggtggctccagaacccgcgcgtggtccccggggacgtggcgctggaccaggcctgcttccggatctcgggcgccgcgcgcaacagcagctccttcatcaccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgtcgccgccgccgcaccgggcgagcgcgcggtgcccgccgggtacggcgccgcggggatcctcgccgccctggggcggctgtccgccgcgcccgcctcccccgcggggggcgacgaccccgacgccgcccgccacgccgacgccgacgacgacgccgggcgccgcgcccaggccggccgcgtggccgtcgagtgcctggccgcctgccgcgggatcctggaggcgctggccgagggcttcgacggcgacctggcggccgtcccggggctggccggggcccggcccgccagccccccgcggccggagggacccgcgggccccgcttccccgccgccgccgcacgccgacgcgccccgcctgcgcgcgtggctgcgcgagctgcggttcgtgcgcgacgcgctggtgctcatgcgcctgcgcggggacctgcgcgtggccggcggcagcgaggccgccgtggccgccgtgcgcgccgtgagcctggtcgccggggccctgggccccgcgctgccgcgggacccgcgcctgccgagctccgcggccgccgccgccgcggacctgctgtttgacaaccagagcctgcgccccctgctggcggcggcggccagcgcaccggacgccgccgacgcgctggcggccgccgccgcctccgccgcgccgcgggaggggcgcaagcgcaagagtcccggcccggcccggccgcccggaggcggcggcccgcgacccccgaagacgaagaagagcggcgcggacgcccccggctcggacgcccgcgcccccctccccgcgcccgcgcccccctccacgcccccggggcccgagcccgcccccgcccagcccgcggcgccccgggccgccgcggcgcaggcccgcccgcgccccgtggccgtgtcgcgccggcccgccgagggccccgaccccctgggcggctggcggcggcagcccccggggcccagccacacggcggcgcccgcggccgccgccctggaggcctactgctccccgcgcgccgtggccgagctcacggaccacccgctgttccccgtcccctggcgaccggccctcatgtttgacccgcgggccctggcctcgatcgccgcgcggtgcgccgggcccgcccccgccgcccaggccgcgtgcggcggcggcgacgacgacgataacccccacccccacggggccgccgggggccgcctctttggccccctgcgcgcctcgggcccgctgcgccgcatggcggcctggatgcgccagatccccgaccccgaggacgtgcgcgtggtggtgctgtactcgccgctgccgggcgaggacctggccggcggcggggcctcgggggggccgccggagtggtccgccgagcgcggcgggctgtcctgcctgctggcggccctggccaaccggctgtgcgggccggacacggccgcctgggcgggcaattggaccggcgcccccgacgtgtcggcgctgggcgcacagggcgtgctgctgctgtccacgcgggacctggccttcgccggggccgtggagtttctggggctgctcgccagcgccggcgaccggcggctcatcgtggtcaacaccgtgcgcgcctgcgactggcccgccgacgggcccgcggtgtcgcggcagcacgcctacctggcgtgcgagctgctgcccgccgtgcagtgcgccgtgcgctggccggcggcgcgggacctgcgccgcacggtgctggcctcgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcacgccgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcgacgacgacggcccgggggccctgccgccggcgccgcccgggattcgctgggcctcggccacgggccgcagcggcaccgtgctggcggcggcgggggccgtggaggtgctgggggcggaggcgggcttggccacgcccccgcggcgggaagttgtggactgggaaggcgcctgggacgaagacgacggcggcgcgttcgagggggacggggtgctgtaacgggccgggacggggcggggcgcttgtgagacccgaagacgcaataaacggcaacaacctgattaagttttgcagtagcgttgtttattcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcggtggtggtgcgcgggcgcccccggagggtttggatctctgacctgagattggcggcactgaggtagagatgcccgaacccccccgagggagcgcgggacgcgccggggagggctggggccggggagggctggggccggggagggctggggccggggagggctggggccggggagggctggggccggggagggctggggccggggagggctggggctggggagggctggggctggggagggggcggtggtgtgtagcaggagcggtgtgttgcgccggggtacgtctggaggagcgggaggtgcgcggtgacgtgtggatgaggaacaggagttgttgcgcggtgagttgtcgctgtgagttgtgttgttgggcaggtgtggtggatgacgtgacgtgtgacgtgcggagtgcgccgtgctctgttggtttcacctgtggcagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggc

SEQ ID NO: 2 - DNA, 170660 bp, Artificial Sequence
  Synco-B18R is a modified oncolytic virus made by incorporating a B18R gene into the Synco-2D gene sequence.
ggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatcccgggcccgcggtaccgtcgactgcagaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactagcggaagagcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggccggcgcggagtcgggcacggcgccagtgctcgcacttcgccctaataatatatatatattgggacgaagtgcgaacgcttcgcgttctcacttcttttacccggcggccccgcccccttggggcggtcccgcccgccggccaatgggggggcggcaaggcgggcggcccttgggccgcccgccgtcccgttggtcccggcgtccggcgggcgggaccggggggcccgggggcgcgccggatccttaattaagtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtagatctgctagcgtttaaacttaagcttggtaccgagctcggatccagtacccttcaccatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggaaaggctagaaaaaaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattagaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaaaagggtcaagacaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctagacttaattaagtctagagtccacgtgacgtgcggagtgcgccgtgctctgttggtttcacctgtggcagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggcagcccgggccccccgcggccgagactagcgagttagagactgtttagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacctccacgcccaactcggaacccgcggtcaggagcgcgcccgcggccgccccgccgccgccccccgccggtgggcccccgccttcttgttcgctgctgctgcgccagtggctccacgttcccgagtccgcgtccgacgacgacgatgacgacgactggccggacagccccccgcccgagccggcgccagaggcccggcccaccgccgccgccccccggccccggcccccaccgcccggcgtgggcccggggggcggggctgacccctcccaccccccctcgcgccccttccgccttccgccgcgcctcgccctccgcctgcgcgtcaccgcggagcacctggcgcgcctgcgcctgcgacgcgcgggcggggagggggcgccggagccccccgcgacccccgcgacccccgcgacccccgcgacccccgcgacccccgcgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaacgttacacccgaggcggcctgggtcttccgcggagctcccgggagctccgcaccaagccgctctccggagagacgatggcaggagccgcgcatatatacgcttggagccagcccgccctcacagggcgggccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccccatggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggtgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacaaggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcgggggggggagagggggaactcgtgggtgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccacccggagcccaccacggacgaggatgacgacgacctggacgacggtgaggcggggggcggcaaggaccctgggggaggaggaggaggagggggggggagggaggaataggcgggcgggcgaggaaagggcgggccggggagggggcgtaacctgatcgcgccccccgttgtctcttgcagcagactacgtaccgcccgccccccgccggacgccccgcgcccccccacgcagaggcgccgccgcgccccccgtgacgggcggggcgtctcacgcagccccccagccggccgcggctcggacagcgcccccctcggcgcccatcgggccacacggcagcagtaacaccaacaccaccaccaacagcagcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggccccccgcgtccgcggccgcgtcgggacccgcgcgcccccgcgcggccgtggccccgtgcgtgcgagcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtgggcacgggccctcccgcggcgccgccccctccggcgccgccccgctcccctccgccgcctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgacccccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagactatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataagggacgccccccgtgtttgtggggaggggggggtcgggcgctgggtggtctctggccgcgcccactacaccagccaatccgtgtcggggaggggaaaagtgaaagacacgggcaccacacaccagcgggtcttttgtgttggccctaataaaaaaaaactcaggggatttttgctgtctgttgggaaataaaggtttacttttgtatcttttccctgtctgtgttggatgtatcgcggggatgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgccatgttgggcaggctctggtgttaaccacagagccgcggcccgggctgcctgaccaccgatccccgaaagcatcctgccactggcatggagccagaaccacagtgggttgggtgtgggtgttaagtttccgcgagcgcctgcccgcccggactgacctggcctctggccgccacaaagggcggggggggttaactacactatagggcaacaaaggatgggaggggtggcggggcgggacggggcgcccaaaagggggtcggccacaccacagacgtgggtgttggggggtggggcggaggggtgggggggggggagacagaaacaggaacatagttagaaaacaagaatgcggtgcagccagagaatcacaggagacgaggggatgggcgtgttggttaccaacccacacccaggcatgctcggtggtatgaaggagggggggcggtgcttcttagagaccgccgggggacgtggggttggtgtgcaaaggcacgcgcacccgcgcggccaggtgggccggtactccatccccccctcccccgacccttcccacccccgcgtgccagagatcaccccggtcccccggcacccgccactcctccgtatcctcgctttaggaacaactttagggggggtacacacgcgccgtgcatttccttccacaccccccctcccccgcactccccccccccaggcagtaagacccaagcatagagagccaggcacaaaaacacaggcggggtgggacacatgccttcttggagtacgtgggtcattggcgtggggggttacagcgacaccggccgaccccctggcggtcttccagccggcccttagataagggggcagttggtggtcggacgggtaagtaacagagtctgactaagggtgggagggggggaaaagaacgggctggtgtgctgtaacacgagcccacccgcgagtggcgtggccgaccttagcctctggggcgccccctgtcgtttgggtcccccccctctattggggagaagcaggtgtctaacctacctggaaacgcggcgtctttgttgaacgacaccggggcgccctcgacgagtgggataacgggggaggaagggagggaggagggtactgggggtgaagaaggggggggggagaagcgagaacaggaaaggcgatggagcccggcagaacaccgaggaaaaaaaaaccacagcgcatgcgccgggccgttgtggggccccgggccggggccccttgggtccgccggggccccgggccgggccgccacgggggccggccgttggcggtaaccccgagtgttcatctcaggccccgggccgggaacccggaaaagcctccggggggcctttttcgcgtcgcgtgccggcgagcgggtccggacggggcccggaccgccgcggtcgggggcccctcgtcccgggccgtacgcggccttcgccccgtgaggggacagacgaacgaaacattccggcgacggaacgaaaaacaccccagacgggttaaagaaacagaaaccgcaacccccaccacccccgaaacggggaaaacgaaaaaacagaccagcggccggccggcgcttagggggaggatgtcgccgacgccccttggccgccccggctgcaggggggcccggagagccgcggcacccggacgcgcccggaaagtctttcgcaccacccgcgatcggcacggccgcgcccccgcttttataaaggctgagatgacgcagcaaaaacaggccacagcaccacgtgggtaggtgatgtaattttattttcctcgtctgcggcctaatggatttccgggcgcggtgcccctgtctgcagagcacttaacggattgatatctcgcgggcacgcgcgcccttaatggaccggcgcggggcggggggccggatacccacacgggcggggggggggtgtcgcgggccgtctgctggcccgcggccacataaacaatgactctgggcctttctgcctctgccgcttgtgagtgcgcgcgccggctctgcggtgtcggcggcggctgcggcggctgcggcggccgccgtgttcggtctcggtagccggccggcgggtggactcgcggggggccggagggtggaaggcaggggggtgtaggatgggtatcaggacttccacttcccgtccttccatcccccgttcccctcggttgttcctcgcctcccccaacaccccgccgctttccgttggggttgttattgttgtcgggatcgtgcgggccgggggtcgccggggcaggggcgggggcgtgggcgggggtgctcgtcgatcgaccgggctcagtgggggcgtggggtgggtgggagaaggcgaggagactggggtgggggtgtcggtgggtggttgttttttgtggttgtttttgtgtctgttcccgtcccccgtcaccccctccctccgtcccctccgtccccccgtcgcgggtgtttgtgtttgtttattccgacattggtttatttaaataaacacagccgttctgcgtgtctgttcttgcgtgtggctgggggcttatatgtggggtcccgggggcgggatggggtttagcggcggggggcggcgcgccggacggggcgctggagataacggcccccggggaacgggggaccggggctgggtatcccgaggtgggtgggtgggcggcggtggccgggccgggccgggccgggccgggccgggtgggcggggtttggaaaaacgaggaggaggaggagaaggcgggggggggggagacggggggaaagcaaggacacggcccggggggtgggagcgcgggccgggccgctcgtaagagccgcgacccggccgccggggagcgttgtcgccgtcggtctgccggcccccgtccctcccttttttgaccaaccagcgccccccccccccctcaccaccattcctactaccaccaccaccaccaccaccgacacctcccgcgcacccccgcccacatccccccccaacccgcaccaccagcacgggttgggggtagcaggggatcaaaggggggcaaagcggcggggcggttcggggggggggggggggggcgggaaaccaagtaggcccgcccatccgcggcccctcccggcagccacgcccccagcgtcgggtgtcacggggaaagagcagaggggagaggggagagggggggagaggggagagggggggagaggggagagggggggagaggggagagggggggagaggggagagggggggagaggggagagggggggagaggggagagggggggagaggggagagggggggagaggggagagggggggagaggggagagggggggagagggggtatataaaccaacgaaaagcgcgggaacggggatacggggcttgtgtggcacgacgtcgtggttgtgttactgggcaaacacttggggactgtaggtttctgtgggtgccgaccctaggcgctatggggattttgggttgggtcgggcttattgccgttggggttttgtgtgtgcgggggggcttgccttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttttggaggacttgagttaccccgcgtttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcagccgaagcccctcaccacgccgccgcccatcatcgccacgtcggaccccaccccgcgacgggacgccgccacaaaaagcagacgccgacgaccccactcccggcgcctctaacgatgcctcgacggaaacccgtccgggttcggggggcgaaccggccgcctgtcgctcgtcagggccggcggcgctcctcgccgccctagaggctggtcccgctggtgtgacgttttcctcgtccgcgccccccgaccctcccatggatttaacaaacgggggggtgtcgcctgcggcgacctcggcgcctctggactggaccacgtttcggcgtgtgtttctgatcgacgacgcgtggcggcccctgatggagcctgagctggcgaaccccttaaccgcccacctcctggccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggcggccgtcaagaactgttatcccgaggcacggatgagcggccacggttgcctggaaaagtgggcgcgggacggcgtcctgttactaaacacgaccctgaccgtcaagcgcggggcggcggcgtcccactctagaatcggttgggaccgtttcgtgggcggagttatccgccggttggccgcgcgccgccccggcctggtgtttatgctctggggcacacacgcccagaatgccatcaggccggaccctcgggtccattgcgtcctcaagttttcgcacccgtcgcccctctccaaggttccgttcggaacctgccagcatttcctcgtggcgaaccgatacctcgagacccggtcgatttcacccatcgactggtcggtttgaaaggcatcgacgtccggggtttttgtcggtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcggggatatcgccagatacgactcccgcagattccattcggggtgccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgccccccccccatgccaccctcactgcccgtcgcgcgtgtttgatgttaataaataacacataaatttggctggttgtttgttgtctttaatggaccgcccgcaagggggggggggcatttcagtgtcgggtgacgagcgcgatccggccgggatcctaggaccccaaaagtttgtctgcgtattccagggcggggctcagttgaatctcccgcagcacctctaccagcaggtccgcggtgggctggagaaactcggccgtcccggggcaggcggttgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtggaactgtaaggggacggcgggttaatagacaatgaccacgttcggatcgcgcagagccgatagtatgtgctcactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgtagtcggcgtttatggccatggccagctccgtagagtcaatggactcgacaaactcgctgatgttggtgttgacgacggacatgaagccgtgttggtcccgcaagaccacgtaaggcaggggggcctcttccagtaactcggccacgttggccgtcgcgtgccgcctccgcagctcgtccgcaaaggcaaacacccgtgcgtacgtgtatcccatgagcgtataattgtccgtctgcagggcgacggacatcagccccccgcgcggcgagccggtcagcatctcgcagccccggaagataacgttgtccacgtacgtgctaaagggggcgacttcaaatgcctccccgaagagctcttggaggattcggaatctcccgaggaaggcccgcttcagcagcgcaaactgggtgtgaacggcggcggtggtctccggttccccgggggtgtagtggcagtaaaacacgtcgagctgttgttcgtccagccccgcgaaaataacgtcgaggtcgtcgtcgggaaaatcgtccgggcccccgtcccgcggccccagttgcttaaaatcaaacgcacgctcgccgggggcgcctgcgtcggccattaccgacgcctgcgtcggcacccccgaagatttggggcgcagagacagaatctccgccgttagttctcccatgcgggcgtaggcgagggtcctctgggtcgcatccaggcccgggcgctgcagaaagttgtaaaaggagataagcccgctaaatatgagccgcgacaggaacctgtaggcaaactccaccgaagtctccccctgagtctttacaaagctgtcgtcacgcaacactgcctcgaaggcccggaacgtcccactaaacccaaaaaccagttttcgcaggcgcgcggtcaccgcgatctggctgttgaggacgtaagtgacgtcgttgcgggccacgaccagctgctgtttgctgtgcacctcgcagcgcatgtgccccgcgtcctggtcctggctctgcgagtagttggtgatgcggctggcgttggccgtgagccacttttcaatcgtcaggccgggctggtgtgtcagccgtcggtattcgtcaaactccttgaccgacacgaacgtaagcacggggagggtgaacacgacgaactccccctcacgggtcaccttcaggtaggcgtggagcttggccatgtacgcgctcacctctttgtgggaggagaacagccgcgtccagccggggaggttggcggggttggtgatgtagttttccgggacgacgaagcgatccacgaactgcatgtgctcctcggtgatgggcaggccgtactccagcaccttcatgaggttaccgaactcgtgctcgacgcaccgtttgttgttaataaaaatggcccagctatacgagaggcgggcgtactcgcgcagcgtgcggttgcagatgaggtacgtgagcacgttctcgctctggcggacggaacaccgcagtttctggtgctcgaaggtcgactccagggacgccgtctgcgtcggcgagcccacacacaccaacacgggccgcaggcgggccgcgtactggggggtgtggtacagggcgttaatcatccaccagcaatacaccacggccgtgaggaggtgacgcccaaggagcccggcctcgtcgatgacgatcacgttgctgcgggtaaaggccggcagcgccccgtgggtggccggggccaaccgcgtcagggcgccctcggccaaccccagggtccgttccagggcggccagggcgcgaaactcgttccgcaactcctcgcccccggaggcggccagggcgcgcttcgtgaggtccaaaatcacctcccagtagtacgtcagatctcgtcgctgcaggtcctccagcgaggcggggttgctggtcagggtgtacgggtactgtcccagttgggcctggacgtgattcccgcgaaacccaaattcatgaaagatggtgttgatgggtcggctgagaaaggcgcccgagagtttggcgtacatgttttgggccgcaatgcgcgtggcgcccgtcaccacacagtccaagacctcgttgattgtctgcacgcacgtgctctttccggagccagcgttgccggtgataagatacaccgcgaacggaaactccctgaggggcaggcctgcgggggactctaaggccgccacgtcccggaaccactgcagatggggcacttgcgctccgtcgagctgttgttgcgagagctctcggatgcgcttaaggattggctgcaccccgtgcatagacgtaaaatttaaaaaggcctcggccctccctggaacggctggtcggtccccgggttgctgaaggtgcggcgggccgggtttctgtccgtctagctggcgctccccgccggccgccgccatgaccgcaccacgctcgcgggcccccactacgcgtgcgcggggggacacggaagcgctgtgctcccccgaggacggctgggtaaaggttcaccccagccccggtacgatgctgttccgcgagattctccacgggcagctggggtataccgagggccagggggtgtacaacgtcgtccggtccagcgaggcgaccacccggcagctgcaggcggcgatctttcacgcgctcctcaacgccaccacttaccgggacctcgaggcggactggctcggccacgtggcggcccgcggtctgcagccccaacggctggttcgccggtacaggaacgcccgggaggcggatatcgccggggtggccgagcgggtgttcgacacgtggcggaacacgcttaggacgacgctgctggactttgcccacgggttggtcgcctgctttgcgccgggcggcccgagcggcccgtcaagcttccccaaatatatcgactggctgacgtgcctggggctggtccccatattacgcaagcgacaagaagggggtgtgacgcagggtctgagggcgtttctcaagcagcacccgctgacccgccagctggccacggtcgcggaggccgcggagcgcgccggccccgggttttttgagctggcgctggccttcgactccacgcgcgtggcggactacgaccgcgtgtatatctactacaaccaccgccggggcgactggctcgtgcgagaccccatcagcgggcagcgcggagaatgtctggtgctgtggccccccttgtggaccggggaccgtctggtcttcgattcgcccgtccagcggctgtttcccgagatcgtcgcgtgtcactccctccgggaacacgcgcacgtctgccggctgcgcaataccgcgtccgtcaaggtgctgctggggcgcaagagcgacagcgagcgcggggtggccggtgccgcgcgggtcgttaacaaggtgttgggggaggacgacgagaccaaggccgggtcggccgcctcgcgcctcgtgcggcttatcatcaacatgaagggcatgcgccacgtaggcgacattaacgacaccgtgcgttcctacctcgacgaggccggggggcacctgatagacgccccggccgtcgacggtaccctccctggattcggcaagggcggaaacagccgcgggtctgcgggccaggaccagggggggcgggcgccgcagcttcgccaggccttccgcacggccgtggttaacaacatcaacggcgtgttggagggctatataaataacctgtttggaaccatcgagcgcctgcgcgagaccaacgcgggcctggcgacccaattgcaggagcgcgaccgcgagctccggcgcgcaacagcgggggccctggagcgccagcagcgcgcggccgacctggcggccgagtccgtgaccggtggatgcggcagccgccctgcgggggcggacctgctccgggccgactatgacattatcgacgtcagcaagtccatggacgacgacacgtacgtcgccaacagctttcagcacccgtacatcccttcgtacgcccaggacctggagcgcctgtcgcgcctctgggagcacgagctggtgcgctgttttaaaattctgtgtcaccgcaacaaccagggccaagagacgtcgatctcgtactccagcggggcgatcgccgcattcgtcgccccctactttgagtcagtgcttcgggccccccgggtaggcgcgcccatcacgggctccgatgtcatcctgggggaggaggagttatgggatgcggtgtttaagaaaacccgcctgcaaacgtacctgacagacatcgcggccctgttcgtcgcggacgtccagcacgcagcgctgcccccgcccccctccccggtcggcgccgatttccggcccggcgcgtccccgcggggccggtccagatcgcggtcgcccggaagaactgcgcgaggcgcgccggaccagggcgggggcatcgggcaccgggatggccgccgcgacggccgacgatgaggggtcggccgccaccatcctcaagcaggccatcgccggggaccgcagcctggtcgaggcggccgaggcgattagccagcagacgctgctccgcctggcctgcgaggtgcgccaggtcggcgaccgccagccgcggtttaccgccaccagcatcgcgcgcgtcgacgtcgcgcctgggtgccggttgcggttcgttctggacgggagtcccgaggacgcctatgtgacgtcggaggattactttaagcgctgctgcggccagtccagttatcgcggcttcgcggtggcggtcctgacggccaacgaggaccacgtgcacagcctggccgtgccccccctcgttctgctgcaccggttctccctgttcaaccccagggacctcctggactttgagcttgcctgtctgctgatgtacctggagaactgcccccgaagccacgccaccccgtcgacctttgccaaggttctggcgtggctcggggtcgcgggtcgccgcacgtccccattcgaacgcgttcgctgccttttcctccgcagttgccactgggtcctaaacacactcatgttcatggtgtacgtaaaaccgttcgacgacgagttcgtcctgccccactggtacatggcccggtacctgctggccaacaacccgccccccgttctctcggccctgttctgtgccaccccgacgagctcctcattccggctgccggggccgcccccccgctccgactgcgtggcctataaccccgccgggatcatggggagctgctgggcgtcggaggaggtgcgcgcgcctctggtctattggtggctttcggagaccccaaaacgacagacgtcgtcgctgttttatcagttttgttgaattttaggaaataaacccggttttgtttctgtggcctcccgacggatgcgcgtgtccttactccgtcttggtgggtgggtggctgtgtatggcgtcccatctgtgcggggaggggggcaagtcggcacgtattcggacagactcaagcacacacgggggagcgctcttgtctcagggcaatgtttttattggtcaaactcaggcaaacagaaacgacatcttgtcgtcaaagggatacacaaacttccccccctcgccccatactcccgccagcaccccggtaaacaccaactcaatctcgcgcaggatttcgcgcaggtgatgagcgcagtccacgggggggagcacaaggggccgcgggtatagatcgacggggacgccgaccgactccccgcctccgggacagacacgcacgacgcgccgccagtagtgctctgcgtccagcaaggcgccgccgcggaaggcagtggggggcaaggggtcgctggcctcaaagggggacacccgaacgctccagtactccgcgtccaaccgtttattaaacgcgtccaagataaggcggtcgcaggcgtcctccataaggccccgggccgtgagtgcgtcctcctccggcacgcatgccgttgtcaggcccaggacccgtcgcagcgtgtcgcgtacgacccctgccgccgtggtgtacgcgggcccgcggagaggaaatcccccaagatggtcagtgttgtcgcgggagttccagaaccacactcccgcctggctccaggcgactgcgtgggtgtagacgccctcgagggccaggcacagtgggtgccgcagccggacggcgttggccctaagcacggctcccacggccgtctcgatggcccgccgggcgtcctcgatcaccccggaagccgcatccgcgtcttgggggtccacgttaaagacaccccagaacgcacccccatcgcccccgcagaccgcgaacttcaccgagctggccgtctcctcgatctgcaggcagacggcggccattaccccacccaggagctgccgcagcgcagggcaggcgttgcacgtgtccgggaccaggcgctccaagacggccccggcccagggctctgagggagcggccaccaccagcgcgtccagtcttgctaggcccgtccggccgtgggggtccgccagcccgctccccccgaggtcggccagggccgccaggagctgggcgcgaagtccggggaagcaaaaccgcgccgtccagacgggcccgacggccgcgggcgggtctaacagttggatgattttagtggcgggatgccaccgcgccaccgcctcccgcaccgcgggcaggaggcatccggctgccgccgaggccacgccgggccaggctcgcggggggaggacgaccctggcccccaccgcgggccaggcccccaggagcgcggcgtaagcggccgcggccccgcgcaccaggtcccgtgccgactcggccgtggccggcacggtgaacgtgggccaacccggaaaccccaggacggcaaagtacgggacgggtcccccccggacctcaaactcgggccccagaaaggcaaagacgggggccagggccccgggggcggcgtggaccgtggtatgccactgccggaaaagggcgacgagcgccggcgcggagaacttctcgccggcgcttacaaagtagtcgtaatcgcggggcagcagcacccgtgccgtgactcgttgcgggtgcccgcgtggccgcaggcccacctcgcacacctcgaccaggtccccgaacgcgccctccttcttgatcggcggaaacgcaagagtctggtattcgcgcgcaaatagcgcggttccggtggtgatgttaacggtcagcgaagcggcggacgcgcactggggggtgtcgcgaatggccgccaggcgcgcccacgccagccgcgcgtcgggatgctcggcaacgcgcgccgccagggccatagggtcgatgtcaatgttggcctccgcgaccaggagagcggcgcgaggggcggcgggcgggccccacgacgctctctcaactttcaccaccagtcccgtgcgtgggtccgagccgatacgcagcggggcgaacagggccaccggcccggtctggcgctccagggccgccaggacgcacgcgtacagcgcccgccacagagtcgggttctccaggggctccagcggggaggcggccggcgtcgtcgcggcgcgggcggccgccacgacggcctggacggagacgtccgcggagccgtagaaatcccgcagctccgtcgcggtgacggagacctccgcaaagcgcgcgcgaccctcccctgcggcgttgcgacatacaaaatacaccagggcgtggaagtactcgcgagcgcgggggggcagccataccgcgtaaagggtaatggcgctgacgctctcctccacccacacgatatctgcggtgtccatcgcacggcccctaaggatcacgggcggtctgtgggtcccatgctgccgtgcctggccgggcccggtgggtcgcggaaaccggtgacggggggggggcggtttttggggttggggtgggggtgggaaacggcccgggtccgggggccaacttggcccctcggtgcgttccggcaacagcgccgccggtccgcggacgaccacgtaccgaacgagtgcggtcccgagacttatagggtgctaaagttcaccgccccctgcatcatgggccaggcctcggtggggagctccgacagcgccgcctccaggatgatgtcagcgttggggttggcgctggatgagtgcgtgcgcaaacagcgcccccacgcgggcacgcgtagcttgaagcgcgcgcccgcaaactcccgcttgtgggccataagcagggcgtacagctgcctgtgggtccggcaggcgctgtggtcgatgtggtgggcgtccaacaaccccacgattgtctgtttggtgaggtttttaacgcgccccgccccgggaaacgtctgcgtgcttttggccatctgcacgccaaacagttcgccccagattatcttgaacagcgccaccgcgtggtccgtctcgctaacggacccgcgcgggggacagccgcttagggcgtcggcgacgcgcttgacggcttcctccgagagcagaagtccgtcggttacgttacagtggcccagttcgaacaccagctgcatgtagcggtcgtagtggggggtcagtaggtccagcacgtcatcggggccgaaggtcctcccagatcccccggccgccgagtcccaatgcaggcgcgcggccatggtgctgcacaggcacaacagctcccagacgggggttacgttcagggtggggggcagggccacgagctccagctctccggtgacgttgatcgtggggatgacgcccgtggcgtagtggtcatagatccgccgaaatatggcgctgctgcgggtggccatgggaacgcggagacaggcctccagcaacgccaggtaaataaaccgcgtgcgtcccatcaggctgttgaggttgcgcatgagcgcgacaatttccgccggcgcgacatcggaccggaggtatttttcgacgaaaagacccacctcctccgtctcggcggcctgggccggcagcgacgcctcgggatcccggcaccgcagctcccgtagatcgcgctgggccctgagggcgtcgaaatgtacgccccgcaaaaacagacagaagtcctttggggtcagggtatcgtcgtgtccccagaagcgcacgcgtatgcagtttagggtcagcagcatgtgaaggatgttaaggctgtccgagagacacgccagcgtgcatctctcaaagtagtgtttgtaacggaatttgttgtagatgcgcgacccccgccccagcgacgtgtcgcatgccgacgcgtcacagcgccccttgaaccggcgacacagcaggtttgtgacctgggagaactgcgcgggccactggccgcaggaactgaccacgtgattaaggagcatgggcgtaaagacgggctccgagcgcgccccggagccgtccatgtaaatcagtagctcccccttgcggagggtgcgcacccgtcccagggactggtacacggacaccatgtccggtccgtagttcatgggtttcacgtaggcgaacatgccatcaaagtgcaggggatcgaagctgaggcccacggttacgaccgtcgtgtatataaccacgcggtattggccccacgtggtcacgtccccgaggggggtgagcgagtgaagcaacagcacgcggtccgtaaactgacggcagaaccgggccacgatctccgcgaaggagaccgtcgacgaaaaaatgcagatgttatcgcccccgccaaggcgcgcttccagctccccaaagaacgtggccccccgggcctccggagaggcgtccggagacgggccgctcggcggcccgggcgggcgcagggcagcctgcaggagctcggtccccagacgcgggagaaacaggcaccggcgcgccgaaaacccgggcatggcgtactcgccgaccaccacatgcacgtttttttcgccccggagaccgcacaggaagtccaccaactgcgcgttggcggttgcgtccatggcgatgatccgaggacagatgcgcagcaggcgtagcattaacgcatccacgcggcccagttgctgcatcgttggcgaatagagctggcccagcgtcgacataacctcgtccagaacgaggacgtcgtagttgttcagaaggttggggcccacgcgatgaaggctttccacctggacgataagtcggtggaaggggcggtcgttcataatgtaattggtggatgagaagtaggtgacaaagtcgaccaggcctgactcagcgaaccgcgtcgctagggtctgggtaaaactccgacgacaggagacgacgagcacactcgtgtccggagagtggatcgcttcccgcagccagcggatcagcgcggtagtttttcccgaccccattggcgcgcggaccacagtcacgcacctggccgtcggggcgctcgcgttggggaaggtgacgggtccgtgctgctgccgctcgatcgttgttttcgggtgaacccggggcacccattcggccaaatcccccccgtacaacatccgcgctagcgatacgctcgacgtgtactgttcgcactcgtcgtccccaatgggacgcccggcccccagaggatctcccgactccgcgccccccacgaaaggcatgaccggggcgcggacggcgtggtgggtctggtgtgtgcaggtggcgacgtttgtggtctctgcggtctgcgtcacggggctcctcgtcctggcctctgtgttccgggcacggtttccctgcttttacgccacggcgagctcttatgccggggtgaactccacggccgaggtgcgcgggggtgtagccgtgcccctcaggttggacacgcagagccttgtgggcacttatgtaatcacggccgtgttgttgttggccgtggccgtgtatgccgtggtcggcgccgtgacctcccgctacgaccgcgccctggacgcgggccgccgtctggctgcggcccgcatggccatgccgcacgccacgctgatcgccggaaacgtctgctcttggttgctgcagatcaccgtcctgttgctggcccatcgcatcagccagctggcccacctggtttacgtcctgcactttgcgtgtctggtgtattttgcggcccatttttgcaccaggggggtcctgagcgggacgtatctgcgtcaggtgcacggcctgatggagctggccccgacccatcatcgcgtcgtcggcccggctcgcgccgtgctgacaaacgccttgctgttgggcgtcttcctgtgcacggccgacgccgcggtatccctgaataccatcgccgcgttcaactttaatttttcggccccgggcatgctcatctgcctgaccgtgctgttcgccattctcgtcgtatcgctgttgttggtggtcgagggggtgttgtgtcactacgtgcgcgtgttggtgggcccccacctgggggccgtggccgccacgggcatcgtcggcctggcctgcgagcactattacaccaacggctactacgttgtggagacgcagtggccgggggctcagacgggagtccgcgtcgccctcgccctggtcgccgcctttgccctcggcatggccgtgctccgctgcacccgcgcctatctgtatcacaggcggcaccacaccaaattttttatgcgcatgcgcgacacgcgacaccgcgcacattccgccctcaagcgcgtacgcagttccatgcgcggatcgcgagacggccgccacaggcccgcacccggcagcccgcccgggattcccgaatatgcggaagacccctacgcgatctcatacggcggccagctcgaccggtacggagattccgacggggagccgatttacgacgaggtggcggacgaccaaaccgacgtattgtacgccaagatacaacacccgcggcacctgcccgacgacgatcccatctatgacaccgttggggggtacgaccccgagcccgccgaggaccccgtgtacagcaccgtccgccgttggtagctgtttggttccgttttaataaaccgtttgtgtttaacccgaccgtggtgtatgtctggtgtgtggcgtccgatcccgttactatcaccgtccccccccccccctcaaccccggcgattgtgggttttttaaaaacgacacgcgtgcgaccgtatacagaacattgttttggtttttattcgctatcggacatggggggtggaaactgggtggcggggcaggcgcctccgggggtccgccggtgagtgtggcgcgagggggggtccgatgaacgcaggcgctgtctccccggggcccgcgtaaccccgcgcatatccgggggcacgtagaaattaccttcctcttcggactcgatatccacgacgtcaaagtcgtgggcggtcagcgagacgacctccccgtcgtcggtgatgaggacgttgtttcggcagcagcagggccgggccccggagaacgagaggcccatagctcggcgagcgtgtcgtcgaatgccaggcggctgcttcgctggatggccttatagatctccggatcgatgcggacgggggtaatgatcagggcgatcggaacggcctggttcgggagaatggacgccttgctgggtcctgcggccccgagagccccggcgccgtcctccaggcggaacgttacgccctcctccgcgctggtgcggtgcctgccgataaacgtcaccagatgcgggtggggggggcagtcggggaagtggctgtcgagcacgtagccctgcaccaagatctgcttaaagttcgggtgacgggggttcgcgaagacgggctcgcggcggaccagatccccggagctccaggacacgggggagatggtgtggcgtccgaggtcgggggcgccaaacagaagcacctccgagacaacgccgctatttaactccaccaaggcccgatccgcggcggagcaccgccttttttcgcccgaggcgtgggcctctgaccaggcctggtcttgcgtgacgagagcctcctccgggccggggacgcgcccgggcgcgaagtatcgcacgctgggcttcgggatcgaccggataaatgcccggaacgcctccggggaccggtgtgccatcaagtcctcgtacgcggaggccgtggggtcgctggggtccatggggtcgaaagcgtacttggcccggcatttgacctcgtaaaaggccaggggggtcttggggactggggccaggtagccgtgaatgtcccgaggacagacgagaatatccagggacgccccgaccatccccgtgtgaccgtccatgaggaccccacacgtatgcacgttctcttcggcgaggtcgctgggttcgtggaagataaagcgccgcgtgtcggcgccggcctcgccgccgtcgtccgcgcggcccacgcagtagcgaaacagcaggcttcgggccgtcggctcgttcacccgcccgaacatcaccgccgaagactgtacatccggccgcaggctggcgttgtgcttcagccactggggcgagaaacacggaccctgggggccccagcggagggtggatgcggtcgtgaggccccgccggagcagggcccatagctggcagtcggcctggttttgcgtggccgcctcgtaaaaccccatgaggggccggggcgccacggcgtccgcggcggccgggggcccgcggcgcgtcaggcgccataggtgccgaccgagtccgcggtccaccatacccgcctcctcgaggaccacggccagggaacacagataatccaggcgggcccagaggggaccgatggccagaggggcgcggacgccgcgcagcaacccgcgcaggtggcgctcgaacgtctcggctagtatatgggagggcagcgcgttggggatcaccgacgccgaccacatagagtcaaggtccggggagtcgggatcggcgtccgggtcgcgggcgtgggtgcccccaggagatagcggaatgtctggggtcggaggccctgaggcgtcagaaagtgccggcgacgcggcccggggcttttcgtctgcggtgtcggtggcgtgctgatcacgtggggggttaacgggcgaatgggagctcgggtccacagctgatgtcgtctggggtggggggggcaggggacggaaggtggttgtcagcggaagactgttagggcgggggcgcttgggggggctgtcggggccacgaggggtgtcctcggccagggcccagggacgcttagtcacggtgcgtcccggcggacatgctgggcctaccgtggactccatttccgagacgacgtggggggagcggtggttgagcgcgccgccgggtgaacgctgattctcacgacagcgcgtgccgcgcgcacgggttggtgtgacacaggcgggacaccagcaccaggagaggcttaagctcgggaggcagcgccaccgacgacagtatcgccttgtgtgtgtgctggtaatttatacaccgatccgtaaacgcgcgccgaatcttgggattgcggaggtggcgccggatgccctctgggacgtcatacgccaggccgtgggtgttggtctcggccgagttgacaaacagggctgggtgcagcacgcagcgataggcgagcagggccagggcgaagtccggcgacagctggttgttaaaatactggtaaccgggaaaccgggtcacgggtacgcccaggctcggggcgacgtacacgctaaccaccaactccagcagcgtctggcccagggcgtacaggtcaaccgctaacccgacgtcgtgcttcaggcggtggttggtaaattcggcccgttcgttgttaaggtatttcaccaacagctccgggggctggttatacccgtgacccaccagggtgtgaaagttggctgtggttagggcggtgggcatgccaaacatccggggggacttgaggtccggctcctggaggcaaaactgcccccgggcgatcgtggagttggagttgagggtgacgaggctaaagtcggcgaggacggcccgccggagcgagacggcgtccgaccgcagcatgacgaggatgttggcgcacttgatatccaggtggctgatcccgcaggtggtgtttaaaaacacaacggcgcgggccagctccgtgaagcactggtggagggccgtcgagaccgaggggtttgttgtgcgcagggacgccagttggccgatatacttaccgaggtccatgtcgtacgcggggaacactatctgtcgttgttgcagcgagaacccgaggggcgcgatgaagccgcggatgttgtgggtgcggccggcgcgtagaacgcactccccgaccaacagggtcgcgatgagctcaacggcaaaccactccttttcctttatggtcttaacggcaagcttatgttcgcgaatcagttggacgtcaccgtatcccccagaccccccgaagcttcgggccccggggatctcgagggtcgtgtagtgtagggcggggttgatggcgaacacggggctgcatagcttgcggatgcgcgtgagggtgaggatgtgcgagggggacgaggggggtgcggttaacgccgcctgggatctgcgcaggggcgggcggttcagtttggccgccgtaccgggcgtctcgggggacgcgcggcgatgagacgagcggctcattcgccatcgggatagtcccgcgcgaagccgctcgcggaggccggatcggtggcgggacccgtgggaggagcgggagacggcggcgtcctggagagaggggccgctggggcgcccggaggccccgtgggggttggagtgtacgtaggatgcgagccaatccttgaaggaccgttggcgtgcaccttgggggctgaggttagctgccacatgaccagcaggtcgctgtctgcgggactcatccatccttcggccaggtcgccgtctccccacagagaagcgttggtcgctgcttcctcgagttgctcctcctggtccgcaagacgatcgtccacggcgtccaggcgctcaccaagcgccggatcgaggtaccgtcggtgtgcggttagaaagtcacgacgcgccgcttgctcctccacgcgaattttaacacaggtcgcgcgctgtcgcatcatctctaagcgcgcgcgggactttagccgcgcctccaattccaagtgggccgcctttgcagccataaaggcgccaacaaaccgaggatcttgggtgctgacgccctcccggtgcagctgcagggtctggtccttgtaaatctcggctcggaggtgcgtctcggccaggcgtcggcgcagggccgcgtgggcggcatctcggtccattccgccaccctgcgggcgacccggggggtgctctgatagtctcgcgtgcccaaggcccgtgatcggggtacttcgccgccgcgacccgccacccggtgtgcgcgatgtttggtcagcagctggcgtccgacgtccagcagtacctggagcgcctcgagaaacagaggcaacttaaggtgggcgcggacgaggcgtcggcgggcctcaccatgggcggcgatgccctacgagtgccctttttagatttcgcgaccgcgacccccaagcgccaccagaccgtggtccctggcgtcgggacgctccacgactgctgcgagcactcgccgctcttctcggccgtggcgcggcggctgctgtttaatagcctggtgccggcgcaactaaaggggcgtgatttcgggggcgaccacacggccaagctggaattcctggcccccgagttggtacgggcggtggcgcgactgcggtttaaggagtgcgcgccggcggacgtggtgcctcagcgtaacgcctactatagcgttctgaatacgtttcaggccctccaccgctccgaagcctttcgccagctggtgcactttgtgcgggactttgcccagctgctcaaaacctccttccgggcctccagcctcacggagaccacgggcccccccaaaaaacgggccaaggtggacgtggccacccacggccggacgtacggcacgctggagctgttccaaaaaatgatccttatgcacgccacctactttctggccgccgtgctcctcggggaccacgcggagcaggtcaacacgttcctgcgtctcgtgtttgagatccccctgtttagcgacgcggccgtgcgccacttccgccagcgcgccaccgtgtttctcgtcccccggcgccacggcaagacctggtttctggtgcccctcatcgcgctgtcgctggcctcctttcgggggatcaagatcggctacacggcgcacatccgcaaggcgaccgagccggtgtttgaggagatcgacgcctgcctgcggggctggttcggttcggcccgagtggaccacgttaaaggggaaaccatctccttctcgtttccggacgggtcgcgcagtaccatcgtgtttgcctccagccacaacacaaacgtaagtcctcttttctttcgcatggctctcccaaggggccccgggtcgacccgacccacacccacccacccacatacacacacaaccagacgcgggaggaaagtctgccccgtgggcactgatttttattcgggatcgcttgaggaggcccgggcaacggcccgggcaacggtggggcaactcgtagcaaataggcgactgatgtacgaagagaagacacacaggcgccacccggcgctggtcggggggatgttgtccgcgccgcaccgtcccccgacgacctcttgcagacggtccgtgatgcaaggacggcggggggcctgcagcagggtgaccgtatccacgggatggccaaagagaagcggacacaggctagcatccccctggaccgccagggtacactgggccatcttggcccacagacacggggcgacgcagggacaggactccgttacgacggaggagagccacagtgcgttggcggaatcgatgtggggcggcggggcgcaggactcgcagccccccgggtggttggtgatcctggccaggagccatcccagatggcgggccctgcttcccggtggacagagcgaccccaggtcgctgtccatggcccagcagtagatctggccgctggggaggtgccaccaggcccccgggcccaaggcgcagcacgcgcccggctccgggggggtcttcgcggggaccagatacgcgccatccagctcgccgaccactggctcctccgcgagctgttcggtggttgggtcgggggtttcctccgggggggtggccgcccgtatgcgtgcgaacgtgagggtgcacaggagcggggtcagggggtgcgtcacgctccggaggtggacgatcgcgcagtagcggcgctcgcggttaaagaaaaagagggcaaagaaggtgttcgggggcaaccgcagcgccttggggcgcgtcagatacagaaaaatctcgcagaagagggcgcgcccggggtctgggttaggaagggccacctgacacagaggctcggtgaggaccgttagacaccgaaagatcttgagccgctcgtccgcccgaacgacgcgccacacaaagacggagttgacaatgcgcgcgatagagtcgacgtccgtccccaggtcgtcgactctatcgcgcgtgccgcgagctccggcccgggaatccggccggggcaaggtccccgggggaccaggcggcgccaggggccgccggggtcccagctgcgccatgccgggggcggggggagggcaaaccccagaggcgggggccaacggcgcggggaggagtgggtgggcgaggtggccgggggaaggcgcccgctagcgagaccggccgttcccggacgacaccttgcgacaaaacctaaggacagcggcccgcgcgacggggtccgagaggctaaggtaggccgcgatgttaatggtgaacgcaaagccgccgggaaagacaactatgccacagaggcggcgattaaaccccaggcagaggtaggcgtagctttccccgggcaggtattgctcgcagaccctgcgtggggctgtggaggggacggcctccatgaagcgacatttactctgctcgcgtttactgacgtcaccatccatcgccacggcgattggacgattgttaagccgcagcgtgtctccgcttgtgctgtagtagtcaaaaacgtaatggccgtcggagtcggcaaagcgggccgggaggtcgtcgccgagcgggacgacccgccgcccccgaccgccccgtccccccaggtgtgccaggacggccagggcatacgcggtgtgaaaaaaggcgtcgggggcggtcccctcgacggcgcgcatcaggttctcgaggagaatggggaagcgcctggtcacctcccccaaccacgcgcgttggtcggggccaaagtcatagcgcaggcgctgtgagattcgcgggccgccctgaagcgcggcccggatggcctggcccagggcccggaggcacgccagatgtatgcgcgcggtaaaggcgacctcggcggcgatgtcaaagggcggcaggacggggcgcgggtggcgcaggggcacctcgagcgcgggaaagcgtagcagcagctccgcctgcccagcgggagacagctggtgggggcgcacgacgcgttctgcggcgcaggcctcggtcagggccgtggccagcgccgaggacagcagcggagggcgggcgcgtcgcccgccccacgccacggagttctcgtaggagacgacgacgaagcgctgcttggttccgtagtggtggcgcaggaccacggagatagaacgacggctccacagccagtccggccggtcgccgccggccagggcttcccatccgcgatccaaccactcgaccagcgaccgcggctttgcggtaccaggggtaagggttagaacgtcgttcaggatgtcctcgcccccgggcccgtggggcgctggggccacaaagcggcccccgccggggggctccagacccgccagcaccgcatctgcgtcagccgcccccatggcgcccccgctgacggcctggtgaaccagggcgccctggcgtagccccgatgcaacgccacaggccgcacgcccggtccgcgctcggaccgggtggcggcgggtgacgtcctgcactgcccgctgaaccaacgcgaggatctcctcgttctcctgtgcgatggacacgtcctgggccgcggtcgtgtcgccgccgggggccgtcagctgctcctccggggagatgggggggtcggacgccccgacgatgggcgggtctgcgggcgcccccgcgtggggccgggccaagggctgcggacgcggggacgcgctttcccccagacccatggacaggtgggccgcagcctccttcgcggccggcggggcggcggcgccaagcagagcgacgtagcggcacaaatgccgacagacgcgcatgatgcgcgtgctgtcggccgcgtagcgcgtgttgggggggacgagctcgtcgtaactaaacagaatcacgcgggcacagctcgcccccgagccccacgcaaggcgcagcgccgccacggcgtacgggtcatagacgccctgcgcgtcacacaccacgggcagggagacgaacaaccccccggcgctggacgcacgcggaaggaggccagggtgtgccggcacgacgggggccagaagctcccccaccgcatccgcgggcacgtaggcggcaaacgccgtgcaccacggggtacagtcgccggtggcatgagcccgagtctggatttcgacctggaagtttgcggccgtcccgagtccggggcggccgcgcatcagggcggccagagggattcccgcggccgccaggcactcgctggatatgatgacgtgaaccaaagaccgagggccgacccgggccgtggccgagatcgtctggacctcgttggccaagtgcgcgttcatggttcgggggtgggtgtgggtgtgtaggcgatgcgggtcccccgagtccgcgggaagggcgtgggtttggcgcgcgtatgcgtattcgccaacggaggcgtgcgtgcttatgcgcggcgcgtttcttctgtctctagggaatccgaggccaggactttaacctgctctttgtcgacgaggccaactttattcgcccggatgcggtccagacgattatgggctttctcaaccaggccaactgcaagattatcttcgtgtcgtccaccaacaccgggaaggccagtacgagctttttgtacaacctccgcggggccgcagacgagcttctcaacgtggtgacctatatatgcgatgatcacatgccgagggtggtgacgcacacaaacgccacggcctgttcttgttatatcctcaacaagcccgttttcatcacgatggacggggcggttcgccggaccgccgatttgtttctggccgattccttcatgcaggagatcatcgggggccaggccagggagaccggcgacgaccggcccgttctgaccaagtctgcgggggagcggtttctgttgtaccgcccctcgaccaccaccaacagcggcctcatggcccccgatttgtacgtgtacgtggatcccgcgttcacggccaacacccgagcctccgggaccggcgtcgctgtcgtcgggcggtaccgcgacgattatatcatcttcgccctggagcacttttttctccgcgcgctcacgggctcggcccccgccgacatcgcccgctgcgtcgtccacagtctgacgcaggtcctggccctgcatcccggggcgtttcgcggcgtccgggtggcggtcgagggaaatagcagccaggactcggccgtcgccatcgccacgcacgtgcacacagagatgcaccgcctactggcctcggagggggccgacgcgggctcgggccccgagcttctcttctaccactgcgagcctcccgggagcgcggtgctgtacccctttttcctgctcaacaaacagaagacgcccgcctttgaacactttattaaaaagtttaactccgggggcgtcatggcctcccaggagatcgtttccgcgacggtgcgcctgcagaccgacccggtcgagtatctgctcgagcagctaaataacctcaccgaaaccgtctcccccaacactgacgtccgtacgtattccggaaaacggaacggcgcctcggatgaccttatggtcgccgtcattatggccatctacctcgcggcccaggccggacctccgcacacattcgctcctatcacacgcgtctcgtgagcgcccaataaacacacccaggtatgctacgcacgaccacggtgtcgtctgttaaggggggggggggaagggggtgttggcgggaagcgtgggaacacgggggattctctcacgaccggcaccagtaccacccccctgtgaacacagaaaccccaacccaaatcccataaacatacgacacacaggcatattttggaatttcttaggtttttatttatttaggtatgctggggtttctccctggatgcccacccccacccccccgtgggtctagccgggccttagggatagcgtataacgggggccatgtctccggaccgcacaacggccgcgccgtcaaaggtgcacacccgaaccacgggagccagggccaaggtgtctcctagttggcccgcgtgggtcagccaggcgacgagcgcctcgtaaagcggcagccttcgctctccatcctgcatcagggccggggcttcggggtgaatgagctgggcggcctcccgcgtgacactctgcatctgcagtagagcgttcacgtacccgtcctgggcacttagcgcaaagagccgggggattagcgtaaggatgatggtggttccctccgtgatcgagtaaaccatgttaaggaccagcgatcgcagctcggcgtttacgggaccgagttgttggacgtccgccagcagcgagaggcgactcccgttgtagtacagcacgttgaggtctggcagccctccggggtttctggggctggggttcaggtcccggatgcccctggccacgagccgcgccacgatttcgcgcgccaggggcgatggaagcggaacgggaaaccgcaacgtgaggtccagcgaatccaggcgcacgtccgtcgcttggccctcgaacacgggcgggacgaggctgatggggtccccgttacagagatctacgggggaggtgttgcgaaggttaacggtgccggcgtgggtgaggcccacgtccagggggcaggcgacgattcgcgtgggaagcacccgggtgatgaccgcggggaagcgccttcggtacgccagcaacaaccccaacgtgtcgggactgacgcctccggagacgaaggattcgtgcgccacgtcggccagcgtcagttgccggcggatggtcggcaggaataccacccgcccttcgcagcgctgcagcgccgccgcatcggggcgcgagatgcccgagggtatcgcgatgtcagtttcaaagccgtccgccagcatggcgccgatccacgcggcagggagtgcagtggtggttcgggtggcgggaggagcgcggtgggggtcagcggcgtagcagagacgggcgaccaacctcgcataggacggggggtgggtcttagggggttgggaggcgacagggaccccagagcatgcgcggggaggtctgtcgggcccagacgcaccgagagcgaatccgtccgcggagtcccggcttgggttttatggggcccggccctcggaatcgcggcttgtcggcggggacaaagggggcggggctaggggcttgcggaaacagaagacgcgtgggataaaagaatcgcactaccccaaggaagggcggggcggtttattacagagccagtcccttgagcggggatgcgtcatagacgagatactgcgcgaagtgggtctcccgcgcgtgggcttccccgttgcgggcactgcggaggagggcggggtcgctggcgcaggtgagcgggtaggcctcctgaaacaggccacacgggtcctccacgagttcgcggcaccccggggggcgcttaaactgtacgtcgctggcggcggtggccgtggacaccgccgaacccgtctccacgatcaggcgctccaggcagcgatgtttggcggcgatgtcggccgacgtaaagaacttaaagcaggggctgagcaccggcgaggccccgttgaggtggtaggccccgttatagagcaggtccccgtacgaaaatcgctgcgacgcccacgggttggccgtggccgcgaaggcccgggacgggtcgctctggccgtggtcgtacatgagggcggtgacatccccctccttgtcccccgcgtaaacgcccccggcggcgcgtccccgggggttgcagggccggcggaagtagttgacgtcggtcgacacgggggtggcgataaactcacacacggcgtcctggccgtggtccatccctgcgcgccgcggcacctgggcgcacccgaacacggggacgggctgggccggccccaggcggtttcccgccacgaccgcgttccgcaggtacacggctgccgcgttgtccaggagagggggagccccgcggcccaggtaaaagttttggggaaggttgcccatgtcggtgacggggttgcggacggttgccgtggccacgacggcggtgtagcccacgcccaggtccacgttcgcgcgcggctgggtgagcgtgaagtttacccccccgccagtttcgtgccgggccacctggagctggcccaggaagtacgcctccgacgcgcgctccgagaacagcacgttctcagtcacaaagcggtcctgtcggacgacggtgaacccaaacccgggatggaggcccgtcttgagctgatgatgcaaggccacgggactgatcttgaagtaccccgccatgagcgcgtaggtcagcgcgttctccccggccgcgctctcgcggacgtgctgcacgacgggctgtcggatcgacgaaaagtagttggcccccagagccggggggaccagggggacctgccgcgacaggtcgcgcagggccggggggaaattgggcgcgttcgccacgtggtcggccccggcgaacagcgcgtggacggggagggggtaaaaatagtcgccattttggatggtatggtccagatgctggggggccatcagcaggattccggcgtgcaacgccccgtcgaatatgcgcatgttggtggtggacgcggtgttggcgcccgcgtcgggcgccgccgagcagagcagcgccgttgtgcgttcggccatgttgtgggccagcacctgcagcgtgagcatggcgggcccgtccactaccacgcgcccgttgtgaaacatggcgttgaccgtgttggccaccagattggccgggtgcagggggtgcgcggggtccgtcacggggtcgctggggcactcctcgccgggggcgatctccgggaccaccatgttctgcagggtggcgtatacgcggtcgaagcgaacccccgcggtgcagcagcggccccgcgagaaggcgggcaccatcacgtagtagtaaatcttgtggtgcacggtccagtccgccccccggtgcggccggtcatccgcggcgtccgcggctcgggcctgggtgttgtgcagcagctggccgtcgttgcggttgaagtccgcggtcgccacgttacatgccgccgcgtacacggggtcgtggccccccgcgctaacccggcagtcgcgatggcggtccagggccgcgcgccgcatcagggcgtcacagtcccacacgaggggtggcagcagcgccgggtctcgcattaggtgattcagctcggcttgcgcctgcccgcccagctccgggccggtcagggtaaagtcatcaaccagctgggccagggcctcgacgtgcgccaccaggtcccggtacacggccatgcactcctcgggaaggtctcccccgaggtaggtcacgacgtacgagaccagcgagtagtcgttcacgaacgccgcgcaccgcgtgttgttccagtagctggtgatgcactggaccacgagccgggccagggcgcagaagacgtgctcgctgccgtgtatggcggcctgcagcaggtaaaacaccgccgggtagttgcggtcgtcgaacgccccgcgaacggcggcgatggtggcgggggccatggcgtggcgtcccacccccagctccaggccccgggcgtcccggaacgccgccggacatagcgccaggggcaagttgccgttcaccacgcgccaggtggcctggatctcccccgggccggccgggggaacgtccccccccggcagctccacgtcggccacccccacaaagaagtcgaacgcggggtgcagctcaagagccaggttggcgttgtcgggctgcataaactgctccggggtcatctggccttccgcgacccatcggacccgcccgtgggccaggcgctgcccccaggcgttcaaaaacagctgctgcatgtctgcggcggggccggccggggccgccacgtacgccccgtacggattggcggcttcgacggggtcgcggttaaggcccccgaccgccgcgtcaacgttcatcagcgaagggtggcacacggtcccgatcgcgtgttccagagacaggcgcagcacctggcggtccttcccccaaaaaaacagctggcggggcgggaaggcgcggggatccgggtggccgggggcggggactaggtccccggcgtgcgcggcaaaccgttccatgaccggattgaacaggcccaggggcaggacgaacgtcaggtccatggcgcccaccagggggtagggaacgttggtggcggcgtagatgcgcttctccagggcctccagaaagaccagcttctcgccgatggacaccagatccgcgcgcacgcgcgtcgtctggggggcgctctcgagctcgtccagcgtctgccggttcaggtcgagctgctcctcctgcatctccagcaggtggcggcccacgtcgtccagacttcgcacggccttgcccatcacgagcgccgtgaccaggttggccccgttcaggaccatctcgccgtacgtcaccggcacgtcggcttcggtgtcctccactttcaggaaggactgcaggaggcgctgtttgatcggggcggtggtgacgagcaccccgtcgaccggccgcccgcgcgtgtcggcatgcgtcagacggggcacggccacggagggctgcgtggccgtggtgaggtccacgagccaggcctcgacggcctcccggcggtggcccgccttgcccaggaaaaagctcgtctcgcagaagcttcgctttagctcggcgaccagggtcgcccgggccaccctggtggccaggcggccgttgtccaggtatcgttgcatcggcaacaacaaagccaggggcggcgccttttccagcagcacgtgcagcatctggtcggccgtgccgcgctcaaacgccccgaggacggcctggacgttgcgagcgagctgttggatggcgcgcaactggcgatgcgcgccgatacccgtcccgtccagggcctcccccgtgagcagggcgatggcctcggtggccaggctgaaggcggcgttcagggcccggcggtcgataatcttggtcatgtaattgtgtgtgggttgctcgatggggtgcgggccgtcgcgggcaatcagcggctggtggacctcgaactgtacgcgcccctcgttcatgtaggccagctccggaaacttggtacacacgcacgccaccgacaacccgagctccagaaagcgcacgagcgacagggtgttgcaatacgaccccagcagggcgtcgaactcgacgtcgtacaggctgtttgcatcggagcgcacgcgggaaaaaaaatcaaacaggcgtcgatgcgacgccacctcgatcgtgctaaggagggacccggtcggcaccatggccgcggcataccggtatcccggagggtcgcggttgggagcggccatggggtcgcgtggagatcggctgtctctagcgatattggcccggggaggctaagatccaccccaacgcccggccacccgtgtacgtgcccgacggcccaaggtccaccgaaagacacgacgggcccggacccaaaaaggcgggggatgctgtgtgagaggccgggtgccggtcgggggggaaaggcaccgggagaaggctgcggcctcgttccaggagaacccagtgtccccaacagacccggggacgtgggatcccaggccttatatacccccccccccgccccacccccgttagaacgcgacgggtgcattcaagatggccctggtccaaaagcgtgccaggaagaaattggcagaggcggcaaagctgtccgccgccgccacccacatcgaggccccggccgcgcaggctatccccagggcccgtgtgcgcaggggatcggtgggcggcagcatttggttggtggcgataaagtggaaaagcccgtccggactgaaggtctcgtgggcggcggcgaacaaggcacacagggccgtgcctcccaaaaacacggacatcccccaaaacacgggcgccgacaacggcagacgatccctcttgatgttaacgtacaggaggagcgcccgcaccgcccacgtaacgtagtagccgacgatggcggccaggatacaggccggcgccaccacccttccggtcagcccgtaatacatgcccgctgccaccatctccaacggcttcaggaccaaaaacgaccaaaggaacagaatcacgcgctttgaaaagaccggctgggtatggggcggaagacgcgagtatgccgaactgacaaaaaaatcagaggtgccgtacgaggacaatgaaaactgttcctccagcggcagttctccctcctcccccccgaaggcggcctcgtcgaccagatctcgatccaccagaggaaggtcatcccgcatggtcatggggtgtgcggtggaggtggggagaccgaaaccgcaaagggtcgcttacgtcagcaggatcccgagatcaaagacacccgggttcttgcacaaacaccacccgggttgcatccgcggaggcgagtgttttgataaggccgttccgcgccttgatataacctttgatgttgaccacaaaacccggaatttacgcctacgccccaatgcccacgcaagatgaggtaggtaacccccccgtgggtgtgacgttgcgtttagttcattggaggccaaggggaaaaatggggtggggaggaaacggaaaacccagtaggccgtgtcgggaacacgcccggggttgtcctcaaaaggcagggtccatactacggaagccgtcgttgtattcgagacctgcctgtgcaacgcacgtcggggttgcctgtgtccggttcggcccccaccgcgtgcggcacgcacgaggacgagtccgcgtgctttattggcgttccaagcgttgccctccagtttctgttgtcggtgttcccccatacccacgcccacatccaccgtagggggcctctgggccgtgttacgtcgccgcccgcgatggagcttagctacgccaccaccatgcactaccgggacgttgtgttttacgtcacaacggaccgaaaccgggcctactttgtgtgcggggggtgtgtttattccgtggggcggccgtgtgcctcgcagcccggggagattgccaagtttggtctggtcgttcgagggacaggcccagacgaccgcgtggtcgccaactatgtacgaagcgagctccgacaacgcggcctgcaggacgtgcgtcccattggggaggacgaggtgtttctggacagcgtgtgtcttctaaacccgaacgtgagctccgagctggatgtgattaacacgaacgacgtggaagtgctggacgaatgtctggccgagtactgcacctcgctgcgaaccagcccgggtgtgctaatatccgggctgcgcgtgcgggcgcaggacagaatcatcgagttgtttgaacacccaacgatagtcaacgtttcctcgcactttgtgtataccccgtccccatacgtgttcgccctggcccaggcgcacctcccccggctcccgagctcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgactcgagttccctggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctagccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctgtttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagccacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgtctcgctcgctcccaacacggacagtctgtgtaaaagaccccaataaacgtatatcgctactacacccttgtgtgtcaatggacgcctctccgggggggggggagggaaagcaaagaggggctgggggagcggcaccaccggggcctgaacaaacaaaccacagacacggttacagtttattcggtcgggcggagaaacggccgaagccacgcccactttattcgcgtctccaaaaaaacgggacacttgtccggagaacctttaggatgccagccagggcggcggtaatcataaccacgcccagcgcagaggcggccagaaacccgggcgcaattgcggccacgggctgcgtgtcaaaggctagcaaatgaatgacggttccgtttggaaatagcaacaaggccgtggacggcacgtcgctcgaaaacacgcttggggcgccctccgtcggcccggcggcgatttgctgctgtgtgttgtccgtatccaccagcaacacagacatgacctccccggccggggtgtagcgcataaacacggcccccacgagccccaggtcgcgctggttttgggtgcgcaccagccgcttggactcgatatcccgggtggagccttcgcatgtcgcggtgaggtaggttaggaacagtgggcgtcggacgtcgacgccggtgagcttgtagccgatcccccggggcagaggggagtgggtgacgacgtagctggcgttgtgggtgatgggtaccaggatccgtggctcgacgttggcagactgccccccgcaccgatgtgaggcctcagggacgaaggcgcggatcagggcgttgtagtgtgcccaacgcgtcagggtcgaggcgaggccgtgggtctgctgggccaggacttcgaccggggtctcggatcgggtggcttgagccagcgcgtccaggataaacacgctctcgtctagatcaaagcgcagggaggccgcgcatggcgaaaagtggtccggaagccaaaagagggttttctggtggtcggcccgggccagcgcggtccggaggtcggcgttggtcgctgcggcgacgtcggacgtacacagggccgaggctatcagaaggctccggcgggcgcgttcccgctgcaccgccgaggggacgccagccaagaacggctgccggaggacagccgaggcgtaaaatagcgcccggtggacgaccggggtggtcagcacgcggccccctagaaactcggcatacagggcgtcgatgagatgggctgcgctgggcgccactgcgtcgtacgccgaggggctatccagcacgaaggccagctgatagcccagcgcgtgtaatgccaagctctgttcgcgctccagaatctcggccaccaggtgctggagccgagcctctagctgcaggcgggccgtgggatccaagactgacacattaaaaaacacagaatccgcggcacagcccgcggccccgcgggcggccaacccggcaagcgcgcgcgagtgggccaaaaagcctagcaggtcggagaggcagaccgcgccgtttgcgtgggcggcgttcacgaaagcaaaacccgacgtcgcgagcagccccgttaggcgccagaagagaggggggcgcgggccctgctcggcgcccgcgtcccccgagaaaaactccgcgtatgcccgcgacaggaactgggcgtagttcgtgccctcctccgggtagccgcccacgcggcggagggcgtccagcgcggagccgttgtcggcccgcgtcagggaccctaggacaaagacccgataccgggggccgcccgggggcccgggaagagcccccggggggttttcgtccgcggggtccccgacccgatctagcgtctggcccgcggggaccaccatcacttccaccggagggctgtcgtgcatggatatcacgagccccatgaattcccgcccgtagcgcgcgcgcaccagcgcggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggtcgggggggcggtgtatcacgcggcccgctcgggtggctcgccgtcgccacgttgtctccccgcgggaacgtcagggcctcggggtcagggacggccgaaaacgttacccaggcccgggaacgcagcaacacggaggcggctggattgtgcaagagacccttaaggggggcgaccgaggggggaggctgggcggtcggctcgaccgtggtgggggcgggcaggctcgcgttcgggggccggccgagcaggtaggtcttcgggatgtaaagcagctggccggggtcccgcggaaactcggccgtggtgaccaatacaaaacaaaagcgctcctcgtaccagcgaagaaggggcagagatgccgtagtcaggtttagttcgtccggcggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggcccgtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggcagccggcgggcgattggtcgtaatccaggataaagacatgcatgggacggaggcgtttggccaagacgtccaaagcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgcccccgaaagctgtccccaatcctcccgccacgacccgccgccctgcagataccgcaccgtattggcaagcagcccataaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccaacacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccaggcggacttccgtggctttttgttgccggcgagggcgcaacgccgtacgtcggttgttatggccgcgagaacgcgcagcctggtcgaacgcagacgcgtgttgatggcaggggtacgaagccatacgcgcttctacaaggcgctggccgaagaggtgcgggagtttcacgccaccaagatctgcggcacgctgttgacgctgttaagcgggtcgctgcagggtcgctcggtattcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccattgagcctaggacccagcgagcccgtcgccgccgcggcggcgccgcccgggggtctgcgagcagaccgaaaaggtcacactctggggcgcgcgacccgcccgagtcagcggcccgccagttaccacccgccgaccaaacccccacctccacggagggcgggggggtgcttaagaggatcgcggcgctcttctgcgtgcccgtggccaccaagaccaaaccccgagccgcctccgaatgagagtgtttcgttccttccccctccccccgcgtcagacaaaccctaaccaccgcttaagcggcccccgcgaggtccgaagactcatttggatccggcgggagccacccgacaacagcccccgggttttcccacgccagacgccggtccgctgtgccatcgcgccccctcatcccaccccccatcttgtccccaaataaaacaaggtctggtagttaggacaacgaccgcagttctcgtgtgttattttcgctctccgcctctcgcagatggacccgtactgcccatttgacgctctggacgtctgggaacacaggcgcttcatagtcgccgattcccgaaacttcatcacccccgagttcccccgggacttttggatgtcgcccgtctttaacctcccccgggagacggcggcggagcaggtggtcgtcctacaggcccagcgcacagcggctgccgctgccctggagaacgccgccatgcaggcggccgagctccccgtcgatatcgagcgccggttacgcccgatcgaacggaacgtgcacgagatcgcaggcgccctggaggcgctggagacggcggcggccgccgccgaagaggcggatgccgcgcgcggggatgagccggcgggtgggggcgacgggggggcgcccccgggtctggccgtcgcggagatggaggtccagatcgtgcgcaacgacccgccgctacgatacgacaccaacctccccgtggatctgctacacatggtgtacgcgggccgcggggcgaccggctcgtcgggggtggtgttcgggacctggtaccgcactatccaggaccgcaccatcacggactttcccctgaccacccgcagtgccgactttcgggacggccgtatgtccaagaccttcatgacggcgctggtactgtccctgcaggcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcggtgatcgggaccgagggcggccggccacagtaccgctaccgcgacgacaagctccccaagacgcagttcgcggccggcgggggccgctacgaacacggagcgctggcgtcgcacatcgtgatcgccacgctgatgcaccacggggtgctcccggcggccccgggggacgtcccccgggacgcgagtacccacgttaaccccgacggcgtggcgcaccacgacgacataaaccgcgccgccgccgcgttcctcagccggggccacaacctattcctgtgggaggaccagactctgctgcgggcaaccgcgaacaccataacggccctgggcgttatccagcggctcctcgcgaacggcaacgtgtacgcggaccgcctcaacaaccgcctgcagctgggcatgctgatccccggagccgtcccttcggaggccatcgcccgtggggcctccgggtccgactcgggggccatcaagagcggagacaacaatctggaggcgctatgtgccaattacgtgcttccgctgtaccgggccgacccggcggtcgagctgacccagctgtttcccggcctggccgccctgtgtcttgacgcccaggcggggcggccggtcgggtcgacgcggcgggtggtggatatgtcatcgggggcccgccaggcggcgctggtgcgcctcaccgccctggaactcatcaaccgcacccgcacaaaccccacccctgtgggggaggttatccacgcccacgacgccctggcgatccaatacgaacaggggcttggcctgctggcgcagcaggcacgcattggcttgggctccaacaccaagcgtttctccgcgttcaacgttagcagcgactacgacatgttgtactttttatgtctggggttcattccacagtacctgtcggcggtttagtgggtggtgggcgaggggggagggggcattagggagaaaaaacaagagcctccgttgggttttctttgtgcctgtactcaaaaggtcataccccgtaaacggcgggctccagtcccggcccggcggttggcgtgaacgcaacggcgggagctgggttagcgtttagtttagcattcgctctcgcctttccgcccgccccccgaccgttgcgcctttttttttttcgtccaccaaagtctctgtgggtgcgcgcatggcagccgatgccccgggagaccggatggaggagcccctgcccgacagggccgtgcccatttacgtggctgggtttttggccctgtatgacagcggggactcgggcgagttggcattggatccggatacggtgcgggcggccctgcctccggataacccactcccgattaacgtggaccaccgcgctggctgcgaggtggggcgggtgctggccgtggtcgacgacccccgcgggccgttttttgtggggctgatcgcctgcgtgcagctggagcgcgtcctcgagacggccgccagcgctgcgattttcgagcgccgcgggccgccgctctcccgggaggagcgcctgttgtacctgatcaccaactacctgccctcggtctccctggccacaaaacgcctggggggcgaggcgcaccccgatcgcacgctgttcgcgcacgtcgcgctgtgcgcgatcgggcggcgcctcggcactatcgtcacctacgacaccggtctcgacgccgccatcgcgccctttcgccacctgtcgccggcgtctcgcgagggggcgcggcgactggccgccgaggccgagctcgcgctgtccgggcgcacctgggcgcccggcgtggaggcgctgacccacacgctgctttccaccgccgttaacaacatgatgctgcgggaccgctggagcctggtggccgagcggcggcggcaggccgggatcgccggacacacctacctccaggcgagcgaaaaattcaaaatgtggggggcggagcctgtttccgcgccggcgcgcgggtataagaacggggccccggagtccacggacataccgcccggctcgatcgctgccgcgccgcagggtgaccggtgcccaatcgtccgtcagcgcggggtcgccttgtccccggtactgccccccatgaaccccgttccgacatcgggcaccccggcccccgcgccgcccggcgacgggagctacctgtggatcccggcctcccattacaaccagctcgtcgccggccatgccgcgccccaaccccagccgcattccgcgtttggtttcccggctgcggcggggtccgtggcctatgggcctcacggtgcgggtctttcccagcattaccctccccacgtcgcccatcagtatcccggggtgctgttctcgggacccagcccactcgaggcgcagatagccgcgttggtgggggccatagccgcggaccgccaggcgggcggtcagccggccgcgggagaccctggggtccgggggtcgggaaagcgtcgccggtacgaggcggggccgtcggagtcctactgcgaccaggacgaaccggacgcggactacccgtactaccccggggaggctcgaggcgcgccgcgcggggtcgactcccggcgcgcggcccgccattctcccgggaccaacgagaccatcacggcgctgatgggggcggtgacgtctctgcagcaggaactggcgcacatgcgggctcggaccagcgccccctatggaatgtacacgccggtggcgcactatcgccctcaggtgggggagccggaaccaacaacgacccacccggccctttgtcccccggaggccgtgtatcgccccccaccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaataagacccattggtgtatgttctttttttattgggaggcgcgggtaggcgggtagctttacaatgcaaaagccttcgacgtggaggaaggcgtggggggggggggaatcggcactgaccaagggggtccgttttgtcacgggaaaggaaagaggaaacaggccgcggacacccgggggagtttgtgttcccttttctttcttcccacacacacaaaaggcgtaccaaacaaacaaaccaaaagatgcacatgcggtttaacacccgtggtttttatttacaacaaaccccccatcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggcagacaccagggccatgtaccgtatcatctcccgggcctcggctagcttggcctcgtcaaagtcgccgccctcctcgccctccccggacgcgtccgggttggtggggttcttgagctccttggtggttagcgggtacagggccttcatggggttgctctgcagccgcatgacgtagcgaaaggcgaagaaggccgccgccaggccggccaggaccaacagacccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcaaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggtgatgttgaggtcgatgaaggtgctgacggtggtgatgtcggcgcggctcagctggtgggagtacgcgtactcctcgaagtacacgtagcccccaccgaaggtgaagtagcgccggtgtcccacggtgcacggctcgatcgcatcgcgcgtcagccgcagctcgttgttctcccccagctgcccctcgaccaacgggccctggtcttcgtaccgaaagctgaccagggggcggctgtagcaggccccgggccgcgagctgatgcgcatcgagttttggacgatcacgttgtccgcggcgaccggcacgcacgtggagacggccatcacgtcgccgagcatccgcgcgctcacccgccggcccacggtggccgaggcgatggcgttggggttcagcttgcgggcctcgttccacagggtcagctcgtgattctgcagctcgcaccacgcgatggcaacgcggcccaacatatcgttgacatggcgctgtatgtggttgtacgtaaactgcagcctggcgaactcgatggaggaggtggtcttgatgcgctccacggacgcgttggcgctggccccgggcggcgggggcgtggggtttgggggcttgcggctctgctcgcggaggtgttcccgcacgtacagctccgcgagcgtgttgctgagaaggggctggtacgcgatcagaaagcccccattggccaggtagtactgcggctggcccaccttgatgtgcgtcgcgttgtacctgcgggcgaagatgcggtccatggcgtcgcgggcgtccttgccgatgcagtcccccaggtccacgcgcgagagcgggtactcggtcaggttggtggtgaaggtggtggatatggcgtcggaagagaatcggaaggagccgccgtactcggagcgcagcatctcgtccacctcctgccacttggtcatggtgcagaccgacgggcgctttggcacccagtcccaggccacggtgaacttgggggtcgtgagcaggttccgggtggtcggcgccgtggcccgggccttggtggtgaggtcgcgcgcgtagaagccgtcgacctgcttgaagcggtcggcggcgtagctggtgtgttcggtgtgcgacccctcccggtagccgtaaaacggggacatgtacacaaagtcgccagtcgccaacacaaactcgtcgtacgggtacaccgagcgcgcgtccacctcctcgacgatgcagtttaccgtcgtcccgtaccggtggaacgcctccacccgcgaggggttgtacttgaggtcggtggtgtgccagccccggctcgtgcgggtcgcggcgttggccggtttcagctccatgtcggtctcgtggtcgtcccggtgaaacgcggtggtctccaggttgttgcgcacgtacttggccgtggaccgacagacccccttggcgttgatcttgtcgatcacctcctcgaaggggacgggggcgcggtcctcaaagatccccataaactgggagtagcggtggccgaaccacacctgcgaaacggtgacgtctttgtagtacatggtggccttgaacttgtacggggcgatgttctccttgaagaccaccgcgatgccctccgtgtagttctgaccctcgggccgggtcgggcagcggcgcggctgctcgaactgcaccaccgtggcgcccgtggggggtgggcacacgtaaaagtttgcatcggtgttctccgccttgatgtcccgcaggtgctcgcgcagggtggcgtggcccgcggcgacggtcgcgttgtcgccggcggggcgcggcggctttgggggtttcggttttctgttcttcttcggtttcgggtcccccgttgggggggcgccaggggcgggcggcgccggagtggcagggcccccgttcgccgcctgggtcgcggccgcgaccccaggcgtgccgggggaactcggagccgccgacgccaccaggacccccagcgtcaaccccaagagcgcccatacgacgaaccaccggcgcccccgcgcgggggcgccctggcgcatggcgggactacgggggcccgtcgtgccccccgtcaggtagcctgggggcgaggtgctggaggaccgagtagaggatcgagaaaacgtctcggtcgtagaccacgaccgaccgggggccgatacagccgtcgggggcgctctcgacgatggccaccagcggacagtcggagtcgtacgtgagatatacgccgggcgggtaacggtaacgaccttcggaggtcgggcggctgcagtccgggcggcgcaactcgagctccccgcaccggtagaccgaggcaaagagtgtggtggcgataatcagctcgcgaatatatcgccaggcggcgcgctgagtgggcgttattccggaaatgccgtcaaaacagtaaaacctctgaaattcgctgacggcccaatcagcacccgagccccccgcccccatgatgaaccgggcgagctcctccttcaggtgcggcaggagccccacgttctcgacgctgtaatacagcgcggtgttggggggctgggcgaagctgtgggtggagtgatcaaagaggggcccgttgacgagctcgaagaagcgatgggtgatgctggggagcagggccgggtccacctggtgtcgcaggagagacgctcgcatgaaccggtgcgcgtcgaacacgcccggcgccgagcggttgtcgatgaccgtgcccgcgcccgccgtcagggcgcagaagcgcgcgcgcgccgcaaagccgttggcgaccgcggcgaacgtcgcgggcagcacctcgccgtggacgctgacccgcagcatcttctcgagctccccgcgctgctcgcggacgcagcgccccaggctggccaacgaccgcttcgtcaggcggtccgcgtacagccgccgtcgctcccgcacgtccgcggccgcttgcgtggcgatgtccccccacgtctcgggcccctgccccccgggcccgcggcgacggtcttcgtcctcgcccccgcccccgggagctcccaacccccgtgccccttcctctacggcgacacggtccccgtcgtcgtcggggcccgcgccgcccttgggcgcgtccgccgcgccccccgcccccatgcgcgccagcacgcgacgcagcgcctcctcgtcgcactgttcggggctgacgaggcgccgcaagagcggcgtcgtcaggtggtggtcgtagcacgcgcggatgagcgcctcgatctgatcgtcgggtgacgtggcctgaccgccgattattagggcgtccaccatatccagcgccgccaggtggctcccgaacgcgcgatcgaaatgctccgcccgccgcccgaacagcgccagttccacggccaccgcggcggtctcctgctgcaactcgcgccgcgccagcgcggtcaggttgctggcaaacgcgtccatggtggtctggccggcgcggtcgccggacgcgagccagaatcgcaattcgctgatggcgtacaggccgggcgtggtggcctgaaacacgtcgtgcgcctccagcagggcgtcggcctccttgcggaccgagtcgttctcgggcgacgggtggggctgcccgtcgccccccgcggtccgggccagcgcatggtccaacacggagagcgcccgcgcgcggtcggcgtccgacagcccggcggcgtggggcaggtaccgccgcagctcgttggcgtccagccgcacctgcgcctgctgggtgacgtggttacagatacggtccgccaggcggcgggcgatcgtcgccccctggttcgccgtcacacacagttcctcgaaacagaccgcgcaggggtgggacgggtcgctaagctccggggggacgataaggcccgaccccaccgcccccaccataaactcccgaacgcgctccagcgcggcggtggcgccgcgcgagggggtgatgaggtggcagtagtttagctgctttagaaagttctcgacgtcgtgcaggaaacacagctccatatggacggtcccgccatacgtatccagcctgacccgttggtgatacggacagggtcgggccaggcccatggtctccgtgaaaaacgccgcgacgtctcccgcggtcgcgaacgtctccaggctgcccaggagccgctcgccctcgcgccacgcgtactctagcagcaactccagggtgaccgacagcggggtgagaaaggccccggcctgggcctccaggcccggcctcagacgacgccgcagcgcccgcacctgaagcgcgttcagcttcagttgggggagcttcccccgtccgatgtgggggtcgcaccgccggagcagctctatctgaaacacataggtctgcacctgcccgagcagggctaacaacttttgacgggccacggtgggctcggacaccggggcggccatctcgcggcgccgatctgtaccgcggccggagtatgcggtggaccgaggcggtccgtacgctacccggcgtctggctgagccccggggtccccctcttcggggcggcctcccgcgggcccgccgaccggcaagccgggagtcggcggcgcgtgcgtttctgctctattcccagacaccgcggagaggaatcacggcccgcccagagatatagacacggaacacaaacaagcacggatgtcgtagcaataatttattttacacacattccccgccccgccctaggttcccccaccccccaacccctcacagcatatccaacgtcaggtctccctttttgtcggggggcccctccccaaacgggtcatccccgtggaacgcccgtttgcggccggcaaatgccggtcccggggcccccgggccgccgaacggcgtcgcgttgtcgtcctcgcagccaaaatccccaaagttaaacacctccccggcgttgccgagttggctgactagggcctcggcctcgtgcgccacctccagggccgcgtccgtcgaccactcgccgttgccgcgctccagggcacgcgcggtcagctccatcatctcctcgcttaggtactcgtcctccaggagcgccagccagtcctcgatctgcagctgctgggtgcggggccccaggcttttcacggtcgccacgaacacgctactggcgacggccgccccgccctcggagataatgccccggagctgctcgcacagcgagctttcgtgcgctccgccgccgaggcttgaggccgcgcacacaaacccggcccggggacaggccaggacgaacttgcgggtgcggtcaaaaataaggagcgggcacgcgtttttgccgcccatcaggctggcccagttcccggcctgaaacacacggtcgttgccggccatgccgtagtacttgctgatgctcaaccccaacacgaccatggggcgcgccgccatgacgggccgcagcaggttgcagctggcgaacatggacgtccacgcgcccggatgcgcgtccacggcgtccatcagcgcgcgggccccggcctccaggcccgccccgccctgcgcggaccacgcggccgcagcctgcacgctggggggacggcgggaccccgcgatgatggccgtaagggtgttgatgaagtatgtcgagtgatcgcagtaccgcagaatctggtttgccatgtagtacatcgccagctcgctcacgttgttgggggccaggttaataaagtttatcgcgccgtagtccagggaaaactttttaatgaacgcgatggtctcgatgtcctcgcgcgacaggagccgggcgggaagctggttgcgttggagggccgtccagaaccactgcgggttcggctggttggaccccgggggcttgccgttggggaagatggccgcgtggaactgcttcagcagaaagcccagcggtccgaggaggatgtccacgcgcttgtcgggcttctggtaggcgctctggaggctggcgacccgcgccttggcggcctcggacgcgttggcgctcgcgcccgcgaacaacacgcggctcttgacgcgcagctccttgggaaaccccagggtcacgcgggcaacgtcgccctcgaagctgctctcggcgggggccgtctggccggccgttaggctgggggcgcagatagccgccccctccgagagcgcgaccgtcagcgttttggccgacagaaacccgttgttaaacatgtccatcacgcgccgccgcagcaccggttggaattgattgcgaaagttgcgcccctcgaccgactgcccggcgaacaccccgtggcactgactcagggccaggtcctggtacacggcgaggttggatcgccgcccgagaagctgaagcagggggcacggcccgcacgcgtacgggtccagcgtcagggacatggcgtggttggcctcgcccagaccgtcgcgaaacttgaagttcctcccctccaccaggttgcgcatcagctgctccacctcgcggtccacgacctgcctgacgttgttcaccaccgtatgcagggcctcgcggttggtgatgatggtctccagccgccccatggccgtggggaccgcctggtccacgtactgcagggtctcgagttcggccatgacgcgctcggtcgccgcgcggtacgtctcctgcatgatggtccgggcggtctcggatccgtccgcgcgcttcagggccgagaaggcggcgtagtttcccagcacgtcgcagtcgctgtacatgctgttcatggtcccgaagacgccgatggctccgcgggcggcgctggcgaactttggatggcgcgcccggaggcgcatgagcgtcgtgtgtacgcaggcgtggcgcgtgtcgaaggtgcataggttacagggcacgtcggtctggttggagtccgcgacgtatcgaaacacgtccatctcctggcgcccgacgatcacggcgccgtcgcagcgctccaggtaaaacagcatcttggccagcagcgccggggaaaacccacacagcatggccaggtgctcgccggcaaattcctgggttccgccgacgaggggcgcggtgggccgaccctcgaacccgggcaccacgtgtccctcgcggtccacctgtgggttggccgccacgtgggtcccgggcacgaggaagaagcggtaaaaggagggtttgctgtggtcctttgggtccgccgggccggcgtcgtccacctcggtgagatggagggccgagttggtgctaaataccatggcccccacgagtcccgcggcgcgcgccaggtacgccccgacggcgttggcgcgggccgcggccgtgtcctggccctcgaacagcggccacgcggagatgtcggtgggcggctcgtcaaagacggccatcgacacgatagactcgagggccagggcggcgtctccggccatgacggaggccaggcgctgttcgaacccgcccgccgcgcccttgccgccgccgtcgcgcccgccccgcggggtcttaccctggctggcttcgaaggccgtgaacgtaatgtcggcggggagggcggcgccctcgtggttttcgtcaaacgccaggtgggcggccgcgcgggccacggcgtccacgtttcggcatcgcagtgccacggcggcgggtcccacgaccgcctcgaacaggaggcggttgagggggcggttaaaaaacggaagcgggtaggtaaatttctccccgatcgatcggtggttggcgttgaacggctctgcgatgacacggctaaaatccggcatgaacagctgcaacgggtacacgggtatgcggtgcacctccgccccgcctatggttaccttgtccgagcctcccaggtgcagaaaggtgttgttgatgcacacggcctccttgaagccctcggtaacgaccagatacaggagggcgcggtccgggtccaggccgaggcgctcacacagcgcctcccccgtcgtctcgtgtttgaggtcgccgggccggggggtgtagtccgaaaagccaaaatggcggcgtgcccgctcgcaaagtcgcgtcaggttcggggcctgggtgctggggtccaggtgccggccgccgtgaaagacgtacacggacgagctgtagtgcgagggcgtcagtttcagggacaccgcggtacccccgagccccgtcgtgcgagaacccacgaccacggccacgttggcctcaaagccgctctccacggtcaggcccacgaccaggggcgccacggcgacgtcggaatcgccgctgcgtgccgacagtaacgccagaagctcgatgccttcggacggacacgcgcgagcgtacacgtatcccaggggcccgggggggaccttgatggtggttgccgtcttgggctttgtctccatgtccttttgtcaatcggtccgcgaacggaggtaatcccggcacgacgacggacgcccgacaaggtatgtctcccgagcgtcaaaatccgggggggggcggcgacggtcaaggggagggttggagaccggggttggggaatgaatccctccccttcaccgacaaccccccgggtaaccacggggtcgccgatgaaccccggcggccggcaacgcggggtccctgcgagaggcacagatgcttacggtcaggtgctccgggtcgggtgcgtctggtatgcggttggtatatgtacactttacctgggggcgtgccggtccgccccagcccctcccacgccccgcgcgtcatcagccggtgggcgtggccgctattataaaaaaagtgagaacgcgaagcgttcgcactttgtcctaataatatatatattattaggacaaagtgcgaacgcttcgcgttctcactttttttataatagcggccacgcccaccggctacgtcactctcctgtcggccgccggcggtccataagcccggccggccgggccgacgcgaataaaccgggccgccggccggggcgccgcgcagcagctcgccgcccggatccgccagacaaacaaggcccttgcacatgccggcccgggcgagcctgggggtccggtaattttgccatcccacccaagcggctttttgggtttttctcttcccccctccccacattcccctctttaggggttcgggtgggaacaaccgcgatgttttccggtggcggcggcccgctgtcccccggaggaaagtcggcggccagggcggcgtccgggttttttgcgcccgccggccctcgcggagccagccggggacccccgccttgtttgaggcaaaacttttacaacccctacctcgccccagtcgggacgcaacagaagccgaccgggccaacccagcgccatacgtactatagcgaatgcgatgaatttcgattcatcgccccgcgggtgctggacgaggatgcccccccggagaagcgcgccggggtgcacgacggtcacctcaagcgcgcccccaaggtgtactgcgggggggacgagcgcgacgtcctccgcgtcgggtcgggcggcttctggccgcggcgctcgcgcctgtggggcggcgtggaccacgccccggcggggttcaaccccaccgtcaccgtctttcacgtgtacgacatcctggagaacgtggagcacgcgtacggcatgcgcgcggcccagttccacgcgcggtttatggacgccatcacaccgacggggaccgtcatcacgctcctgggcctgactccggaaggccaccgggtggccgttcacgtttacggcacgcggcagtacttttacatgaacaaggaggaggtcgacaggcacctacaatgccgcgccccacgagatctctgcgagcgcatggccgcggccctgcgcgagtccccgggcgcgtcgttccgcggcatctccgcggaccacttcgaggcggaggtggtggagcgcaccgacgtgtactactacgagacgcgccccgctctgttttaccgcgtctacgtccgaagcgggcgtgtgctgtcgtacctgtgcgacaacttctgcccggccatcaagaagtacgagggtggggtcgacgccaccacccggttcatcctggacaaccccgggttcgtcaccttcggctggtaccgtctcaaaccgggccggaacaacacgctagcccagccggcggccccgatggccttcgggacatccagcgacgtcgagtttaactgtacggcggacaacctggccatcgaggggggcatgagcgacctaccggcatacaagctcatgtgcttcgatatcgaatgcaaggcggggggggaggacgagctggcctttccggtggccgggcacccggaggacctggtcatccagatatcctgtctgctctacgacctgtccaccaccgccctggagcacgtcctcctgttttcgctcggttcctgcgacctccccgaatcccacctgaacgagctggcggccaggggcctgcccacgcccgtggttctggaattcgacagcgaattcgagatgctgttggccttcatgacccttgtgaaacagtacggccccgagttcgtgaccgggtacaacatcatcaacttcgactggcccttcttgctggccaagctgacggacatttacaaggtccccctggacgggtacggccgcatgaacggccggggcgtgtttcgcgtgtgggacataggccagagccacttccagaagcgcagcaagataaaggtgaacggcatggtgaacatcgacatgtacgggattataaccgacaagatcaagctctcgagctacaagctcaacgccgtggccgaagccgtcctgaaggacaagaagaaggacctgagctatcgcgacatccccgcctactacgccgccgggcccgcgcaacgcggggtgatcggcgagtactgcatacaggattccctgctggtgggccagctgttttttaagtttttgccccatctggagctctcggccgtcgcgcgcttggcgggtattaacatcacccgcaccatctacgacggccagcagatccgcgtctttacgtgcctgctgcgcctggccgaccagaagggctttattctgccggacacccaggggcgatttaggggcgccgggggggaggcgcccaagcgtccggccgcagcccgggaggacgaggagcggccagaggaggagggggaggacgaggacgaacgcgaggagggcgggggcgagcgggagccggagggcgcgcgggagaccgccggcaggcacgtggggtaccagggggccagggtccttgaccccacttccgggtttcacgtgaaccccgtggtggtgttcgactttgccagcctgtaccccagcatcatccaggcccacaacctgtgcttcagcacgctctccctgagggccgacgcagtggcgcacctggaggcgggcaaggactacctggagatcgaggtgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtgtacgggttcacgggagtgcagcacggactcctgccgtgcctgcacgttgccgcgacggtgacgaccatcggccgcgagatgctgctcgcgacccgcgagtacgtccacgcgcgctgggcggccttcgaacagctcctggccgatttcccggaggcggccgacatgcgcgcccccgggccctattccatgcgcatcatctacggggacacggactccatctttgtgctgtgccgcggcctcacggccgccgggctgacggccgtgggcgacaagatggcgagccacatctcgcgcgcgctgtttctgccccccatcaaactcgagtgcgaaaagacgttcaccaagctgctgctgatcgccaagaaaaagtacatcggcgtcatctacgggggtaagatgctcatcaagggcgtggatctggtgcgcaaaaacaactgcgcgtttatcaaccgcacctccagggccctggtcgacctgctgttttacgacgataccgtctccggagcggccgccgcgttagccgagcgccccgcggaggagtggctggcgcgacccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtattacaagctcatggcccgccgcgcgcaggtcccgtccatcaaggaccggatcccgtacgtgatcgtggcccagacccgcgaggtagaggagacggtcgcgcggctggccgccctccgcgagctagacgccgccgccccaggggacgagcccgccccccccgcggccctgccctccccggccaagcgcccccgggagacgccgtcgcctgccgaccccccgggaggcgcgtccaagccccgcaagctgctggtgtccgagctggccgaggatcccgcatacgccattgcccacggcgtcgccctgaacacggactattacttctcccacctgttgggggcggcgtgcgtgacattcaaggccctgtttgggaataacgccaagatcaccgagagtctgttaaaaaggtttattcccgaagtgtggcaccccccggacgacgtggccgcgcggctccggaccgcagggttcggggcggtgggtgccggcgctacggcggaggaaactcgtcgaatgttgcatagagcctttgatactctagcatgagccccccgtcgaagctgatgtccctcattttacaataaatgtctgcggccgacacggtcggaatctccgcgtccgtgggtttctctgcgttgcgccggaccacgagcacaaacgtgctctgccacacgtgggcgacgaaccggtaccccgggcacgcggtgagcatccggtctatgagccggtagtgcaggtgggcggacgtgccgggaaagatgacgtacagcatgtggcccccgtaagtggggtccgggtaaaacaacagccgcgggtcgcacgccccgcctccgcgcaggatcgtgtggacgaaaaaaagctcgggttggccaagaatcccggccaagaggtcctggaggggggcgttgtggcggtcggccaacacgaccaaggaggccaggaaggcgcgatgctcgaatatcgtgttgatctgctgcacgaaggccaggattagggcctcgcggctggtggcggcgaaccgcccgtctcccgcgttgcacgcgggacagcaacccccgatgcctaggtagtagcccatcccggagagggtcaggcagttgtcggccacggtctggtccagacagaagggcagcgagacgggagtggtcttcaccaggggcaccgagagcgagcgcacgatggcgatctcctcggagggcgtctgggcgagggcggcgaaaaggccccgatagcgctggcgctcgtgtaaacacagctcctgtttgcgggcgtgaggcggcaggctcttccgggaggcccgacgcaccacgcccagagtcccgccggccgcagaggagcgcgaccgccggcgctccttgccgtgatagggcccgggccgggagccgcggcgatgggggtcggtgtcatacataggtacacagggtgtgctccagggacaggagcgagatcgagtggcgtctaagcagcgcgcccgcctcacggacaaatgtggcgagcgcggtgggctttggtacaaatacctgatacgtcttgaaggtgtagatgagggcacgcaacgctatgcagacacgcccctcgaactcgttcccgcaggccagcttggccttgtggagcagcagctcgtcgggatgggtggcggggggatggccgaacagaacccaggggtcaacctccatctccgtaatggcgcacatggggtcacagaacatgtgcttaaagatggcctcgggccccgcggcccgaagcaggctcacaaaccggcccccgtccccgggctgcgtctcggggtcagcctcgagctggtcgacgacgggtacgatacagtcgaagaggctcgtgttgttttccgagtagcggaccacggaggcccggagtctgcgcagggccagccagtaagcacgcaccagtaacaggttacacagcaggcattctccgccggtgcgcccgcgcccccggccgtgtttcagcacggtggccatcagagggcccaggtcgaggtcgggctgggcatcgggttcggtaaactgcgcaaagcgcggagccacgtcgcgcgtgcgtgccccgcgatgcgcttcccaggactggcggaccgtggcgcgacgggcctccgcggcagcgcgcagctggggccccgactcccagacggcgggggtgccggcgaggagcaacaggaccagatccgcgtacgcccacgtatccggcgactcctccggctcgcggtccccggcgaccgtctcgaattccccgttgcgagcggcggcgcgagtacagcagctgtccccgcccccgcgccgaccctccgtgcagtccaggagacgggcgcaatccttccagttcatcagcgcggtggtgagcgacggctgcgtgccggatcccgccgccgaccccgccccctcctcgcccccggaggccaaggttccgatgagggcccgggtggcagactgcgccaggaacgagtagttggagtactgcaccttggcggctcccggggagggcgagggcttgggttgcttctgggcatgccgcccgggcaccccgccgtcggtacggaagcagcagtggagaaaaaagtgccggtggatgtcgtttatggtgagggcaaagcgtgcgaaggagccgaccagggtcgccttcttggtgcgcagaaagtggcggtccatgacgtacacaaactcgaacgcggccacgaagatgctagcggcgcagtggggcgcccccaggcatttggcacagagaaacgcgtaatcggccacccactgaggcgagaggcggtaggtttgcttgtacagctcgatggtgcggcagaccagacagggccggtccagcgcgaaggtgtcgatggccgccgcggaaaagggcccggtgtccaaaagcccctccccacagggatccgggggcgggttgcggggtcctccgcgcccgcccgaaccccctccgtcgcccgcccccccgcgggcccttgagggggcggtgaccacgtcggcggcgacgtcctcgtcgagcgtaccgacgggcggcacacctatcacgtgactggccgtcaggagctcggcgcagagagcctcgttaagagccaggaggctgggatcgaaggccacatacgcgcgctcgaacgcccccgccttccagctgctgccgggggactcttcgcacaccgcgacgctcgccaggaccccggggggcgaagttgccatggctgggcgggaggggcgcacgcgccagcgaactttacgggacacaatccccgactgcgcgctgcggtcccagaccctggagagtctagacgcgcgctacgtctcgcgagacggcgcgcatgacgcggccgtctggttcgaggatatgacccccgccgagctggaggttgtcttcccgactacggacgccaagctgaactacctgtcgcggacgcagcggctggcctccctcctgacgtacgccgggcctataaaagcgcccgacgacgccgccgccccgcagaccccggacaccgcgtgtgtgcacggcgagctgctcgccgccaagcgggaaagattcgcggcggtcattaaccggttcctggacctgcaccagattctgcggggctgacgcgcgtgctgttgggcgggacggttcgcgaaccctttggtgggtttacgcgggcacgcacgctcccatcgcgggcgccatggcgggactgggcaagccctacaccggccacccaggtgacgccttcgagggtctcgttcagcgaattcggcttatcgtcccatctacgttgcggggcggggacggggaggcgggcccctactctccctccagcctcccctccaggtgcgcctttcagtttcatggccatgacgggtccgacgagtcgtttcccatcgagtatgtactgcggcttatgaacgactgggccgaggtcccgtgcaacccttacctgcgcatacagaacaccggcgtgtcggtgctgtttcaggggttttttcatcgcccacacaacgcccccgggggcgcgattacgccagagcggaccaatgtgatcctgggctccaccgagacgacggggctgtccctcggcgacctggacaccatcaaggggcggctcggcctggatgcccggccgatgatggccagcatgtggatcagctgctttgtgcgcatgccccgcgtgcagctcgcgtttcggttcatgggccccgaagatgccggacggacgagacggatcctgtgccgcgccgccgagcaggctattacccgtcgccgccgaacccggcggtcccgggaggcgtacggggccgaggccgggctgggggtggccggaacgggtttccgggccaggggggacggttttggcccgctccccttgttaacccaagggccctcccgcccgtggcaccaggccctgcggggtcttaagcacctacggattggcccccccgcgctcgttttggcggcgggactcgtcctgggggccgctatttggtgggtggttggtgctggcgcgcgcctataaaaaaggacgcaccgccgccctaatcgccagtgcgttccggacgccttcgccccacacagccctcccgaccgacacccccatatcgcttcccgacctccggtcccgatggccgtcccgcaatttcaccgccccagcaccgttaccaccgatagcgtccgggcgcttggcatgcgcgggctcgtcttggccaccaataactctcagtttatcatggataacaaccacccgcacccccagggcacccaaggggccgtgcgggagtttctccgcggtcaggcggcggcgctgacggaccttggtctggcccacgcaaacaacacgtttaccccgcagcctatgttcgcgggcgacgccccggccgcctggttgcggcccgcgtttggcctgcggcgcacctattcaccgtttgtcgttcgagaaccttcgacgcccgggaccccgtgaggcccggggagttccttctggggtgttttaatcaataaaagaccacaccaacgcacgagccttgcgtttaatgtcgtgtttattcaagggagtgggatagggttcgacggttcgaaacttaacacaccaaataatcgagcgcgtctagcccagtaacatgcgcacgtgatgtaggctggtcagcacggcgtcgctgtgatgaagcagcgcccggcgggtccgctgtaactgctgttgtaggcggtaacaggcgcggatcagtaccgccagggcgctacgaccggtgcgttgcacgtagcgtcgcgacagaactgcgtttgccgatacgggcggggggccgaattgtaagcgcgtcacctcttgggagtcatcggcggataacgcactgaatggttcgttggttatgggggagtgtggttccccagggagtgggtcgagcgcctcggcctcggaatccgagaggaacaacgaggtggcgtcggagtcttcgtcgtcagagacatacagggtctgaagcagcgacacgggcgggggggtagcgtcgatgtgtagcgcgagggaggatgcccacgaagacaccccagacaaggagctgcccgtgcgtggatttgtggaagacgcggaagccgggacggatgggcggttttgcggtgcccggaaccgaaccgccggatactccccgggtgctacatgcccgttttggggctggggttggggctggggttggggctggggttggggctggggttggggctggggttggggctggggttggggttggggttggggctggggttggggttggggctggggctggggctggggctggggctggggctggggctggggctggggctggggctggggctggggctggggctggggctggggctggggctggggttggggcgcggacaggcggctgacggtcaaatgcccccgggggcgcgcagatgtggtgggcgtggccaccggctgccgtgtagtggggcggcgggaaaccgggcctccgggcgcaacaccgccctccagcgtcaagtatgtggggggcgggcctgacgtcgggggcggggcgacgggttggaccgcgggaggcgggggagagggacctgcgggagaggatgaggtcggctcggccgggttgcggcctaaaacaggggccgtggggtcggcggggtcccagggtgaagggagggattcccgcgattcggacagcgacgcgacagcggggcgcgtaaggcgccgctgcggcccgcctacgggaaccctggggggggttggcgcgggacccgaggttagcggggggcggcggttttcgcccccgggcaaaaccgtgccggttgcgaccgggggcggaacgggatcgatagggagagcgggagaagcctggccggcggcctggggcccgagcgggaggggcacaccagacaccaaagcgtggggcgctggctctgggggtttgggaggggccggggggcgcgcgaaatcggtaaccggggcgaccgtgtcggggagggcaggcggccgccaaccctgggtggtcgcggaagcctgggtggcgcgcgccagggagcgtgcccggcggtgtcggcgcgcgcgcgacccggacgaagaagcggcagaagcgcgggaggaggcgggggggcggggggcggtggcatcggggggcgccggggaactttggggggacggcaagcgccggaagtcgtcgcgggggcccacgggcgccggccgcgtgctttcggccgggacgcccggtcgtgcttcgcgagccgtgactgccggcccagggggccgcggtgcacactgggacgtggggacggactgatcggcggtgggcgaaagggggtccggggcaaggaggggcgcggggccgccggagtcgtcagacgcgagctcctccaggccgtgaatccatgcccacatgcgaggggggacgggctcgccgggggtggcgtcggtgaatagcgtgggggccaggcttccgggccccaacgagccctccgccccaacaaggtccacagggccgggggtcgggtttgggaccgaggggctctggtcgtcgggggcgcgctggtacaccggatgccccgggaatagctcccccgacaggagggaggcgtcgaacggccgcccgaggatagctcgcgcgaggaaggggtcctcgtcggtggcgctggcggcgaggacgtcctcgccgcccgccacaaacgggagctcctcggtggcctcgctgccaacaaaccgcacgtcgggggggccgggggggtccgggttttcccacaacaccgcgaccggggtcatggagatgtccacgagcaccagacacggcgggccccgggcgaggggccgctcggcgatgagcgcggacaggcgcgggagctgtgccgccagacacgcgttttcgatcgggttcaggtcggcgtgcaggaggcggacggcccacgtctcgatgtcggacgacacggcatcgcgcaaggcggcgtccggcccgcgagcgcgtgagtcaaacagcgtgagacacagctccagctccgactcgcgggaaaaggccgtggtgttgcggagcgccacgacgacgggcgcgcccaggagcactgccgccagcaccaggtccatggccgtaacgcgcgccgcgggggtgcggtgggtggcggcggccggcacggcgacgtgctggcccgtgggccggtagagggcgttggggggagcggggggtgacgcctcgcgcccccccgaggggctcagcgtctgcccagattccagacgcgcggtcagaagggcgtcgaaactgtcatactctgtgtagtcgtccggaaacatgcaggtccaaagagcgaccagagcggtgcttgggagacacatgcgcccgaggacgctcaccgccgccagcgcctgggcgggactcagctttcccagcgcggcgccgcgctcggttcccagctcggggaccgagcgccagggcgccagggggtcggtttcggacaacttgccgcggcgccagtctgccagccgcgtgccgaacatgaggccccgggtcggagggcctccggccgaaaacgctggcagcacgcggatgcgggcgtctggatgcggggtcaggcgctgcacgaatagcatggaatctgctgcgttctgaaacgcacgggggagggtgagatgcatgtactcgtgttggcgaaccagatccaggcgccaaaaggtgtaaatgtgttccggggagctggccaccagcgccaccagcacgtcgttctcgttaaaggaaacgcggtgcctagtggagctctggggtccgagcggcggccccggggccgccgcgtcacccccccattccagctgggcccagcgacacccaaactcgcgcgtgagagtggtcgcgacgagggcgacgtagagctcggccgccgcatccatcgaggccccccatctcgcctggcggtggcgcacaaagcgtccgaagagctgaaagttggcggcctgggcgtcgctgagggccagctgaagccggttgatgacggtgaggacgtacatggccgtgacggtcgaggccgactccagggtgtccgtcggaagcggggggcgaatgcatgccgcctcgggacacatcagcagcgcgccgagcttgtcggtcacggccgggaagcagagcgcgtactgcagtggcgttccatccgggaccaaaaagctgggggcgaacggccgatccagcgtactggtggcctcgcgcagcaccaggggccccgggcctccgctcactcgcaggtacgcctcgccccggcggcgcagcatctgcgggtcggcctcttggccgggtggggcggacgcccgggcgcgggcgtctagggcgcgaagatccacgagcaggggcgcgggcgcggccgccgcgcccgcgcccgtctggcctgtggccttggcgtacgcgctatataagcccatgcggcgttggatgagctcccgcgcgccccggaactcctccaccgcccatggggccaggtccccggccaccgcgtcgaattccgccaacaggccccccagggtgtcaaagttcatctcccaggccacccttggcaccacctcgtcccgcagccgggcgctcaggtcggcgtgttgggccacgcgccccccgagctcctccacggccccggcccgctcggcgctcttggcgcccaggacgccctggtacttggcgggaaggcgctcgtagtcccgctgggctcgcagccccgacacagtgttggtggtgtcctgcagggcgcgaagctgctcgcatgccgcgcgaaatccctcgggcgatttccaggcccccccgcgaacgcggccgaagcgaccccatacctcgtcccactccgcctcggcctcctcgagagacctccgcagggcctcgacgcggcgacgggtgtcgaagagcgcctgcaggcgcgcgccctgtcgcgtcaggaggcccgggccgtcgccgctggccgcgcttagcgggtgcgtctcaaaggtacgctgggcatgttccaaccaggcgaccgcctgcacgtcgagctcgcgcgccttctccgtctggtccaccagaatttcgacctgatccgcgatctcctccgccgagcgcgcctggtccagcgtcttggccacggtcgccgggacggcgaccaccttcagcagggtcttcagattggccagaccctcggcctcgagctgggcccggcgctcgcgcgcggccagcacctcccgcagccccgccgtgacccgctcggtggcttcggcgcgctgctgtttggcgcgcaccacggcgtccttggtatcggccaggtcctgtcgggtcacgaatgcgacgtagtgcgcgtacgccgtgtccttcacggggctctggtccacgcgctccagcgccgccacgcacgccaccagcgcgtcctcgctcgggcagggcagggtgacccctgcccggacaagctcggcggccgccgccgggtcgttgcgcaccgcggatatctcctccgcggcggcggccaggtccagcgccacgcttccgatcgcgcgccgcgcgtcggcccggagggcgtccaggcgatcgcggatatccacgtactcggcgtagcccttttgaaaaaacggcacgtactggcgcagggccggcacgccccccaagtcttccgacaggtgtaggacggcctcgtggtagtcgataaacccgtcgttcgcctgggcccgctccagcagcccccccgccagccgcagaagccgcgccaggggctcggtgtccacccgaaacatgtcggcgtacgtgtcggccgcggccccgaaggccgcgctccagtcgatgcggtgaatggctgcgagcggggggagcatggggtggcgctggttctcgggggtgtatgggttaaacgcaagggccgtctccagggcaagggtcaccgccttggcgttggttcccagcgcctgttcggcccgctttcggaagtcccgggggttgtagccgtgcgtgcccgccagcgcctgcaggcgacggagctcgaccacgtcaaactcggcaccgctttccacgcggtccagcacggcctccacgtcggcggcccagcgctcgtggctactgcgggcgcgctgggccgccatcttctctctgaggtcggcggtggcggcctcaagttcgtcggcgcggcgtcgcgtggcgccgatgacctttcccagctcctgcagggcgcgcccgctgggggagtggtccccggccgtcccttcggcgtgcaacaggcccccgaacctgccctcgtggcccgcgaggctttcccgcgcgccggtggtcgcgcgcgtcgcggcctggatcagggaggcatgctctccctccggttggttggcggcccggcgcacctggacgacaaggtcggcggcagccgaccctaaggtcgtgagctgggcgatggccacccgcgcgtccagggccaaccgagtcgccttgacgtatcccgcggcgctgtcggccatggccgctaggaaggccaggggggaggccgggtcgctggcggccgcgcccagggccgtcaccgcgtcgaccaggacgcggtgcgcccgcacggccgcatccaccgtcgacgcagggtctgccgttgcgacggcggcgctgccggcgttgatggcgttcgagacggcgtgggctatgatcggggcgtgatcggcgaagaactgcaagagaaacggagtctctggggcgtcggcgaacaggttcttcagcaccaccacgaagctgggatgcaagccagacagagccgtcgccgtgtccggagtcgggtgctccagggcatctcggtactgccccagcagcccccacatgtccgcccgcagcgccgccgtaacctcagggggcgccccccgaacggcctcggggaggtccgaccagcccgccggcagggaggcccgcagggtcgccaggacggccggacaggcctttagccccacaaagtcagggagggggcgcaggaccccctggagtttgtgcaagaacttctcccgggcgtcgcgggccaccttcgcccgctcccgcgctccctcgagcattgcctccagggagcgcgcgcgctcccgcaaacgggcacgcgcatcgggggcgagctctgccgtcagcttggcggcatccatggcccgcgcctgccgcagcgcttcctcggccatgcgcgtggcctctggcgacagcccgccgtcgtcggggtagggcgacgcgccgggcgcaggaacaaaggccgcgtcgctgtccagctgctggcccagggccgcatctagggcgtcgaagcgccgcagctcggccagacccgagctgcggcgcgcctgttggtcgttaatgtcgcggatgctgcgcgccagctcgtccagtggcttgcgttctatcagcccttggttggcggcgtccgtcaggacggagagccaggccgccaggtcctcgggggcgtccagcgtctggccccgctggatcagatcccgcaacaggatggccgtggggctggtcgcgatcgggggcggggcgggaatggcggcgcgctgcgcgatgtcccgcgtgtgctggtcgaagacaggcagggactcgagcagctggaccacgggcacgacggcggccgaagccacgtgaaaccggcggtcgttgttgtcgctggcctgtagagccttggcgctgtatacggccccccggtaaaagtactccttaaccgcgccctcgatcgcccgacgggcctgggtccgcacctcctccagccgaacctgaacggcctcggggcccagggggggtgggcgcggagccccctgcggggccgccccggccggggcgggcattacgccgaggggcccggcgtgctgtgagaccgcgtcgaccccgcgagcgagggcgtcgagggcctcgcgcatctggcgatcctccgcctccaccctaatctcttcgccacgggcaaatttggccagagcctggactctatacagaagcggttctgggtgcgtcggggtggcgggggcaaaaagggtgtccgggtgggcctgcgagcgctccagaagccactcgccgaggcgtgtatacagattggccggcggggccgcgcgaagctgcagctccaggtccgcgagttccccgtaaaaggcgtccgtctcccgaatgacatccctagccacaaggatcagcttcgccagcgccaggcgaccgatcagagagttttcgtccagcacgtgctggacgaggggcagatgggcggccacgtcggccaggctcaggcgcgtggaggccagaaagtcccccacggccgttttccagggcagcatgttcagggtaaactccagcagggcggcggccgggccggccaccccggcctgggtgtgcgtccgggccccgttctcgatgagaaaggcgaggacgcgttcaaagaaaaaaataacacagagctccagcagccccggagaggccggatacggcgaccgtaaggcgctgatggtgagccgcgaacacgcggcgacctcgcgggccagggcggcggagcacgcggtgaacttaaccgccgtggcggccacgtttgggtgggcctcgaacagctgggcgaggtctgcgcccgggggctcgggcgagcggcgagtcttcagcgcctcgagggcctgtgaggacgccggaaccgtgggcccgtcgtcctcgcccgcctcggcgaccggcggcccggccgggtcggggggtgccgaggcgaggacaggctccggaacggaggcggggaccgcggccccgacgggggttttgcctttgggggtggatttcttcttggttttggcagggggggccgagcgtttcgttttctcccccgaagtcaggtcttcgacgctggaaggcggagtccaggtgggtcggcggcgcttgggaaggccggccgagtagcgtgcccggtgccgaccaaccgggacgacgcccatctccaggacccgcatgtcgtcgtcatcttcttcggccgcctctgcggcgggggtcttgggggcggagggaggcggtggtgggatcgcggagggtgggtcggcggagggtgggtcggcggaggggggatccgtgggtggggtacccttcagggccaccgcccatacatcgtcgggcgcccgattcgggcgcttggcctctggttttgccgacggaccggccgtcccccgggatgtctcggaggccctgtcgtcgcgacgggcccgggtcggtggcggcgactgggcggctgtgggcgggtgtggccccgggccccctaccccctcccgggggcccacgccgacgcagggctcccccaggcccgcgatctcgccccgcagggggtgcgtgatggccacgcgccgttcgctgaacgcttcgtcctgcaggtaagtctcgctggccccgtaaagatgcagagccgcggccgtcaagtccgcaggagccgcgggttccgggcccgacggcacgaaaaacaccatggctcccgcccaccgtacgtccgggcgatcgcgggtgtaatacgtcaggtatggatacatgtcccccgcccgcactttggcgatgaacgcgggggtgccctccggaaggccgtgcgggtcaaaaaggtatgcggtgtcgccgtccctgaacagccccatccctagggggccaatggttaggagcgtgtacgacagggggcgcagggcccacgggccggcgaagaacgtgtgtgcggggcattgtgtctccagcaggcccgccgcgggctccccgaagaagcccacctcgccgtatacgcgcgagaagacacagcgcagtccgccgcgcgcccctgggtactcgaggaagttggggagctcgacgatcgaacacatgcgcggcggcccagggcccgcggtcgcgcgcgtccactcgcccccctcgaccaaacatccctcgatggcctccgcggacaggacgtcgcgagggcccacatcaaatatgaggctgagaaaggacagcgacgagcgcatgcacgataccgacccccccggctccaggtcgggcgcgaactggttccgagcaccggtgaccacgatgtcgcgatcccccccgcgttccatcgtggagtgcggtggggtgcccgcgatcatatgtgccctgctggccagagacccggcctgtttatggaccggacccccggggttagtgttgtttccgccacccatgcccccgtaccatggccccggttcccctgattaggctacgagtcgcggtgatcgcttcccaaaaaccgagctgcgtttgtctgtcttggtcttccacccccccccccgcccgcccgcacaccataacaccgagaacaacacacgggggtgggcgtaacataataaagctttattggtaactagttaacggcaagtccgtgggtggcgcgacggtgtcctccgggatcatctcgtcgtcctcgacgggggtgttggaatgaggcgccccctcgcggtccgcctggcgtgggccgtgcccataggcctccggcttctgtgcgtccatgggcataggcgcggggagactgtttccggcgtcgcggacctccaggtccctgggagactccggtccggctaacggacgaaacgcggaagcgcgaaacacgccgtcggtgacccgcaggagctcgttcatcagtaaccaatccatactcagcgtaacggccagcccctggcgagacagatccacggagtccggaaccgcggtcgtctggcccagggggccgaggctgtagtccccccaggcccctaggtcgcgacggctcgtaagcacgacgcggtcggccgcggggctttgcgggggggcgtcctcgggcgcatgcgccattacctctcggatggccgcggcgcgctggtcggccgagctgaccaagggcgccacgaccacggcgcgctccgtctgcaggcccttccacgtgtcgtggagttcctggacaaactcggccacgggctcgggtcccgcggccgcgcgcgcggcttgatagcaggccgacagacgccgccagcgcgctagaaactgacccatgaagcaaaacccggggacctggtctcccgacagcagcttcgacgcccgggcgtgaatgccggacacgacggacagaaacccgtgaatttcgcgccggaccacggccagcacgttgtcctcgtgcgacacctgggccgccagctcgtcgcacacccccaggtgcgccgtggtttcggtgatgacggaacgcaggctcgcgagggacgcgaccagcgcgcgcttggcgtcgtgatacatgctgcagtactgactcaccgcgtcccccatggcctcggggggccagggccccaggcggtcgggcgtgtccccgaccaccgcatacaggcggcgcccgtcgctctcgaaccgacactcgaaaaaggcggagagcgtgcgcatgtgcagccgcagcagcacgatggcgtcctccagttggcgaatcagggggtctgcgcgctcggcgaggtcctgcagcaccccccgggcggccagggcgtacatgctaatcaacaggaggctggtgcccacctcggggggcggggggggctgcagctggaccaggggccgcagctgctcgacggcacccctggagatcacgtacagctcccggagcagctgctctatgttgtcggccatctgcatagtggggccgaggccgccccgggcggccggttcgaggagggtgatcagcgcgcccagtttggtgcgatggccctcaaccgtggggagatagcccagcccaaagtcccgggcccaggccaacacacgcagggcgaactcgaccgggcggggaaggtaggccgcgctacacgtggccctcagcgcgtccccgaccaccagggccagaacgtaggggacgaagcccgggtcggcgaggacgttggggtgaatgccctcgagggcggggaagcggatctgggtcgccgcggccaggtggacagagggggcgtggctgggctgcccgacggggagaagcgcggacagcggcgtggccggggtggtgggggtgatgtcccagtgggtctgaccatacacgtcgatccagatgagcgccgtctcgcggagaaggctgggttgaccggaactaaagcggcgctcggccgtctcaaactcccccacgagcgcccgccgcaggctcgccagatgttccgtcggcacggccggacccatgatacgcgccagcgtctggcttagaacgccccccgacaggccgaccgcctcgcagagccgcccgtgcgtgtgctcgctggcgccctggacccgcctgaaagtttttacgtagttggcatagtacccgtattcccgcgccaacccaaacacgttcgaccccgcgagggcaatgcacccaaagagctgctggacttcgccgagtccgtggccggtgggcgtccgcgcggggacgcccgccgccagaaacccctccagggccgaaaggtagtgcgtgcagtgcgagggcgtgaacccagcgtcgatcagggtgttgatcaccacggagggcgaattggtattctggatcaacgtccacgtctgctgcagcagagccagcagccgctgctgggcgccggcggagggctgctccccgagctgcagcaggctggagacggcaggctggaagactgccagtgccgacgaactcaggaacggcacgtcgggatcaaacacggccacgtccgtccgcacgcgcgccattagcgtccccgggggcgcacaggccgagcgcgggctgacgcggctgagggccgtcgacacgcgcacctcctcgcggctgcgaaccatcttgttggcctcgagcggcggaatcattatggccgggtcgatctcccgcacggtgtgctgaaactgcgccaacaggggcggcgggaccacagccccccgctcgggggtcgtcaggtactcgtccaccagggccaacgtaaagagggcccgtgtgaggggagtgagggtcgcgtcgtctatgcgctggaggtgcgccgagaacagcgtcacccgattactcaccagggccaagaaccggaggccctcttgcacgaacggggcggggaagagcaggctgtacgccggggtggtaaggttcgcgctgggctgccccaacgggaccggcgccatcttgagcgacgtctccccaagggcctcgatggaggtccgcgggctcatggccaagcagctcttggtgacggtttgccagcggtctatccactccacggcgcactggcggacgcggaccggccccagggccgccgcggtgcgcaggccggcggaatccagcgcatgggacgtgtcggagccggtgaccgcgaggatggtgtccttgatgacctccatctcccggaaggcctggtcgggggcctcggggagagccaccaccaagcggtgtacgagcaacccggggaggttctcggccaagagcgccgtctccggaagcccgtgggcccggtggagcgcgcacaggtgttccagcagcggccgccagcagtcccgcgcgtctgccggggcgatggccgttcccgacaacagaaacgccgccatggcggcgcgcagcttggccgtggccagaaacgccgggtcgtccgccccgtttgccgtctcggccgtgggggttggcggttggcgaaggccggctaggctcgccaataggcgctgcataggtccgtccgagggcggaccggcgggtgaggtcgtgacgacgggggcctcggacgggagaccgcggtctgccatgacgcccggctcgcgtgggtgggggacagcgtagaccaacgacgagaccgggcgggaatgactgtcgtgcgctgtagggagcggcgaattatcgatcccccgcggccctccaggacccccgcaggcgttgcgagtaccccgcgtcttcgcggggtgttatacggccacttaagtcccggcatcccgttcgcggacccaggcccgggggattgtccggatgtgcgggcagcccggacggcgtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgacccccccccccctccctcccccgggtcttgctaggtcgcgatctggggtcgcaatgaagaccaatccgctacccgcaaccccttccgtgtggggcgggagtaccgtggaactcccccccacaacacgcgataccgcgggacagggcctgcttcggcgcgtcctgcgccccccgatctctcgccgcgacggcccagggctccccagggggtcgggaccccggagggcggccagcacgctgtggttgcttggcctggacggcacagacgcgccccctggggcgctgacccccaacgacgataccgaacaggccctggacaagatcctgcggggcaccatgcgcgggggggcggccctgatcggctccccgcgccatcatctaacccgccaagtgatcctgacggatctgtgccaacccaacgcggatcgtgctgggacgctgcttctggcgctgcggcaccccgccgacctgcctcacctggcccaccagcgcgccccgccaggccggcagaccgagcggctgggcgaggcctggggccagctgatggaggcgaccgccctggggtcggggcgagccgagagcgggtgcacgcgcgcgggcctagtgtcgtttaacttcctggtggcggcgtgtgccgcctcgtacgacgcgcgcgacgccgccgatgcggtacgggcccacgtcacggccaactaccgcgggacgcgggtgggggcgcgcctggatcgtttttccgagtgtctgcgcgccatggttcacacgcacgtcttcccccacgaggtcatgcggtttttcggggggctggtgtcgtgggtcacccaggacgagctagcgagcgtcaccgccgtgtgcgccgggccccaggaggcggcgcacaccggccacccgggccggccccgctcggccgtgatcctcccggcatgtgcgttcgtggacctggacgccgagctggggctggggggcccgggcgcggcgtttctgtacctggtattcacttaccgccagcgccgggaccaggagctgtgttgtgtgtacgtgatcaagagccagctccccccgcgcgggttggagccggccctggagcggctgtttgggcgcctccggatcaccaacacgattcacggcaccgaggacatgacgcccccggccccaaaccgaaaccccgacttccccctcgcgggcctggccgccaatccccaaaccccgcgttgctcggctggccaggtcacgaacccccagttcgccgacaggctgtaccgctggcagccggacctgcgggggcgccccaccgcacgcacctgtacgtacgccgcctttgcagagctcggcatgatgcccgaggatagtccccgctgcctgcaccgcaccgagcgctttggggcggtcagcgtccccgttgtcattctggaaggcgtggtgtggcgccccggcgagtggcgggcatgcgcgtgagcgtagcaaacgccccgcccacacaacgctccgcccccaaccccttccccgctgtcactcgttgttcgttgacccgggcgtccgccaaataaagccactgaaacccgaaacgcgagtgttgtaacgtcctttgggcgggaggaagccacaaaatgcaaatgggatacatggaaggaacacacccccgtgactcaggacatcggtgtgtccttttgggtttcactgaaactggcccgcgccccacccctgcgcgatgtggataaaaagccagcgcgggtggtttagggtaccacaggtgggtgctttggaaacttgccggtcgccgtgctcctgtgagcttgcgtccctccccggtttcctttgcgctcccgccttccggacctgctctcgcctactcttctttggctctcggtgcgattcgtcaggcagcggccttgtcgaatctcgaccccaccactcgccggacccgccgacgtcccctctcgagcccgccgaaacccgccgcgtctgttgaaatggccagccgcccagccgcatcctctcccgtcgaagcgcgggccccggttgggggacaggaggccggcggccccagcgcagccacccagggggaggccgccggggcccctctcgcccacggccaccacgtgtactgccagcgagtcaatggcgtgatggtgctttccgacaagacgcccgggtccgcgtcctaccgcatcagcgataacaactttgtccaatgtggttccaactgcaccatgatcatcgacggagacgtggtgcgcgggcgcccccaggacccgggggccgcggcatcccccgctcccttcgttgcggtgacaaacatcggagccggcagcgacggcgggaccgccgtcgtggcattcgggggaaccccacgtcgctcggcggggacgtctaccggtacccagacggccgacgtccccaccgaggcccttgggggcccccctcctcctccccgcttcaccctgggtggcggctgttgttcctgtcgcgacacacggcgccgctctgcggtattcgggggggagggggatccagtcggccccgcggagttcgtctcggacgaccggtcgtccgattccgactcggatgactcggaggacacggactcggagacgctgtcacacgcctcctcggacgtgtccggcggggccacgtacgacgacgcccttgactccgattcgtcatcggatgactccctgcagatagatggccccgtgtgtcgcccgtggagcaatgacaccgcgcccctggatgtttgccccgggacccccggcccgggcgccgacgccggtggtccctcagcggtagacccacacgcgccgacgccagaggccggcgctggtcttgcggccgatcccgccgtggcccgggacgacgcggaggggctttcggacccccggccacgtctgggaacgggcacggcctaccccgtccccctggaactcacgcccgagaacgcggaggccgtggcgcgctttctgggagatgccgtgaaccgcgaacccgcgctcatgctggagtacttttgccggtgcgcccgcgaggaaaccaagcgtgtcccccccaggacattcggcagcccccctcgcctcacggaggacgactttgggcttctcaactacgcgctcgtggagatgcagcgcctgtgtctggacgttcctccggtcccgccgaacgcatacatgccctattatctcagggagtatgtgacgcggctggtcaacgggttcaagccgctggtgagccggtccgctcgcctttaccgcatcctgggggttctggtgcacctgcggatccggacccgggaggcctcctttgaggagtggctgcgatccaaggaagtggccctggattttggcctgacggaaaggcttcgcgagcacgaagcccagctggtgatcctggcccaggctctggaccattacgactgtctgatccacagcacaccgcacacgctggtcgagcgggggctgcaatcggccctgaagtatgaggagttttacctaaagcgttttggcgggcactacatggagtccgtcttccagatgtacacccgcatcgccggctttttggcctgccgggccacgcgcggcatgcgccacatcgccctggggcgagaggggtcgtggtgggaaatgttcaagttctttttccaccgcctctacgaccaccagatcgtaccgtcgacccccgccatgctgaacctggggacccgcaactactacacctccagctgctacctggtaaacccccaggccaccacaaacaaggcgaccctgcgggccatcaccagcaacgtcagtgccatcctcgcccgcaacgggggcatcgggctatgcgtgcaggcgtttaacgactccggccccgggaccgccagcgtcatgcccgccctcaaggtccttgactcgctggtggcggcgcacaacaaagagagcgcgcgtccgaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagtgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaacctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgccgaatttcaggacctgaacaaacacatcgccgaggtgatgctgctgtcggcgatgaagaccagcaacgcgctgtgcgttcgcggggcccgtcccttcaaccactttaagcgcagcatgtatcgcgccggccgctttcactgggagcgctttccggacgcccggccgcggtacgagggcgagtgggagatgctacgccagagcatgatgaaacacggcctgcgcaacagccagtttgtcgcgctgatgcccaccgccgcctcggcgcagatctcggacgtcagcgagggctttgcccccctgttcaccaacctgttcagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgctgatcgacctgtgtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcatgagctgcgcgctgtgaccgacaaaccccctccgcgccaggcccgccgccactgtcgtcgccgtcccacgctctcccctgctgccatggattccgcggccccagccctctcccccgctctgacggcccttacggaccagagcgcgacggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgcttttccacaacaacgaccaggcgcgccgcgagtacgtggccggtaccatcaaccacccggccatccgcgccaaggtggactggttggaagcgcgggtgcgggaatgcgcctccgttccggaaaagttcattctcatgatcctcatcgagggcatcttttttgccgcctcgtttgccgccatcgcctaccttcgcaccaacaaccttctgcgggtcacctgccagtcaaacgacctcatcagccgggacgaggccgtgcacacgacggcctcgtgttacatctacaacaactacctcggcgggcacgccaagcccccgcccgaccgcgtgtacgggctgttccgccaggcggtcgagatcgagatcggatttatccgatcccaggcgccgacggacagccatatcctgagcccggcggcgctggcggccatcgaaaactacgtgcgattcagcgcggatcgcctgttgggccttatccacatgaagccactgttttccgccccaccccccgacgccagctttccgctgagcctcatgtccaccgacaaacacaccaattttttcgagtgtcgcagcacctcctacgccggggcggtcgtcaacgatctgtgagtgtcgcggcgcgcttctacccgtgtttgcccataataaacctctgaaccaaactttgggtctcattgtgattcttgtcagggacgcgggggtgggagaggataaaaggcggcgcaaaaagcagtaaccaggtccgtccagattctgcgggcataggataccataattttattggtgggtcgtttgttcggggacaagcgcgctcgtctgacgtttgggctactcgtcccagaatttggccaggacgtccttgtagaacgcgggtgggggggcctgggtccgcaactgctccagaaacctgtcggcgatatcaggggccgtgatatgccgggtcacgatagatcgcgccaggttttcgtcgcggatgtcctggtagataggcaggcgtttcagaagagtccacggcccccgctccttggggccgataagcgatatgacgtacttaatgtagcggtgttccaccagctcggtgatggtcatgggatcggggagccagtccagggactctggggcgtcgtggatgacgtggcgtcgcgggttggccacataactgcggtgctcttccagcagctgcgcgttcgggacctggacgagctcgggcggggtgagtatctccgaggaggacgacctggggccggggtggcccccggtaacgtcccggggatccagggggaggtcctcgtcgtcttcgtatccgccggcgatctgttgggttagaatttcggtccacgagacgcgcgtctcggtgccgccggcggccggcggcagagggggcctggtttccgtggagcgcgagctggtgtgttcccggcggatggcccgccgggtctgagagcgactcgggggggtccagtgacattcgcgcagcacatcctccacggaggcgtaggtgttattgggatggaggtcggtgtggcagcggacaaagagggccaggaactgggggtagctcatcttaaagtactttagtatatcgcgacagttgatcgtgggaatgtagcaggcgctaatatccaacacaatatcacagcccatcaacaggaggtcagtgtccgtggtgtacacgtacgcgaccgtgttggtgtgatagaggttggcgcaggcatcgtccgcctccagctgacccgagttaatgtaggcgtaccccagggcccggagaacgcgaatacagaacagatgcgccagacgcagggccggcttcgagggcgcggcggacggcagcgcggctccggacccggccgtcccccgggtccccgaggccagagaggtgccgcgccggcgcatgttggaaaaggcagagctgggtctggagtcggtgatgggggaaggcggtggagaggcgtccacgtcactggcctcctcgtccgtccggcactgggccgtcgtgcgggccaggatggccttggctccaaacacaaccggctccatacaattgaccccgcgatcggtaacgaagatggggaaaagggacttttgggtaaacacctttaataagcgacagaggcagtgtagcgtaatggcctcgcggtcgtaactggggtatcggcgctgatatttgaccaccaacgtgtacatgacgttccacaggtccacggcaatgggggtgaagtacccggccggggccccaaggccccggcgcttgaccagatggtgtgtgtgggcaaacttcatcatcccgaacaaacccatgtcaggtcaattgtaactgcggatcggcctaactaaggcgtggttggtgcgacggtccgggacacccgagcctgtctctctgtgtatggtgacccagacaacaacaccgacacaagaggacaataatccgttaggggacgctctttataatttcgatggcccaactccacgcggattggtgcagcaccctgcatgcgccggtgcgggccaaccttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccatcaaaaccccaccctcgcagcggcatacgccccctccgcgtcccgcacccgagaccccggcccggctgccctcaccaccgaagcccacctcgtcactgtggggtgttcccagcccgcgttgggatgacggattcccctggcggtgtggcccccgcctcccccgtggaggacgcgtcggacgcgtccctcgggcagccggaggagggggcgccctgccaggtggtcctgcagggcgccgaacttaatggaatcctacaggcgtttgccccgctgcgcacgagccttctggactcgcttctggttatgggcgaccggggcatccttatccataacacgatctttggggagcaggtgttcctgcccctggaacactcgcaattcagtcggtatcgctggcgcggacccacggcggcgttcctgtctctcgtggaccagaagcgctccctcctgagcgtgtttcgcgccaaccagtacccggacctacgtcgggtggagttggcgatcacgggccaggccccgtttcgcacgctggttcagcgcatatggacgacgacgtccgacggcgaggccgttgagctagccagcgagacgctgatgaagcgcgaactgacgagctttgtggtgctggttccccagggaacccccgacgttcagttgcgcctgacgaggccgcagctcaccaaggtccttaacgcgaccggggccgatagtgccacgcccaccacgttcgagctcggggttaacggcaaattttccgtgttcaccacgagtacctgcgtcacctttgctgcccgcgaggagggcgtgtcgtccagcaccagcacccaggtccagatcctgtccaacgcgctcaccaaggcgggccaggcggccgccaacgccaagacggtgtacggggaaaatacccatcgcaccttctctgtggtcgtcgacgattgcagcatgcgggcggtgctccggcgactgcaggtcggcgggggcaccctcaagttcttcctcacgacccccgtccccagtctgtgcgtcaccgccaccggtcccaacgcggtatcggcggtatttctcctgaaaccccagaagatttgcctggactggctgggtcatagccaggggtctccttcagccgggagctcggcctcccgggcctctgggagcgagccaacagacagccaggactccgcgtcggacgcggtcagccacggcgatccggaagacctcgatggcgctgcccgggcgggagaggcgggggccttgcatgcctgtccgatgccgtcgtcgaccacgcgggtcactcccacgaccaagcgggggcgctcggggggcgaggatgcgcgcgcggacacggccctaaagaaacctaagacggggtcgcccaccgcacccccgcccgcagatccagtccccctggacacggaggacgactccgatgcggcggacgggacggcggcccgtcccgccgctccagacgcccggagcggaagccgttacgcgtgttactttcgcgacctcccgaccggagaagcaagccccggcgccttctccgccttccgggggggcccccaaaccccgtatggttttggattcccctgacggggcggggccttggcggccgcccaactctcgcaccatcccgggttaatgtaaataaacttggtattgcccaacactttcccgcgtgtcgcgtgtggttcatgtgtgtgcctggcgcccccaccctcgggttcgtgtatttcctttccctgtccttataaaagccgtatgtggggcgtgacggaaccaccccgcgtgccatcacggccaaggcgcgggatgctccgcaacgacagccaccgggccgtgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgaatgccgcgtctggggcgttcgccgtcgggtgcgccgtgctgggctttatgcgcgcaccccctcccctcgcgcggcccaccgcgcggatatacgcctggctcaaactggcggccggtggagcggcccttgttctgtggagtctcggggagcccggcacgcagccgggggccccggccccgggcccggccacccagtgcctggcactgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccctggccgcggcggtgttggcgggcccgggggcgaccaccgccagggactgcttttccagggcttgccccgaccaccgccgcgtctgtgtcatcaccgcaggcgagtctctttcccgccgccccccggaggacccagagcgacccggggttcccgggcccccgtcccccccgaccccccaacgatcccacgggccgccggccgatgaggtcgcaccggccagggtcgcgcggcccgaaaacgtctgggtgcccgtggtcacctttctgggggcgggcgcgcttgccgtcaagacggtgcgagaacatgcccggggaacgccgggcccgggcctgccgctgtggccccaggtgtttctcggaggccatgtggcggtggccctgacggagctgtgtcaggcgcttccgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggtgctgctgtgccctccggggtcaacgggcgggcggtcgggcgattgatatatttttcaataaaaggcattagtcccgaagaccgccggtgtgtgatgatttcgccataacacccaaaccccggatggggcccgggtataaattccggaaggggacacgggctaccctcactatcgagggcgcttggtcgggaggccgcatcgaacgcacacccccatccggtggtccgtgtggaggtcgttttcatgcccggtctcgctttgccgggaacgctagccgatccctcgcgagggggaggcgtcgggcatggccccggggcgggtgggccttgccgtggtcctgtggagcctgttgtggctcggggcgggggtgtccgggggctcggaaactgcctccaccgggcccacgatcaccgcgggagcggtgacgaacgcgagcgaggcccccacatcggggtcccccgggtcagccgccagcccggaggtcacccccacatcgaccccaaaccccaacaatgtcacacaaaacaaaaccacccccaccgagccggccagccccccaacaacccccaagcccacctccacgcccaaaagcccccccacgtccacccccgaccccaaacccaagaacaacaccacccccgccaagtcgggccgccccactaaaccccccgggcccgtgtggtgcgaccgccgcgacccattggcccggtacggctcgcgggtgcagatccgatgccggtttcggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctaacggacccccacgtgctctgggcggagggggccggcccgggcgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccaccaggtgtttaacccgggccagatcgacacgcagacgcacgagcaccccgacgggttcaccacagtctctaccgtgacctccgaggctgtcggcggccaggtccccccgcggaccttcacctgccagatgacgtggcatcgcgactccgtgacgttctcgcgacgcaatgccaccgggctggccctggtgctgccgcggccaaccatcaccatggaatttggggtccgcattgtggtctgcacggccggctgcgtccccgagggcgtgacgtttgcctggttcctgggggacgacccctcaccggcggctaagtcggccgttacggcccaggagtcgtgcgaccaccccgggctggctacggtccggtccaccctgcccatttcgtacgactacagcgagtacatctgtcggttgaccggatatccggccgggattcccgttctagaacaccacggcagtcaccagcccccacccagggaccccaccgagcggcaggtgatcgaggcgatcgagtgggtggggattggaatcggggttctcgcggcgggggtcctggtcgtaacggcaatcgtgtacgtcgtccgcacatcacagtcgcggcagcgtcatcggcggtaacgcaagacccccccgttacctttttaatatctatatagtttggtcccccctctatcccgcccaccgctgggcgctataaagccgccaccctctcttccctcaggtcatccttggtcgatcccgaacgacacacggcgtggagcaaaacgcctccccctgagccgctttcctaccaacacaacggcatgcctctgcgggcatcggaacacgcctaccggcccctgggccccgggacaccccccatgcgggctcggctccccgccgcggcctgggttggcgtcgggaccatcatcgggggagttgtgatcattgccgcgttggtcctcgtgccctcgcgggcctcgtgggcactttccccatgcgacagcggatggcacgagttcaacctcgggtgcatatcctgggatccgacccccatggagcacgagcaggcggtcggcggctgtagcgccccggcgaccctgatcccccgcgcggctgccaaacagctggccgccgtcgcacgcgtccagtcggcaagatcctcgggctactggtgggtgagcggagacggcattcgggcctgcctgcggctcgtcgacggcgtcggcggtattgaccagttttgcgaggagcccgcccttcgcatatgctactatccccgcagtcccgggggctttgttcagtttgtaacttcgacccgcaacgcgctggggctgccgtgaggcgcgtgtactgcggtctgtctcgtctcctcttctccccttccctccccctccgcatcccaggatcacaccggtcaacgagggttgggggggtccggcacggacccaaaataataaacacacaatcacgtgcgataaaaagaacacgcggtcccctgtggtgtttttggttatttttattaaatctcgtcgacaaacagggggaaaggggcgtggtctagcgacggcagcacgggcggaggcgttcaccggctccggcgtccttcgcgtttaagcttggtcaggagggcgctcagggcggcgacgttggtcgggccgtcgttggtcagggcgttggctcgatggcgggcgaggacgggcgaggggctcaacggcgggggcgggggcccggtgcggcccgggggggaaaatagggcggatcccccccagtcgtacaggggattttccgcctcaatgtacggggaggccggcgctgcattcgccgtgttcacgcagacgttttcgtagacccgcatccatggtatttcctcgtagacacgccccccgtcctcgctcaccgtctcgtatattgactcgtcgtcctcgtagggggcgtgccgttcgcgggccgaggcggcgtgggtggctttgcggcgggcgtcgtcgtcgtcgtcgtcggccgtcagatacgtggcttccatctggtcgggttctccctccggggcgggtccccacacccgtggccgatcgaggctccccagagacgcgcgccggacgaggagggggcacgtcgccgccggcggtcgcctgtcgggtcccgcgacgttacgggccgggaggcgcgggggcacctcccccatgtgcgtgtaatacgtggccggctgtgtggccgcagcggggggctcggcgaccgggtcgttcgcatccggaagcgggggccccgcgccgtccgcgcggcgcctccggaacctccgggtggacgcgggggtcgagtgtaggcgaggtcgggggaggggcgggggctcgttgtcgcgccgcgcccgctgaatcttttcccgacaggtcccaccccccgcgcgatgcccccccgggccgctggccatgtcgtccgggggaggccccgcggaccacgtcgtccggcgagacgccacgagccgcaggatggactcgtagtggagcgacggcgccccgttgcggagcagatccgcggccagggcggccccgaaccaagccttgatgctcaactccatccgggcccagctgggggcggtcatcgtggggaacaggggggcggtggtccgacagaaacgctcctggctgtccaccgcggcccgcagatactcgttgttcaggctgtcggtggcccagacgccgtacccggtgagggtcgcgttgatgatatactgggcgtggtgatggacgatcgacagaacctccaccgtggatacgacggtatccacggtcccgtacgtaccgccgctccgcttgccggtctgccacaggttggctaggcgcgtcaggtggcccaggacgtcgctgaccgccgccctgagcgccatgcactgcatggagccggtcgtgccgctgggaccccggtccagatggcgcgcgaacgtttccgcgggcgcctccgggctgccgccgagcgggaggaaccggcgattggagggactcagccggtgacatacgtgcttgtccgtcgtccacagcatccaggacgcccaccggtacagcacggagacgtaggccaggagctcgttgagccgcagtgcggtgtcggtgctggggcggcttgggtccgccgggcgcataaagaacatgtactgctgaatccgatggagggcgtcgcgcaggccggccacggtggcggcgtacttggccgccacggccccgctcttgaacggggtgcgcgccagcagctttggcgccagggtgggccgcagcagcacgtgaaggctggggtcgcagtcgcccacggggtcctcggggacgtccaggccgctgggcaccaccgtctgcaggtacttccagtactgcgtgaggatggcgcggctcaactggccgccgggcagctccacctcgcccagcgcctgggtggcggccgaagcgtagtgccggatgtactcgtagtgcgggtcgctggcgagcccgtccacgatcaaactctcgggaaccgtgttgtgttgccgcgcggccaaccggacgctgcgatcggtgcaggtcagaaacgccggctgcgcgtcgtcggagcgctgccgcaaggcgcccacggccgcgctaaggagcccctccggggtggggagcagacacccgccgaagatgcgccgctcgggaacgcccgcgttgtcgccgcggatcaggttggcaggcgtcaggcaccgcgccagccgcagggagctcgcgccgcgcgtccggcgctgcatggtgacgcccgttcggtcgggacccgccggtcggagttatgccgcgtccagggccatcggggcgctttttatcgggaggagcttatgggcgtggcgggcctcccagcccggtcgcgcgcctccccgacacgtgcgcccgcagggcggcggccccctcgtctcccatcagcagtttcctaaactgggacatgatgtccaccacgcggacccgcgggcccaacacggacccgccgcttacgggggcgggggggaagggctccaggtccttgagaagaaaggcggggtctgccgtcccggacacgggggcccggggcgctgaggaggcggggcgcagatccacgtgctccgcggccgcgcggacgtccgcccagaacttggcgggggtggtgcgcgcgtacaggggctgggtcgctcggaggacgcacgcgtagcgcaggggggtgtacgtgcccacctcgggggccgtgaatcccccgtcaaacgcggccagtgtcacgcacgccaccacggtgtcggcaaagcccagcagccgctgcaggacgagcccggcggccagaatggcgcgcgtggccgccgcgtcgtcccggcgccggtgcgcgtccccgcacgcccgggcgtactttaaggtcacggtcgccagggccgtgtgcagcgcgtacaccgcagcgcccagcacggcgttgagcccgctgttggcgagcagccggcgcgctgcggtgtcgcccagcgcctcgtgctcggcccccacgaccgcggggcttcccaggggcagggcgcgaaacagctcctcccgcgccacgtccgcaaaggcggggtggtgcacgtgcgggtgcaggcgcgcccccacgaccaccgagagccactggaccgtctgctccgccatcaccgccagcacatccagcacgcgccccaggaaggcggcctcccgcgtcaaaacgcaccggacggcgtcgggattgaagcgggcgagcagggccccggtggccaggtacgtcatgcggccggcatagcgggcggccacgcgacagtcgcggtccagcagcgcgcgcaccccgggccagtacagcagggaccccagcgagctgcggaacaccgcggcgtcggggccggattggggggacactaacccccccgcgctcagtaacggcacggccgcggccccgacgggacgcaacgccgtgaggctcgcgaactgccgcctcagctcggccgccctgtcgtccaggtcagacccgcgcgcctccgcgtgaaggcgcgtcccgcacacccacccgttgatggccagccgcacgacggcatccgccaaaaagctcatcgcctgggcggggctggtttttgttcgacgatccgtcaggtcaagaatcccatcgcccgtgatataccaggccaacgcctcgccctgctgcagggtttggcggaaaaacaccgcggggttgtcgggggaggcgaagtgcatgacccccacgcgcgataacccgaacgcgctatccggacacgggtaaaacccggccggatgccccagggctagggcggagcgcacggactcgtcccacacggcaacctgaggggccagtcgatccaacgggaatgccgcccggagctccgggcccggcacgcgtccctccagaacctccaccttgggcggggaacgggccccgccgccgtcctccggcccgacggcttccgggtagtcgtcctcctcgtactgcagctcctctaggaacagcggcgacggcgccacccgcgaaccgccgacccgccccaaaatagcccgcgcgtcgatcgatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagttatcgacttaattaagtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtagatctgctagcgtttaaacttaagcttggtaccgagctcggatccactagtccagtgtggtggaattcggctttaagcctggtaccgtaacaatccctcacccgttccaggtcggggatcaagtgcttgtcagacgccatcgacccagcagccttgagcctcggtggaaaggcccatacctggtgttgctgactaccccgaccgcggtaaaagtcgatggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaataagttagttcaattcatcaatgataggataagtgcatgttaaggcgcctagtgttgacaattaatcatcggcatagtatacggcatagtataatacgactcactataggagggccaccaagccgaattctgcagatatccagcacagtggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaataaacagtctctaactcgctagtctcggccgcggggggcccgggctgcccgccgccaccgctttaaagggccgcgcgcgacccccggggggtgtgttttggggggggcccgttttcggcgtctggccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcccgcggccccgccccccacgcccgccgcgcgcgcgcacgccgcccggaccgccgcccgccttttttgcgcgcgcgcgcgcccgcggggggcccgggctgccacaggtgaaaccaacagagcacggcgcactccgcacgtcacgtggactctagacttaattaaggatccggcgcgaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataattaactcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggacgggacccaggtatcccccctgccgggcctgcggaggaccgcggggaacctcatcatcatcgtccaggcgaccgcgcaccgactggctacgggccgcatcgggcccggggcgctgccgggacgctcggcgatgggatgtgggcggggcttccgacgcgcgccgtcgtcgggctcgcgggccttcccgtcgacggcgcacgggcggctcgtcgcccgccatctcctccagagcctctagctcgctgtcgtcatccccgcggaacaccgcacgcaggtaccccatgaaccccaccccatcgcccgctggctcgtccgccacgggcgaggcgcgggggcgggtggatgcgcgcctcctgcgccccgcgggttcgcgagccgacatggtggcgatagacgcgggttatcggatgtccgctaccccccaaaaaagaaaaagaccccacagcgcggatggaggccggggtaggtgccgccggaccccctcgcgatgggaatggacgggagcgacggggccggcgcaaaaaaacgcagtatctcccgcgaaggctacccgccgccccagcccccggccaaatgcggaaacggtcccgcgctctcgcctttatacgcgggccgccctgcgacacaatcacccgtccgtggtttcgaatctacacgacaggcccgcagacgcggctaacacacacgccggcaacccagaccccagtgggttggttgcgcggtcccgtctcctggctagttctttcccccaccaccaaataatcagacgacaaccgcaggttttgtaatgtatgtgctcgtgtttattgtggatacgaaccggtgacgggaggggaaaacccagacgggggatgcgggtccggtcgcgccccctacccaccgtactcgtcaattccaagggcatcggtaaacatctgctcaaactcgaagtcggccatatccagagcgccgtagggggcggagtcgtggggggtaaatcccggccccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcgtgttcccgcatgaccgcctcggagggcgaggtcgtgaagctggaatacgagtccaacttcgcccgaatcaacaccataaagtacccagaggcgcgggcctggttgccatgcagggtgggaggggtcgtcaacggcgcccctggctcctccgtagccgcgctgcgcaccagcgggaggttaaggtgctcgcgaatgtggtttagctcccgcagccggcgggcctcgattggcactccccggacggtgagcgctccgttgacgaacatgaagggctggaacagacccgccaactgacgccagctctccaggtcgcaacagaggcagtcaaacaggtcgggccgcatcatctgctcggcgtacgcggcccataggatctcgcgggtcaaaaatagatacaaatgcaaaaacaaaacacgcgccagacgagcggtctctcggtagtacctgtccgcgatcgtggcgcgcagcatttctcccaggtcgcgatcgcgtccgcgcatgtgcgcctggcggtgcagctgccggacgctggcgcgcaggtaccggtacagggccgagcagaagttggccaacacggttcgatagctctcctcccgcgcccgtagctcggcgtggaagaaacgagagagcgcttcgtagtagagcccgaggccgtcgcgggtggccggaagcgtagggaaggccacgtcgccgtgggcgcgaatgtcgatttgggtgcgttcggggacgtacgcgtccccccattccaccacatcgctgggcagcgttgataggaatttacactcccggtacaggtcggcgttggtcggtagcgccgaaaacagatcctcgttccaggtatcgagcatggtacatagcgcggggcccgcgctaaagcccaagtcgtcgaggagacggttaaagagggcggcgggggggacgggcatgggtggggagggcatgagctgggcctggctcaggcgccccgttgcgtacagcgggggggccgccggggtgtttttgggacccccggccgggcgggggggcggtggcgaagcgccgtccgcgttcatgtcggcaaacagctcgtcgaccaagaggtccattgggtggggttgatacgggaaagacgatatcgggcttttgatgcgatcgtccccgcccgcccagagagtgtgggacgcccgacggcgcgggaagagaaaacccccaaacgcgttagaggaccggacggaccttatggggggaagtgggcagcgggaaccccgtccgttcccgaggaatgacagcccgtggtcgccaccacgcatttaagcaacccgcacgggccgccccgtacctcgtgacttccccccacattggctcctgtcacgtgaaggcgaaccgagggcggctgtccaacccaccccccgccacccagtcccggtccccgtcggattgggaaacaaaggcacgcaacgccaacaccgaatgaacccctgttggtgctttattgtctgggtacggaagttttcactcgacgggccgtctggggcgagaagcggagcgggctggggctcgaggtcgctcggtggggcgcgacgccgcagaacgccctcgagtcgccgtggccgcgtcgacgtcctgcaccacgtctggattcaccaactcgttggcgcgctgaagcaggtttttgccctcgcagaccgtcacgcggatggtggtgatgccaaggagttcgttgaggtcttcgtctgtgcgcggacgcgacatgtcccagagctggaccgccgccatccgggcatgcatggccgccaggcgcccgaccgcggcgcagaagacgcgcttgttaaagccggccacccggggggtccatggcgcgtcggggtttgggggggcggtgctaaagtgcagctttctggccagcccctgcgcgggtgtcttggatcgggttggcgccgtcgacgcgggggcgtctgggagtgcggcggattctggctgggccgatttcctgccgcgggtggtctccgccgccggggccgcgggggccttagtcgccacccgctgggttcggggggcccggggggcggtggtgggtgtgcgtccggcccctccggacccagcgggtggcggaggcgcccgcgcaggccccgggccggacaaaaccgccccggaaacgggacgccgcgtccgggggacctccgggtgttcgtcgtcttcggatgacgagcccccgtagagggcataatccgactcgtcgtactggacgaaacggacctcgcccctctggcgcgagcgtgtctgtagggcgccacggcgggaggtgtcaggcggactatcgggactcgccatacctgaagacggggtgtagtacagatcctcgtactcatcgcgcggaacctcccgcggacccgacttcacggagcggcgagaggtcatggttccacgaacacgctagggtcggatgcgcggacaattaggcctgggttcggacggcgggggtggtgcaggtgtggagaggtcgagcgataggggcggcccgggagagaagagagggtccgcaaaacccactggggatgcgtgagtggccctctgtgggcggtgggggagagtcttataggaagtgcatataaccacaacccatgggtctaaccaatccccaggggccaagaaacagacacgccccaaacggtctcggtttccgcgaggaaggggaagtcctgggacaccctccacccccacccctcaccccacacagggcgggttcaggcgtgcccggcagccagtagcctctggcagatctgacagacgtgtgcgataatacacacgcccatcgaggccatgcctacataaaagggcaccagggcccccggggcagacatttggccagtgttttgggtctcgcaccgcgcgcccccgatcccatcgcgcccgccctcctcgccgggcggctccccgcgcgggcccgcgtctcccgccgctaaggcgacgagcaagacaaacaacaggcccgcccgacagacccttctgggggggcccatcgtccctaacaggaagatgagtcagtggggatccggggcgatccttgtccagccggacagcttgggtcgggggtacgatggcgactggcacacggccgtcgctactcgcgggggcggagtcgtgcaactgaacctggtcaacaggcgcgcggtggcttttatgccgaaggttagcggggactccggatgggccgtcgggcgcgtctctctggacctgcgaatggctatgccggctgacttttgcgcgattattcacgcccccgcgctagccagccccgggcaccacgtaatactgggtcttatcgactcggggtaccgcggaaccgttatggccgtggtcgtagcgcctaaaaggacgcgggaatttgcccccgggaccctgcgggtcgacgtgacgttcctggacatcctggcgacccccccggccctcaccgagccgatttccctgcggcagttcccgcaactggcgcccccccctccaaccggggccgggatacgcgaagatccttggttggagggggcgctcggggccccaagcgtgactacggccctaccggcgcgacgccgagggcggtccctcgtctatgccggcgagctgacgccggttcagacggaacacggggacggcgtacgagaagccatcgccttccttccaaaacgcgaggaggatgccggtttcgacattgtcgtccgtcgcccggtcaccgtcccggcaaacggcaccacggtcgtgcagccatccctccgcatgctccacgcggacgccgggcccgcggcctgctatgtgttggggcggtcgtcgctcaacgcccgcggcctcctggtcgttcctacgcgctggctccccgggcacgtatgtgcgtttgttgtttacaaccttacgggggttcctgtgaccctcgaggccggcgccaaggtcgcccagctcctggttgcgggggcggacgctcttccttggatccccccggacaactttcacgggaccaaagcgcttcgaaactaccccaggggtgttccggactcaaccgccgaacccaggaacccgccgctcctggtgtttacgaacgagtttgacgcggaggcccccccgagcgagcgcgggaccgggggttttggttctaccggtatttagcccacagctttgggttcgttccgggcaataaaaaacgtttgtatcgcatctttcctgtgtgtagttgtttatgttggatgcctgtgggtctatcacacccgcccctccatcccacaaacacaaaacacacgggttggatgaaaacacgcatttattgacccaaaacacacggagctgctcgagatgggccagggcgaggtgcggttggggaggctgtaggtctgggaacggacacgcggggacacgattccggtttggggtccgggagggcgtcgccgtttcgggcggcaggcgccagcgtaacctccgggggcggcgtgtgggggtgccccaaggagggcgcctcggtcaccccaatcccccccgaccgggttcccccggcaaccccgaaggcggagaggccaagggcccgttcggcgatggccacatcctccatgaccacgtcactctcggccatgctccgaatagcctgggagacgagcacatccgcggacttgtcagccgcccccacggacatgtacatctgcaggatggtggccatacacgtgtccgccaggcgccgcatcttgtcctgatgggccgccacggccccgtcgatcgtgggggcctcgaacccggggtggtggcgcgccagtcgttctaggttcaccatgcaggcgtggtacgtgcgggccaaggcgcgggccttcacgaggcgtcgggtgtcgtccagggaccccagggcgtcatcgagcgtgatgggggcgggaagtagcgcgttaacgaccgccagggcctcctgcagccgcggctccgcctccgagggcggaacggccgcgcggatcatctcatattgttcctcggggcgcgctccccagccacatatagccccgagaagagaagccatcgcgggcgggtactggcccttgggcgcgcggacgcaatggggcaggaagacgggaaccgcggggagaggcgggcggccgggactcccgtggaggtgaccgcgctttatgcgaccgacgggtgcgttattacctcttcgatcgccctcctcacaaactctctactgggggccgagccggtttatatattcagctacgacgcatacacgcacgatggccgtgccgacgggcccacggagcaagacaggttcgaagagagtcgggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgatcctggctctcaccgtggccatcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggtgcgcgtgcttgccgcgtaccgcagggcgtattatggaagcgcgcagagtcccttctggtttcttagcaaattcgggccggacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgctgcgtccctgacctcgtttgccgccatcacgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtacatctacctggcccattttgagtgtttcagccccccgcgcctagccacgcatcttcgggccgtgacgacccacgaccccaaccccgcggccagcacggagcagccctcgcccctgggcagggaggccgtggaacaatttttttgtcacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgatggggcgtttggcggacgccgaaaagctcctggtcccccgcgggtggcccgcgtttgcgcccgccagtcccggggaggacacggcgggcggcacgccgcccccacagacctgcggaattgtcaagcgcctcctgagactggccgccacggaacagcagggccccacacccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatattcaacggcgcgctggcaatcacaaacatcatcctggatctcgacatcgccctgaaggagcccgtcccctttcgccggctccacgaggccctgggccactttaggcgcggggctctggctgcggttcagctcctgtttcccgcggcccgcgtggaccccgacgcatatccctgttattttttcaaaagcgcatgtcggcccggcccggcgtccgtgggttccggcagcggactcggcaacgacgacgacggggactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggcacgccatgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccgtaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggctgccgatggtttgtcggacccccgccggcgtgcccccagaaccaatcgcaaccctgggggaccccgccccggggcggggtggacggacggccccggcgccccccatggcgaggcgtggcgcggcagtgagcagcccgacccacccggaggccagcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacgaccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggtacaataaaaacaaaacatttcaaacaaatcgcccctcgtgttgtccttctttgctcatggccggcggggcgtgggtcacggcagatggcgggggtgggcccggcgtacggcctgggtgggcggagggaactaacccaacgtataaatccgtccccgttccaaggccggtgtcatagtgcccttaggagcttcccgcccgggcgcatccccccttttgcactatgacagcgacccccctcaccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacacccaccacacccctccgctgattaccgagctccgcaactttgcggacctggttaaccacccgccggtcctacgcgaactggaggataagcgcggggtgcggctgcggtgtgcgcggccgtttagcgtcgggacgattaaggacgtctctgggtccggcgcgtcctcggcgggagagtacacgataaacgggatcgtgtaccactgccactgtcggtatccgttctcaaaaacatgctggatgggggcctccgcggccctacagcacctgcgctccatcagctccagcggcatggccgcccgcgcggcagagcatcgacgcgtcaagattaaaattaaggcgtgatctccaacccccccatgaatgtgtgtaaccccccccaaaaaaataaagagccgtaacccaaccaaaccaggcgtggtgtgagtttgtggacccaaagccctcagagacaacgcgacaggccagtatggaccgtgatacttttatttattaactcacaggggcgcttaccgccacaggaataccagaataatgaccaccacaatcgcgaccaccccaaatacagcatggcgccacaccacgccacaacagccctgtcgccggtatggggcatgatcagacgagccgcgcgccgcgcgttgggccctgtacagctcgcgcgaattgaccctaggaggccgccacgcgcccgagttttgcgttcgtcgctggtcgtcgggcgccaaagccccggacggctgttcggtcgaacgaacggccacgacagtggcataggttggggggtggtccgacatagcctcggcgtacgtcgggaggcccgacaagaggtcccttgtgatgtcgggtggggccacaagcctggtttccggaagaaacaggggggttgccaataacccgccagggccaaaactccggcgctgcgcacgtcgttcggcgcggcgccgggcgcgccgagcggctcgctgggcggcttggcgtgagcggccccgctccgacgcctcgccctctccggaggaggttggcggaattggcacggacaacaggggcccagcagagtacggtggaggtgggtccgtgggggtgtccagatcaataacgacaaacggcccctcgttcctaccagacaagctatcgtaggggggcgggggatcagcaaacgcgttccccgcgctccataaacccgcgtcgggttgcgccgcctccgaagccatggatgcgccccaaagccacgactcccgcgcgctaggtccttggggtaatggaaaaggccctactccccatccaagccagccaagttaacgggctacgccttcgggaatgggactggcaccccggcggattttgttgggctggcatgcgtcgcccaaccgagggccgcgtccacgggacgcgccttttataaccccgggggtcattcccaacgatcacatgcaatctaactggctcccctctccccccctctcccctctccccccctctcccctctccccccctctcccctctccccccctctcccctctccccccctctcccctctccccccctctcccctctccccccctctcccctctccccccctctcccctctccccccctctcccctctccccccctctcccctctcccctctgctctttccccgtgacacccgacgctgggggcgtggctgccgggaggggccgcggatgggcgggcctacttggtttcccgccccccccccccccccccgaaccgccccgccggctttgcccccctttgatcccctgctacccccaacccgtgctggtggtgcgggttggggggggatgtgggcgggggtgcgcgggaggtgtcggtggtggtggtggtggtggtagtaggaatggtggtgagggggggggggcgctggttggtcaaaaaagggagggacgggggccggcagaccgacggcgacaacgctccccggcggccgggtcgcggctcttacgagcggcccggcccgcgctcccaccccccgggccgtgtccttgctttccccccgtctcccccccccccgccttctcctcctcctcctcgtttttccaaaccccgcccacccggcccggcccggcccggcccggcccggccaccgccgcccacccacccacctcgggatacccagccccggtcccccgttccccgggggccgttatctccagcgccccgtccggcgcgccgccccccgccgctaaaccccatcccgcccccgggaccccacatataagcccccagccacacgcaagaacagacacgcagaacggctgtgtttatttaaataaaccaatgtcggaataaacaaacacaaacacccgcgacggggggacggaggggacggagggagggggtgacgggggacgggaacagacacaaaaacaaccacaaaaaacaaccacccaccgacacccccaccccagtctcctcgccttctcccacccaccccacgcccccactgagcccggtcgatcgacgagcacccccgcccacgcccccgcccctgccccggcgacccccggcccgcacgatcccgacaacaataacaaccccaacggaaagcggcggggtgttgggggaggcgaggaacaaccgaggggaacgggggatggaaggacgggaagtggaagtcctgatacccatcctacacccccctgccttccaccctccggccccccgcgagtccacccgccggccggctaccgagaccgaacacggcggccgccgcagccgccgcagccgccgccgacaccgcagagccggcgcgcgcactcacaagcggcagaggcagaaaggcccagagtcattgtttatgtggccgcgggccagcagacggcccgcgacaccccccccccgcccgtgtgggtatccggccccccgccccgcgccggtccattaagggcgcgcgtgcccgcgagatatcaatccgttaagtgctctgcagacaggggcaccgcgcccggaaatccattaggccgcagacgaggaaaataaaattacatcacctacccacgtggtgctgtggcctgtttttgctgcgtcatctcagcctttataaaagcgggggcgcggccgtgccgatcgcgggtggtgcgaaagactttccgggcgcgtccgggtgccgcggctctccgggcccccctgcagccggggcggccaaggggcgtcggcgacatcctccccctaagcgccggccggccgctggtctgttttttcgttttccccgtttcgggggtggtgggggttgcggtttctgtttctttaacccgtctggggtgtttttcgttccgtcgccggaatgtttcgttcgtctgtcccctcacggggcgaaggccgcgtacggcccgggacgaggggcccccgaccgcggcggtccgggccccgtccggacccgctcgccggcacgcgacgcgaaaaaggccccccggaggcttttccgggttcccggcccggggcctgagatgaacactcggggttaccgccaacggccggcccccgtggcggcccggcccggggccccggcggacccaaggggccccggcccggggccccacaacggcccggcgcatgcgctgtggtttttttttcctcggtgttctgccgggctccatcgcctttcctgttctcgcttctccccccccccttcttcacccccagtaccctcctccctcccttcctcccccgttatcccactcgtcgagggcgccccggtgtcgttcaacaaagacgccgcgtttccaggtaggttagacacctgcttctccccaatagaggggggggacccaaacgacagggggcgccccagaggctaaggtcggccacgccactcgcgggtgggctcgtgttacagcacaccagcccgttcttttccccccctcccacccttagtcagactctgttacttacccgtccgaccaccaactgcccccttatctaagggccggctggaagaccgccagggggtcggccggtgtcgctgtaaccccccacgccaatgacccacgtactccaagaaggcatgtgtcccaccccgcctgtgtttttgtgcctggctctctatgcttgggtcttactgcctggggggggggagtgcgggggagggggggtgtggaaggaaatgcacggcgcgtgtgtaccccccctaaagttgttcctaaagcgaggatacggaggagtggcgggtgccgggggaccggggtgatctctggcacgcgggggtgggaagggtcgggggagggggggatggagtaccggcccacctggccgcgcgggtgcgcgtgcctttgcacaccaaccccacgtcccccggcggtctctaagaagcaccgccccccctccttcataccaccgagcatgcctgggtgtgggttggtaaccaacacgcccatcccctcgtctcctgtgattctctggctgcaccgcattcttgttttctaactatgttcctgtttctgtctcccccccccccacccctccgccccaccccccaacacccacgtctgtggtgtggccgacccccttttgggcgccccgtcccgccccgccacccctcccatcctttgttgccctatagtgtagttaaccccccccgccctttgtggcggccagaggccaggtcagtccgggcgggcaggcgctcgcggaaacttaacacccacacccaacccactgtggttctggctccatgccagtggcaggatgctttcggggatcggtggtcaggcagcccgggccgcggctctgtggttaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcatccccgcgatacatccaacacagacagggaaaagatacaaaagtaaacctttatttcccaacagacagcaaaaatcccctgagtttttttttattagggccaacacaaaagacccgctggtgtgtggtgcccgtgtctttcacttttcccctccccgacacggattggctggtgtagtgggcgcggccagagaccacccagcgcccgacccccccctccccacaaacacggggggcgtcccttattgttttccctcgtcccgggtcgacgccccctgctccccggaccacgggtgccgagaccgcaggctgcggaagtccagggcgcccactagggtgccctggtcgaacagcatgttccccacgggggtcatccagaggctgttccactccgacgcgggggccgtcgggtactcggggggcatcacgtggttacccgcggtctcggggagcagggtgcggcggctccagccggggaccgcggcccgcagccgggtcgccatgtttcccgtctggtccaccaggaccacgtacgccccgatgttccccgtctccatgtccaggatgggcaggcagtcccccgtgatagtcttgttcacgtaaggcgacagggcgaccacgctagagacccccgagatgggcaggtagcgcgtgaggccgcccgcggggacggccccggaagtctccgcgtggcgcgtcttccgggcacacttcctcggcccccgcggcccagaagcagcgcgggggccgagggaggtttcctcttgtctccctcccagggcaccgacggccccgcccgaggaggcggaagcggaggaggacgcggccccggcggcggaagaggcggcccccgcgggggtcggggccgaggaggaagaggcagaggaggaagaggcggaggccgccgaggacgtcaggggggtcccgggcccaccctggccgcgcccccccggccctgagtcggagggggggtgcgtcgccgccctcttggcccctgccggcgcgagggggggacgcgtggactggggggaggggttttcctggcccgacccgcgcctcttcctcggacgcaccgccgcctcctgctcgacagaggcggcggaggggagcggggcggcgccggagggggcggcgccgcgggagggcccgtgcccaccctccacgcccggcccccccgagccgcgcgccaccgtcgcacgcgcccggcacagactctgttcttggttcgcggcctgagccagggacgagtgcgactggggcacacggcgcgcgtccgcggggcgggcggccggctccgccccgggggccggggcgcgggggccgggccccggaggcggcgctcgcacgcacggggccacggccgcgcgggggcgcgcgggtcccgacgcggccgcggacgcggggggcccggggcggggggcggagcctggcatgggcgccgcggggggcctgtggggagaggccgggggggagtcgctgatcactatggggtctctgttgtttgcaaggggggcgggtctgttgacaagggggcccgtccggcccctcggccgccccgcctccgcttcaacaaccccaaccccaaccccaacccccccggaggggccagacgccccccgcggcgccgcggctcgcgactggcgggagccgccgccgccgctgctgttggtggtggtgttggtgttactgctgccgtgtggcccgatgggcgccgaggggggcgctgtccgagccgcggccggctggggggctgcgtgagacgccccgcccgtcacggggggcgcggcggcgcctctgcgtgggggggcgcggggcgtccggcggggggcgggcggtacgtagtctgctgcaagagacaacggggggcgcgatcaggttacgccccctccccggcccgccctttcctcgcccgcccgcctattcctccctccccccccctcctcctcctcctcccccagggtccttgccgccccccgcctcaccgtcgtccaggtcgtcgtcatcctcgtccgtggtgggctccgggtgggtgggcgacagggccctcaccgtgtgcccccccagggtcaggtaccgcggggcgaaccgctgattgcccgtccagataaagtccacggccgtgcccgccctgacggcctcctcggcctccatgcgggtctgggggtcgttcacgatcgggatggtgctgaacgacccgctgggcgtcacgcccactatcaggtacaccagcttggcgttgcacagcgggcaggtgttgcgcaattgcatccaggttttcatgcacgggatgcagaagcggtgcatgcacgggaaggtgtcgcagcgcaggtggggcgcgatctcatccgtgcacacggcgcacacgtcgccctcgtcgctccccccgtcctctcgagggggggcgcccccgcaactgccggggtcttcctcgcggggggggctcccccccgagaccgcccccccatccacgccctgcggccccagcagccccgtctcgaacagttccgtgtccgtgctgtccgcctcggaggcggagtcgtcgtcatggtggtcggcgtccccccgcccccccacttcggtctccgcctcagagtcgctgctgtccggcaggtctcggtcgcagggaaacacccagacatccggggcgggctaaggggaaaaaaggggggcgggtaagaatggggggggatttcccgcgtcaatcagcacccacgagttccccctctcccccccccgcctcacaaagtcctgcccccctgctggcctcggaagaggggggagaaaggggtctgcaaccaaaggtggtctgggtccgtcctttggatcccgacccctcttcttccctcttctcccgccctccagacgcaccggagtcgggggtcccacggcgtcccccaaatatggcgggcggctcctccccacccccctagatgcgtgtgagtaaggggggcctgcgtatgagtcagtggggaccacgcccccaacacggcgaccccggtccttgtgtgtttgttgtgggggcgtgtctctgtgtatgagtcagggggtcccacggcgaccccgggccctgcgtctgagtcaaaggggccatgtgtatgtgttgggggtctgtatatataaagtcagggggtcacatggcgacccccaacagggcgaccccggtccctgtatatatagggtcagggggttccgcaccccctaacatggcgcccccggtccctgtatatatagtgtcacggggttccacgccccctaacatggcgccccaacatggcgcccggctcccgtgtatgagtgggggtcccccaacatggcggccggttccagtgtaagggtcgggggtcccccaacatggcgccccccaatatggcgccccccaatatggcgccccagacatggcgcccggcccctcacctcgcgctgggggcggccctcaggccggcgggtactcgctccggggcggggctccatgggggtcgtatgcggctggagggtcgcggacggagggtccctgggggtcgcaacgtaggcggggcttctgtggtgatgcggagagggggcggcccgagtctgcctggctgctgcgtctcgctccgagtgccgaggtgcaaatgcgaccagactgtcgggccagggctaacttataccccacgcctttcccctccccaaaggggcggcagtgacgattcccccaatggccgcgcgtcccaggggaggcaggcccaccgcggggcggccccgtccccggggaccaacccggcgcccccaaagaatatcattagcatgcacggcccggcccccgatttgggggcccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattaatgagtttctaattaccataccgggaagcggaacaaggcctcttgcaagtttttaattaccataccgggaagtgggcggcccggcccattgggcggtaactcccgcccaatgggccgggccccgaagactcggcggacgctggttggccgggccccgccgcgctggcggccgccgattggccagtcccgcccccgaggcggcccgccctgtgagggcgggctggctccaagcgtatatatgcgcggctcctgccatcgtctctccggagagcggcttggtgcggagctcccgggagctccgcggaagacccaggccgcctcgggtgtaacgttagaccgagttcgccgggccggctccgcgggccagggcccgggcacgggcctcgggccccaggcacggcccgatgaccgcctcggcctccgccacccggcgccggaaccgagcccggtcggcccgctcgcgggcccacgagccgcggcgcgccaggcgggcggccgaggcccagaccaccaggtggcgcacccggacgtggggcgagaagcgcacccgcgcgggggtcgcgggggtcgcgggggtcgcgggggtcgcgggggtcgcggggggctccggcgccccctccccgcccgcgcgtcgcaggcgcaggcgcgccaggtgctccgcggtgacgcgcaggcggagggcgaggcgcggcggaaggcggaaggggcgcgagggggggtgggaggggtcagccccgccccccgggcccacgccgggcggtgggggccggggccggggggcggcggcggtgggccgggcctctggcgccggctcgggcggggggctgtccggccagtcgtcgtcatcgtcgtcgtcggacgcggactcgggaacgtggagccactggcgcagcagcagcgaacaagaaggcgggggcccaccggcggggggcggcggcggggcggccgcgggcgcgctcctgaccgcgggttccgagttgggcgtggaggttacctgggactgtgcggttgggacggcgcccgtgggcccgggcggccgggggcggcgggggccgcgatggcggcggcggcgggccatggagacagagagcgtgccggggtggtagagtttgacaggcaagcatgtgcgtgcagaggcgagtagtgcttgcctgtctaactcgctagtctcggccgcggggggcccgggctgcccgccgccaccgctttaaagggccgcgcgcgacccccggggggtgtgttttggggggggcccgttttcggcgtctggccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcccgcggccccgccccccacgcccgccgcgcgcgcgcacgccgcccggaccgccgcccgccttttttgcgcgcgcgcgcgcccgcggggggcccgggctgccacaggtgaaaccaacagagcacggcgcactccgcacgtcacacgtcacgtcatccaccacacctgcccaacaacacaactcacagcgacaactcaccgcgcaacaactcctgttcctcatccacacgtcaccgcgcacctcccgctcctccagacgtaccccggcgcaacacaccgctcctgctacacaccaccgccccctccccagccccagccctccccagccccagccctccccggccccagccctccccggccccagccctccccggccccagccctccccggccccagccctccccggccccagccctccccggccccagccctccccggcgcgtcccgcgctccctcgggggggttcgggcatctctacctcagtgccgccaatctcaggtcagagatccaaaccctccgggggcgcccgcgcaccaccaccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgaataaacaacgctactgcaaaacttaatcaggttgttgccgtttattgcgtcttcgggtctcacaagcgccccgccccgtcccggcccgttacagcaccccgtccccctcgaacgcgccgccgtcgtcttcgtcccaggcgccttcccagtccacaacttcccgccgcgggggcgtggccaagcccgcctccgcccccagcacctccacggcccccgccgccgccagcacggtgccgctgcggcccgtggccgaggcccagcgaatcccgggcggcgccggcggcagggcccccgggccgtcgtcgtcgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcggcgtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgaagcgcgtgcgcacgcggtagcgcacgttgccgccgcggcacaggcgcagcggcggcgcgtcggggtacaggcgcgcgtgcgcggcctccacgcgcgcgaagacccccgggccgaacacgcggcccgaggccagcaccgtgcggcgcaggtcccgcgccgccggccagcgcacggcgcactgcacggcgggcagcagctcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgtgcgcccagcgccgacacgtcgggggcgccggtccaattgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttatcgtcgtcgtcgccgccgccgcacgcggcctgggcggcgggggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgtggctgggccccgggggctgccgccgccagccgcccagggggtcggggccctcggcgggccggcgcgacacggccacggggcgcgggcgggcctgcgccgcggcggcccggggcgccgcgggctgggcgggggcgggctcgggccccgggggcgtggaggggggcgcgggcgcggggaggggggcgcgggcgtccgagccgggggcgtccgcgccgctcttcttcgtcttcgggggtcgcgggccgccgcctccgggcggccgggccgggccgggactcttgcgcttgcgcccctcccgcggcgcggcggaggcggcggcggccgccagcgcgtcggcggcgtccggtgcgctggccgccgccgccagcagggggcgcaggctctggttgtcaaacagcaggtccgcggcggcggcggccgcggagctcggcaggcgcgggtcccgcggcagcgcggggcccagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggcccgcgggtccctccggccgcggggggctggcgggccgggccccggccagccccgggacggccgccaggtcgccgtcgaagccctcggccagcgcctccaggatcccgcggcaggcggccaggcactcgacggccacgcggccggcctgggcgcggcgcccggcgtcgtcgtcggcgtcggcgtggcgggcggcgtcggggtcgtcgccccccgcgggggaggcgggcgcggcggacagccgccccagggcggcgaggatccccgcggcgccgtacccggcgggcaccgcgcgctcgcccggtgcggcggcggcgacggcggcgaccccctcgtcatctgcgccggcgccggggctccccgcggcccccgtcagcgccgcgttctcgcgcgccaacaggggcgcgtaggcgcggcgcaggctggtcagcaggaagcccttctgcgcgcggtcgtatcggcggctcatggccacggcggccgccgcgtgcgccaggccccagccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccaccccatggcctccgcgtccggggtgtacagcagccgcgtgatcagggcgtactgctgcgcggcgtcgcccagctcgggcgcccacacggccgccggggcgcccgaggcctcgaaccggcgtcgcgcctcctccgcctcgggcgccccccagaggcccgggcggctgtcgcccaggccgccgtacagcacccgccccgggggcgggggcccggcgccgggccacggctccccgctgacgtacccgtcgcgatagcgcgcgtagaaggcgccggaggtcgcgtcggcgtccagctcgacccgccggggctgcccggccgtgaagcggcccgtggcgtcgcggccggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggctgccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccaccggccgtgacgacgtctccgcggcggctgggccggcgggcgcggcgacaggcggtccgtggggtccggatacgcgccgcgtagcggggcctcccgttcgcggccccgggccggggcccggtcgccggcggcgtcggctgcgtcgtcgtactcgtccccgtcatcgtcgtcggctcgaaaggcgggggtccggggcggcgaggccgcggggtcgggcgtcgggatcgtccggacggcctcctctaccatggaggccagcagagccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctccggggatacggctgcgacggcggacgtagcacggtaggtcacctacggactctcgatggggggagggggcgagacccacggaccccgacgacccccgccgtcgacgcggaactagcgcggaccggtcgatgcttgggtgggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccaggggccggcgggaaggaggagcagcggaggccgccggccccccgcccccccggcgggcccaccccgaacggaattccattatgcacgaccccgccccgacgccggcacgccgggggcccgtggccgcggcccgttggtcgaacccccggccccgcccatccgcgccatctgccatgggcggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggccccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgaggcgaccggcggcgaccgttgcgtggaccgcttcctgctcgtcgggcggggggaagccactgtggtcctccgggacgttttctggatggccgacatttccccaggcgcttttgcgccttgtgtaaaagcgcggcgtcccgctctccgatccccgcccctgggcacgcgcaagcgcaagcgcccttcccgccccctctcatcggagtctgaggtagaatccgatacagccttggagtctgaggtcgaatccgagacagcatcggattcgaccgagtctggggaccaggatgaagccccccgcatcggtggccgtagggccccccggaggcttggggggcggttttttctggacatgtcggcggaatccaccacggggacggaaacggatgcgtcggtgtcggacgaccccgacgacacgtccgactggtcttatgacgacattcccccacgacccaagcgggcccgggtaaacctgcggctcacgagctctcccgatcggcgggatggggttatttttcctaagatggggcgggtccggtctacccgggaaacgcagccccgggcccccaccccgtcggccccaagcccaaatgcaatgctacggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgtttgctgcaggtgtccggcggaacctggggcatgcacctgcgcaacaccatacgggaggtggaggctcgattcgacgccaccgcggaacccgtgtgcaagcttccttgtttggagaccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgccgcagaagaccgcaagtgtctggacggctgccggaaaatgcgcttctccaccgcctgcccctatccgtgcagcgacacgtttctccggccgtgagtccggtcgccccgacccccttgtatgtccccaaaataaaagaccaaaatcaaagcgtttgtcccagcgtcttaatggcgggaagggcggagagaaacagaccacgcggacatggggggtgtttgggggtttattggcaccgggggctaaagggtggtaaccggatagcagatgtgaggaagtcggggccgttcgccgcgaacggcgatcagagggtcagtttcttgcggaccacggcccggcgatgtgggttgctcgtctgggacctcgggcatgcccatacacgcacaacacggacgccgcaccggatgggacgtcgtaagggggcctggggtagctgggtggggtttgtgcagagcaatcagggaccgcagccagcgcatacaatcgcgctcccgtccgtttgtcccgggcagtaccacgccgtactggtattcgtaccggctgagcagggtctccagggggtggttgggggccgcggggaacggggtccacgccacggtccactcgggcaaaaaccgagtcggcacggcccacggttctcccacccacgcgtctggggtcttgatggcgataaatcttaccccgagccggattttttgggcgtattcgagaaacggcacacacagatccgccgcgcctaccacccacaagtggtagaggcgaggggggctgggttggtctcggtgcagcagtcggaagcacgccacggcgtccacgacctcggtgctctccaaggggctgtcctccgcaaacaggcccgtggtggtgtttggggggcagcgacaggacctagtgcgcacgatcgggcgggtgggtttgggtaagtccatcagcggctcggccaaccgtcgaaggttggccggacgaacgacgaccggggtacccaggggttctgatgccaaaatgcggcactgcctaagcaggaagctccacagggccgggcttgcgtcgacggaagtccggggcagggcgttgttctggtcaaggagggtcattacgttgacgacaacaacgcccatgttggtatattacaggcccgtgtccgatttggggcacttgcagatttgtaaggccacgcacggcggggagacaggccgacgcgggggctgctctaaaaatttaagggccctacggtccacagacccgccttcccgggggggcccttggagcgaccggcagcggaggcgtccgggggaggggagggtgatttacgggggggtaggtcagggggtgggtcgtcaaactgccgctccttaaaaccccggggcccgtcgttcggggtgctcgttggttggcactcacggtgcggcgaatggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccagcgaggccgagcgcctgtgtcatctgcaggagatccttgcccagatgtacggaaaccaggactaccccatagaggacgaccccagcgcggatgccgcggacgatgtcgacgaggacgccccggacgacgtggcctatccggaggaatacgcagaggagctttttctgcccggggacgcgaccggtccccttatcggggccaacgaccacatccctcccccgtgtggcgcatctccccccggtatacgacgacgcagccgggatgagattggggccacgggatttaccgcggaagagctggacgccatggacagggaggcggctcgagccatcagccgcggcggcaagcccccctcgaccatggccaagctggtgactggcatgggctttacgatccacggagcgctcaccccaggatcggaggggtgtgtctttgacagcagccatccagattacccccaacgggtaatcgtgaaggcggggtggtacacgagcacgagccacgaggcgcgactgctgaggcgactggaccacccggcgatcctgcccctcctggacctgcatgtcgtctccggggtcacgtgtctggtcctccccaagtaccaggccgacctgtatacctatctgagtaggcgcctgaacccactgggacgcccgcagatcgcagcggtctcccggcagctcctaagcgccgttgactacattcaccgccagggcattatccaccgcgacattaagaccgaaaatatttttattaacacccccgaggacatttgcctgggggactttggcgccgcgtgcttcgtgcagggttcccgatcaagccccttcccctacggaatcgccggaaccatcgacaccaacgcccccgaggtcctggccggggatccgtataccacgaccgtcgacatttggagcgccggtctggtgatcttcgagactgccgtccacaacgcgtccttgttctcggccccccgcggccccaaaaggggcccgtgcgacagtcagatcacccgcatcatccgacaggcccaggtccacgttgacgagttttccccgcatccagaatcgcgcctcacctcgcgctaccgctcccgcgcggccgggaacaatcgcccgccgtacacccgaccggcctggacccgctactacaagatggacatagacgtcgaatatctggtttgcaaagccctcaccttcgacggcgcgcttcgccccagcgccgcagagctgctttgtttgccgctgtttcaacagaaatgaccgccccctgggggcggtgctgtttgcgggttggcacaaaaagaccccgatccgcgtctgtggtgtttttggcatcatgtcgcagggcgccatgcgtgccgttgttcccattatcccattccttttggttcttgtcggtgtatcgggggttcccaccaacgtctcctccaccacccaaccccaactccagaccaccggtcgtccctcgcatgaagcccccaacatgacccagaccggcaccaccgactctcccaccgccatcagccttaccacgcccgaccacacaccccccatgccaagtattggactggaggaggaggaagaggaggagggggccggggacggcgaacatcttgaggggggagatgggacccgtgacaccctaccccagtccccgggcccagccttcccgttggctgaggacgtcgagaaggacaaacccaaccgtcccgtagtcccatcccccgatcccaacaactcccccgcgcgccccgagaccagtcgcccgaagacaccccccaccattatcgggccgctggcaactcgccccacgacccgactcacctcaaagggacgacccttggttccgacgcctcaacataccccgctgttctcgttcctcactgcctcccccgccctggacaccctcttcgtcgtcagcaccgtcatccacaccttatcgtttttgtgtattggtgcgatggcgacacacctgtgtggcggttggtccagacgcgggcgacgcacacaccctagcgtgcgttacgtgtgcctgccgtccgaacgcgggtagggtatggggcgggggatggggagagcccacatgcggaaagcaagaacaataaaggcggtggtatctagttgatatgcatctctgggtgtttttggggtgtggcggacgcggggcggtcattggacggggtgcagttaaatacatgcccgggacccatgaagcatgcgcgacttccgggcctcagaacccacccgaaacggccaacggacgtctgagccaggcctggctatccggagaaacagcacacgacttggcgttctgtgtgtcgcgatgtctctgcgcgcagtctggcatctggggcttttgggaagcctcgtgggggctgttcttgccgccacccatcggggacctgcggccaacacaacggaccccttaacgcacgccccagtgtcccctcaccccagccccctggggggctttgccgtccccctcgtagtcggtgggctgtgcgccgtagtcctgggggcggcatgtctgcttgagctcctgcgtcgtacgtgccgcgggtgggggcgttaccatccctacatggacccagttgtcgtataatttcccccccccccccccttctccgcgtgggtgatgtcgggtccaaactcccgacaccaccagctggcatggtataaatcaccggtgcgccccccaaaccatgtccggcagggggatgggggggcaatgcggagggcacccaacaacaccgggctaaccaggaaatccgtggccccggcccccaataaagatcgcggtagcccggccgtgtgacactatcgtccataccgaccacaccgacgaatcccccaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcagcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactagatacccccccttaatgggtgcgggggggtcaggtctgcggggttgggatgggaccttaactccatataaagcgagtctggaaggggggaaaggtggacagtcgataagtcggtagcgggggacgcgcacctgttccgcctgtcgcacccacagctttttttgcgaaccgtcccgttccgggatgccgtgccgcccgttgcagggcctggtgctcgtgggcctctgggtctgtgccaccagcctggttgtccgtggccccacggtcagtctggtatcaaactcatttgtggacgccggggccttggggcccgacggcgtagtggaggaagacctgcttattctcggggagcttcgctttgtgggggaccaggtcccccacaccacctactacgatgggggcgtagagctgtggcactaccccatgggacacaaatgcccacgggtcgtgcatgtcgtcacggtgaccgcgtgcccacgtcgccccgccgtggcattcgccctgtgtcgcgcgaccgacagcactcacagccccgcatatcccaccctggagctcaatctggcccaacagccgcttttgcgggtccagagggcaacgcgggactatgccggggtgtacgtgttacgcgtatgggtcggtgacgcgccaaacgccagcctgtttgtcctggggatggccatagccgccgaagggactctggcgtacaacggctcggcctatggctcctgcgacccgaaactgcttccgtcttcggccccgcgtctggccccggcgagcgtataccaacccgcccctaaccaggcctccaccccctcgaccaccacctccaccccctcgaccaccatccccgctccctcgaccaccatccccgctccccaagcatcgaccacgcccttccccacgggagatccaaaaccacaacctcccggggtcaaccacgaacccccatctaatgccacgcgagcgacccgcgactcgcgatacgcgctaacggtgacccagataatccagatagccatccccgcgtccatcatagccctggtgtttctggggagctgtatttgctttatacacagatgtcaacgccgctaccgacgctcccgtcgcccgatttacagcccccagatgcccacgggcatctcatgcgcggtgaacgaagcggccatggcccgcctcggagccgagctcaaatcgcatccgagcaccccccccaaatcccggcgccggtcgtcacgcacgccaatgccctccctgacggccatcgccgaagagtcggagcccgctggggcggctgggcttccgacgccccccgtggaccccacgacacccaccccaacgcctcccctgttggtataggtccacggccactggccgggagcaccacataaccgaccgcagtccctgagttgggaataaaccggtattatttacctatatccgtgtatgtcgatttctttccccccctccccggaaaccaaagaaggaagcaaagaatggatgggaggagttcaggaagccggggagagggcccgcggcgcatttaaggcgttgttgtgttgactttgcctcttctggcgggttggtgcggtgctgtttgttgggctcccattttacccgaagatcggctgctatccccgggacatggatcgcggggcggtggtggggtttcttctcggtgtttgtgttgtatcgtgcttggcgggaacgcccaaaacgtcctggagacgggtgagtgtcggcgaggacgtttcgttgcttccagctccggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacgaacggacttcgtgtggcaggagcgcgcggccgtggttaaccggagtctggttattcacggggtccgagagacggacagcggcctgtataccctgtccgtgggcgacataaaggacccggctcgccaagtggcctcggtggtcctggtggtgcaaccggccccagttccgaccccacccccgaccccagccgattacgacgaggatgacaatgacgagggcgaggacgaaagtctcgccggcactcccgccagcgggaccccccggctcccgcctccccccgcccccccgaggtcttggcccagcgcccccgaagtctcacatgtgcgtggggtgaccgtgcgtatggagactccggaagctatcctgttttcccccggggagacgttcagcacgaacgtctccatccatgccatcgcccacgacgaccagacctactccatggacgtcgtctggttgaggttcgacgtgccgacctcgtgtgccgagatgcgaatatacgaatcgtgtctgtatcacccgcagctcccagaatgtctgtccccggccgacgcgccgtgcgccgcgagtacgtggacgtctcgcctggccgtccgcagctacgcggggtgttccagaacaaaccccccaccgcgctgttcggccgaggctcacatggagcccgtcccggggctggcgtggcaggcggcctccgtcaatctggagttccgggacgcgtccccacaacactccggcctgtatctgtgtgtggtgtacgtcaacgaccatattcacgcctggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcactggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatacccccgtagcgatgggcatcaatctcgccgccagctcacaacctttggatccggaaggcccgatcgccgttactcccaggcctccgattcgtccgtcttctggtaaggcgccccatcccgaggccccacgtcggtcgccgaactgggcgaccgccggcgaggtggacgtcggagacgagctaatcgcgatttccgacgaacgcggacccccccgacatgaccgcccgcccctcgccacgtcgaccgcgccctcgccacacccgcgacccccgggctacacggccgttgtctccccgatggccctccaggctgtcgacgccccctccctgtttgtcgcctggctggccgctcggtggctccggggggcttccggcctgggggccgtcctgtgtgggattgcgtggtatgtgacgtcaattgcccgaggcgcataaagggccggtggtccgcctagccgcagcaaattaaaaatcgtgagtcacagcgaccgcaacttcccacccggagctttcttccggcctcgatgacgtcccggctctccgatcccaactcctcagcgcgatccgacatgtccgtgccgctttatcccacggcctcgccagtttcggtcgaagcctactactcggaaagcgaagacgaggcggccaacgacttcctcgtacgcatgggccgccaacagtcggtattaaggcgtcgacgcagacgcacccgctgcgtcggcatggtgatcgcctgtctcctcgtggccgttctgtcgggcggatttggggcgctcctgatgtggctgctccgctaaaagaccgcatcgacacgcgcgtccttcttgtcgtctctcttcccccccatcaccccgcaatttgcacccagcctttaactacattaaattgggttcgattggcaatgttgtctcccggttgatttttgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtggcaaggaagaaacaagcccgaccaccagacagaaaatgtaaccatacccaaaccgactctgggggctgtttgtggggtcggaaccataggatgaacaaaccaccccgtaccacccgcacccaagggtgcggtggctcatcggcatctgtccggtatgggttgttccccacccactcgcgttcggacgtcttagaatcatggcggttttctatgccgacatcggttttctcccccgcaataagacacgatgcgataaaatctgtttgtaaaatttattaagggtacaaattgccctagcacaggggtggggttagggccgggtccccacacccaaacgcaccaaacagatgcaggcagtgggtcgagtacagccccgcgtacgaacacgtcgatgcgtgtgtcagacagcaccagaaagcacaggccatcaacaggtcgtgcatgtgtcggtgggtttggacgcggggggccatggtggtgataaagttaatggccgccgtccgccagggccacaggggcgacgtctcttggttggcccggagccactgggtgtggaccagccgcgcgtggcggcccaacatggcccctgtagccgggggcgggggatcgcgcacgtttgcagcgcacatgcgagacacctcgaccacggttcgaaagaaggcccggtggtccgcgggcaacatcaccaggtgcgcaagcgcccgggcgtccagagggtagagccctgagtcatccgaggttggctcatcgcccgggtcttgccgcaagtgcgtgtgggttgggcttccggtgggcgggacgcgaaccgcggtgtggatcccgacgcgggcccgagcgtatgctccatgttgtggggagaaggggtctgggctcgccaggggggcatacttgcccgggctatacagacccgcgagccgtacgtggttcgcggggggtgcgtggggtccggggctcccggggagaccggggctcccggggagaccggggctccctgggagaccggggttgtcgtggatccctggggtcacgcggtaccctggggtctctgggagctcgcggtactctgggttccctaggttctcggggtggtcgcggaacccggggctcccggggaacacgcggtgtcctggggattgttggcggtcggacggcttcagatggcttcgagatcgtagtgtccgcaccgactcgtagtagacccgaatctccacattgccccgccgcttgatcattatcaccccgttgcgggggtccggagatcatgcgcgggtgtcctcgaggtgcgtgaacacctctggggtgcatgccggcggacggcacgccttttaagtaaacatctgggtcgcccggcccaactggggccgggggttgggtctggctcatctcgagagccacgggggggaaccaccctccgcccagagactcgggtgatggtcgtacccgggactcaacgggttaccggattacggggactgtcggtcacggtcccgccggttcttcgatgtgccacacccaaggatgcgttgggggcgatttcgggcagcagcccgggagagcgcagcaggggacgctccgggtcgtgcacggcggttctggccgcctcccggtcctcacgcccccttttattgatctcatcgcgtacgtcggcgtacgtcctgggcccaacccgcatggtgtccaggaaggtgtccgccatttccagggcccacgacatgctcccccccgacgagcaggaagcggtccacgcaacggtcgccgccggtcgcctcgacgaggacgttcctcctgcgggaaggcacgaacgcgggtgagccccctcctccgcccccgcgtcccccctcctccgcccccgcgtcccccctcctccgcccccgcgtcccccctcctccgcccccgcgtcccccctcctccgcccccgcgtcccccctcctccgcccccgcgtcccccctcctccacccccgcgtccccccctcctccgcccacccaaggtgcttacccgtgcaaaaaaggcggaccggtgggtttctgtcgtcggaggcccccggggtgcgtcccctgtgtttcgtgggtggggtgggcgggtctttcccccccgcgtccgcgtgtccctttccgatgcgatcccgatcccgagccggggcgtcgcgatgccgacgccgtccgctccgacggccctctgcgactcccgctcccggtccgcgtgctccgcagccgctcccgtcgttcgtggccggcgccgtctgcgggcgtcggtcgcgccgggcctttatgtgcgccggagagacccgccccccgccgcccgggcccgcccccggggccggcgcggagtcgggcacggcgccagtgctcgcacttcgccctaataatatatatatattgggacgaagtgcgaacgcttcgcgttctcacttcttttacccggcggccccgcccccttggggcggtcccgcccgccggccaatgggggggcggcaaggcgggcggcccttgggccgcccgccgtcccgttggtcccggcgtccggcgggcgggaccggggggcccggggacggccaacgggcgcgcggggctcgtatctcattaccgccgaaccgggaagtcggggcccgggccccgccccctgcccgttcctcgttagcatgcggaacggaagcggaaaccgccggatcgggcggtaatgagatgccatgcggggcggggcgcggacccacccgccctcgcgccccgcccatggcagatggcgcggatgggcggggccgggggttcgaccaacgggccgcggccacgggcccccggcgtgccggcgtcggggcggggtcgtgcataatggaattccgttcggggtgggcccgccgggggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttcccacccaagcatcgaccggtccgcgctagttccgcgtcgacggcgggggtcgtcggggtccgtgggtctcgccccctccccccatcgagagtccgtaggtgacctaccgtgctacgtccgccgtcgcagccgtatccccggaggatcgccccgcatcggcgatggcgtcggagaacaagcagcgccccggctccccgggccccaccgacgggccgccgcccaccccgagcccagaccgcgacgagcggggggccctcgggtggggcgcggagacggaggagggcggggacgaccccgaccacgaccccgaccacccccacgacctcgacgacgcccggcgggacgggagggcccccgcggcgggcaccgacgccggcgaggacgccggggacgccgtctcgccgcgacagctggctctgctggcctccatggtagaggaggccgtccggacgatcccgacgcccgaccccgcggcctcgccgccccggacccccgcctttcgagccgacgacgatgacggggacgagtacgacgacgcagccgacgccgccggcgaccgggccccggcccggggccgcgaacgggaggccccgctacgcggcgcgtatccggaccccacggaccgcctgtcgccgcgcccgccggcccagccgccgcggagacgtcgtcacggccggtggcggccatcggcgtcatcgacctcgtcggactccgggtcctcgtcctcgtcgtccgcatcctcttcgtcctcgtcgtccgacgaggacgaggacgacgacggcaacgacgcggccgaccacgcacgcgaggcgcgggccgtcgggcggggtccgtcgagcgcggcgccggcagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgagctggacgccgacgcgacctccggcgccttctacgcgcgctatcgcgacgggtacgtcagcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggcggccgtgtgggcgcccgagctgggcgacgccgcgcagcagtacgccctgatcacgcggctgctgtacaccccggacgcggaggccatggggtggctccagaacccgcgcgtggtccccggggacgtggcgctggaccaggcctgcttccggatctcgggcgccgcgcgcaacagcagctccttcatcaccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgtcgccgccgccgcaccgggcgagcgcgcggtgcccgccgggtacggcgccgcggggatcctcgccgccctggggcggctgtccgccgcgcccgcctcccccgcggggggcgacgaccccgacgccgcccgccacgccgacgccgacgacgacgccgggcgccgcgcccaggccggccgcgtggccgtcgagtgcctggccgcctgccgcgggatcctggaggcgctggccgagggcttcgacggcgacctggcggccgtcccggggctggccggggcccggcccgccagccccccgcggccggagggacccgcgggccccgcttccccgccgccgccgcacgccgacgcgccccgcctgcgcgcgtggctgcgcgagctgcggttcgtgcgcgacgcgctggtgctcatgcgcctgcgcggggacctgcgcgtggccggcggcagcgaggccgccgtggccgccgtgcgcgccgtgagcctggtcgccggggccctgggccccgcgctgccgcgggacccgcgcctgccgagctccgcggccgccgccgccgcggacctgctgtttgacaaccagagcctgcgccccctgctggcggcggcggccagcgcaccggacgccgccgacgcgctggcggccgccgccgcctccgccgcgccgcgggaggggcgcaagcgcaagagtcccggcccggcccggccgcccggaggcggcggcccgcgacccccgaagacgaagaagagcggcgcggacgcccccggctcggacgcccgcgcccccctccccgcgcccgcgcccccctccacgcccccggggcccgagcccgcccccgcccagcccgcggcgccccgggccgccgcggcgcaggcccgcccgcgccccgtggccgtgtcgcgccggcccgccgagggccccgaccccctgggcggctggcggcggcagcccccggggcccagccacacggcggcgcccgcggccgccgccctggaggcctactgctccccgcgcgccgtggccgagctcacggaccacccgctgttccccgtcccctggcgaccggccctcatgtttgacccgcgggccctggcctcgatcgccgcgcggtgcgccgggcccgcccccgccgcccaggccgcgtgcggcggcggcgacgacgacgataacccccacccccacggggccgccgggggccgcctctttggccccctgcgcgcctcgggcccgctgcgccgcatggcggcctggatgcgccagatccccgaccccgaggacgtgcgcgtggtggtgctgtactcgccgctgccgggcgaggacctggccggcggcggggcctcgggggggccgccggagtggtccgccgagcgcggcgggctgtcctgcctgctggcggccctggccaaccggctgtgcgggccggacacggccgcctgggcgggcaattggaccggcgcccccgacgtgtcggcgctgggcgcacagggcgtgctgctgctgtccacgcgggacctggccttcgccggggccgtggagtttctggggctgctcgccagcgccggcgaccggcggctcatcgtggtcaacaccgtgcgcgcctgcgactggcccgccgacgggcccgcggtgtcgcggcagcacgcctacctggcgtgcgagctgctgcccgccgtgcagtgcgccgtgcgctggccggcggcgcgggacctgcgccgcacggtgctggcctcgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcacgccgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcgacgacgacggcccgggggccctgccgccggcgccgcccgggattcgctgggcctcggccacgggccgcagcggcaccgtgctggcggcggcgggggccgtggaggtgctgggggcggaggcgggcttggccacgcccccgcggcgggaagttgtggactgggaaggcgcctgggacgaagacgacggcggcgcgttcgagggggacggggtgctgtaacgggccgggacggggcggggcgcttgtgagacccgaagacgcaataaacggcaacaacctgattaagttttgcagtagcgttgtttattcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcggtggtggtgcgcgggcgcccccggagggtttggatctctgacctgagattggcggcactgaggtagagatgcccgaacccccccgagggagcgcgggacgcgccggggagggctggggccggggagggctggggccggggagggctggggccggggagggctggggccggggagggctggggccggggagggctggggccggggagggctggggctggggagggctggggctggggagggggcggtggtgtgtagcaggagcggtgtgttgcgccggggtacgtctggaggagcgggaggtgcgcggtgacgtgtggatgaggaacaggagttgttgcgcggtgagttgtcgctgtgagttgtgttgttgggcaggtgtggtggatgacgtgacgtgtgacgtgcggagtgcgccgtgctctgttggtttcacctgtggcagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggc